(19) United States
(12) Patent Application Publication
(10) Pub. No.: US 2022/0409593 A1
(43) Pub. Date: Dec. 29, 2022

(54) PROGRANULIN MODULATORS AND METHODS OF USING THE SAME

(71) Applicant: ARKUDA THERAPEUTICS, Watertown, MA (US)

(72) Inventors: Duane A. Burnett, Cambridge, MA (US); James C. Lanter, Cambridge, MA (US)

(21) Appl. No.: 17/770,469

(22) PCT Filed: Oct. 23, 2020
(86) PCT No.: PCT/US20/56973

§ 371 Date: Apr. 20, 2022

Related U.S. Application Data
(60) Provisional application No. 62/926,013, filed on Oct. 25, 2019.

(51) Int. Cl. A61K 31/435; A61P 25/28

(52) U.S. Cl. CPC A61K 31/435; A61P 25/28

(57) ABSTRACT

Provided herein are compounds of formula (I) that modulate progranulin and methods of using the compounds in progranulin-associated disorders, such as Frontotemperal lobe dementia (FTLD).

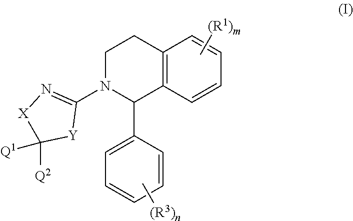

### BACKGROUND

Provided herein are compounds that modulate progranulin levels and can be useful as therapeutics for granulin (GRN)- and/or progranulin (PGRN)-associated disorders. Mutations in the GRN gene cause Frontotemporal lobar degeneration (FTLD) (see, e.g., Cruts et al., Granulin Mutations Associated with Frontotemporal Lobar Degeneration and Related Disorders: An Update, Hu Mutation, 2008 and Baker et al., Nature, 2006.) FTLD-associated mutations in GRN result in a reduction of progranulin protein expression, which suggests that haploinsufficiency of progranulin is the critical pathogenic factor in FTLD-GRN. Plasma and CSF progranulin levels are reduced by up to 70% in pathogenic GRN mutation carriers (Ghidoni, et al., Neurodegen Dis, 2012). More than 60 non-sense mutations in the GRN gene have been described. Plasma can be easily monitored for PGRN (see e.g., Meeter, Nature Neurology, volume 13, 2017). Thus, granulin- and/or progranulin-associated disorders can be modulated by compounds which increase progranulin secretion and/or activity.

All known FTLD-GRN-associated mutations cause haploinsufficiency of progranulin, suggesting that restoration of proper progranulin levels or progranulin protein function will be therapeutically beneficial for FTLD-GRN patients. Several studies have shown that even subtle reductions in progranulin levels by genetic modifiers (e.g., TMEM106B, SLPI, Rs5848) have significant effects on the age-of-onset of FTLD, increase the risk of developing FTLD, or worsen the course of autoimmune diseases such as osteoarthritis (see, e.g., Nicholson et al., J Neurochem, 2013; Cruchaga et al., Arch Neurol, 2012; and Wei et al, Plos One, 2014). Polymorphisms that affect progranulin levels have also been identified as genetic modifiers of several other neurodegenerative diseases, such as Alzheimer's disease and C9orf72-linked FTLD (see, e.g., Sheng et al., Gene, 2014 and van Blitterswijk et al., Mol Neurodegen, 2014). As such, it is contemplated herein that progranulin-targeted therapeutics are effective across multiple neurodegenerative and autoimmune disorders.

Granulins are a family of secreted and glycosylated proteins. They are cleaved from a common precursor protein called progranulin (PGRN). Progranulin is a secreted glycoprotein and is expressed in neurons, neuroglia, chondrocytes, epithelial cells and leukocytes (Toh H et al. J Mol Neurosci 201 1 November; 45(3):538-48). It is a precursor protein with an N-terminal signal peptide and seven granulin motifs. Each of these granulin motifs contains 12 cysteines, which are responsible for 6 disulfide bridges in every granulin (Bateman A et al. Bioessays 2009:1245-54). Progranulin is coded by the GRN gene. Mutations in the GRN gene have been implicated in up to 25% of frontotemporal lobar degeneration, inherited in an autosomal dominant fashion with high penetrance (see, e.g., Mackenzie, Acta Neuropathologica, 114(1): 49-54 (2007)). Thus, modulation of progranulin activity is an attractive target for treating disorders associated with GRN activity or GRN-gene mutations.

### SUMMARY

Provided herein are compounds and methods for modulating progranulin, e.g., increasing the level of progranulin or granulin in a subject. More particularly, provided are modulators of progranulin and the uses of such modulators in treating progranulin-associated disorders, e.g., Alzheimer [symbol] disease (AD), Parkinson [symbol] disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington [symbol] disease (HD), Spinocerebellar ataxia (SCA), Spinal muscular atrophy (SMA), lysosomal storage diseases, diseases associated with inclusions and/or misfunction of C9orf72, TDP-43, FUS, UBQLN2, VCP, CHMP28, and/or MAPT, acute neurological disorders, glioblastoma, or neuroblastoma.

In one aspect, the disclosure provides compounds of Formula (I):

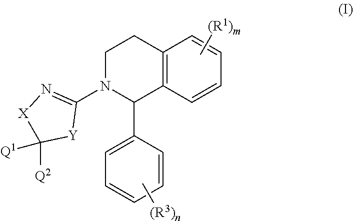

wherein

one of X and Y is O and the other is CH₂ or CH₂CH₂, or X is null or CH₂ and Y is CH₂O or (C═O)NH;

one of Q¹ and Q² is H and the other is C₀₋₃alkylene-NR⁶₂ or ring A,

or Q¹ and Q² together with the atom to which they are attached form ring A;

ring A comprises a 4- to 8-membered monocyclic or bicyclic carbocycle or heterocycle optionally substituted with 1-3 R² groups, wherein the heterocycle comprises a ring nitrogen or oxygen, or both;

each R¹ is independently C₁₋₆alkyl, O—C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SOₚR⁵, O—C₀₋₆alkylene-Ar, oxo, and C₀₋₆alkylene-Ar;

each R³ is independently halo, C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SO₂R⁵, O—C₀₋₆alkylene-Ar, and C₀₋₆alkylene-Ar;

each R² is independently halo, OH, C₁₋₆alkyl, C₁₋₆hydroxyalkyl, or C₀₋₆alkylene-N(R^{N})₂;

each R^{N} is independently H or C₁₋₆alkyl;

each R⁴ is independently H, C₁₋₆alkyl, or C(O)C₁₋₆alkyl;

each R⁵ is independently C₁₋₆alkyl, C₁₋₆haloalkyl, or Ar;

each R⁶ is independently H or C₁₋₆alkyl;

Ar is 3-8-membered carbocycle or heterocycle, wherein the heterocycle comprises 1-4 ring heteroatoms selected from N, O, and S; C₆₋₁₀aryl; or 5-10 membered heteroaryl comprising 1-4 ring heteroatoms selected from N, O, and S and Ar is optionally substituted with 1-3 groups independently selected from halo, C₁₋₆alkyl, C₁₋₆alkoxy, C₁₋₆haloalkoxy, CN, and SO₂C₁₋₃alkyl;

m is 0-2;

n is 0-3; and

p is 0-2. In some cases, one of X and Y is O and the other is CH₂ or CH₂CH₂, or X is null or CH₂ and Y is CH₂O, and each R² is independently halo, C₁₋₆alkyl, or C₀₋₆alkylene-N(R^{N})₂.

In some cases, the compounds have a structure of Formula (II), (IIa), or (IIb):

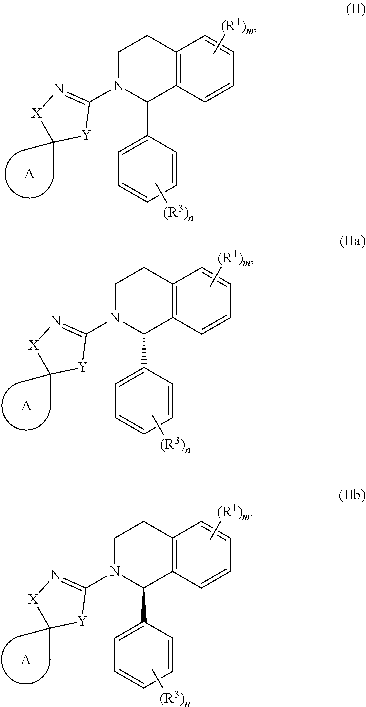

In some cases, the compound has a structure of Formula (III):

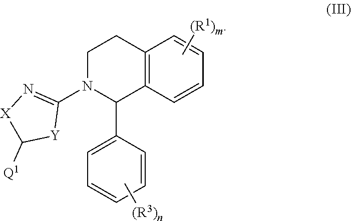

In some cases, ring A is

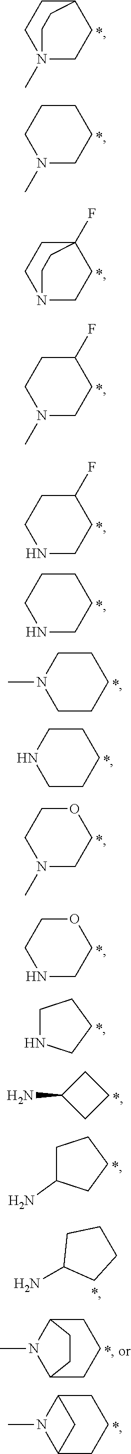

wherein * indicates the point of attachment.

Further provided are methods of modulating progranulin in a subject. In some embodiments, provided are methods of treating a progranulin-associated disorder in a subject.

Other aspects of the disclosure include a compound as disclosed herein for use in the preparation of a medicament for the modulation of progranulin, and the use of a compound as disclosed herein in a method of treating or preventing a progranulin-associated disorder in a subject.

### DETAILED DESCRIPTION

### Compounds as Progranulin Modulators

Provided herein are compounds that can modulate progranulin production and/or secretion. In some cases, the compounds can increase the level of progranulin or granulin in a subject.

The disclosure provides compounds of Formula (I):

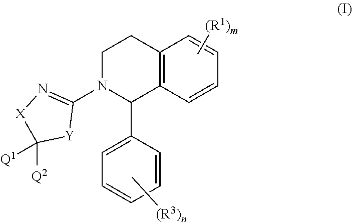

wherein

one of X and Y is O and the other is CH₂ or CH₂CH₂, or X is null or CH₂ and Y is CH₂O or (C═O)NH;

one of Q¹ and Q² is H and the other is C₀₋₃alkylene-NR⁶₂ or ring A,

or Q¹ and Q² together with the atom to which they are attached form ring A;

ring A comprises a 4- to 8-membered monocyclic or bicyclic carbocycle or heterocycle optionally substituted with 1-3 R² groups, wherein the heterocycle comprises a ring nitrogen or oxygen, or both;

each R¹ is independently C₁₋₆alkyl, O—C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SOₚR⁵, O—C₀₋₆alkylene-Ar, oxo, and C₀₋₆alkylene-Ar;

each R³ is independently halo, C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SO₂R⁵, O—C₀₋₆alkylene-Ar, and C₀₋₆alkylene-Ar;

each R² is independently halo, OH, C₁₋₆alkyl, C₁₋₆hydroxyalkyl, or C₀₋₆alkylene-N(R^{N})₂;

each R^{N} is independently H or C₁₋₆alkyl;

each R⁴ is independently H, C₁₋₆alkyl, or C(O)C₁₋₆alkyl;

each R⁵ is independently C₁₋₆alkyl, C₁₋₆haloalkyl, or Ar;

each R⁶ is independently H or C₁₋₆alkyl;

Ar is 3-8-membered carbocycle or heterocycle, wherein the heterocycle comprises 1-4 ring heteroatoms selected from N, O, and S; C₆₋₁₀aryl; or 5-10 membered heteroaryl comprising 1-4 ring heteroatoms selected from N, O, and S and Ar is optionally substituted with 1-3 groups independently selected from halo, C₁₋₆alkyl, C₁₋₆alkoxy, C₁₋₆haloalkoxy, CN, and SO₂C₁₋₃alkyl;

m is 0-2;

n is 0-3; and

p is 0-2. In some cases, one of X and Y is O and the other is CH₂ or CH₂CH₂, or X is null or CH₂ and Y is CH₂O, and each R² is independently halo, C₁₋₆alkyl, or C₀₋₆alkylene-N(R^{N})₂.

In some cases, the compound has a structure of Formula (II):

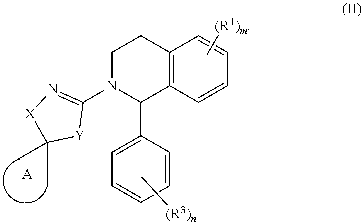

In some cases, the compound has a structure of Formula (IIa):

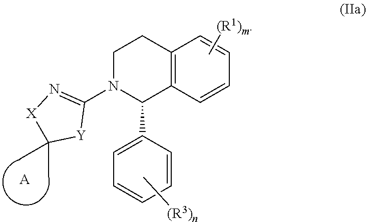

In some cases, the compound has a structure of Formula (IIb):

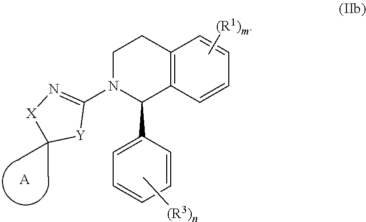

In some cases, one of X and Y is O and the other is CH₂ or CH₂CH₂, or X is null or CH₂ and Y is CH₂O. In some cases, each R² is independently halo, C₁₋₆alkyl, or —N(R^{N})₂. In some cases, one of X and Y is O and the other is CH₂ or CH₂CH₂, or X is null or CH₂ and Y is CH₂O and each R² is independently halo, C₁₋₆alkyl, or —N(R^{N})₂.

In some cases, Y is O. In some cases, Y is CH₂ or CH₂CH₂. In some cases, Y is CH₂. In some cases, Y is CH₂CH₂. In some cases, Y is CH₂O.

In some cases, X is CH₂. In some cases, X is CH₂CH₂. In some cases, X is O.

In some cases, X is CH₂ and Y is O. In some cases, X is CH₂CH₂ and Y is O. In some cases, X is CH₂ and Y is CH₂O. In some cases, X is null and Y is CH₂O. In some cases, X is null and Y is (C═O)NH.

In some cases, R² is halo. In some cases, R² is F. In some cases, R² is C₁₋₆alkyl. In some cases, R² is methyl. In some cases, R² is C₀₋₆alkylene-N(R^{N})₂. In some cases, R² is C₀alkylene-N(R^{N})₂, i.e., N(R^{N})₂. In some cases, R² is NH₂. In some cases, R² is OH. In some cases, R² is C₁₋₆haloalkyl. In some cases, R² is C₁₋₆hydroxyalkyl.

In some cases, R³ is halo. In some cases, R³ is F.

In some cases, m is 0 or 1. In some cases, m is O. In some cases, m is 1. In some cases, m is 2.

In some cases, n is 0. In some cases, n is 1, 2, or 3. In some cases, n is 1. In some cases, n is 2. In some cases, n is 3.

In some cases, the compound has a structure of Formula (III):

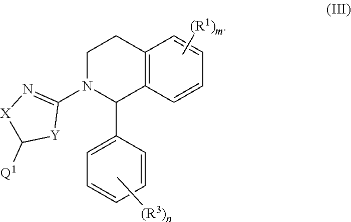

In some cases, Q¹ is C₀₋₃alkylene-NR⁶₂. In some cases, Q¹ is C₁₋₃alkylene-NR⁶₂. In some cases, Q¹ is CH₂NH₂.

In some cases, Q¹ is a 4- to 8-membered monocyclic or bicyclic carbocycle or heterocycle optionally substituted with 1-3 R² groups, wherein the heterocycle comprises a ring nitrogen or oxygen. In some cases, Q¹ comprises a quinuclidine, piperidine, pyrrolidine, azetidine, or cyclobutane moiety. In some cases, Q¹ is substituted with 1-3 R² groups. In some cases, Q¹ is

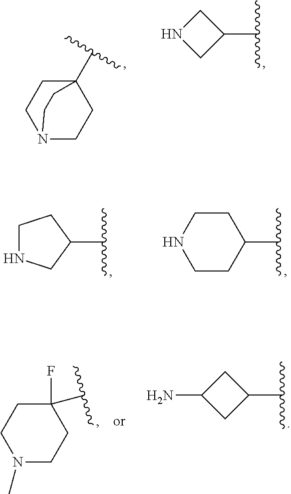

In some cases, Q¹ is

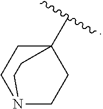 [SMILES: *C12CCN(CC1)CC2]

In some cases, Q¹ is

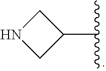 [SMILES: *C1CNC1]

In some cases, Q¹ is

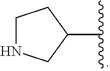 [SMILES: *C1CCNC1]

In some cases, Q¹ is

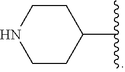 [SMILES: *C1CCNCC1]

In some cases, ring A comprises a quinuclidine, piperidine, pyrrolidine, 8-azabicyclo[3.2.1]octane, 6-azabicyclo[3.1.1]heptane, cyclobutane, cyclopentane, cyclohexane, cycloheptane, furan, pyran, oxepin, morpholine, or thiomorpholine moiety.

In some cases, ring A is substituted with 1-3 R² groups. In some cases, ring A is

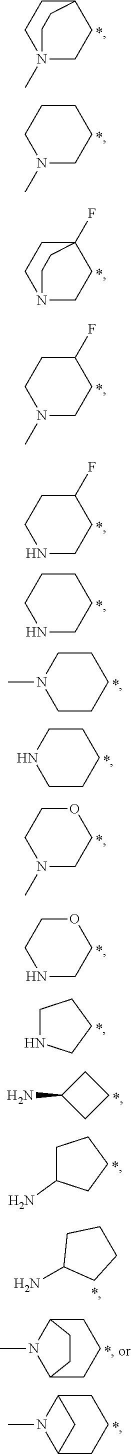

wherein * indicates the point of attachment. In some cases, ring A is

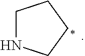

In some cases, ring A is

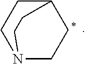

In some cases, ring A is

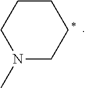

In some cases, ring A is

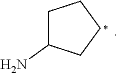

In some cases, ring A is

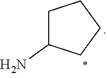

In some cases, R¹ is F, Cl, OH, OMe, OiPr, OBn, O-cyclopropyl, CF₃, OCF₃, CN, SO₂Me, SO₂-cyclopropyl, SO₂iPr, oxo, imidazolyl, triazolyl, pyrrolidinyl, pyrrolidinonyl, thiadiazolyl, methyl-thiadiazolyl, trifluoromethyl-thiadiazolyl, oxadiazolyl, methyl-oxadiazolyl, trifluoromethyl-oxadiazolyl, or phenyl. In some cases, R¹ is F or Cl. In some cases, R¹ is F. In some cases, R¹ is Cl.

In some cases, p is 0. In some cases, p is 1. In some cases, p is 2.

Specific compounds contemplated include those listed in Table A, or a pharmaceutically acceptable salt thereof:

TABLE A

| | Compound |
|---|---|
| Structure | No. |
| 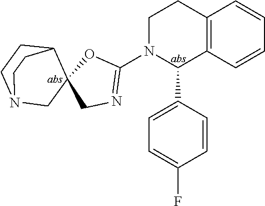 [SMILES: Fc1ccc([C@H]2c3ccccc3CCN2C2=NC[C@]3(CN4CCC3CC4)O2)cc1] | 3000 |
| 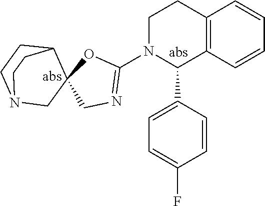 [SMILES: Fc1ccc([C@H]2c3ccccc3CCN2C2=NC[C@@]3(CN4CCC3CC4)O2)cc1] | 3001 |
| 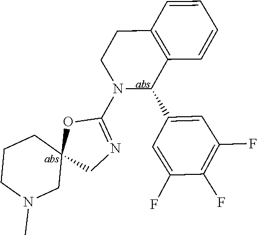 [SMILES: CN1CCC[C@]2(CN=C(N3CCc4ccccc4[C@@H]3c3cc(F)c(F)c(F)c3)O2)C1] | 3002 |
| 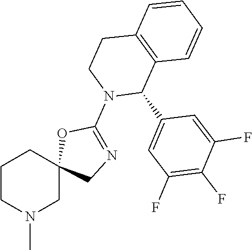 [SMILES: CN1CCC[C@@]2(CN=C(N3CCc4ccccc4[C@@H]3c3cc(F)c(F)c(F)c3)O2)C1] | 3003 |
| 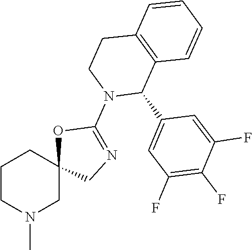 [SMILES: CN1CCC[C@]2(CN=C(N3CCc4ccccc4[C@@H]3c3cc(F)c(F)c(F)c3)O2)C1] | 3004 |
| 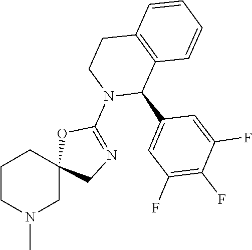 [SMILES: CN1CCC[C@@]2(CN=C(N3CCc4ccccc4[C@H]3c3cc(F)c(F)c(F)c3)O2)C1] | 3005 |
| 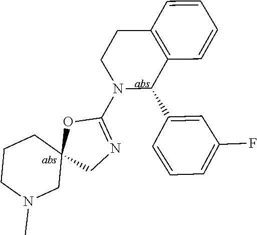 [SMILES: CN1CCC[C@]2(CN=C(N3CCc4ccccc4[C@@H]3c3cccc(F)c3)O2)C1] | 3006 |
| 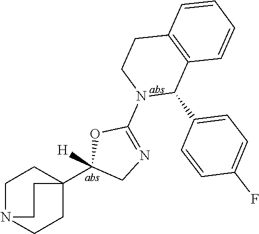 [SMILES: Fc1ccc([C@H]2c3ccccc3CCN2C2=NC[C@H](C34CCN(CC3)CC4)O2)cc1] | 3200 |
| 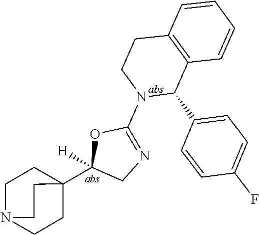 [SMILES: Fc1ccc([C@H]2c3ccccc3CCN2C2=NC[C@@H](C34CCN(CC3)CC4)O2)cc1] | 3201 |
| 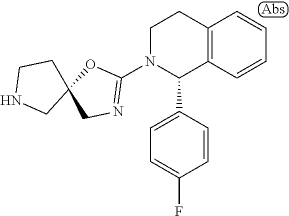 [SMILES: Fc1ccc([C@H]2c3ccccc3CCN2C2=NC[C@@]3(CCNC3)O2)cc1] | 3202 |
| 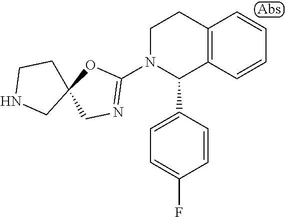 [SMILES: Fc1ccc([C@H]2c3ccccc3CCN2C2=NC[C@]3(CCNC3)O2)cc1] | 3203 |
| 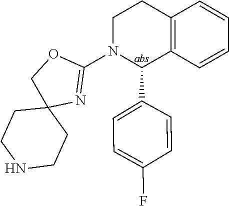 [SMILES: Fc1ccc([C@H]2c3ccccc3CCN2C2=NC3(CCNCC3)CO2)cc1] | 3204 |
| 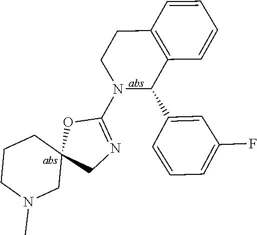 [SMILES: CN1CCC[C@@]2(CN=C(N3CCc4ccccc4[C@@H]3c3cccc(F)c3)O2)C1] | 3207 |
| 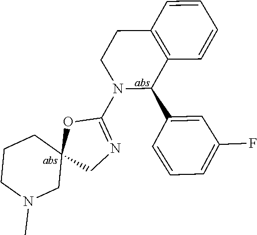 [SMILES: CN1CCC[C@]2(CN=C(N3CCc4ccccc4[C@H]3c3cccc(F)c3)O2)C1] | 3008 |
| 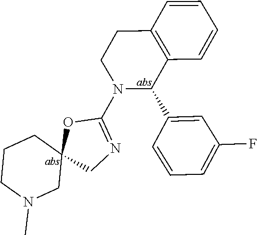 [SMILES: CN1CCC[C@]2(CN=C(N3CCc4ccccc4[C@@H]3c3cccc(F)c3)O2)C1] | 3009 |
| 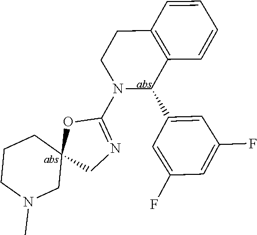 [SMILES: CN1CCC[C@]2(CN=C(N3CCc4ccccc4[C@@H]3c3cc(F)cc(F)c3)O2)C1] | 3010 |
| 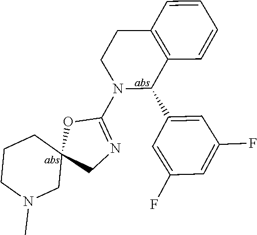 [SMILES: CN1CCC[C@@]2(CN=C(N3CCc4ccccc4[C@@H]3c3cc(F)cc(F)c3)O2)C1] | 3011 |
| 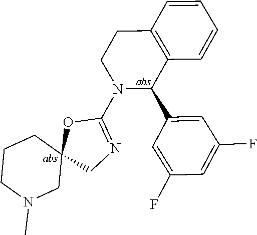 [SMILES: CN1CCC[C@]2(CN=C(N3CCc4ccccc4[C@H]3c3cc(F)cc(F)c3)O2)C1] | 3012 |
| 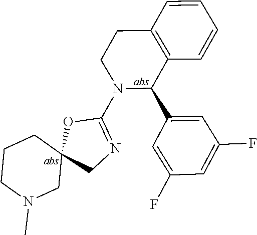 [SMILES: CN1CCC[C@@]2(CN=C(N3CCc4ccccc4[C@H]3c3cc(F)cc(F)c3)O2)C1] | 3013 |
| 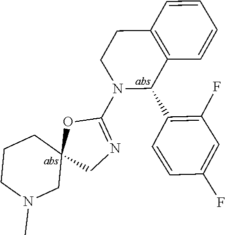 [SMILES: CN1CCC[C@]2(CN=C(N3CCc4ccccc4[C@@H]3c3ccc(F)cc3F)O2)C1] | 3014 |
| 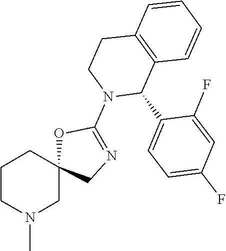 [SMILES: CN1CCC[C@@]2(CN=C(N3CCc4ccccc4[C@@H]3c3ccc(F)cc3F)O2)C1] | 3015 |
| 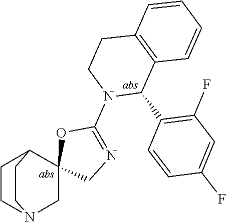 [SMILES: Fc1ccc([C@H]2c3ccccc3CCN2C2=NC[C@@]3(CN4CCC3CC4)O2)c(F)c1] | 3016 |
| 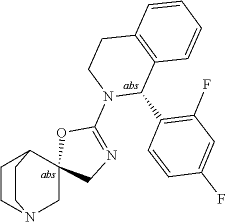 [SMILES: Fc1ccc([C@H]2c3ccccc3CCN2C2=NC[C@]3(CN4CCC3CC4)O2)c(F)c1] | 3017 |
| 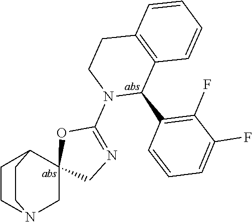 [SMILES: Fc1cccc([C@@H]2c3ccccc3CCN2C2=NC[C@@]3(CN4CCC3CC4)O2)c1F] | 3018 |
| 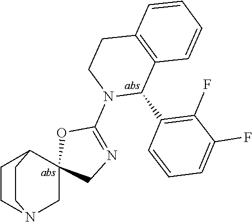 [SMILES: Fc1cccc([C@H]2c3ccccc3CCN2C2=NC[C@]3(CN4CCC3CC4)O2)c1F] | 3019 |
| 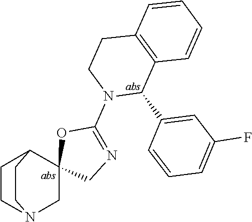 [SMILES: Fc1cccc([C@H]2c3ccccc3CCN2C2=NC[C@@]3(CN4CCC3CC4)O2)c1] | 3020 |
| 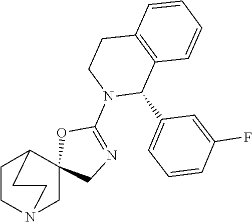 [SMILES: Fc1cccc([C@H]2c3ccccc3CCN2C2=NC[C@]3(CN4CCC3CC4)O2)c1] | 3021 |
| 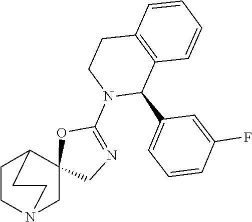 [SMILES: Fc1cccc([C@@H]2c3ccccc3CCN2C2=NC[C@@]3(CN4CCC3CC4)O2)c1] | 3022 |
| 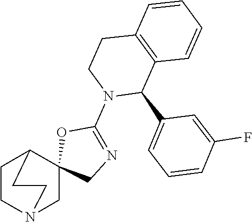 [SMILES: Fc1cccc([C@@H]2c3ccccc3CCN2C2=NC[C@]3(CN4CCC3CC4)O2)c1] | 3023 |
| 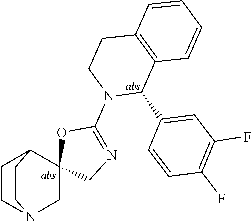 [SMILES: Fc1ccc([C@H]2c3ccccc3CCN2C2=NC[C@@]3(CN4CCC3CC4)O2)cc1F] | 3024 |
| 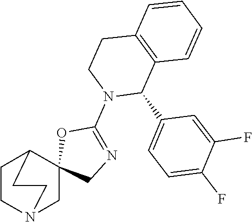 [SMILES: Fc1ccc([C@H]2c3ccccc3CCN2C2=NC[C@]3(CN4CCC3CC4)O2)cc1F] | 3025 |
| 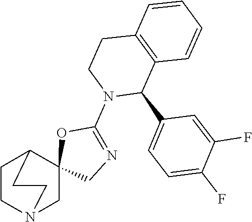 [SMILES: Fc1ccc([C@@H]2c3ccccc3CCN2C2=NC[C@@]3(CN4CCC3CC4)O2)cc1F] | 3026 |
| 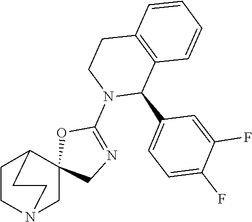 [SMILES: Fc1ccc([C@@H]2c3ccccc3CCN2C2=NC[C@]3(CN4CCC3CC4)O2)cc1F] | 3027 |
| 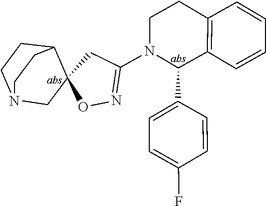 [SMILES: Fc1ccc([C@H]2c3ccccc3CCN2C2=NO[C@@]3(C2)CN2CCC3CC2)cc1] | 3028 |
| 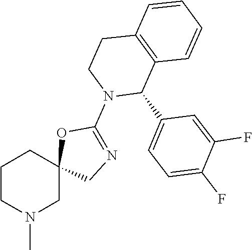 [SMILES: CN1CCC[C@]2(CN=C(N3CCc4ccccc4[C@@H]3c3ccc(F)c(F)c3)O2)C1] | 3029 |
| 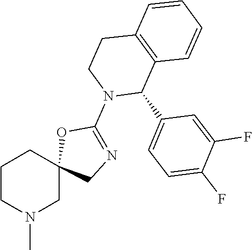 [SMILES: CN1CCC[C@@]2(CN=C(N3CCc4ccccc4[C@@H]3c3ccc(F)c(F)c3)O2)C1] | 3030 |
| 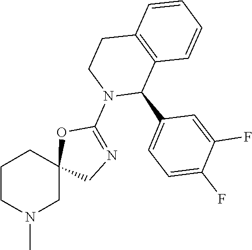 [SMILES: CN1CCC[C@]2(CN=C(N3CCc4ccccc4[C@H]3c3ccc(F)c(F)c3)O2)C1] | 3031 |
| 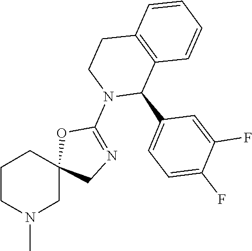 [SMILES: CN1CCC[C@@]2(CN=C(N3CCc4ccccc4[C@H]3c3ccc(F)c(F)c3)O2)C1] | 3032 |
| 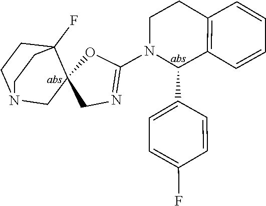 [SMILES: Fc1ccc([C@H]2c3ccccc3CCN2C2=NC[C@]3(CN4CCC3(F)CC4)O2)cc1] | 3033 |
| 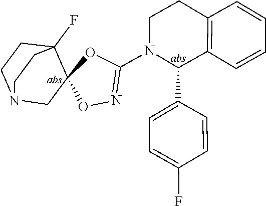 [SMILES: Fc1ccc([C@H]2c3ccccc3CCN2C2=NO[C@@]3(CN4CCC3(F)CC4)O2)cc1] | 3034 |
| 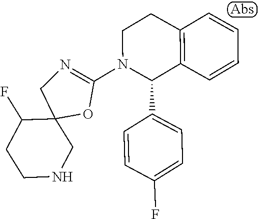 [SMILES: Fc1ccc([C@H]2c3ccccc3CCN2C2=NCC3(CNCCC3F)O2)cc1] | 3035 |
| 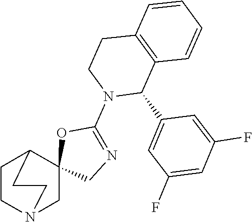 [SMILES: Fc1cc(F)cc([C@H]2c3ccccc3CCN2C2=NC[C@@]3(CN4CCC3CC4)O2)c1] | 3036 |
| 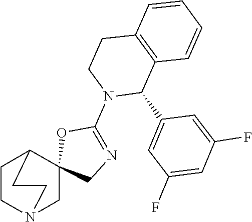 [SMILES: Fc1cc(F)cc([C@H]2c3ccccc3CCN2C2=NC[C@]3(CN4CCC3CC4)O2)c1] | 3037 |
| 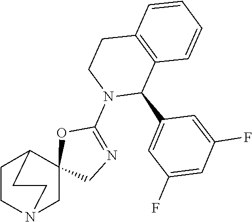 [SMILES: Fc1cc(F)cc([C@@H]2c3ccccc3CCN2C2=NC[C@@]3(CN4CCC3CC4)O2)c1] | 3038 |
| 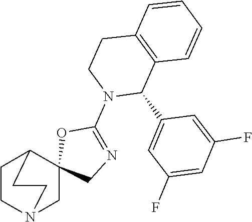 [SMILES: Fc1cc(F)cc([C@H]2c3ccccc3CCN2C2=NC[C@]3(CN4CCC3CC4)O2)c1] | 3039 |
| 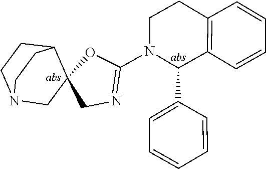 [SMILES: c1ccc([C@H]2c3ccccc3CCN2C2=NC[C@]3(CN4CCC3CC4)O2)cc1] | 3040 |
| 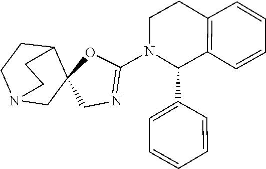 [SMILES: c1ccc([C@H]2c3ccccc3CCN2C2=NC[C@@]3(CN4CCC3CC4)O2)cc1] | 3041 |
| 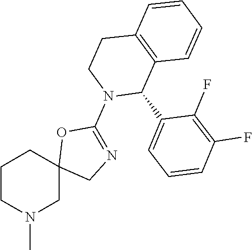 [SMILES: CN1CCCC2(CN=C(N3CCc4ccccc4[C@@H]3c3cccc(F)c3F)O2)C1] | 3042 |
| 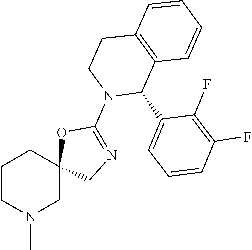 [SMILES: CN1CCC[C@]2(CN=C(N3CCc4ccccc4[C@@H]3c3cccc(F)c3F)O2)C1] | 3043 |
| 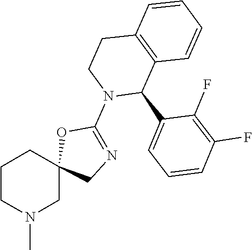 [SMILES: CN1CCC[C@@]2(CN=C(N3CCc4ccccc4[C@H]3c3cccc(F)c3F)O2)C1] | 3044 |
| 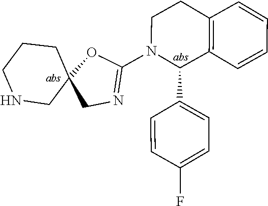 | 3051 |
| 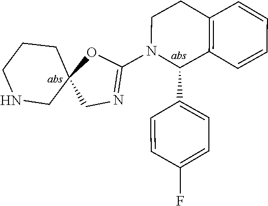 [SMILES: Fc1ccc([C@H]2c3ccccc3CCN2C2=NC[C@]3(CCCNC3)O2)cc1] | 3052 |
| 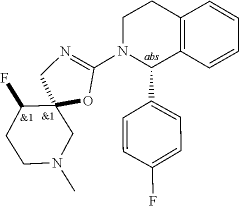 | 3045 |
| 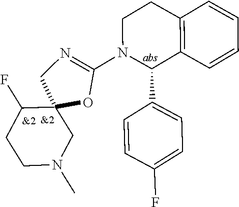 | 3046 |
| 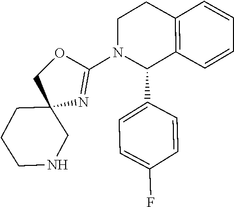 [SMILES: Fc1ccc([C@H]2c3ccccc3CCN2C2=N[C@]3(CCCNC3)CO2)cc1] | 3053 |
| 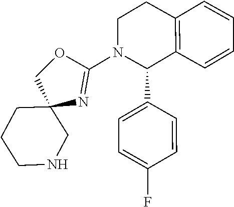 [SMILES: Fc1ccc([C@H]2c3ccccc3CCN2C2=N[C@@]3(CCCNC3)CO2)cc1] | 3054 |
| 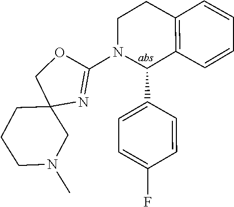 [SMILES: CN1CCCC2(COC(N3CCc4ccccc4[C@@H]3c3ccc(F)cc3)=N2)C1] | 3055 |
| 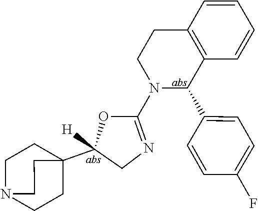 [SMILES: Fc1ccc([C@H]2c3ccccc3CCN2C2=NC[C@H](C34CCN(CC3)CC4)O2)cc1] | 3047 |
| 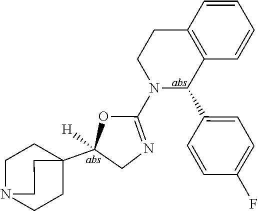 [SMILES: Fc1ccc([C@H]2c3ccccc3CCN2C2=NC[C@@H](C34CCN(CC3)CC4)O2)cc1] | 3048 |
| 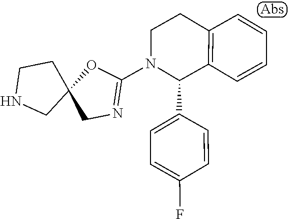 [SMILES: Fc1ccc([C@H]2c3ccccc3CCN2C2=NC[C@@]3(CCNC3)O2)cc1] | 3049 |
| 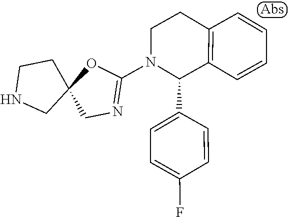 [SMILES: Fc1ccc([C@H]2c3ccccc3CCN2C2=NC[C@]3(CCNC3)O2)cc1] | 3050 |
| 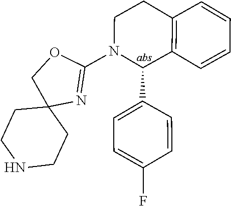 [SMILES: Fc1ccc([C@H]2c3ccccc3CCN2C2=NC3(CCNCC3)CO2)cc1] | 3056 |

In some cases, the compound of Table A, or salt thereof, is selected from Compounds 3000-3056. In some cases, the compound of Table A, or salt thereof, is selected from Compounds 3007-3056 and 3200-3204.

Additional compounds contemplated include those listed in Table B, or a pharmaceutically acceptable salt thereof:

TABLE B

| | Compound |
|---|---|
| Structure | No. |
| 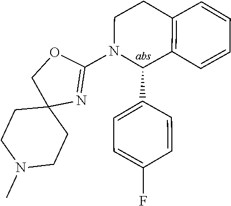 [SMILES: CN1CCC2(CC1)COC(N1CCc3ccccc3[C@@H]1c1ccc(F)cc1)=N2] | 3058 |
| 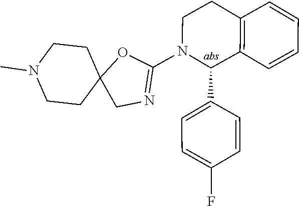 [SMILES: CN1CCC2(CC1)CN=C(N1CCc3ccccc3[C@@H]1c1ccc(F)cc1)O2] | 3059 |
| 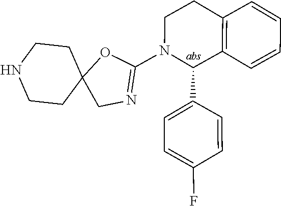 [SMILES: Fc1ccc([C@H]2c3ccccc3CCN2C2=NCC3(CCNCC3)O2)cc1] | 3060 |
| 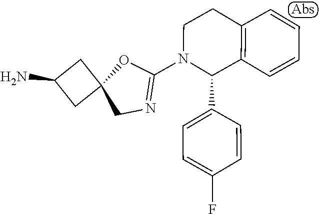 [SMILES: N[C@H]1C[C@@]2(CN=C(N3CCc4ccccc4[C@@H]3c3ccc(F)cc3)O2)C1] | 3061 |
| 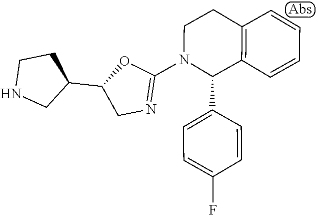 [SMILES: Fc1ccc([C@H]2c3ccccc3CCN2C2=NC[C@H]([C@@H]3CCNC3)O2)cc1] | 3063 |
| 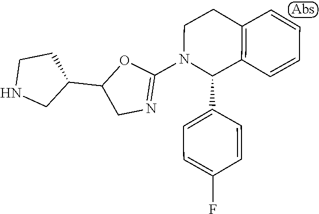 [SMILES: Fc1ccc([C@H]2c3ccccc3CCN2C2=NCC([C@H]3CCNC3)O2)cc1] | 3064 |
| 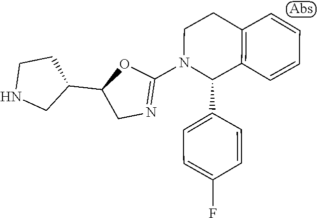 [SMILES: Fc1ccc([C@H]2c3ccccc3CCN2C2=NC[C@@H]([C@H]3CCNC3)O2)cc1] | 3065 |
| 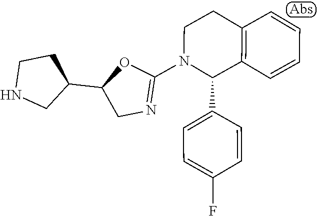 [SMILES: Fc1ccc([C@H]2c3ccccc3CCN2C2=NC[C@@H]([C@@H]3CCNC3)O2)cc1] | 3066 |
| 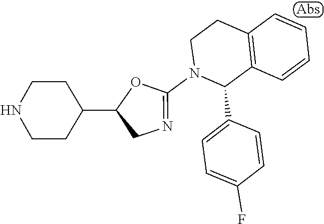 [SMILES: Fc1ccc([C@H]2c3ccccc3CCN2C2=NC[C@@H](C3CCNCC3)O2)cc1] | 3067 |
| 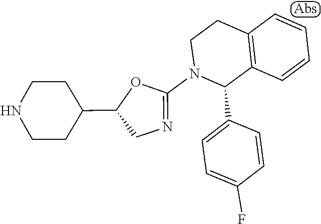 [SMILES: Fc1ccc([C@H]2c3ccccc3CCN2C2=NC[C@H](C3CCNCC3)O2)cc1] | 3068 |
| 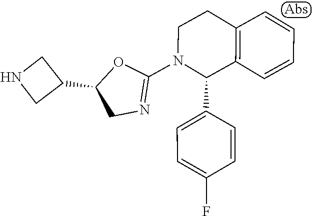 | 3069 |
| 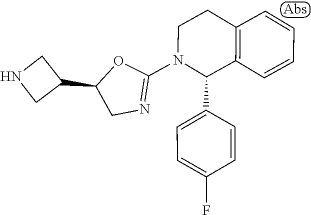 [SMILES: Fc1ccc([C@H]2c3ccccc3CCN2C2=NC[C@@H](C3CNC3)O2)cc1] | 3070 |
| 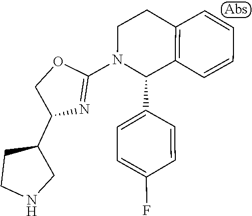 [SMILES: Fc1ccc([C@H]2c3ccccc3CCN2C2=N[C@H]([C@@H]3CCNC3)CO2)cc1] | 3071 |
| 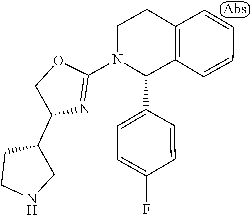 [SMILES: Fc1ccc([C@H]2c3ccccc3CCN2C2=N[C@H]([C@H]3CCNC3)CO2)cc1] | 3072 |
| 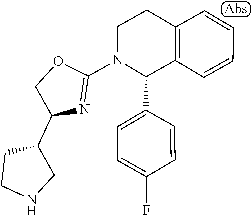 [SMILES: Fc1ccc([C@H]2c3ccccc3CCN2C2=N[C@@H]([C@H]3CCNC3)CO2)cc1] | 3073 |
| 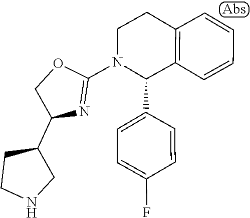 [SMILES: Fc1ccc([C@H]2c3ccccc3CCN2C2=N[C@@H]([C@@H]3CCNC3)CO2)cc1] | 3074 |
| 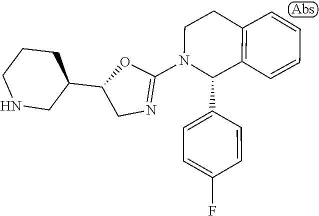 [SMILES: Fc1ccc([C@H]2c3ccccc3CCN2C2=NC[C@H]([C@@H]3CCCNC3)O2)cc1] | 3075 |
| 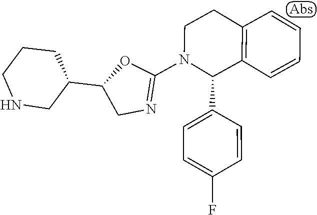 [SMILES: Fc1ccc([C@H]2c3ccccc3CCN2C2=NC[C@H]([C@H]3CCCNC3)O2)cc1] | 3076 |
| 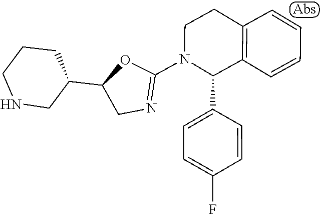 [SMILES: Fc1ccc([C@H]2c3ccccc3CCN2C2=NC[C@@H]([C@H]3CCCNC3)O2)cc1] | 3077 |
| 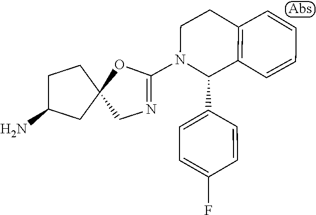 [SMILES: N[C@H]1CC[C@]2(CN=C(N3CCc4ccccc4[C@@H]3c3ccc(F)cc3)O2)C1] | 3078 |
| 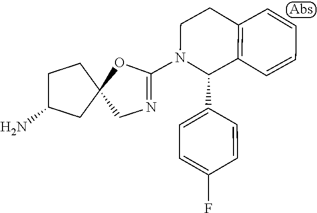 [SMILES: N[C@@H]1CC[C@]2(CN=C(N3CCc4ccccc4[C@@H]3c3ccc(F)cc3)O2)C1] | 3079 |
| 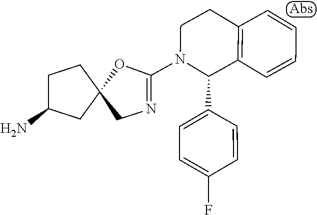 [SMILES: N[C@H]1CC[C@@]2(CN=C(N3CCc4ccccc4[C@@H]3c3ccc(F)cc3)O2)C1] | 3080 |
| 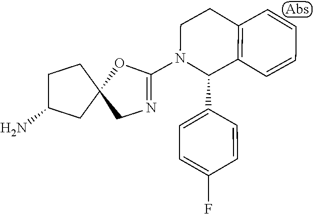 [SMILES: N[C@@H]1CC[C@@]2(CN=C(N3CCc4ccccc4[C@@H]3c3ccc(F)cc3)O2)C1] | 3081 |
| 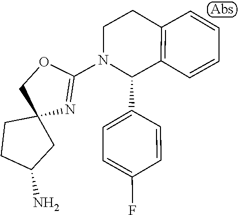 [SMILES: N[C@@H]1CC[C@@]2(COC(N3CCc4ccccc4[C@@H]3c3ccc(F)cc3)=N2)C1] | 3082 |
| 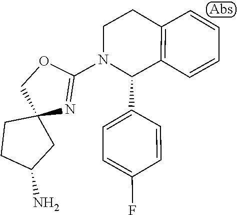 [SMILES: N[C@@H]1CC[C@]2(COC(N3CCc4ccccc4[C@@H]3c3ccc(F)cc3)=N2)C1] | 3083 |
| 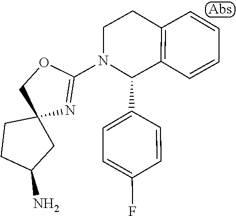 [SMILES: N[C@H]1CC[C@@]2(COC(N3CCc4ccccc4[C@@H]3c3ccc(F)cc3)=N2)C1] | 3084 |
| 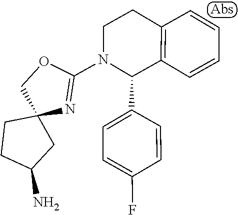 [SMILES: N[C@H]1CC[C@]2(COC(N3CCc4ccccc4[C@@H]3c3ccc(F)cc3)=N2)C1] | 3085 |
| 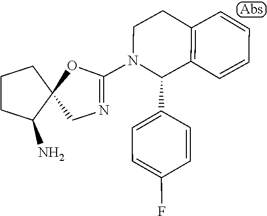 [SMILES: N[C@H]1CCC[C@]12CN=C(N1CCc3ccccc3[C@@H]1c1ccc(F)cc1)O2] | 3086 |
| 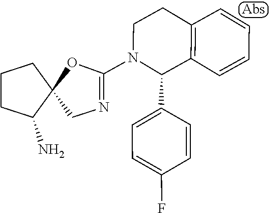 [SMILES: N[C@@H]1CCC[C@]12CN=C(N1CCc3ccccc3[C@@H]1c1ccc(F)cc1)O2] | 3087 |
| 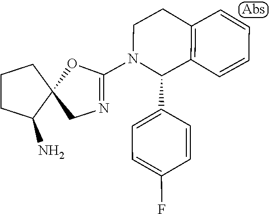 [SMILES: N[C@H]1CCC[C@@]12CN=C(N1CCc3ccccc3[C@@H]1c1ccc(F)cc1)O2] | 3088 |
| 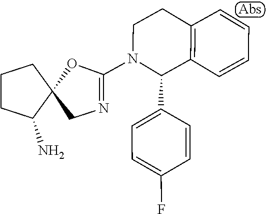 [SMILES: N[C@@H]1CCC[C@@]12CN=C(N1CCc3ccccc3[C@@H]1c1ccc(F)cc1)O2] | 3089 |
| 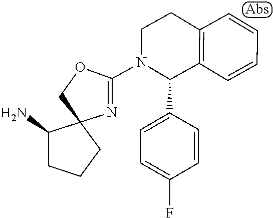 [SMILES: N[C@@H]1CCC[C@]12COC(N1CCc3ccccc3[C@@H]1c1ccc(F)cc1)=N2] | 3090 |
| 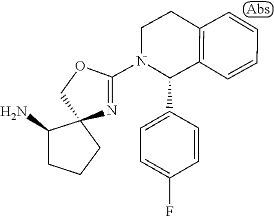 [SMILES: N[C@@H]1CCC[C@@]12COC(N1CCc3ccccc3[C@@H]1c1ccc(F)cc1)=N2] | 3091 |
| 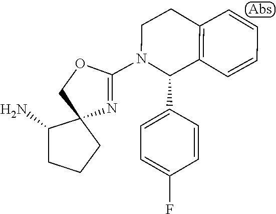 [SMILES: N[C@H]1CCC[C@]12COC(N1CCc3ccccc3[C@@H]1c1ccc(F)cc1)=N2] | 3092 |
| 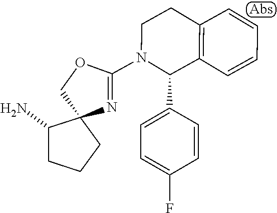 [SMILES: N[C@H]1CCC[C@@]12COC(N1CCc3ccccc3[C@@H]1c1ccc(F)cc1)=N2] | 3093 |
| 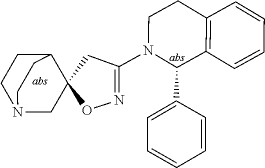 [SMILES: c1ccc([C@H]2c3ccccc3CCN2C2=NO[C@@]3(C2)CN2CCC3CC2)cc1] | 3094 |
| 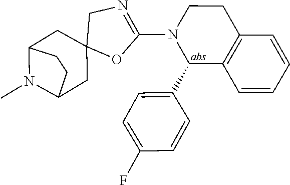 [SMILES: CN1C2CCC1CC1(CN=C(N3CCc4ccccc4[C@@H]3c3ccc(F)cc3)O1)C2] | 3095 |
| 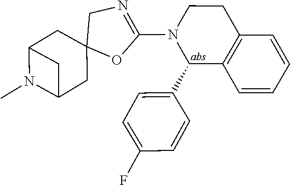 [SMILES: CN1C2CC1CC1(CN=C(N3CCc4ccccc4[C@@H]3c3ccc(F)cc3)O1)C2] | 3096 |
| 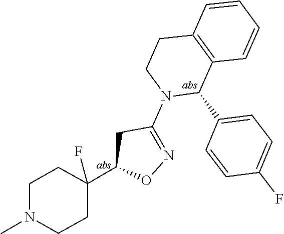 | 3097 |
| 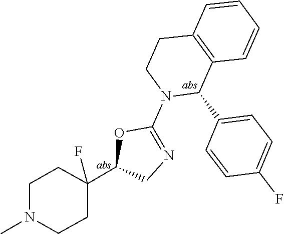 | 3098 |
| 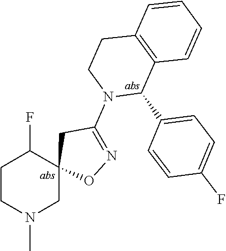 [SMILES: CN1CCC(F)[C@@]2(CC(N3CCc4ccccc4[C@@H]3c3ccc(F)cc3)=NO2)C1] | 3099 |
| 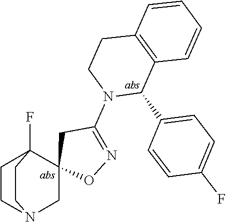 [SMILES: Fc1ccc([C@H]2c3ccccc3CCN2C2=NO[C@]3(C2)CN2CCC3(F)CC2)cc1] | 3100 |
| 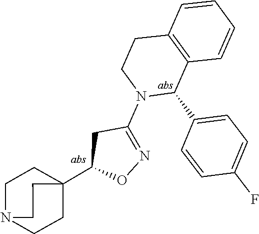 | 3101 |
| 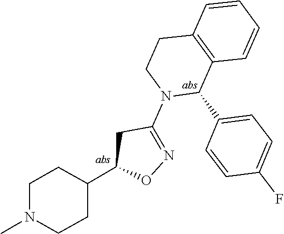 | 3102 |
| 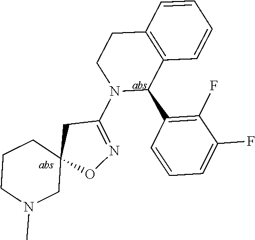 [SMILES: CN1CCC[C@@]2(CC(N3CCc4ccccc4[C@H]3c3cccc(F)c3F)=NO2)C1] | 3103 |
| 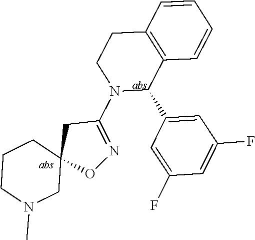 [SMILES: CN1CCC[C@@]2(CC(N3CCc4ccccc4[C@@H]3c3cc(F)cc(F)c3)=NO2)C1] | 3104 |
| 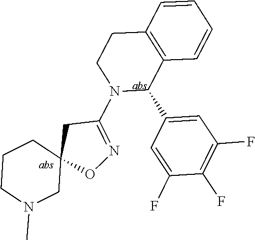 [SMILES: CN1CCC[C@@]2(CC(N3CCc4ccccc4[C@@H]3c3cc(F)c(F)c(F)c3)=NO2)C1] | 3105 |
| 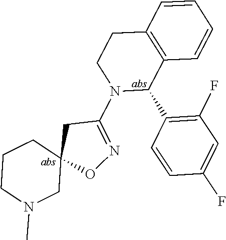 [SMILES: CN1CCC[C@@]2(CC(N3CCc4ccccc4[C@@H]3c3ccc(F)cc3F)=NO2)C1] | 3106 |
| 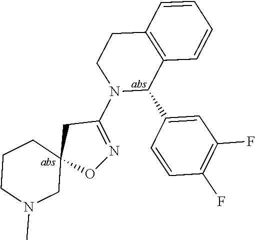 [SMILES: CN1CCC[C@@]2(CC(N3CCc4ccccc4[C@@H]3c3ccc(F)c(F)c3)=NO2)C1] | 3107 |
| 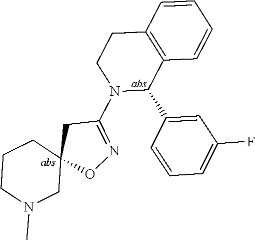 [SMILES: CN1CCC[C@@]2(CC(N3CCc4ccccc4[C@@H]3c3cccc(F)c3)=NO2)C1] | 3108 |
| 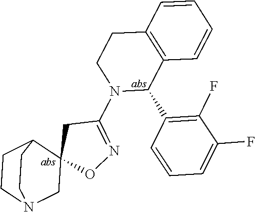 [SMILES: Fc1cccc([C@H]2c3ccccc3CCN2C2=NO[C@]3(C2)CN2CCC3CC2)c1F] | 3109 |
| 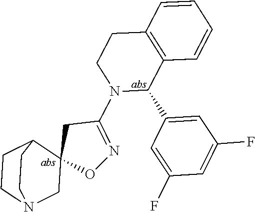 [SMILES: Fc1cc(F)cc([C@H]2c3ccccc3CCN2C2=NO[C@]3(C2)CN2CCC3CC2)c1] | 3110 |
| 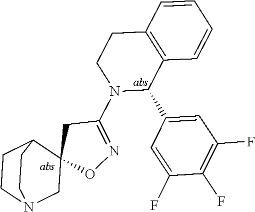 [SMILES: Fc1cc([C@H]2c3ccccc3CCN2C2=NO[C@]3(C2)CN2CCC3CC2)cc(F)c1F] | 3111 |
| 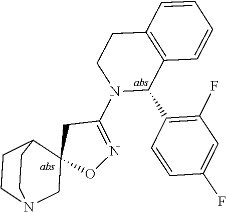 [SMILES: Fc1ccc([C@H]2c3ccccc3CCN2C2=NO[C@]3(C2)CN2CCC3CC2)c(F)c1] | 3112 |
| 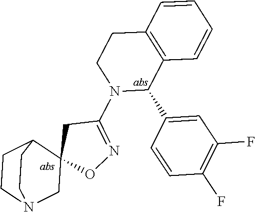 [SMILES: Fc1ccc([C@H]2c3ccccc3CCN2C2=NO[C@]3(C2)CN2CCC3CC2)cc1F] | 3113 |
| 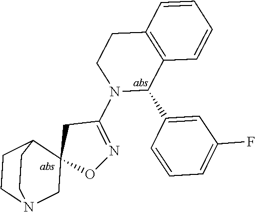 [SMILES: Fc1cccc([C@H]2c3ccccc3CCN2C2=NO[C@]3(C2)CN2CCC3CC2)c1] | 3114 |
| 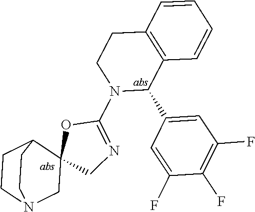 [SMILES: Fc1cc([C@H]2c3ccccc3CCN2C2=NC[C@@]3(CN4CCC3CC4)O2)cc(F)c1F] | 3115 |
| 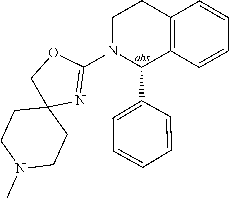 [SMILES: CN1CCC2(CC1)COC(N1CCc3ccccc3[C@@H]1c1ccccc1)=N2] | 3116 |
| 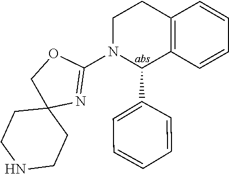 [SMILES: c1ccc([C@H]2c3ccccc3CCN2C2=NC3(CCNCC3)CO2)cc1] | 3117 |
| 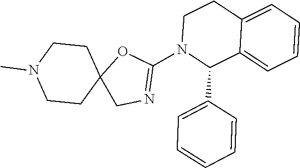 [SMILES: CN1CCC2(CC1)CN=C(N1CCc3ccccc3[C@@H]1c1ccccc1)O2] | 3118 |
| 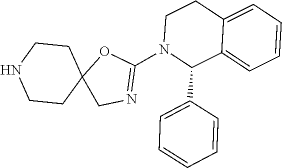 [SMILES: c1ccc([C@H]2c3ccccc3CCN2C2=NCC3(CCNCC3)O2)cc1] | 3119 |
| 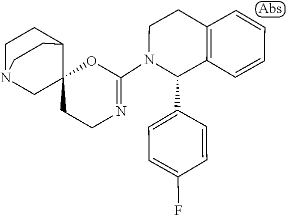 [SMILES: Fc1ccc([C@H]2c3ccccc3CCN2C2=NCC[C@]3(CN4CCC3CC4)O2)cc1] | 3120 |
| 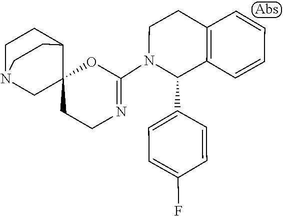 [SMILES: Fc1ccc([C@H]2c3ccccc3CCN2C2=NCC[C@]3(CN4CCC3CC4)O2)cc1] | 3121 |
| 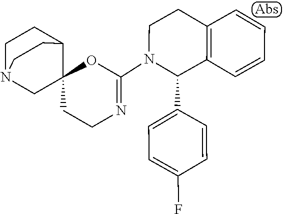 [SMILES: Fc1ccc([C@H]2c3ccccc3CCN2C2=NCC[C@@]3(CN4CCC3CC4)O2)cc1] | 3122 |
| 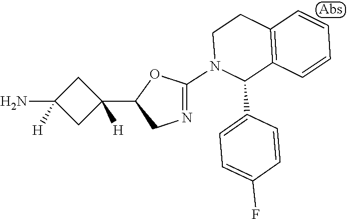 [SMILES: N[C@H]1C[C@H]([C@@H]2CN=C(N3CCc4ccccc4[C@@H]3c3ccc(F)cc3)O2)C1] | 3123 |
| 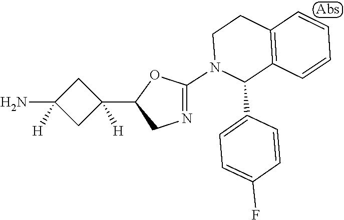 [SMILES: N[C@H]1C[C@@H]([C@@H]2CN=C(N3CCc4ccccc4[C@@H]3c3ccc(F)cc3)O2)C1] | 3124 |
| 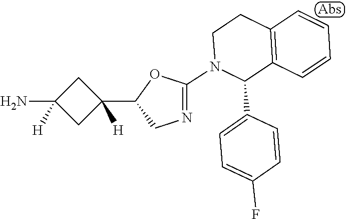 [SMILES: N[C@H]1C[C@H]([C@H]2CN=C(N3CCc4ccccc4[C@@H]3c3ccc(F)cc3)O2)C1] | 3125 |
| 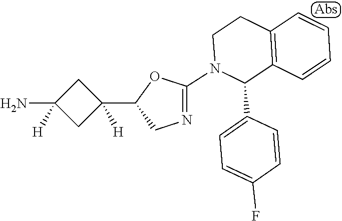 [SMILES: N[C@H]1C[C@@H]([C@H]2CN=C(N3CCc4ccccc4[C@@H]3c3ccc(F)cc3)O2)C1] | 3126 |
| 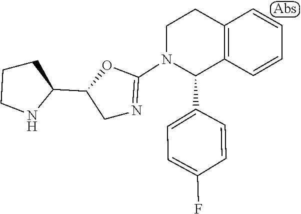 [SMILES: Fc1ccc([C@H]2c3ccccc3CCN2C2=NC[C@H]([C@@H]3CCCN3)O2)cc1] | 3127 |
| 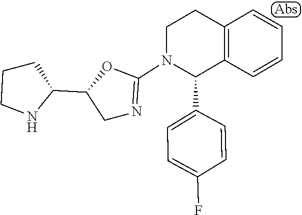 [SMILES: Fc1ccc([C@H]2c3ccccc3CCN2C2=NC[C@H]([C@H]3CCCN3)O2)cc1] | 3128 |
| 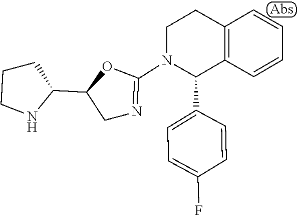 [SMILES: Fc1ccc([C@H]2c3ccccc3CCN2C2=NC[C@@H]([C@H]3CCCN3)O2)cc1] | 3129 |
| 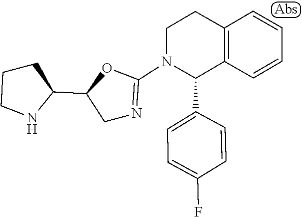 [SMILES: Fc1ccc([C@H]2c3ccccc3CCN2C2=NC[C@@H]([C@@H]3CCCN3)O2)cc1] | 3130 |
| 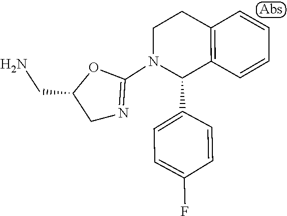 [SMILES: NC[C@H]1CN=C(N2CCc3ccccc3[C@@H]2c2ccc(F)cc2)O1] | 3131 |
| 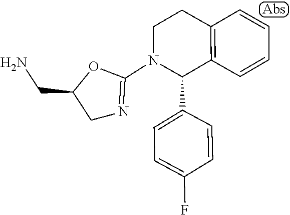 [SMILES: NC[C@@H]1CN=C(N2CCc3ccccc3[C@@H]2c2ccc(F)cc2)O1] | 3132 |
| 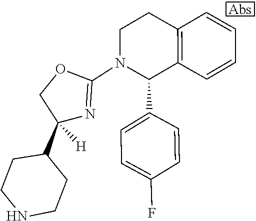 [SMILES: Fc1ccc([C@H]2c3ccccc3CCN2C2=N[C@@H](C3CCNCC3)CO2)cc1] | 3133 |
| 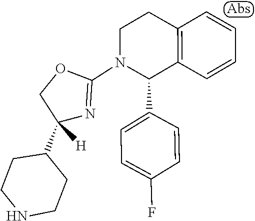 [SMILES: Fc1ccc([C@H]2c3ccccc3CCN2C2=N[C@H](C3CCNCC3)CO2)cc1] | 3134 |
| 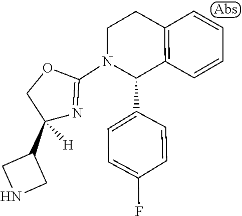 [SMILES: Fc1ccc([C@H]2c3ccccc3CCN2C2=N[C@@H](C3CNC3)CO2)cc1] | 3135 |
| 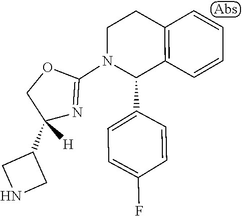 [SMILES: Fc1ccc([C@H]2c3ccccc3CCN2C2=N[C@H](C3CNC3)CO2)cc1] | 3136 |
| 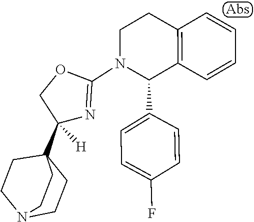 [SMILES: Fc1ccc([C@H]2c3ccccc3CCN2C2=N[C@@H](C34CCN(CC3)CC4)CO2)cc1] | 3137 |
| 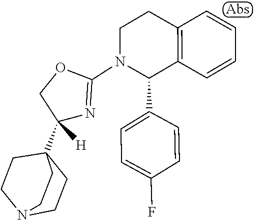 [SMILES: Fc1ccc([C@H]2c3ccccc3CCN2C2=N[C@H](C34CCN(CC3)CC4)CO2)cc1] | 3138 |
| 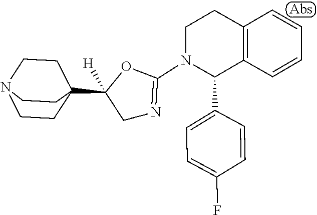 [SMILES: Fc1ccc([C@H]2c3ccccc3CCN2C2=NC[C@@H](C34CCN(CC3)CC4)O2)cc1] | 3139 |
| 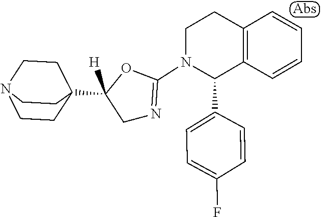 [SMILES: Fc1ccc([C@H]2c3ccccc3CCN2C2=NC[C@H](C34CCN(CC3)CC4)O2)cc1] | 3140 |
| 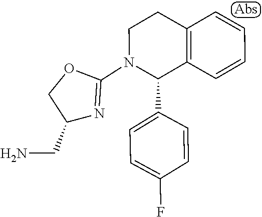 [SMILES: NC[C@@H]1COC(N2CCc3ccccc3[C@@H]2c2ccc(F)cc2)=N1] | 3141 |
| 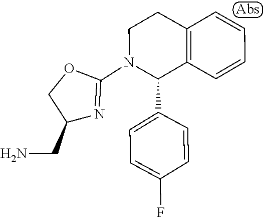 [SMILES: NC[C@H]1COC(N2CCc3ccccc3[C@@H]2c2ccc(F)cc2)=N1] | 3142 |

Additional compounds contemplated include those listed in Table C, or a pharmaceutically acceptable salt thereof:

TABLE C

| | Compound |
|---|---|
| Structure | No. |
| 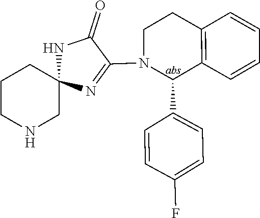 [SMILES: O=C1N[C@]2(CCCNC2)N=C1N1CCc2ccccc2[C@@H]1c1ccc(F)cc1] | 3147 |
| 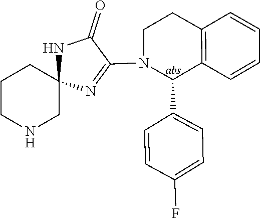 [SMILES: O=C1N[C@@]2(CCCNC2)N=C1N1CCc2ccccc2[C@@H]1c1ccc(F)cc1] | 3148 |
| 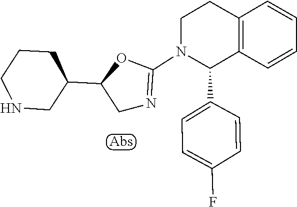 [SMILES: Fc1ccc([C@H]2c3ccccc3CCN2C2=NC[C@@H]([C@@H]3CCCNC3)O2)cc1] | 3149 |
| 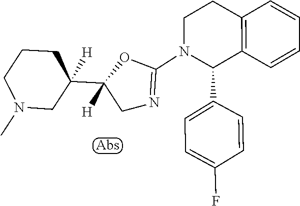 [SMILES: CN1CCC[C@@H]([C@@H]2CN=C(N3CCc4ccccc4[C@@H]3c3ccc(F)cc3)O2)C1] | 3150 |
| 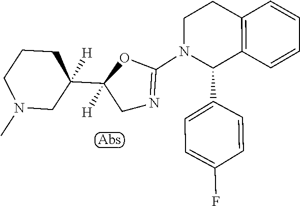 [SMILES: CN1CCC[C@@H]([C@H]2CN=C(N3CCc4ccccc4[C@@H]3c3ccc(F)cc3)O2)C1] | 3151 |
| 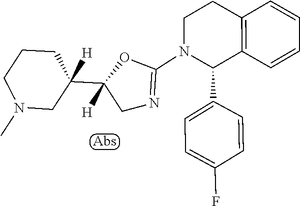 [SMILES: CN1CCC[C@H]([C@@H]2CN=C(N3CCc4ccccc4[C@@H]3c3ccc(F)cc3)O2)C1] | 3152 |
| 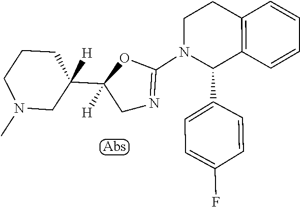 [SMILES: CN1CCC[C@H]([C@H]2CN=C(N3CCc4ccccc4[C@@H]3c3ccc(F)cc3)O2)C1] | 3153 |
| 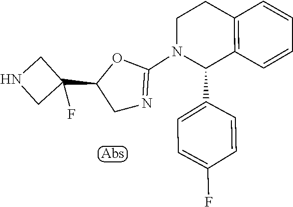 [SMILES: Fc1ccc([C@H]2c3ccccc3CCN2C2=NC[C@@H](C3(F)CNC3)O2)cc1] | 3154 |
| 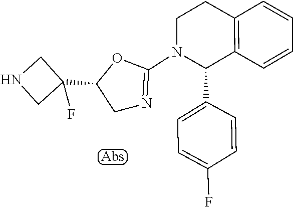 [SMILES: Fc1ccc([C@H]2c3ccccc3CCN2C2=NC[C@H](C3(F)CNC3)O2)cc1] | 3155 |
| 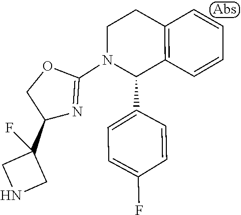 [SMILES: Fc1ccc([C@H]2c3ccccc3CCN2C2=N[C@@H](C3(F)CNC3)CO2)cc1] | 3156 |
| 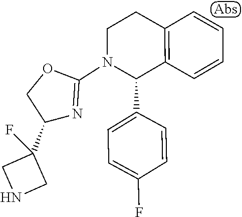 [SMILES: Fc1ccc([C@H]2c3ccccc3CCN2C2=N[C@H](C3(F)CNC3)CO2)cc1] | 3157 |
| 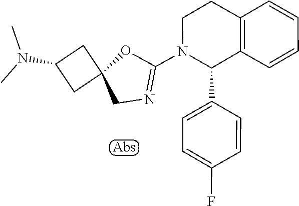 [SMILES: CN(C)[C@H]1C[C@]2(CN=C(N3CCc4ccccc4[C@@H]3c3ccc(F)cc3)O2)C1] | 3158 |
| 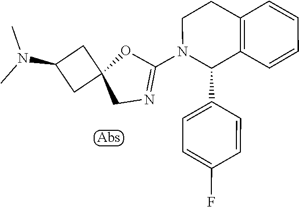 [SMILES: CN(C)[C@H]1C[C@@]2(CN=C(N3CCc4ccccc4[C@@H]3c3ccc(F)cc3)O2)C1] | 3159 |
| 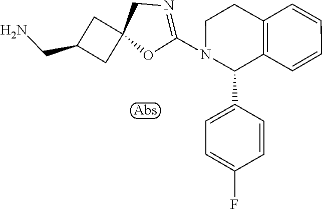 [SMILES: NC[C@H]1C[C@@]2(CN=C(N3CCc4ccccc4[C@@H]3c3ccc(F)cc3)O2)C1] | 3160 |
| 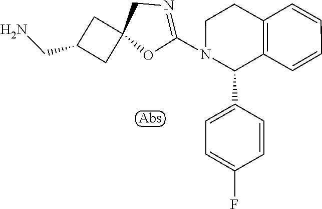 [SMILES: NC[C@H]1C[C@]2(CN=C(N3CCc4ccccc4[C@@H]3c3ccc(F)cc3)O2)C1] | 3161 |
| 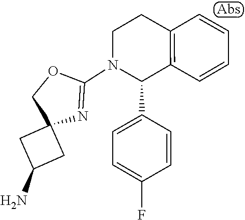 | 3162 |
| 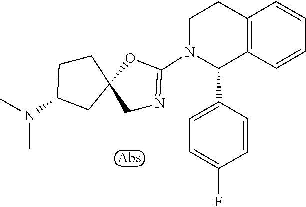 [SMILES: CN(C)[C@@H]1CC[C@@]2(CN=C(N3CCc4ccccc4[C@@H]3c3ccc(F)cc3)O2)C1] | 3163 |
| 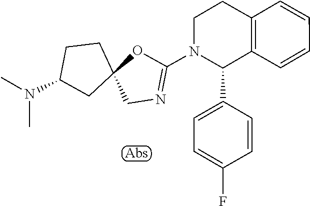 [SMILES: CN(C)[C@@H]1CC[C@]2(CN=C(N3CCc4ccccc4[C@@H]3c3ccc(F)cc3)O2)C1] | 3164 |
| 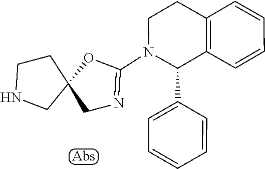 [SMILES: c1ccc([C@H]2c3ccccc3CCN2C2=NC[C@@]3(CCNC3)O2)cc1] | 3165 |
| 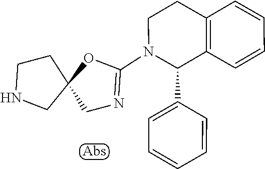 [SMILES: c1ccc([C@H]2c3ccccc3CCN2C2=NC[C@]3(CCNC3)O2)cc1] | 3166 |
| 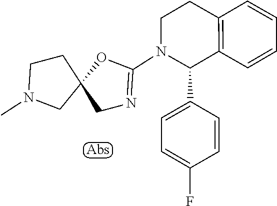 [SMILES: CN1CC[C@@]2(CN=C(N3CCc4ccccc4[C@@H]3c3ccc(F)cc3)O2)C1] | 3167 |
| 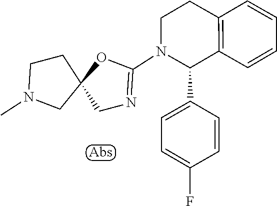 [SMILES: CN1CC[C@]2(CN=C(N3CCc4ccccc4[C@@H]3c3ccc(F)cc3)O2)C1] | 3168 |
| 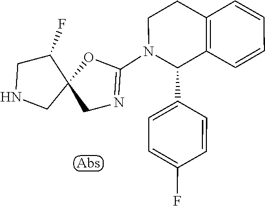 [SMILES: Fc1ccc([C@H]2c3ccccc3CCN2C2=NC[C@]3(CNC[C@@H]3F)O2)cc1] | 3169 |
| 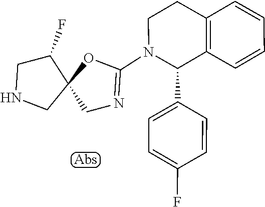 [SMILES: Fc1ccc([C@H]2c3ccccc3CCN2C2=NC[C@@]3(CNC[C@@H]3F)O2)cc1] | 3170 |
| 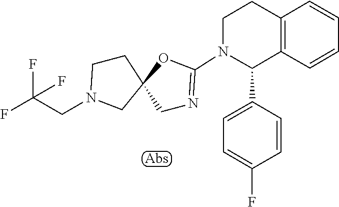 [SMILES: Fc1ccc([C@H]2c3ccccc3CCN2C2=NC[C@]3(CCN(CC(F)(F)F)C3)O2)cc1] | 3171 |
| 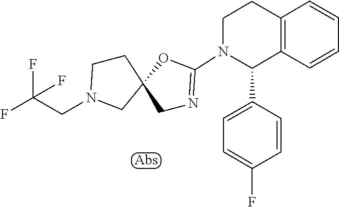 [SMILES: Fc1ccc([C@H]2c3ccccc3CCN2C2=NC[C@@]3(CCN(CC(F)(F)F)C3)O2)cc1] | 3172 |
| 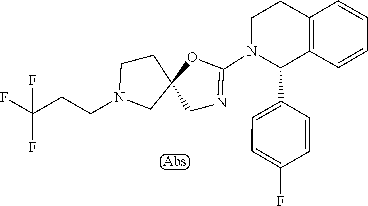 [SMILES: Fc1ccc([C@H]2c3ccccc3CCN2C2=NC[C@]3(CCN(CCC(F)(F)F)C3)O2)cc1] | 3173 |
| 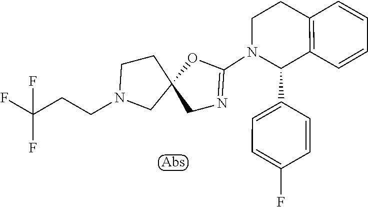 [SMILES: Fc1ccc([C@H]2c3ccccc3CCN2C2=NC[C@@]3(CCN(CCC(F)(F)F)C3)O2)cc1] | 3174 |
| 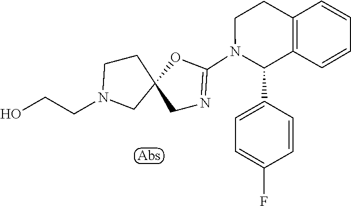 [SMILES: OCCN1CC[C@@]2(CN=C(N3CCc4ccccc4[C@@H]3c3ccc(F)cc3)O2)C1] | 3175 |
| 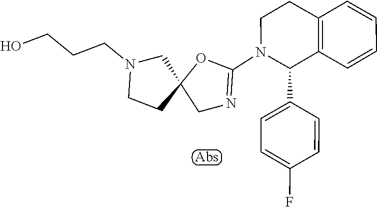 [SMILES: OCCCN1CC[C@@]2(CN=C(N3CCc4ccccc4[C@@H]3c3ccc(F)cc3)O2)C1] | 3176 |
| 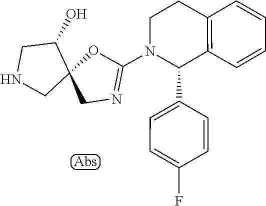 [SMILES: O[C@H]1CNC[C@]12CN=C(N1CCc3ccccc3[C@@H]1c1ccc(F)cc1)O2] | 3177 |
| 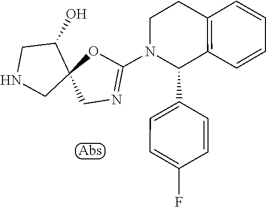 [SMILES: O[C@H]1CNC[C@@]12CN=C(N1CCc3ccccc3[C@@H]1c1ccc(F)cc1)O2] | 3178 |
| 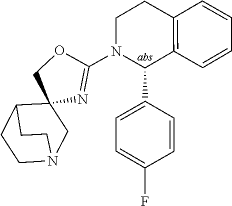 [SMILES: Fc1ccc([C@H]2c3ccccc3CCN2C2=N[C@]3(CO2)CN2CCC3CC2)cc1] | 3057A |
| 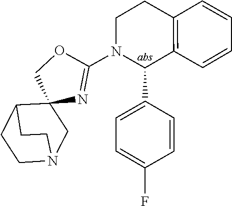 [SMILES: Fc1ccc([C@H]2c3ccccc3CCN2C2=N[C@@]3(CO2)CN2CCC3CC2)cc1] | 3057B |
| 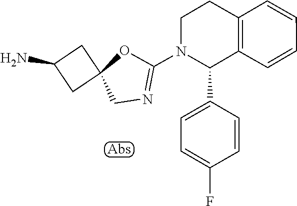 [SMILES: N[C@H]1C[C@]2(CN=C(N3CCc4ccccc4[C@@H]3c3ccc(F)cc3)O2)C1] | 3061B |

In some cases, a compound as disclosed herein is one as shown in any one of Tables A, B, or C, or a pharmaceutically acceptable salt thereof. In some cases, the compound, or salt thereof, is one as shown in Table A or B. In some cases, the compound, or salt thereof, is one as shown in Table A. In some cases, the compound, or salt thereof, is one as shown in Table B. In some cases, the compound, or salt thereof, is one as shown in Table C. In some cases, the compound, or salt thereof, is selected from the group consisting of Compound 3143, 3144, 3145, 3146, 3147, 3148, 3162, 3057A, and 3057B. In some cases, the compound, or salt thereof, is selected from the group consisting of Compound 3000, 3001, 3049, 3050, 3057A, 3057B, 3064, 3073, 3147, 3148, 3154, 3155, 3156, and 3157.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₆ alkyl refers to an alkyl group having a number of carbon atoms encompassing the entire range (e.g., 1 to 6 carbon atoms), as well as all subgroups (e.g., 1-6, 2-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), t-butyl (1,1-dimethylethyl), and 3-methylpentyl. Unless otherwise indicated, an alkyl group can be an unsubstituted alkyl group or a substituted alkyl group.

The term “alkylene” used herein refers to an alkyl group having a substituent. For example, an alkylene group can be —CH₂CH₂— or —CH₂—. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups. A C₀ alkylene indicates a direct bond. Unless otherwise indicated, an alkylene group can be an unsubstituted alkylene group or a substituted alkylene group. Particular substitutions on the alkylene group can be specified, e.g., alkylene-halo, alkylene-CN, alkylene-Ar, or the like.

As used herein, the term “haloalkyl” refers to an alkyl group substituted with one or more halogen substituents. Haloalkyl is alternatively referred to as “alkylene-halo.” For example, C₁-C₆haloalkyl refers to a C₁-C₆ alkyl group substituted with one or more halogen atoms, e.g., 1, 2, 3, 4, 5, or 6 halogen atoms. Non-limiting examples of haloalkyl groups include fluoromethyl, difluoromethyl, trifluoromethyl, chloromethyl, dichloromethyl, and trichloromethyl groups.

As used herein, the term “hydroxyalkyl” refers to an alkyl group substituted with one or more OH substituents. Hydroxyalkyl is alternatively referred to as “alkylene-OH.” For example, C₁-C₆hydroxyalkyl refers to a C₁-C₆ alkyl group substituted with one or more OH groups, e.g., 1, 2, 3, 4, 5, or 6 OH groups. Non-limiting examples of hydroxyalkyl groups include 2-hydroxyethyl and 3-hydroxypropyl groups.

As used herein, the term “alkoxy” or “alkoxyl” refers to a “—O-alkyl” group. The alkoxy or alkoxyl group can be unsubstituted or substituted.

As used herein, the term “haloalkoxy” or “haloalkoxyl” refers to a “—O-haloalkyl” group. The haloalkoxy or haloalkoxyl group can be unsubstituted or substituted.

As used herein, the term “halo” or “halogen” refers to fluorine, chlorine, bromine, or iodine.

As used herein, the term “oxo” refers to ═O substituent, e.g., a carbon can be substituted with an oxo to form a carbonyl (C═O) group.

As used herein, the term “carbocycle” or “carbocyclyl” refers to a cyclic hydrocarbon group containing three to eight carbon atoms (e.g., 3, 4, 5, 6, 7, or 8 carbon atoms). The term Cₙ means the carbocycle group has “n” carbon atoms. For example, C₅ carbocycle refers to a carbocycle group that has 5 carbon atoms in the ring. C₆-C₈ carbocycle refers to carbocycle groups having a number of carbon atoms encompassing the entire range (e.g., 6 to 8 carbon atoms), as well as all subgroups (e.g., 6-7, 6-8, 7-8, 6, 7, and 8 carbon atoms). Nonlimiting examples of carbocycle groups include cyclopropyl, cyclobutyl, cyclopentyl, cyclohexyl, cycloheptyl, and cyclooctyl. Unless otherwise indicated, a carbocycle group can be an unsubstituted carbocycle group or a substituted carbocycle group. The carbocycle groups described herein can be isolated or fused to another carbocycle group, a heterocycle group, an aryl group and/or a heteroaryl group. When a carbocycle group is fused to another carbocycle group, then each of the carbocycle groups can contain three to eight carbon atoms unless specified otherwise.

As used herein, the term “heterocycle” is defined similarly as carbocycle, except the ring contains one to four heteroatoms independently selected from oxygen, nitrogen, and sulfur. In particular, the term “heterocycle” refers to a ring containing a total of three to ten atoms (e.g., three to eight, or four to ten), of which 1, 2, 3 or 4 of those atoms are heteroatoms independently selected from the group consisting of oxygen, nitrogen, and sulfur, and the remaining atoms in the ring are carbon atoms. Nonlimiting examples of heterocycle groups include azetidine, piperidine, piperazine, pyrazolidine, tetrahydrofuran, tetrahydropyran, dihydrofuran, morpholine, quinuclidine, and the like. Heterocycle groups can be saturated or partially unsaturated ring systems optionally substituted with, for example, one to three groups, such as halo, C₁₋₆alkyl, C₁₋₆ haloalkyl, C₁₋₆alkoxy, C₁₋₆haloalkoxy, CN, and SO₂C₁₋₃alkyl. The heterocycle groups described herein can be isolated or fused to another heterocycle group and/or a carbocycle group. In particular, the heterocycles described herein can have a fused, bridged, or spiro structure. When a heterocycle group is fused to another heterocycle group, then each of the heterocycle groups can contain three to ten total ring atoms, and one to four heteroatoms.

As used herein, the term “aryl” refers to an aromatic group, such as phenyl. Aryl groups can be e.g., monocyclic or polycyclic. Unless otherwise indicated, an aryl group can be unsubstituted or substituted with one or more, and in particular one group selected from, for example, alkoxy and alkoxyalkyl. Aryl groups can be isolated (e.g., phenyl) or fused to another aryl group (e.g., naphthyl, anthracenyl), a carbocycle group (e.g. tetraydronaphthyl), a heterocycle group, and/or a heteroaryl group. Exemplary aryl groups include, but are not limited to, phenyl, fluorophenyl, difluorophenyl, trifluorophenyl, and the like.

As used herein, the term “heteroaryl” refers to a monocyclic aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to three, substituents selected from, for example, halo, C₁₋₆alkyl, C₁₋₆ haloalkyl, C₁₋₆alkoxy, C₁₋₆haloalkoxy, CN, and SO₂C₁₋₃ alkyl. Examples of heteroaryl groups include, but are not limited to, thienyl, furyl, pyridyl, pyrrolyl, oxazolyl, triazinyl, triazolyl, isothiazolyl, isoxazolyl, imidazolyl, pyrazinyl, pyrimidinyl, thiazolyl, and thiadiazolyl.

As used herein, the term “substituted,” when used to modify a chemical functional group, refers to the replacement of at least one hydrogen radical on the functional group with a substituent. Unless otherwise specified for a particular moiety, substituents can include, but are not limited to, alkyl, cycloalkyl, alkenyl, cycloalkenyl, alkynyl (e.g., propargyl), heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

Compounds of the present disclosure can exist in particular geometric or stereoisomeric forms having one or more asymmetric carbon atoms. The present disclosure contemplates such forms, including cis- and trans-isomers, R- and S-enantiomers, diastereomers, racemic mixtures thereof, and other mixtures thereof, as falling within the scope of the disclosed compounds. Additional asymmetric carbon atoms may be present in substituents such as alkyl groups. All such isomers, as well as mixtures thereof, are intended for inclusion herein.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

The compounds disclosed herein can be as a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N [symbol] dibenzylethylenediamine, 2-hydroxyethylamine, bis-(2-hydroxyethyl)amine, tri-(2-hydroxyethyl)amine, procaine, dibenzylpiperidine, dehydroabietylamine, N,N [symbol] bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Pharmaceutical Formulations, Dosing, and Routes of Administration

Further provided are pharmaceutical formulations comprising a compound as described herein or pharmaceutically acceptable salt thereof, and a pharmaceutically acceptable excipient.

The compounds described herein can be administered to a subject in a therapeutically effective amount, alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to affect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, for example, from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by physico-chemical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

The compositions used in the methods of the invention may be formulated in micelles or liposomes. Such formulations include sterically stabilized micelles or liposomes and sterically stabilized mixed micelles or liposomes. Such formulations can facilitate intracellular delivery, since lipid bilayers of liposomes and micelles are known to fuse with the plasma membrane of cells and deliver entrapped contents into the intracellular compartment.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, Pa., pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the invention are useful in fields of human medicine and veterinary medicine. Thus, the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds disclosed herein (e.g., compounds of Formula I, II, IIa, IIb, III, and as shown in Tables A and B) can increase the amount of progranulin or granulin in a subject. In some cases, the compounds increase the amount of progranulin in a subject. In some cases, the compounds increase the amount of granulin in a subject. In some cases, the compounds affect cells to increase secretion of progranulin. Solifenacin is a drug currently used for urinary incontinence. It has been found that this compound also causes the secretion of progranulin from mouse BV2 cells. As such, the compounds disclosed herein, (e.g., compounds of Formula I, II, IIa, IIb, III, and as shown in Tables A and B) can be useful in treating disorders associated with aberrant (e.g., reduced) progranulin secretion or activity.

Specifically contemplated are methods of using a therapeutically effective amount of a compound disclosed herein to modulate progranulin (e.g., to increase secretion of progranulin), for use as a therapeutic in a subject. As used herein, the term “therapeutically effective amount” means an amount of a compound or combination of therapeutically active compounds (e.g., a progranulin modulator or combination of modulators) that ameliorates, attenuates or eliminates one or more symptoms of a particular disease or condition (e.g., progranulin- or granulin-associated disorders), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

Contemplated disorders associated with aberrant progranulin activity include Alzheimer® disease (AD), Parkinson disease (PD) and PD-related disorders, Amytrophic lateral sclerosis (ALS), Frontotemperal lobe dementia (FTLD), Lewy body dementia (LBD), Prion disease, Motor neurone diseases (MND), Huntington disease (HD), Spinocerebellar ataxia (SCA), Spinal muscular atrophy (SMA) and other neurodegenerative diseases. Other disorders contemplated include lysosomal dys- or misfunction disorders, such lysosomal storage diseases (e.g., Paget's disease, Gaucher's disease, Nieman's Pick disease, Tay-Sachs Disease, Fabry Disease, Pompes disease, and Naso-Hakula disease). Other diseases contemplated include those associated with inclusions and/or misfunction of C9orf72, TDP-43, FUS, UBQLN2, VCP, CHMP28, and/or MAPT. Other diseases include acute neurological disorders such as stroke, cerebral hemorrhage, traumatic brain injury and other head traumas as well as diseases of the brain such as glioblastoma and neuroblastomas.

In some cases, the progranulin-associated disorder is Alzheimer® disease (AD), Parkinson disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington disease (HD), Spinocerebellar ataxia (SCA), Spinal muscular atrophy (SMA), a lysosomal storage disease, nephropathy, a disease associated with inclusions and/or misfunction of C9orf72, TDP-43, FUS, UBQLN2, VCP, CHMP28, and/or MAPT, an acute neurological disorder, glioblastoma, or neuroblastoma. In some cases, the lysosomal storage disease is Paget's disease, Gaucher's disease, Nieman's Pick disease, Tay-Sachs Disease, Fabry Disease, Pompes disease, or Naso-Hakula disease. In some cases, the acute neurological disorder is stroke, cerebral hemorrhage, traumatic brain injury or head trauma. In some cases, the progranulin-associated disorder is Frontotemporal dementia (FTD). In some cases, the progranulin-associated disorder is Frontotemporal dementia-Granulin subtype (FTD-GRN).

### Synthesis of Compounds Disclosed Herein

Compounds can be synthesized in using typical synthetic chemistry techniques using commercially available starting materials, compounds known in the literature, or from readily prepared intermediates, by employing standard synthetic methods and procedures either known to those of skill in the art, or in light of the teachings herein. Generally, the synthesis of the disclosed compounds can be achieved following similar syntheses as detailed in Schemes A and B below and in the Examples.

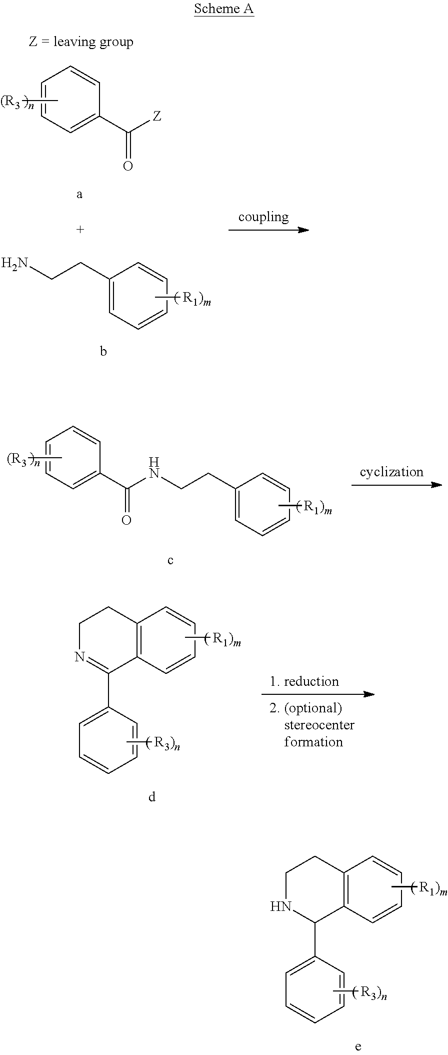

Compounds having structure e can be synthesized using the procedure shown in Scheme A. For example, reaction of an optionally substituted benzoic acid derivative having structure a with an optionally substituted 2-phenylethan-1-amine b produces optionally substituted N-phenethylbenzamide compounds having structure c. Cyclization under appropriate conditions gives optionally substituted 1-phenyl-3,4-dihydroisoquinoline compounds having structure d. Subsequent reduction followed by optional stereocenter formation gives substituted tetrahydroquinoline compounds having structure e.

The coupling of compounds a and b can be catalyzed by appropriate reagents selected based on the precise nature of compounds a and b. For example, when compound a is an acid chloride compound (i.e., when Z is Cl), the coupling of compounds a and b can be catalyzed by e.g., triethylamine. Compounds a and b can be purchased commercially or prepared by a variety of methods from commercially-available starting materials.

Cyclization of compound c can be effected with the use of various reactions known in the art. For example, the cyclization can involve an acid-catalyzed electrophilic aromatic substitution reaction, e.g., cyclization under Bischler-Napieralski reaction conditions. For example, c can be cyclized by treatment with triflic anhydride in the presence of e.g., chloropyridine in a solvent, e.g., dichloromethane. Alternately, compound c can be cyclized by treatment with polyphosphoric acid (PPA).

Compound d can be reduced to form compound e with or without formation of a stereocenter. For example, compound d can be treated with a reducing agent, e.g., sodium borohydride, in a solvent, e.g., methanol. Reduction of compound d can be followed by the formation of a desired stereoisomer, e.g., by crystallization in the presence of D-tartaric acid. Alternately, compound d can be reduced via asymmetric hydrogenation to directly produce substituted tetrahydroquinoline compound e as the desired stereoisomer. For example, compound d can be reduced with H₂ gas in the presence of an iridium catalyst, such as [{Ir(H)[(S,S)-(f)-binaphane]}₂(μ-l)₃]⁺I⁻.

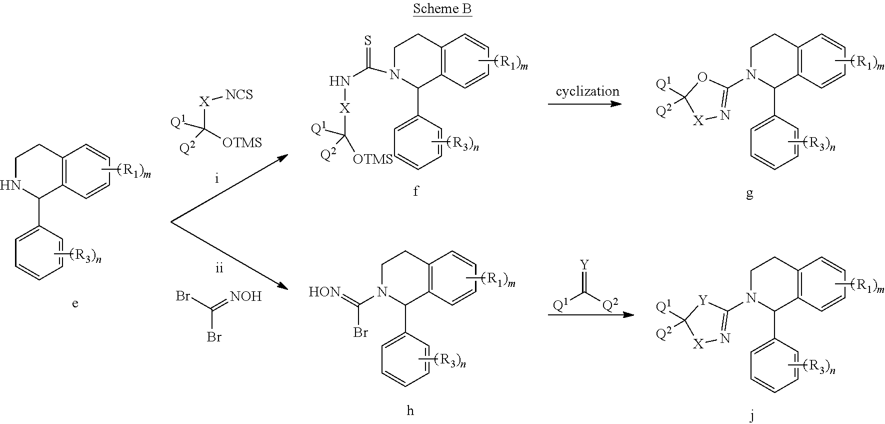

Compounds described herein, e.g., compounds of Formula I, can be synthesized from compounds of structure e using the procedures shown in Scheme B. For example, reaction of a compound having structure e with an O-protected hydroxyl-bearing isothiocyanate compound having structure i produces a substituted tetrahydroquinolinyl thiourea compound having structure f. Deprotection of the hydroxyl group followed by cyclization produced compounds described herein, e.g., compounds of Formula I having structure g. Deprotection can be carried out under appropriate conditions known to the skilled artisan. For example, when the O-protecting group is a trimethyl silyl (TMS) group, the hydroxyl group can be deprotected by treatment with a fluoride compound such as tetrabutylammonium fluoride (TBAF). The deprotected compound can then be cyclized using appropriate reaction conditions, e.g., by treatment with phosphorus oxychloride (POCl₃) to produce a compound having structure g.

Alternately, reaction of a compound having structure e with an oxime compound having structure ii produces a substituted tetrahydroquinolinyl oxime compound having structure h. Intermolecular cyclization of h via e.g., 1,3 dipolar cycloaddition, produces compounds described herein, e.g., compounds of Formula I having structure j. The cycloaddition can be carried out in the presence of appropriate catalysts known to the skilled artisan, e.g., bases such as potassium bicarbonate.

### Examples

General Methods

All ¹HNMR experiments were run in Bruker Avance III 400, at 25° C.

### Analytical Methods:

All CP Analytical-SFC experiments were run on SFC Method Station (Thar, Waters), Column temperature: 40° C., Mobile phase: CO₂/Methanol (0.2% Methanol Ammonia)=Flow: 4.0 ml/min, Back Pressure: 120 Bar, Detection wavelength: 214 nm;

### Preparative Methods:

All CP Preparative-SFC experiments were run on SFC-80 (Thar, Waters), Column temperature: 35° C., Mobile phase (example): CO₂/Methanol (0.2% Methanol Ammonia)=Flow rate: 80 g/min, Back pressure: 100 bar, Detection wavelength: 214 nm.

Preparative CP Method B: Acidic reversed phase MPLC: Instrument type: Reveleris™ prep MPLC; Column: Phenomenex LUNA C18(3) (150×25 mm, 10p); Flow: 40 mL/min; Column temp: room temperature; Eluent A: 0.1% (v/v) Formic acid in water, Eluent B: 0.1% (v/v) Formic acid in acetonitrile; using the indicated gradient and wavelength.

### LCMS Experiments:

All CP LCMS experiments were run on an Agilent 1200 system, with a column temperature of 40° C., monitoring UV absorption at 214 nm and scanning a mass range from 100-1000. Individual conditions varied slightly as described in the methods below:

LCMS CP Method A: Column: ZORBAX SB-C18 3.0×50 mm, 3.5 μm; Mobile Phase: A: Water (0.1% v/v TFA), B: ACN (0.1% v/v TFA); Gradient: 5% B increasing to 95% B over 1.3 min, stopping at 3 min. Flow Rate: 1.8 mL/min

LCMS CP Method A1: Column: XBridge SB-C18 3.0×50 mm, 3.5 μm; Mobile Phase: A: Water (0.01% v/v TFA), B: ACN (0.01% v/v TFA); Gradient: 5% B increasing to 95% B over 1.3 min, stopping at 3 min. Flow Rate: 2.0 mL/min

LCMS CP Method B: Column: XBridge C18 50×4.6 mm, 3.5 μm; Mobile Phase: A: Water (0.1% v/v TFA), B: ACN (0.1% v/v TFA); Gradient: 5% B increasing to 95% B over 1.2 min, stopping at 3 min. Flow Rate: 2.0 mL/min

LCMS CP Method C: Column: XBridge SB-C18 3.0×50 mm, 3.5 μm; Mobile Phase: A: Water (10 mM NH₄HCO₃), B: ACN; Gradient: 5% B increasing to 95% B over 1.2 min. Flow Rate: 2.0 mL/min;

LCMS CP Method C1: Column: XBridge SB-C18 3.0×50 mm, 3.5 μm; Mobile Phase: A: Water (10 mM NH₄HCO₃), B: ACN; Gradient: 5% B increasing to 95% B over 1.4 min. Flow Rate: 2.0 mL/min;

LCMS CP Method D: Column: XBridge SB-C18 3.0×50 mm, 3.5 μm; Mobile Phase: A: Water (0.1% v/v TFA), B: ACN (0.1% v/v TFA); Gradient: 5% B increasing to 95% B over 3.1 min. Flow Rate: 1.8 mL/min;

LCMS CP Method E: Column: XBridge SB-C18 3.0×50 mm, 3.5 μm; Mobile Phase: A: Water (0.1% v/v TFA), B: ACN (0.1% v/v TFA); Gradient: 5% B increasing to 95% B over 1.8 min, stopping at 3 min. Flow Rate: 1.8 mL/min;

LCMS MC1: MC LCMS experiments were run as follows: Apparatus: Agilent 1260 Bin. Pump: G1312B, degasser; autosampler, ColCom, DAD: Agilent G1315D, 220-320 nm, MSD: Agilent LC/MSD G6130B ESI, pos/neg 100-1000, ELSD Alltech 3300 gas flow 1.5 ml/min, gas temp: 40° C., Eluent A: 0.1% formic acid in acetonitrile, Eluent B: 0.1% formic acid in water).

MC Method A: column: Waters XSelect™ C18, 30×2.1 mm, 3.5μ, Temp: 35° C., Flow: 1 mL/min, Gradient: t₀=5% A, t_{1.6 min}=98% A, t_{3 min}=98% A, Posttime: 1.3 min.

MC Method C: column: Waters XSelect™ C18, 50×2.1 mm, 3.5μ, Temp: 35° C., Flow: 0.8 mL/min, Gradient: t₀=5% A, t_{3.5 min}=98% A, t_{6 min}=98% A, Posttime: 2 min.

LCMS MC2: MC LCMS experiments were run as follows: Apparatus: Agilent 1260 Bin. Pump: G1312B, degasser; autosampler, ColCom, DAD: Agilent G1315C, 220-320 nm, MSD: Agilent LC/MSD G6130B ESI, pos/neg 100-1000, Eluent A: acetonitrile, Eluent B: 10 mM ammoniumbicarbonate in water (pH=9.5).

Individual conditions varied slightly as described in the methods below:

MC Method B: column: Waters XSelect™ CSH C18, 30×2.1 mm, 3.5μ, Temp: 25° C., Flow: 1 mL/min, Gradient: t₀=5% A, t_{1.6 min}=98% A, t_{3 min}=98% A, Posttime: 1.3 min.

LCMS MC3: Apparatus: Agilent 1290 series with UV detector (220 nm, 270 nm (band width 100 nm)), and HP 6130 MSD mass detector (API-ES positive and negative).

MC Method E: column: Waters XBridge BEH XP (2.1×50 mm; 2.5 μm; 1034 bar), Temp: 35° C., Flow: 0.6 mL/min, t₀=80% A, t_{1.5 min}=0% A, t_{3 min}=0% A. Eluent A: 100% water, Eluent B: 100% methanol/acetonitrile 1:1.

MC Method K: column: Waters XBridge BEH XP (2.1×50 mm; 2.5 μm; 1034 bar), Temp: 35° C., Flow: 0.6 mL/min, t₀=80% A, t_{1.5 min}=0% A, t_{4 min}=0% A. Eluent A: ammonium acetate (10 mM); water/methanol/acetonitrile (90:6:4), Eluent B: ammonium acetate (10 mM); water/methanol/acetonitrile (10:54:36).

MC Chiral LC: Apparatus: Agilent 1260 Quat. Pump: G1311C, degasser; autosampler, ColCom, DAD: Agilent G1315D (210 nm, 220 nm, 220-320 nm).

MC Method L: column: Chiralcel OD-H (250×4.6 mm, 5 μm); Column temp: 25° C.; flow: 1.0 mL/min; isocratic gradient of 0.1% diethylamine in heptane/isopropanol 90/10.

MC Chiral SFC: Apparatus: Waters Acquity UPC²: Waters ACQ-ccBSM Binary Pump; Waters ACQ-CCM Convergence Manager; Waters ACQ-SM Sample Manager—Fixed Loop; Waters ACQ-CM Column Manager—30S; Waters ACQ-PDA Photodiode Array Detector (210-400 nm); Waters ACQ-ISM Make Up Pump, Waters Acquity QDa MS Detector (pos 100-650).

MC Method N: column: Daicel a Chiral® IG-3 (3.0×150 mm; 3 μm), Temp: 40° C., BPR: 126 bar, Flow: 2.0 mL/min, Pump program: 30% B isocratic, Eluent A: CO₂, Eluent B: 0.2% ammonia in methanol.

MC Method T: Column: Phenomenex Lux Amylose-1 (250×21 mm, 5 μm); Column temp: 35° C.; flow: 70 mL/min; ABPR: 120 bar; Linear gradient: t=0 min 5% B, t=3 min 10% B; t=8.5 min 10% B; Detection: PDA (210-400 nm)/TIC.

MC Method U: Column: Phenomenex Lux Cellulose-1 (250×21.2 mm, 5 μm); Column temp: 35° C.; flow: 70 mL/min; ABPR: 120 bar; Linear gradient: t=0 min 10% B, t=6.5 min 30% B, t=8 min 30% B; Detection: PDA (210-400 nm)/TIC.

MC Method V: column: Phenomenex Cellulose-1 (100×4.6 mm 5 μm), Temp: 35° C., BPR: 170 bar, Flow: 2.5 mL/min, Gradient: t₀=5% B, t_{5 min}=50% B, t_{6 min}=50% B, Posttime: 0.5 min; Eluent A: CO₂, Eluent B: 20 mM ammonia in methanol

### Synthetic Methods:

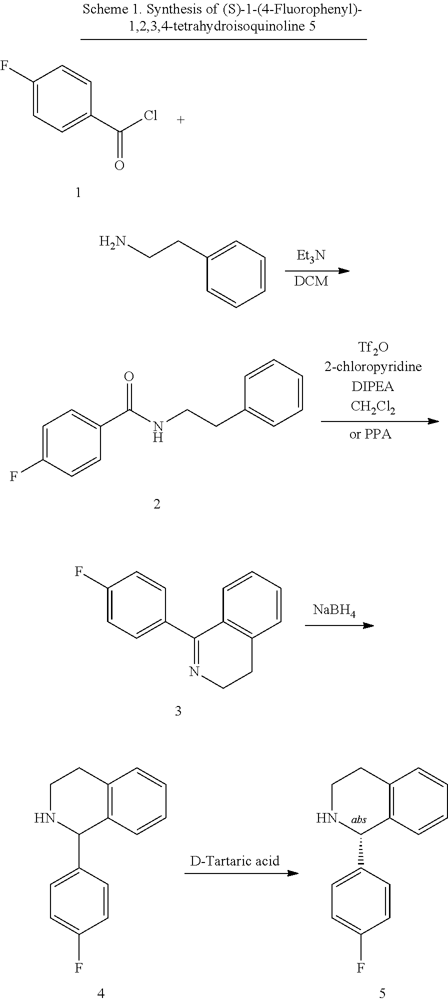

Step 1: At 0° C., a solution of 4-fluorobenzoyl chloride (312 g, 1.96 mol) in tetrahydrofuran (1.0 L) was added dropwise to a solution of 2-phenylethan-1-amine (250 g, 2.06 mol) and N,N-diisopropylethylamine (513 mL, 2.95 mol) in tetrahydrofuran (4.0 L). The resulting white suspension was stirred at room temperature overnight. The mixture was concentrated under reduced pressure and the residue was partitioned between ethyl acetate (4.0 L) and saturated aqueous NH₄Cl (2.0 L). Additional water (2.0 L) was added. The layers were separated and the aqueous phase was extracted with ethyl acetate (2.0 L). The combined organic layers were washed with brine (2 L), dried with Na₂SO₄, and concentrated under reduced pressure to obtain 4-fluoro-N-phenethylbenzamide 2. ¹H NMR (300 MHz, chloroform-d) δ 7.75-7.64 (m, 2H), 7.39-7.28 (m, 2H), 7.25 (tt, J=7.4, 1.4 Hz, 3H), 7.08 (t, J=8.5 Hz, 2H), 6.09 (s, 1H), 3.78-3.65 (m, 2H), 2.93 (t, J=6.9 Hz, 2H).

Step 2: At −78° C., triflic anhydride (274.5 mL, 1.6317 mol) was added dropwise to a suspension of 4-fluoro-N-phenethylbenzamide 2 (330.80 g, 1.3598 mol) and 2-chloropyridine (167 mL, 1.7677 mol)) in dichloromethane (4.5 L). After the addition was complete, the reaction was stirred for 1 hour, then allowed to warm to room temperature and stirred overnight. The reaction mixture was quenched with aqueous NaOH (1 M, 4.5 L). The layers were separated and the aqueous layer was extracted with dichloromethane (4.5 L). The combined organic layers were washed with brine (4.5 L), dried with Na₂SO₄, and concentrated under reduced pressure to obtain 1-(4-fluorophenyl)-3,4-dihydroisoquinoline 3. ¹H NMR (300 MHz, chloroform-d) δ, 7.71-7.53 (m, 2H), 7.46-7.32 (m, 1H) 7.27-7.16 (m, 3H), 7.16-7.01 (m, 2H), 3.87-3.77 (m, 2H), 2.84-2.73 (m, 2H).

Alternate step 2: A round bottomed flask was charged with 500 mL of PPA. The material was heated to 160° C., then 4-fluoro-N-phenethylbenzamide 2 (350 g, 1.44 mol) was added. The mixture was stirred at 160° C. for 3 hours. The mixture was cooled to 25° C. and 3 L of water was added. The mixture was alkalized with NaOH (20% aq.) to pH 11 and extracted with three 1 L portions of ethyl acetate. The combine organic layers were washed three times with brine, dried and concentrated in vacuo to give crude product. The crude product was purified by column chromatography eluting with petroleum ether/ethyl acetate (3:1) to give 273 g of 1-(4-fluorophenyl)-3,4-dihydroisoquinoline 3.

Step 3: At 0° C., sodium borohydride (77.16 g, 2.0396 mol) was added in portions to a solution of 1-(4-fluorophenyl)-3,4-dihydroisoquinoline 3 (306.31 g, 1.36 mol) in methanol (4.0 L), causing the reaction mixture to warm to 42° C. The reaction mixture was stirred at room temperature for 2 hours. Then, aqueous hydrochloric acid (1 M, 4.0 L) was added and the mixture was stirred for 30 minutes at room temperature. The mixture was made basic by addition of aqueous NaOH (2 M) to pH 8-9. The mixture was stirred at room temperature for 30 minutes. The solids were filtered off and dried under reduced pressure to obtain 1-(4-fluorophenyl)-1,2,3,4-tetrahydroisoquinoline 4. ¹H NMR (300 MHz, chloroform-d) δ 7.29-7.20 (m, 2H), 7.20-6.93 (m, 5H), 6.72 (dd, J=7.7, 1.1 Hz, 1H), 5.09 (s, 1H), 3.33-3.18 (m, 1H), 3.17-2.96 (m, 2H), 2.90-2.64 (m, 1H).

Note: Additional related racemic 1-aryl tetrahydroisoquinolines were prepared analogously.

Step 4: 1-(4-Fluorophenyl)-1,2,3,4-tetrahydroisoquinoline (287.98 g, 1.2671 mol) was dissolved in methanol (1.3 L) at 40° C. D-Tartaric acid (190.17 g, 1.2671 mol) was added and the mixture was stirred at reflux for 2 hours. The mixture was allowed to cool to room temperature overnight. Ethyl acetate (650 mL) was added and the mixture was stirred at room temperature for 30 minutes. The solids were filtered off, washed with methanol (325 mL), and dried under reduced pressure. The solids were suspended in water (3.0 L) and stirred for 10 minutes at room temperature. The suspension was basified with aqueous NaOH (2 M) to pH 8-9 and stirred for 1.5 hours. The solids were filtered off and washed with water (400 ml). The solid was partitioned between dichloromethane (3.0 L) and water (2.0 L). The layers were separated, and the organic layer was dried with Na₂SO₄ and concentrated under reduced pressure to obtain (S)-1-(4-fluorophenyl)-1,2,3,4-tetrahydroisoquinoline 5. LCMS MC3: (M+H)⁺=227; purity=99.45%, retention time=1.25 min (MC Method E). MC Chiral LC:, retention time=6.72 min., 100% ee (MC Method L). ¹H NMR (300 MHz, chloroform-d) δ 7.29-7.20 (m, 2H), 7.19-7.10 (m, 2H), 7.09-6.93 (m, 3H), 6.72 (d, J=7.7 Hz, 1H), 5.09 (s, 1H), 3.33-3.19 (m, 1H), 3.18-2.96 (m, 2H), 2.91-2.75 (m, 1H).

Alternate Step 4: To a solution of racemic 1-(4-fluorophenyl)-1,2,3,4-tetrahydroisoquinoline 4 (232 g, 1022 mmol) in isopropanol (2 L) was added dropwise a solution of D-Tartaric acid (200 g, 1329 mmol) in isopropanol (1 L) at room temperature. The mixture was stirred at room temperature overnight. The precipitate was filtered and the cake was washed with isopropanol (200 mL) to give a solid. The solid was added into isopropanol (2 L) and heated to 100° C. Water was added dropwise (0.6 L) at 100° C. until the solid was dissolved. The mixture was allowed to crystallize at room temperature overnight. The precipitate was isolated by filtration and the cake was washed with isopropanol (200 mL) to give a solid (190 g). A second recrystallization from isopropanol and water (˜3/1, 100° C. to room temperature overnight) afforded a solid. The solid was dissolved in water (500 mL), alkalized with NaOH (20% aq.) to pH 11 and extracted with three 200 mL portions of ethyl acetate. The combine organic layers were washed with brine (0.5 L), dried and concentrated in vacuo to give 70 g of (S)-1-(4-fluorophenyl)-1,2,3,4-tetrahydroisoquinoline 5.

Chiral SFC: CO₂/MeOH containing 0.2% ammonia over a Chiral® IG column (4.6×100 mm, 5 μm), retention time=6.08 min), 100% ee.

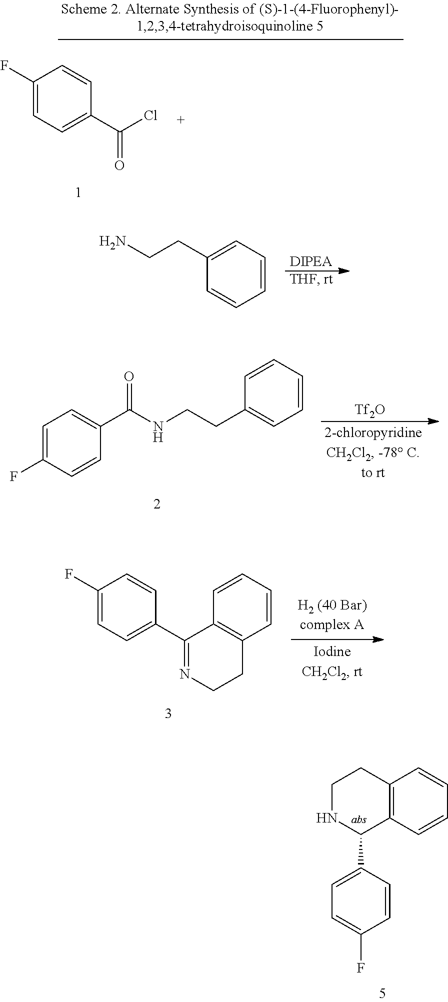

Step 1: To a stirred solution of phenethylamine (4.17 mL, 33.1 mmol) and N,N-diisopropylethylamine (8.24 mL, 47.3 mmol) in anhydrous tetrahydrofuran (80 mL) was added dropwise a solution of 4-fluorobenzoyl chloride (3.73 mL, 31.5 mmol) in anhydrous tetrahydrofuran (20 mL) at room temperature under nitrogen atmosphere. The reaction was kept at room temperature in a water bath. After addition was complete, the reaction was stirred for 1 hour and then concentrated under reduced pressure. The residue was partitioned between ethyl acetate (200 mL) and half saturated aqueous NH₄Cl (100 mL). The aqueous layer was extracted with ethyl acetate (50 mL). The combined organics were washed with brine (50 mL), dried on Na₂SO₄, and concentrated under reduced pressure to give 4-fluoro-N-phenethylbenzamide 2. LCMS MC2: (M+H)⁺=244; purity=100%, retention time=1.990 min. (MC method B). ¹H NMR (400 MHz, chloroform-d) δ 7.74-7.65 (m, 2H), 7.38-7.29 (m, 2H), 7.29-7.19 (m, 3H), 7.12-7.02 (m, 2H), 6.15 (br s, 1H), 3.70 (q, J=6.7 Hz, 2H), 2.93 (t, J=6.9 Hz, 2H).

Step 2: At −78° C. under nitrogen atmosphere, triflic anhydride (5.69 mL, 34.3 mmol) was added dropwise to a suspension of 4-fluoro-N-phenethylbenzamide (6.95 g, 28.6 mmol) and 2-chloropyridine (3.49 mL, 37.1 mmol) in dichloromethane (100 mL). After the addition was complete, the reaction was stirred for 1 hour, then allowed to warm to room temperature and stirred for an additional 64 hours. The reaction mixture was washed with aqueous NaOH (1 M, 75 mL). The aqueous layer was extracted with dichloromethane (25 mL). The combined organics were washed with brine (50 mL), dried on Na₂SO₄, and concentrated under reduced pressure. The residue was purified by flash silica gel column chromatography (0 to 30% ethyl acetate in heptane) to yield 1-(4-fluorophenyl)-3,4-dihydroisoquinoline 3. LCMS MC2: (M+H)⁺=226; purity=99%, retention time=2.051 min., (MC method B). ¹H NMR (400 MHz, chloroform-d) δ 7.64-7.55 (m, 2H), 7.40 (ddd, J=8.3, 6.2, 2.4 Hz, 1H), 7.31-7.21 (m, 3H), 7.16-7.06 (m, 2H), 3.88-3.78 (m, 2H), 2.85-2.75 (m, 2H).

Step 3: To a solution of 1-(4-fluorophenyl)-3,4-dihydroisoquinoline 3 (5.975 g, 26.5 mmol) in dichloromethane (100 mL), iodine (0.168 g, 0.663 mmol) and [{Ir(H)[(S,S)-(f)-Binaphane]}₂(μ-l)₃]⁺I⁻ (Complex A (0.060 g, 0.024 mmol) were added. The resulting suspension was stirred in an autoclave charged with 40 bars of hydrogen gas at room temperature for 16 hours. The reaction mixture was concentrated under reduced pressure. The residue was dissolved in warm 2-propanol (25 mL) and diluted with heptane (12.5 mL). Residual undissolved material was filtered off. The filtrate was partly concentrated under reduced pressure until about 10 mL of solvents were removed. Then, the solution was allowed to crystallize. The formed crystals were filtered off, washed with heptane (2×4 mL), and dried under reduced pressure to yield a first crop of (S)-1-(4-fluorophenyl)-1,2,3,4-tetrahydroisoquinoline 5 (ee=100%). The mother liquid was concentrated under reduced pressure and crystallized again from 2-propanol (8.0 mL) to yield a second crop of (S)-1-(4-fluorophenyl)-1,2,3,4-tetrahydroisoquinoline 5 (ee=100%). LCMS MC2: (M+H)⁺=228; purity=99%, retention time=2.027 min., (MC method B). MC Chiral LC: retention time=6.120 min., 100% ee (MC method L). ¹H NMR (400 MHz, chloroform-d) δ 7.29-7.20 (m, 2H), 7.19-7.12 (m, 2H), 7.09-6.96 (m, 3H), 6.72 (d, J=7.7 Hz, 1H), 5.12 (s, 1H), 3.31-3.20 (m, 1H), 3.15-3.01 (m, 2H), 2.90-2.78 (m, 1H), 2.41 (br s, 1H).

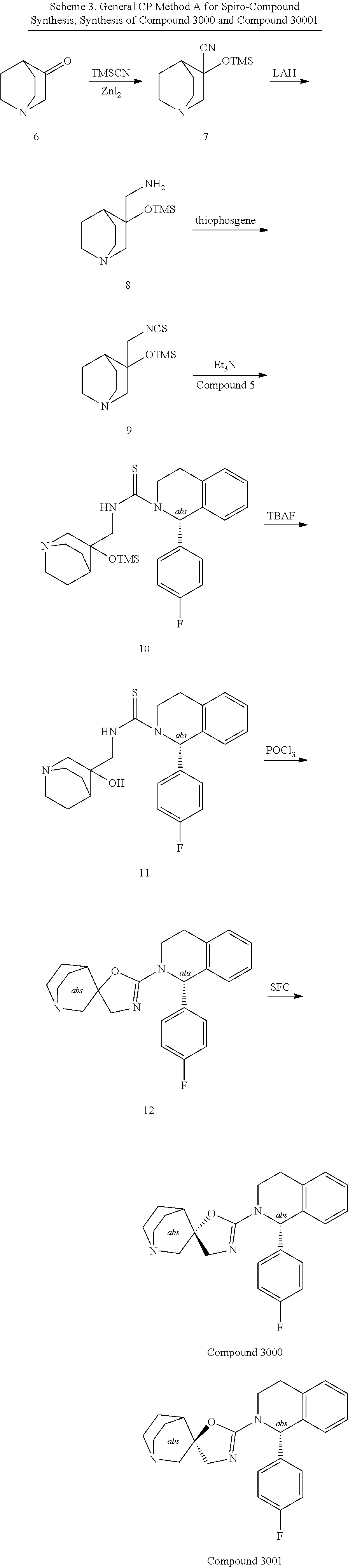

Step 1: A solution of quinuclidin-3-one 6 (2.5 g, 20 mmol), TMSCN (1.98 g, 20 mmol) and ZnI₂ (319 mg 1 mmol) in tetrahydrofuran (100 mL) was stirred at 60° C. When monitoring the reaction for disappearance of the quinuclidin-3-one starting material by LC-MS indicated completion, the mixture was cooled to ambient temperature and filtered to remove the solids. The filtrate was concentrated in vacuo to give crude 3-(trimethylsilyloxy)quinuclidine-3-carbonitrile, 7 (4.48 g).

LCMS: (M+H)⁺=225 (UV 214 nm); Retention time=1.208 min. CP Method C

Step 2: To a solution of 3-(trimethylsilyloxy)quinuclidine-3-carbonitrile 7 (4.48 g, 20 mmol), in THF (100 mL) cooled to 0° C. was added lithium aluminum hydride (20 mL, 20 mmol, 1M in THF). The reaction was stirred at room temperature until the starting material was consumed (TLC). The reaction mixture was quenched with saturated Na₂SO₄, filtered, and concentrated to give crude 3-(trimethylsilyloxy)quinuclidin-3-yl)methanamine 8.

LCMS: (M+H)⁺=229 (UV 214 nm); Retention time=0.903 min. CP Method C

Step 3: To a solution of (3-(trimethylsilyloxy)quinuclidin-3-yl)methanamine 8 (4.56 g, 20 mmol), and TEA (3.03 g 30 mmol) in CH₃CN or tetrahydrofuran (100 mL) was cooled to 0° C. Thiophosgene (2.30 g, 20 mmol) was added and the reaction was stirred at room temperature until the starting material was consumed (TLC). Concentration of the reaction mixture yielded crude of 2-(3-((trimethylsilyl)oxy)quinuclidin-3-yl)ethene-1-thione 9 which was used without purification.

Step 4: To a solution of 2-(3-((trimethylsilyl)oxy)quinuclidin-3-yl)ethene-1-thione 9 (5.4 g, 20 mmol) and TEA (3.03 g 30 mmol) in tetrahydrofuran (100 mL) cooled to 0° C. was added (S)-1-(4-fluorophenyl)-1,2,3,4-tetrahydroisoquinoline 5 (4.54 g, 20 mmol). The reaction mixture was stirred at 60° C. until 5 was consumed (as monitored by TLC). The solvent was removed in vacuo and the residue purified by prep-HPLC to afford (1S)-1-(4-fluorophenyl)-N-((3-(trimethylsilyloxy)quinuclidin-3-yl)methyl)-3,4-dihydroisoquinoline-2(1H)-carbothioamide 10.

LCMS: (M+H)⁺=498 (UV 214 nm); Retention time=1.592 min. CP Method C

Step 5: A solution of (1S)-1-(4-fluorophenyl)-N-((3-(trimethylsilyloxy)quinuclidin-3-yl)methyl)-3,4-dihydroisoquinoline-2(1H)-carbothioamide 10 (994 mg, 2.0 mmol) in THF (50 mL) was cooled to 0° C. and then TBAF (3 mL, 3 mmol, 1M in THF) was added. The reaction was stirred at room temperature until the starting material was consumed (TLC) and then concentrated in vacuo. Purification of the residue by prep-HPLC yielded (1S)-1-(4-fluorophenyl)-N-((3-hydroxyquinuclidin-3-yl)methyl)-3,4-dihydroisoquinoline-2(1H)-carbothioamide 11.

LCMS: (M+H)⁺=426 (UV 214 nm); Retention time=1.608 min. CP Method C

Step 6: A solution of (1S)-1-(4-fluorophenyl)-N-((3-hydroxyquinuclidin-3-yl)methyl)-3,4-dihydroisoquinoline-2(1H)-carbothioamide 11 (850 mg, 20 mmol) in THF (50 mL) was cooled to 0° C., then phosphorus oxychloride (1.53 g, 10 mmol) was introduced. The reaction mixture was stirred at 60° C. until the starting material was consumed (TLC) and then concentrated in vacuo. Purification of the residue by prep-HPLC yielded 2 [symbol] ((S)-1-(4-fluorophenyl)-3,4-dihydroisoquinolin-2(1H)-yl)-4 [symbol] -4-azaspiro[bicyclo[2.2.2]octane-2,5 [symbol] oxazole] 12.

LCMS: (M+H)⁺=392 (UV 214 nm); Retention time=0.4 min. CP Method C

The diastereomers were separated by chiral SFC eluting with CO₂/MeOH containing 0.2% methanolic ammonia over a chiral Column (4.6×100 mm, 5 μm) to give compound 3000 (15 mg, retention time=4.225 min) and compound 3001 (20 mg, retention time=4.563 min). Stereochemical assignment of (S) at the 1 position of the tetrahydroisoquinoline is based on enantiomerically pure starting materials; the stereochemical assignment of (S) at quinuclidine spirocyclic center of the quinuclidine is assigned based on chromatographic elution order as compared to related analogues of known configuration and therefore is assumed.

Compound 3000: LCMS: (M+H)⁺=392; (214 nm); retention time=1.276 min. CP Method D

¹H NMR (400 MHz, DMSO-d₆) δ 7.29-7.10 (m, 7H), 7.07 (d, J=7.5 Hz, 1H), 6.10 (s, 1H), 3.77 (t, J=13.9 Hz, 2H), 3.40 (d, J=12.6 Hz, 1H), 3.32-3.26 (m, 1H), 2.92 (s, 1H), 2.76 (dt, J=13.4, 7.9 Hz, 5H), 2.64-2.53 (m, 2H), 1.83 (d, J=2.2 Hz, 2H), 1.50 (t, J=7.6 Hz, 2H), 1.46-1.34 (m, 1H).

Chiral SFC: CO₂/MeOH containing 0.2% ammonia over a chiral IG column (4.6×100 mm, 5 μm), retention time=5.237 min), 100% ee.

Compound 3001: LCMS: (M+H)⁺=392; purity=100% (214 nm); retention time=1.278 min. CP Method D

¹H NMR (400 MHz, DMSO-d₆) δ 7.23-7.10 (m, 7H), 6.13 (s, 1H), 3.75 (t, J=14.2 Hz, 2H), 3.40 (d, J=12.7 Hz, 1H), 3.00-2.68 (m, 6H), 2.66-2.53 (m, 2H), 1.76 (s, 2H), 1.50 (t, J=6.1 Hz, 2H), 1.35 (s, 1H), 1.23 (s, 1H).

Chiral SFC: CO₂/MeOH containing 0.2% ammonia over a chiral IG column (4.6×100 mm, 5 μm), retention time=6.510 min), 100% ee.

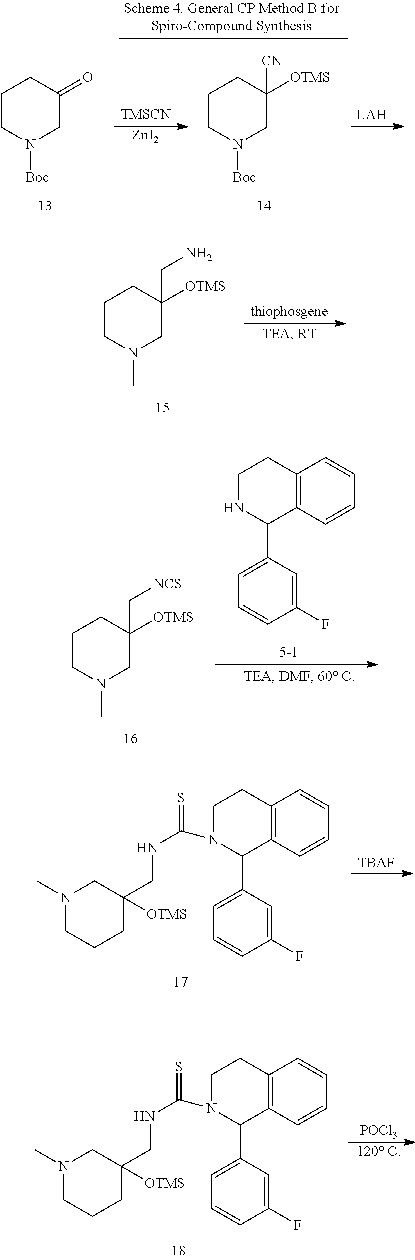

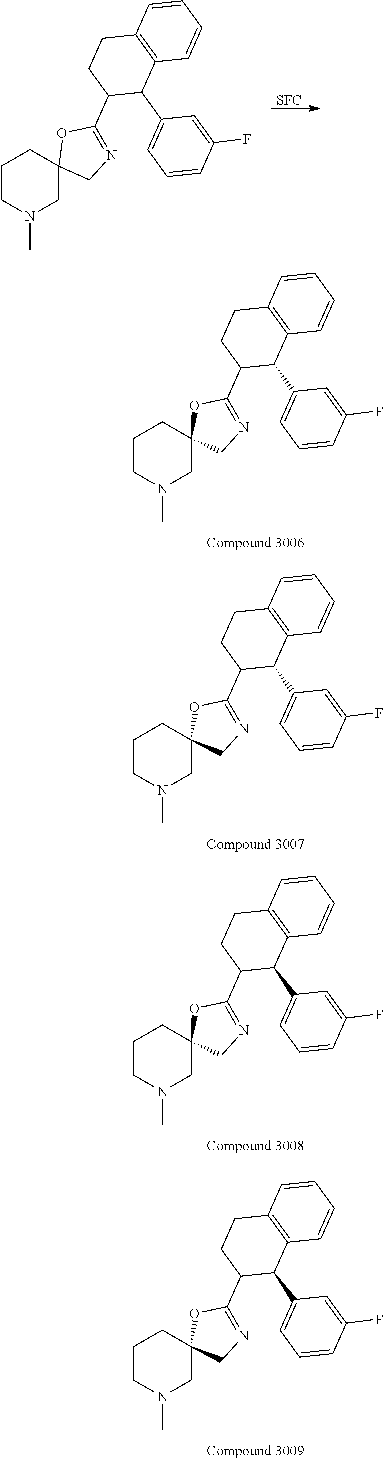 [SMILES: CN1CCCC2(CN=C(C3CCc4ccccc4C3c3cccc(F)c3)O2)C1 ; "SFC" ; CN1CCC[C@]2(CN=C(C3CCc4ccccc4[C@@H]3c3cccc(F)c3)O2)C1 ; CN1CCC[C@@]2(CN=C(C3CCc4ccccc4[C@@H]3c3cccc(F)c3)O2)C1 ; CN1CCC[C@]2(CN=C(C3CCc4ccccc4[C@H]3c3cccc(F)c3)O2)C1 ; CN1CCC[C@@]2(CN=C(C3CCc4ccccc4[C@H]3c3cccc(F)c3)O2)C1]

Step 1: To a solution of tert-butyl 3-oxopiperidine-1-carboxylate 13 (4 g, 20 mmol) and TMSCN (5 mL, 40 mmol) in dry THF (40 mL) was added ZnI₂ (0.32 g, 1 mmol) at room temperature. The reaction was heated at reflux for 16 h and then cooled and filtered to remove the solids. The filtrate was concentrated to give crude tert-butyl 3-cyano-3-(trimethylsilyloxy)piperidine-1-carboxylate 14 which was used without further purification.

LCMS: (M+H—Si(CH₃)₃—Bu-t)⁺ 171; Retention time 1.65 min. CP Method A1

Step 2: To a cooled (0° C.) solution of tert-butyl 3-cyano-3-(trimethylsilyloxy)piperidine-1-carboxylate 14 (5.1 g, 17 mmol) in dry THF (100 mL) was added LiAlH₄ (17 mL, 17 mmol, 1M in THF) dropwise. The reaction mixture was heated to reflux for 16 h and then cooled to 0° C. and quenched by saturated aqueous sodium sulfate solution. The mixture was diluted with THF (100 mL) and anhydrous Na₂SO₄ was added. The mixture was stirred for 20 min at room temperature, filtered and the filtrate concentrated to give crude (1-methyl-3-(trimethylsilyloxy)piperidin-3-yl)methanamine 15.

LCMS: (M+1)+217; Retention time 1.31 min. CP Method C

Step 3: To a solution of crude (1-methyl-3-(trimethylsilyloxy)piperidin-3-yl)methanamine 15 (3 g, 13.9 mmol) in dry CH₃CN (50 mL) was added thiophosgene (1.1 mL, 13.9 mmol) at 0° C. After stirring the reaction mixture at room temperature for 2 hours, the solvent was evaporated to give yield 2-(3-((trimethylsilyl)oxy)quinuclidin-3-yl)ethene-1-thione 16.

Step 4: A solution of 2-(3-((trimethylsilyl)oxy)quinuclidin-3-yl)ethene-1-thione 16 (3.55 g, 13.9 mmol), and TEA (2.11 g 20.85 mmol) in DMF (80 mL) was cooled to 0° C. and racemic 1-(3-fluorophenyl)-1,2,3,4-tetrahydroisoquinoline 5-1 (3.16 g, 13.9 mmol) was added. The reaction mixture was stirred at 60° C. until the starting material was consumed (TLC) and then concentrated in vacuo. Purification of the residue by prep-HPLC afforded (1S)-1-(3-fluorophenyl)-N-((3-hydroxy-1-methylpiperidin-3-yl)methyl)-3,4-dihydroisoquinoline-2(1H)-carbothioamide 17.

LCMS: (M+H)⁺=414 (UV 214 nm); Retention time=1.483 min. CP Method C

Step 5: A solution of (1S)-1-(3-fluorophenyl)-N-((3-hydroxy-1-methylpiperidin-3-yl)methyl)-3,4-dihydroisoquinoline-2(1H)-carbothioamide 17 (970 mg, 2.0 mmol) in THF (50 mL) was cooled to 0° C. and then tetrabutylammonium fluoride (3 mL, 3 mmol, 1M in THF) was added. The reaction was stirred at room temperature until the starting material was consumed (TLC) and then concentrated in vacuo. The residue was purified by prep-HPLC to give (1S)-1-(3-fluorophenyl)-N-((3-hydroxy-1-methylpiperidin-3-yl)methyl)-3,4-dihydroisoquinoline-2(1H)-carbothioamide 18.

LCMS: (M+H)⁺ 432 (UV 214 nm); Retention time 1.653 min. CP Method C

Step 6: A solution of (1S)-1-(3-fluorophenyl)-N-((3-hydroxy-1-methylpiperidin-3-yl)methyl)-3,4-dihydroisoquinoline-2(1H)-carbothioamide 18 (850 mg, 20 mmol) in THF (50 mL) was cooled to 0° C. then phosphorus oxychloride (1.53 g, 10 mmol) was added. The reaction was stirred at 60° C. until the starting material was consumed (TLC) and then concentrated in vacuo. Purification of the residue by prep-HPLC yielded 2-((S)-1-(3-fluorophenyl)-3,4-dihydroisoquinolin-2(1H)-yl)-7-methyl-1-oxa-3,7-diazaspiro[4.5]dec-2-ene 19.

LCMS: (M+H)⁺ 380 (UV 214 nm); Retention time 1.273 min. CP Method C

The diastereomers were separated by chiral SFC eluting with CO₂/MeOH containing 0.2% methanolic ammonia over a Chiral® Column (4.6×100 mm, 5 μm) to give compound 3006 (10.3 mg, retention time=7.455 min), compound 3007 (7.6 mg, retention time=8.941 min), compound 3008 (10.3 mg, retention time=10.037 min) and compound 3009 (9.7 mg, retention time=11.067 min). Stereochemical assignment of the chiral centers at both the spiropiperidine and the tetrahydroisoquinoline is tentative and based on chromatographic elution order on the IG column.

Compound 3006: LCMS: (M+H)⁺=380; purity=100% (214 nm); retention time=1.273 min. LCMS CP Method C

¹H NMR (400 MHz, CDCl₃) δ 7.19 (dd, J=17.8, 6.3 Hz, 4H), 7.05-6.89 (m, 4H), 6.26 (s, 1H), 4.02 (d, J=11.2 Hz, 1H), 3.67 (d, J=12.0 Hz, 1H), 3.54 (d, J=12.1 Hz, 1H), 3.39-3.26 (m, 1H), 3.09-3.01 (m, 1H), 2.77 (d, J=16.2 Hz, 1H), 2.55-2.47 (m, 1H), 2.37 (d, J=10.0 Hz, 2H), 2.31 (s, 3H), 2.07-1.93 (m, 1H), 1.81 (d, J=11.5 Hz, 2H), 1.63 (d, J=6.4 Hz, 2H).

Chiral SFC: CO₂/MeOH containing 0.2% ammonia over a Chiral® IG column (4.6×100 mm, 5 μm), retention time=7.455 min), 100% ee.

Compound 3007: LCMS: (M+H)⁺=380; purity=100% (214 nm); retention time=1.303 min. LCMS CP Method C

¹H NMR (400 MHz, CDCl₃) δ 7.25-7.16 (m, 4H), 7.06 (d, J=7.4 Hz, 1H), 6.99 (d, J=7.7 Hz, 1H), 6.94 (d, J=9.1 Hz, 2H), 6.25 (s, 1H), 4.03-3.94 (m, 1H), 3.66 (d, J=12.1 Hz, 1H), 3.55 (d, J=12.1 Hz, 1H), 3.43-3.33 (m, 1H), 3.04 (ddd, J=16.2, 10.1, 6.1 Hz, 1H), 2.76 (dt, J=16.3, 4.1 Hz, 1H), 2.58-2.48 (m, 1H), 2.44-2.34 (m, 2H), 2.29 (s, 3H), 2.02 (dt, J=15.9, 7.3 Hz, 1H), 1.91-1.76 (m, 2H), 1.67-1.59 (m, 2H).

Chiral SFC: CO₂/MeOH containing 0.2% ammonia over a Chiral® IG column (4.6×100 mm, 5 μm), retention time=8.941 min), 100% ee.

Compound 3008: LCMS: (M+H)⁺=380; purity=100% (214 nm); retention time=1.320 min. LCMS CP Method C

¹H NMR (400 MHz, CDCl₃) δ 7.19 (ddd, J=17.0, 8.9, 2.7 Hz, 4H), 7.05-6.92 (m, 4H), 6.26 (s, 1H), 4.02 (dd, J=7.3, 3.9 Hz, 1H), 3.67 (d, J=12.1 Hz, 1H), 3.56 (t, J=10.0 Hz, 1H), 3.40-3.28 (m, 1H), 3.09-3.01 (m, 1H), 2.81-2.71 (m, 1H), 2.53 (d, J=10.3 Hz, 1H), 2.37 (d, J=10.8 Hz, 2H), 2.31 (s, 3H), 2.02 (dd, J=14.8, 7.7 Hz, 1H), 1.89-1.77 (m, 2H), 1.63 (dd, J=12.6, 6.0 Hz, 2H).

Chiral SFC: CO₂/MeOH containing 0.2% ammonia over a Chiral® IG column (4.6×100 mm, 5 μm), retention time=10.037 min), 100% ee.

Compound 3009: LCMS: (M+H)⁺=380; purity=100% (214 nm); retention time=1.323 min. LCMS CP Method C

¹H NMR (400 MHz, CDCl₃) δ 7.25-7.16 (m, 4H), 7.10-7.03 (m, 1H), 6.99 (d, J=7.8 Hz, 1H), 6.93 (d, J=8.7 Hz, 2H), 6.26 (s, 1H), 4.07-3.95 (m, 1H), 3.67 (d, J=12.1 Hz, 1H), 3.56 (d, J=12.1 Hz, 1H), 3.43-3.33 (m, 1H), 3.05 (ddd, J=16.1, 10.0, 6.0 Hz, 1H), 2.77 (dt, J=16.2, 4.1 Hz, 1H), 2.56 (d, J=11.1 Hz, 1H), 2.44-2.36 (m, 2H), 2.31 (d, J=8.7 Hz, 3H), 2.02 (dt, J=14.5, 6.5 Hz, 1H), 1.90-1.76 (m, 2H), 1.63 (d, J=6.8 Hz, 2H).

Chiral SFC: CO₂/MeOH containing 0.2% ammonia over a Chiral® IG column (4.6×100 mm, 5 μm), retention time=11.067 min), 100% ee.

Compound 3002, compound 3003, compound 3004 and compound 3005 were prepared following the General CP Method B for spiro-compound synthesis using racemic 1-(3,4,5-trifluorophenyl)-tetrahydroisoquinoline.

The stereoisomers were separated by chiral SFC over multiple passes eluting with EtOH containing 1% ammonia in methanol over a Chiral® OX-H column (4.6×100 mm, 5 μm) to give Compound 3002 (retention time=1.21 min), compound 3003 (retention time=1.81 min), compound 3004 (retention time=2.5 min) and compound 3005 (retention time=2.9 min). Compound numbers for these isomers were sequential based on elution order from the OX-H SFC column. Stereochemical assignments of the chiral centers at the piperidine and tetrahydroisoquinoline are tentative.

Compound 3002: LCMS: (M+H)⁺=416; purity=95.6% (214 nm); Retention time=2.035 min. CP Method C

¹H NMR (400 MHz, CDCl₃) δ 7.23 (d, J=9.7 Hz, 2H), 7.19 (d, J=7.3 Hz, 1H), 7.00 (d, J=7.5 Hz, 1H), 6.95-6.86 (m, 2H), 6.19 (s, 1H), 4.02-3.91 (m, 1H), 3.61 (d, J=12.3 Hz, 1H), 3.52 (d, J=12.4 Hz, 1H), 3.29-3.17 (m, 1H), 3.03 (ddd, J=16.6, 10.6, 6.1 Hz, 1H), 2.74 (dt, J=16.3, 3.8 Hz, 1H), 2.55 (s, 1H), 2.48 (s, 1H), 2.31 (s, 5H), 1.93-1.76 (m, 2H), 1.63 (s, 2H).

Chiral SFC: n-hexane (0.1% DEA):EtOH (0.1% DEA)=80:20 over a Chiral® IG column (4.6×250 mm, 5 μm), retention time=4.669 min).

Compound 3003: LCMS: (M+H)⁺=416; purity=97.1% (214 nm); Retention time=2.035 min. CP Method C

¹H NMR (400 MHz, CDCl₃) δ 8.35 (s, 1H), 7.24 (s, 1H), 7.22 (t, J=7.4 Hz, 2H), 7.06 (d, J=7.6 Hz, 1H), 6.87 (dd, J=8.1, 6.7 Hz, 2H), 6.30 (s, 1H), 4.06-3.97 (m, 2H), 3.71 (d, J=11.9 Hz, 1H), 3.61 (d, J=11.9 Hz, 1H), 3.45-3.35 (m, 1H), 3.03 (dd, J=10.0, 6.1 Hz, 1H), 2.79 (dt, J=16.3, 4.4 Hz, 2H), 2.69 (d, J=20.4 Hz, 1H), 2.43 (d, J=11.8 Hz, 1H), 2.37 (s, 3H), 1.99-1.84 (m, 2H), 1.77-1.57 (m, 2H).

Chiral SFC: n-hexane (0.1% DEA):EtOH (0.1% DEA)=80:20 over a Chiral® IG column (4.6×250 mm, 5 μm), retention time=5.555 min).

Compound 3004: LCMS: (M+H)⁺=416; purity=78.7% (214 nm); Retention time=1.955 min. CP Method C

¹H NMR (400 MHz, CDCl₃) δ 7.24 (d, J=1.3 Hz, 1H), 7.20 (dd, J=6.2, 4.0 Hz, 2H), 7.03 (d, J=7.2 Hz, 1H), 6.86 (dd, J=8.4, 6.8 Hz, 2H), 6.17 (s, 1H), 3.95-3.87 (m, 1H), 3.63 (d, J=12.4 Hz, 1H), 3.52 (d, J=12.4 Hz, 1H), 3.32-3.24 (m, 1H), 3.00 (ddd, J=16.1, 10.0, 5.9 Hz, 1H), 2.72 (dt, J=16.3, 4.2 Hz, 1H), 2.51 (s, 1H), 2.37 (d, J=12.7 Hz, 3H), 2.29 (s, 3H), 1.79 (dd, J=20.2, 8.1 Hz, 2H), 1.63 (s, 2H).

Chiral SFC: n-hexane (0.1% DEA):EtOH (0.1% DEA)=80:20 over a Chiral® IG column (4.6×250 mm, 5 μm), retention time=5.402 min).

Compound 3005: LCMS: (M+H)⁺=416; purity=100% (214 nm); Retention time=2.037 min. CP Method A

¹H NMR (400 MHz, CDCl₃) δ 7.26-7.21 (m, 2H), 7.19 (d, J=7.3 Hz, 1H), 7.04-6.96 (m, 1H), 6.91 (dd, J=8.3, 6.8 Hz, 2H), 6.20 (s, 1H), 3.98 (s, 1H), 3.63 (d, J=12.3 Hz, 1H), 3.53 (d, J=12.3 Hz, 1H), 3.33-3.17 (m, 1H), 3.04 (ddd, J=16.2, 10.5, 6.0 Hz, 1H), 2.76 (dt, J=16.3, 3.7 Hz, 1H), 2.56 (s, 1H), 2.48 (s, 1H), 2.31 (s, 5H), 1.93-1.70 (m, 3H), 1.69-1.63 (m, 1H).

Chiral SFC: n-hexane (0.1% DEA):EtOH (0.1% DEA)=80:20 over a Chiral® IG column (4.6×250 mm, 5 μm), retention time=4.989 min).

Compounds 3010-3013 were prepared following the General CP Method B for spiro-compound synthesis using racemic 1-(3,5-difluorophenyl)tetrahydroisoquinoline.

The diastereomers were separated by chiral SFC eluting with CO₂/MeOH containing 0.2% methanolic ammonia over a Chiral® Column (4.6×100 mm, 5 μm) to give compound 3010 (retention time=2.24 min), compound 3011 (retention time=2.32 min), compound 3012 (retention time=3.2 min) and compound 3013 (retention time=3.77 min). Stereochemical assignment of the chiral centers at the piperidine and tetrahydroisoquinoline is tentative and based on chromatographic elution order on the IG column.

Compound 3010: LCMS: (M+H)⁺=398; purity=100% (214 nm); retention time=1.995 min. CP Method C

¹H NMR (400 MHz, DMSO-d₆) δ 7.27-7.18 (m, 4H), 7.14 (tt, J=9.2, 2.3 Hz, 1H), 6.86 (d, J=6.6 Hz, 2H), 6.09 (s, 1H), 3.76-3.66 (m, 1H), 3.46 (d, J=12.5 Hz, 1H), 3.43-3.37 (m, 2H), 2.92-2.75 (m, 2H), 2.38-2.23 (brs, 3H), 2.16 (s, 4H), 1.68-1.59 (m, 1H), 1.56-1.48 (m, 2H), 1.47-1.39 (m, 1H).

Chiral SFC: IPA containing 1% ammonia over a Chiral® IG column (4.6×100 mm, 5 μm), retention time=2.24 min)

Compound 3011: LCMS: (M+H)⁺=398; purity=98.8% (214 nm); retention time=2.086 min. CP Method C

¹H NMR (400 MHz, DMSO-d₆) δ 7.27-7.23 (m, 2H), 7.22-7.11 (m, 3H), 6.86 (d, J=6.5 Hz, 2H), 6.10 (s, 1H), 3.80-3.72 (m, 1H), 3.44 (d, J=12.5 Hz, 1H), 3.33-3.24 (m, 2H), 2.95-2.85 (m, 1H), 2.84-2.75 (m, 1H), 2.35-2.20 (m, 4H), 2.18 (s, 3H), 1.78-1.70 (m, 1H), 1.69-1.54 (m, 2H), 1.53-1.43 (m, 1H).

Chiral SFC: IPA containing 1% ammonia over a Chiral® IG column (4.6×100 mm, 5 μm), retention time=2.32 min)

Compound 3012: LCMS: (M+H)⁺=398; purity=100% (214 nm); retention time=1.942 min. CP Method C

¹H NMR (400 MHz, DMSO-d₆) δ 7.27-7.18 (m, 4H), 7.18-7.10 (m, 1H), 6.86 (d, J=6.5 Hz, 2H), 6.09 (s, 1H), 3.74-3.66 (m, 1H), 3.46 (d, J=12.5 Hz, 1H), 3.43-3.36 (m, 2H), 2.93-2.84 (m, 1H), 2.79 (dt, J=16.3, 4.9 Hz, 1H), 2.39-2.24 (brs, 3H), 2.16 (s, 4H), 1.68-1.59 (m, 1H), 1.56-1.50 (m, 2H), 1.49-1.39 (s, 1H).

Chiral SFC: IPA containing 1% ammonia over a Chiral® IG column (4.6×100 mm, 5 μm), retention time=3.20 min)

Compound 3013: LCMS: (M+H)⁺=398; purity=100% (214 nm); retention time=1.996 min. CP Method C

¹H NMR (400 MHz, DMSO-d₆) δ 7.27-7.24 (m, 2H), 7.23-7.12 (m, 3H), 6.87 (d, J=6.5 Hz, 2H), 6.12 (s, 1H), 3.77 (dt, J=12.7, 5.0 Hz, 1H), 3.45 (d, J=12.4 Hz, 1H), 3.33-3.27 (m, 2H), 2.96-2.86 (m, 1H), 2.84-2.75 (m, 1H), 2.35-2.23 (brs, 3H), 2.20 (s, 3H), 1.80-1.71 (m, 1H), 1.69-1.56 (m, 2H), 1.54-1.45 (m, 1H).

Chiral SFC: IPA containing 1% ammonia over a Chiral® IG column (4.6×100 mm, 5 μm), retention time=3.77 min).

Compound 3014 and compound 3015 were prepared following the General CP Method B for spiro-compound synthesis using N-methylpiperidin-3-one and enantiomerically pure (R)-1-(2,4-difluorophenyl)-1,2,3,4-tetrahydroisoquinoline.

The diastereomers were separated by chiral SFC eluting with EtOH containing 1% methanolic ammonia over a Chiral® OZ-H column (4.6×100 mm, 5 μm) to give Compound 3014 (retention time=1.4 min) and Compound 3015 (retention time=1.57 min). Stereochemical assignment of (R) at tetrahydroisoquinoline 1-position is absolute based on starting materials, stereochemical assignment at spiropiperidine is assigned randomly based on chromatographic elution order.

Compound 3014: LCMS: (M+H)⁺=398; purity=100% (214 nm); Retention time=1.866 min. CP Method C

¹H NMR (400 MHz, CDCl₃) δ 7.23-7.10 (m, 3H), 6.96 (ddd, J=15.0, 8.2, 5.7 Hz, 2H), 6.84-6.68 (m, 2H), 6.40 (s, 1H), 4.01 (ddd, J=13.3, 5.7, 3.1 Hz, 1H), 3.59 (d, J=12.4 Hz, 1H), 3.52-3.47 (m, 1H), 3.44-3.33 (m, 1H), 3.07 (ddd, J=16.5, 10.8, 5.8 Hz, 1H), 2.79 (dt, J=16.2, 3.5 Hz, 1H), 2.49 (d, J=10.5 Hz, 1H), 2.36 (d, J=20.2 Hz, 3H), 2.27 (s, 3H), 1.87-1.77 (m, 1H), 1.58 (d, J=6.2 Hz, 1H), 1.57-1.43 (m, 2H).

Chiral SFC: MeOH (0.2% methanol ammonia) over a Chiral® IG column (4.6×100 mm, 5 μm), retention time=1.40 min).

Compound 3015: LCMS: (M+H)⁺=398; purity=100% (214 nm); Retention time=1.92 min. CP Method C

¹H NMR (400 MHz, CDCl₃) δ 7.25-7.11 (m, 3H), 7.04-6.90 (m, 2H), 6.78 (ddd, J=23.9, 13.8, 5.4 Hz, 2H), 6.41 (s, 1H), 4.09 (s, 1H), 3.70 (d, J=12.0 Hz, 1H), 3.51 (d, J=12.2 Hz, 1H), 3.43-3.33 (m, 1H), 3.08 (ddd, J=16.7, 11.0, 6.0 Hz, 1H), 2.82 (d, J=16.4 Hz, 1H), 2.39 (d, J=11.3 Hz, 2H), 2.34-2.16 (m, 5H), 1.79-1.65 (m, 3H), 1.60-1.52 (m, 1H).

Chiral SFC: MeOH (0.2% methanol ammonia) over a chiral IG column (4.6×100 mm, 5 μm), retention time=1.57 min).

Compound 3016 and compound 3017 were prepared following the General CP Method A for spiro-compound synthesis using quinuclidine-3-one and enantiomerically pure (R)-1-(2,4-difluorophenyl)-1,2,3,4-tetrahydroisoquinoline.

The diastereomers were separated by chiral SFC eluting with CO₂/EtOH containing 0.2% methanolic ammonia over a chiral IG column (20×250 mm, 10 μm) to give Compound 3016 (retention time=1.697 min) and Compound 3017 (retention time=2.465 min). Stereochemical assignment of (R) at the tetrahydroisoquinoline is absolute based on starting materials; stereochemical assignment at quinuclidine is assigned based on chromatographic elution order on the IG column.

Compound 3016: LCMS: (M+H)⁺=410; purity=100% (214 nm); retention time=1.449 min. LCMS CP Method B

¹H NMR (400 MHz, CDCl₃) δ 7.22-7.18 (m, 2H), 7.18-7.12 (m, 1H), 7.04-6.95 (m, 2H), 6.84 (ddd, J=11.2, 8.8, 2.4 Hz, 1H), 6.76 (td, J=8.0, 1.6 Hz, 1H), 6.34 (s, 1H), 4.04 (ddd, J=13.2, 5.2, 3.6 Hz, 1H), 3.90 (d, J=12.4 Hz, 1H), 3.59 (dd, J=24.0, 10.8 Hz, 2H), 3.48 (ddd, J=13.2, 10.4, 4.0 Hz, 1H), 3.14-3.04 (m, 1H), 3.01-2.88 (m, 4H), 2.86-2.78 (m, 2H), 2.08-2.00 (m, 2H), 1.72-1.50 (m, 3H).

Chiral SFC: MeOH (0.2% methanol ammonia) over a chiral IG column (4.6×100 mm, 5 μm), retention time=1.697 min).

Compound 3017: LCMS: (M+H)⁺=410; purity=100% (214 nm); retention time=1.466 min. LCMS CP Method B

¹H NMR (400 MHz, CDCl₃) δ 7.24-7.13 (m, 3H), 7.03 (d, J=7.6 Hz, 1H), 6.97 (dd, J=14.8, 4.8 Hz, 1H), 6.81 (ddd, J=11.2, 8.8, 2.4 Hz, 1H), 6.74 (td, J=8.0, 1.6 Hz, 1H), 6.38 (s, 1H), 4.00-3.90 (m, 2H), 3.54-3.44 (m, 2H), 3.22 (d, J=6.8 Hz, 1H), 3.06 (ddd, J=16.0, 10.0, 1.6 Hz, 1H), 2.95-2.85 (m, 3H), 2.75 (q, J=8.0 Hz, 2H), 2.60 (s, 1H), 1.88 (s, 1H), 1.86-1.76 (m, 1H), 1.64-1.54 (m, 2H), 1.46-1.36 (m, 1H).

Chiral SFC: MeOH (0.2% methanol ammonia) over a chiral IG column (4.6×100 mm, 5 μm), retention time=2.465 min).

Compound 3018 and compound 3019 were prepared following the General CP Method A for spiro-compound synthesis using quinuclidine-3-one and racemic difluorophenyl)-1,2,3,4-tetrahydroisoquinoline.

The diastereomers were separated by chiral SFC eluting with CO₂/MeOH containing 0.2% methanolic ammonia over a chiral IG column (4.6×100 mm, 5 μm) to give Compound 3018 (retention time=1.52 min) and Compound 3019 (retention time=3.56 min). Stereochemical assignment at quinuclidine and the tetrahydroisoquinoline is random based on starting materials and synthesis method. These compounds were peaks 3 and 4 in elution order from the SFC column. Peaks 1-2 were not isolated in pure form.

Compound 3018: LCMS: (M+H)⁺=410; purity=100% (214 nm); retention time=1.145 min. CP Method E

¹H NMR (400 MHz, CDCl₃) δ 7.24-7.13 (m, 3H), 7.10-7.01 (m, 2H), 6.97-6.91 (m, 1H), 6.82-6.74 (m, 1H), 6.42 (s, 1H), 4.01-3.89 (m, 2H), 3.60-3.46 (m, 2H), 3.23 (d, J=14.8 Hz, 1H), 3.11-3.00 (m, 1H), 2.95-2.83 (m, 4H), 2.81-2.68 (m, 2H), 2.09-1.91 (m, 1H), 1.89-1.82 (m, 1H), 1.78-1.68 (m, 1H), 1.62-1.48 (m, 2H).

Chiral SFC: MeOH (0.2% methanol ammonia) over a chiral IG column (4.6×100 mm, 5 μm), retention time=1.52 min).

Compound 3019: LCMS: (M+H)⁺=410; purity=100% (214 nm); retention time=1.532 min. CP Method C1

¹H NMR (400 MHz, CDCl₃) δ 7.24-7.13 (m, 3H), 7.10-7.02 (m, 2H), 6.97-6.90 (m, 1H), 6.84-6.72 (m, 1H), 6.42 (s, 1H), 3.98-3.89 (m, 2H), 3.59-3.43 (m, 2H), 3.21 (d, J=14.8 Hz, 1H), 3.11-3.01 (m, 1H), 2.93-2.83 (m, 4H), 2.82-2.65 (m, 2H), 2.07-1.91 (m, 1H), 1.87-1.82 (m, 1H), 1.77-1.72 (m, 1H), 1.60-1.52 (m, 2H).

Chiral SFC: MeOH (0.2% methanol ammonia) over a chiral IG column (4.6×100 mm, 5 μm), retention time=3.56 min).

Compounds 3020-3023 were prepared following the General CP Method A for spiro-compound synthesis using quinuclidine-3-one and racemic 1-(3-fluorophenyl)-1,2,3,4-tetrahydroisoquinoline.

Two of four stereoisomers were separated by chiral SFC eluting with CO₂/EtOH containing 0.2% methanolic ammonia over a chiral IG column (20×250 mm, 10 μm) to give Compound 3020 (retention time=1.75 min) and Compound 3021 (retention time=2.24 min). The other two stereoisomers were separated by chiral SFC eluting with CO₂/EtOH containing 0.2% methanol ammonia over a chiral IC column (20×250 mm, 10 μm) to give Compound 3022 (retention time=2.19 min) and Compound 3023 (retention time=3.77 min). Stereochemical assignments are random at both the quinuclidine and the tetrahydroisoquinoline and are based on chromatographic elution order on the IG column.

Compound 3020: LCMS: (M+H)⁺=392; purity=99% (214 nm); retention time=1.477 min. LCMS CP Method B

¹H NMR (400 MHz, CDCl₃) δ 7.25-7.17 (m, 4H), 7.10 (d, J=7.6 Hz, 1H), 7.00 (d, J=7.6 Hz, 1H), 6.95-6.89 (m, 2H), 6.18 (s, 1H), 3.95 (d, J=12.8 Hz, 1H), 3.87 (dt, J=12.8, 5.6 Hz, 1H), 3.54 (d, J=12.4 Hz, 1H), 3.41 (ddd, J=13.6, 9.2, 4.4 Hz, 1H), 3.22 (d, J=14.8 Hz, 1H), 3.04-2.95 (m, 1H), 2.94-2.86 (m, 3H), 2.81-2.72 (m, 3H), 2.00-1.96 (m, 1H), 1.92-1.82 (br, 1H), 1.66-1.58 (m, 2H), 1.52-1.42 (m, 1H).

Chiral SFC: MeOH (0.2% methanol ammonia) over a chiral IG column (4.6×100 mm, 5 μm), retention time=1.76 min).

Compound 3021: LCMS: (M+H)⁺=392; purity=97% (214 nm); retention time=1.448 min. LCMS CP Method B

¹H NMR (400 MHz, CDCl₃) δ 7.22-7.16 (m, 4H), 7.05 (t, J=8.4 Hz, 2H), 6.97-6.91 (m, 2H), 6.16 (s, 1H), 3.97-3.84 (m, 2H), 3.54 (d, J=12.4 Hz, 1H), 3.38 (ddd, J=13.6, 10.0, 4.4 Hz, 1H), 3.11 (d, J=11.2 Hz, 1H), 3.09-2.97 (m, 1H), 2.92 (d, J=5.2 Hz, 2H), 2.90 (s, 1H), 2.81-2.72 (m, 3H), 2.03-1.96 (m, 1H), 1.95-1.75 (br, 1H), 1.66-1.58 (m, 2H), 1.52-1.45 (m, 1H).

Chiral SFC: MeOH (0.2% methanol ammonia) over a chiral IG column (4.6×100 mm, 5 μm), retention time=1.83 min).

Compound 3022: LCMS: (M+H)⁺=392; purity=100% (214 nm); retention time=1.507 min. LCMS CP Method B

¹H NMR (400 MHz, CDCl₃) δ 7.25-7.17 (m, 4H), 7.05 (t, J=8.8 Hz, 2H), 6.97-6.89 (m, 2H), 6.16 (s, 1H), 3.99-3.90 (m, 2H), 3.56 (d, J=12.4 Hz, 1H), 3.39 (ddd, J=13.6, 10.0, 4.4 Hz, 1H), 3.15 (d, J=14.8 Hz, 1H), 3.09-3.00 (m, 1H), 2.96-2.90 (m, 3H), 2.85-2.73 (m, 3H), 2.05-1.95 (br, 2H), 1.70-1.58 (m, 2H), 1.57-1.48 (m, 1H).

Chiral SFC: MeOH (0.2% methanol ammonia) over a chiral IG column (4.6×100 mm, 5 μm), retention time=2.24 min).

Compound 3023: LCMS: (M+H)⁺=392; purity=98% (214 nm); retention time=1.443 min. LCMS CP Method B

¹H NMR (400 MHz, CDCl₃) δ 7.25-7.17 (m, 4H), 7.05 (t, J=8.0 Hz, 2H), 6.97-6.90 (m, 2H), 6.16 (s, 1H), 3.97-3.88 (m, 2H), 3.55 (d, J=12.4 Hz, 1H), 3.38 (ddd, J=17.6, 9.2, 4.4 Hz, 1H), 3.12 (d, J=15.2 Hz, 1H), 3.08-2.98 (m, 1H), 2.95-2.87 (m, 3H), 2.81-2.72 (m, 3H), 2.03-1.96 (m, 1H), 1.90-1.70 (br, 2H), 1.66-1.58 (m, 1H), 1.55-1.45 (m, 1H).

Chiral SFC: MeOH (0.2% methanol ammonia) over a chiral IG column (4.6×100 mm, 5 μm), retention time=2.86 min).

Compounds 3024-3027 were prepared following the General CP Method A for spiro-compound synthesis using quinuclidine-3-one and racemic 1-(3,4-difluorophenyl)-1,2,3,4-tetrahydroisoquinoline.

The stereoisomers were separated by chiral SFC eluting with EtOH containing 1% methanolic ammonia) over a chiral AD (4.6×100 mm, 5 μm) to give Compound 3024 (retention time=1.86 min), Compound 3025 (retention time=2.32 min), Compound 3026 (retention time=3.04 min), and Compound 3027 (retention time=3.95 min). Stereochemical assignment at both the quinuclidine and the tetrahydroisoquinoline is tentative and based on chromatographic elution order on the IG column.

Compound 3024: LCMS: (M+H)⁺=410.2; purity=100% (214 nm); retention time=2.045 min. CP Method C

¹H NMR (400 MHz, CDCl₃) δ 7.26-7.19 (m, 3H), 7.11-7.04 (m, 3H), 6.99-6.97 (m, 1H), 6.16 (s, 1H), 3.98-3.90 (m, 2H), 3.55 (d, J=12.8 Hz, 1H), 3.37-3.30 (m, 1H), 3.16 (d, J=14.8 Hz, 1H), 3.09-3.01 (m, 1H), 2.95-2.92 (m, 3H), 2.83-2.73 (m, 3H), 2.00-1.96 (m, 2H), 1.80-1.61 (m, 2H), 1.55-1.48 (m, 1H).

Chiral SFC: EtOH (1% methanol ammonia) over a chiral IG column (4.6×100 mm, 5 μm), retention time=1.99 min).

Compound 3025: LCMS: (M+H)⁺=410.2; purity=100% (214 nm); retention time=1.190 min. CP Method C

¹H NMR (400 MHz, CDCl₃) δ 7.26-7.20 (m, 3H), 7.10-7.03 (m, 3H), 6.96-6.93 (m, 1H), 6.18 (s, 1H), 3.97 (d, J=12.4 Hz, 1H), 3.91-3.86 (m, 1H), 3.57 (d, J=12.4 Hz, 1H), 3.41-3.34 (m, 1H), 3.25 (d, J=14.8 Hz, 1H), 3.41-3.34 (m, 4H), 3.27-3.23 (m, 3H), 2.00 (s, 1H), 1.66-1.47 (m, 4H).

Chiral SFC: EtOH (1% methanol ammonia) over a chiral IG column (4.6×100 mm, 5 μm), retention time=2.13 min).

Compound 3026: LCMS: (M+H)⁺=410.2; purity=100% (214 nm); retention time=2.029 min. CP Method C

¹H NMR (400 MHz, CDCl₃) δ 7.27-7.20 (m, 3H), 7.10-7.04 (m, 3H), 6.96-6.94 (m, 1H), 6.18 (s, 1H), 3.97 (d, J=12.4 Hz, 1H), 3.91-3.86 (m, 1H), 3.58 (d, J=12.4 Hz, 1H), 3.42-3.35 (m, 1H), 3.26 (d, J=14.8 Hz, 1H), 3.01-2.93 (m, 4H), 2.84-2.74 (m, 3H), 2.02 (s, 1H), 1.92-1.90 (m, 1H), 1.66-1.63 (m, 2H), 1.57-1.51 (m, 1H).

Chiral SFC: EtOH (1% methanol ammonia) over a chiral IG column (4.6×100 mm, 5 μm), retention time=2.51 min).

Compound 3027: LCMS: (M+H)⁺=410.2; purity=100% (214 nm); retention time=2.047 min. CP Method C

¹H NMR (400 MHz, CDCl₃) δ 7.26-7.19 (m, 3H), 7.11-7.04 (m, 3H), 6.99-6.97 (m, 1H), 6.16 (s, 1H), 3.98-3.89 (m, 2H), 3.56 (d, J=12.4 Hz, 1H), 3.37-3.30 (m, 1H), 3.17 (d, J=14.8 Hz, 1H), 3.09-3.01 (m, 1H), 2.97-2.93 (m, 3H), 2.84-2.74 (m, 3H), 2.02-1.98 (m, 2H), 1.66-1.60 (m, 2H), 1.56-1.48 (m, 1H).

Chiral SFC: EtOH (1% methanol ammonia) over a chiral IG column (4.6×100 mm, 5 μm), retention time=3.60 min).

Compounds 3029-3032 were prepared following the General Method B for spiro-compound synthesis starting with 1-methyl-3-piperidinone and racemic difluorophenyl)-1,2,3,4-tetrahydroisoquinoline.

The stereoisomers were separated in two stages starting with chiral SFC eluting with EtOH containing 1% methanolic ammonia over a chiral OZ-H column (4.6×100 mm, 5 μm) to give Compound 3029 (retention time=3.46 min) and Compound 3030 (retention time=2.74 min). Stereochemical assignment at the quinuclidine and the tetrahydroisoquinoline is based on chromatographic elution order as compared to stereoisomers of related analogues of known configuration.

Compound 3029: LCMS: (M+H)⁺=398; purity=87.3% (214 nm); Retention time=1.89 min. CP Method C

¹H NMR (400 MHz, CDCl₃) δ 7.20 (dt, J=14.2, 6.3 Hz, 3H), 7.05 (ddt, J=14.3, 10.0, 5.1 Hz, 3H), 6.93 (dd, J=6.4, 4.4 Hz, 1H), 6.20 (s, 1H), 3.93 (ddd, J=13.1, 5.7, 3.9 Hz, 1H), 3.63 (d, J=12.4 Hz, 1H), 3.52 (d, J=12.4 Hz, 1H), 3.33-3.24 (m, 1H), 3.02 (ddd, J=16.4, 10.5, 6.0 Hz, 1H), 2.73 (dt, J=16.3, 4.1 Hz, 1H), 2.51 (s, 1H), 2.37 (d, J=11.2 Hz, 2H), 2.28 (d, J=3.5 Hz, 3H), 1.85-1.73 (m, 2H), 1.63 (s, 2H).

Chiral SFC: MeOH (0.2% methanolic ammonia) over a chiral IG column (4.6×100 mm, 5 μm), retention time=1.76 min).

Compound 3030: LCMS: (M+H)⁺=398; purity=88.2% (214 nm); Retention time=1.31 min. CP Method C

¹H NMR (400 MHz, CDCl₃) δ 7.13 (dt, J=14.3, 6.4 Hz, 3H), 7.03-6.91 (m, 3H), 6.86 (s, 1H), 6.13 (s, 1H), 3.90-3.82 (m, 1H), 3.56 (d, J=12.4 Hz, 1H), 3.45 (d, J=12.4 Hz, 1H), 3.25-3.17 (m, 1H), 2.95 (ddd, J=16.4, 10.4, 6.1 Hz, 1H), 2.66 (dt, J=16.2, 4.1 Hz, 1H), 2.44 (s, 1H), 2.30 (d, J=10.9 Hz, 3H), 2.22 (s, 3H), 1.81-1.67 (m, 2H), 1.55 (s, 2H).

Chiral SFC: MeOH (0.2% methanolic ammonia) over a chiral IG column (4.6×100 mm, 5 μm), retention time=1.77 min).

Secondly, the remaining stereoisomers were separated by chiral SFC eluting with EtOH containing 1% methanolic ammonia over a chiral OZ-H column (4.6×100 mm, 5 μm) to give Compound 3031 (retention time=2.42 min) and Compound 3032 (retention time=3.19 min). Stereochemical assignment at the quinuclidine and the tetrahydroisoquinoline is based on chromatographic elution order as compared to stereoisomers of related analogues of known configuration.

Compound 3031: LCMS: (M+H)⁺=398; purity=65.4% (214 nm); Retention time=1.33 min. CP Method C

¹H NMR (400 MHz, CDCl₃) δ 7.26-7.10 (m, 4H), 7.02 (ddd, J=12.2, 8.8, 6.4 Hz, 2H), 6.92 (dd, J=9.7, 4.9 Hz, 1H), 6.23 (s, 1H), 4.22 (dt, J=14.2, 6.4 Hz, 1H), 4.00 (s, 1H), 3.65 (d, J=12.2 Hz, 1H), 3.53 (d, J=12.2 Hz, 1H), 3.34-3.19 (m, 1H), 3.15-2.98 (m, 1H), 2.76 (d, J=16.5 Hz, 1H), 2.53 (s, 1H), 2.43 (s, 1H), 2.37-2.18 (m, 4H), 1.90-1.78 (m, 2H), 1.70-1.61 (m, 2H).

Chiral SFC: MeOH (0.2% methanolic ammonia) over a chiral IG column (4.6×100 mm, 5 μm), retention time=1.77 min).

Compound 3032: LCMS: (M+H)⁺=398; purity=100% (214 nm); Retention time=1.35 min. CP Method C

¹H NMR (400 MHz, CDCl₃) δ 7.25-7.09 (m, 4H), 7.07-6.98 (m, 2H), 6.93 (dd, J=6.3, 4.3 Hz, 1H), 6.21 (s, 1H), 3.95 (ddd, J=13.0, 5.9, 3.2 Hz, 1H), 3.62 (d, J=12.4 Hz, 1H), 3.51 (d, J=12.4 Hz, 1H), 3.24 (ddd, J=13.2, 10.8, 4.4 Hz, 1H), 3.04 (ddd, J=16.6, 10.8, 6.2 Hz, 1H), 2.74 (dt, J=16.2, 3.8 Hz, 1H), 2.50 (t, J=11.5 Hz, 1H), 2.42 (s, 1H), 2.33 (d, J=11.8 Hz, 5H), 1.90-1.74 (m, 2H), 1.72-1.60 (s, 2H).

Chiral SFC: MeOH (0.2% methanolic ammonia) over a chiral IG column (4.6×100 mm, 5 μm), retention time=1.90 min).

Compounds 3036-3039 were prepared following the General CP Method A for spiro-compound synthesis using racemic 1-(3,5-difluorophenyl)-1,2,3,4-tetrahydroisoquinoline.

The diastereomers were separated by chiral SFC eluting with MeOH containing 0.2% methanolic ammonia over a chiral AD-H column (4.6×100 mm, 5 μm) to give Compound 3036 and Compound 3037 as a mixture (retention time=1.72 min), Compound 3038 (retention time=2.32 min) and Compound 3039 (retention time=2.84 min). The mixture of diastereomers Compound 3036 and Compound 3037 was further separated by chiral SFC eluting with IPA containing 1% methanolic ammonia over a chiral IC column (4.6×100 mm, 5 μm) to give Compound 3036 (retention time=2.15 min) and Compound 3037 (retention time=4.64 min). Stereochemical assignment at both the quinuclidine and the tetrahydroisoquinoline is tentative and based on chromatographic elution order on the IG column.

Compound 3036: LCMS: (M+H)⁺=410; purity=100% (214 nm); retention time=1.167 min. LCMS CP Method C

¹H NMR (400 MHz, CDCl₃) δ 7.27-7.09 (m, 3H), 7.08 (d, J=6.8 Hz, 1H), 6.79 (d, J=6.4 Hz, 2H), 6.73-6.68 (m, 1H), 6.14 (s, 1H), 4.01-3.88 (m, 2H), 3.56 (d, J=12.4 Hz, 1H), 3.42-3.35 (m, 1H), 3.15 (d, J=14.8 Hz, 1H), 3.07-2.99 (m, 1H), 2.93 (d, J=12.4 Hz, 3H), 2.87-2.72 (m, 3H), 2.01 (s, 2H), 1.69-1.61 (m, 2H), 1.57-1.49 (m, 1H).

Chiral SFC: MeOH (0.2% methanol ammonia) over a chiral IG column (4.6×100 mm, 5 μm), retention time=1.73 min).

Compound 3037: LCMS: (M+H)⁺=410; purity=100% (214 nm); retention time=1.168 min. LCMS CP Method C

¹H NMR (400 MHz, CDCl₃) δ 7.28-7.21 (m, 3H), 7.12 (d, J=6.8 Hz, 1H), 6.77 (d, J=9.6 Hz, 2H), 6.72-6.67 (m, 1H), 6.16 (s, 1H), 3.98 (d, J=12.8 Hz, 1H), 3.90-3.84 (m, 1H), 3.58 (d, J=12.4 Hz, 1H), 3.47-3.40 (m, 1H), 3.27 (d, J=14.8 Hz, 1H), 3.00-2.93 (m, 4H), 2.84-2.74 (m, 3H), 2.02-2.01 (m, 2H), 1.68-1.63 (m, 2H), 1.54-1.51 (m, 1H).

Chiral SFC: MeOH (0.2% methanol ammonia) over a chiral IG column (4.6×100 mm, 5 μm), retention time=1.86 min).

Compound 3038: LCMS: (M+H)⁺=410; purity=100% (214 nm); retention time=1.164 min. LCMS CP Method C

¹H NMR (400 MHz, CDCl₃) δ 7.28-7.21 (m, 3H), 7.08 (d, J=7.6 Hz, 1H), 6.81-6.78 (dd, J=8.4 Hz, J=2 Hz, 2H), 6.73-6.68 (m, 1H), 6.14 (s, 1H), 3.97 (d, J=12.4 Hz, 1H), 3.94-3.88 (m, 1H), 3.56 (d, J=12.8 Hz, 1H), 3.43-3.36 (m, 1H), 3.15 (d, J=16 Hz, 1H), 3.08-3.00 (m, 1H), 2.94 (d, J=12.8 Hz, 3H), 2.81-2.75 (m, 3H), 2.02-1.97 (m, 2H), 1.58-1.49 (m, 2H), 1.29-1.25 (m, 1H).

Chiral SFC: MeOH (0.2% methanol ammonia) over a chiral IG column (4.6×100 mm, 5 μm), retention time=2.32 min).

Compound 3039: LCMS: (M+H)⁺=410; purity=100% (214 nm); retention time=1.163 min. LCMS CP Method C

¹H NMR (400 MHz, CDCl₃) δ 7.26-7.20 (m, 3H), 7.12 (d, J=7.6 Hz, 1H), 6.77 (d, J=7.6, 2H), 6.71-6.67 (m, 1H), 6.16 (s, 1H), 3.97 (d, J=12.4 Hz, 1H), 3.88-3.84 (m, 1H), 3.56 (d, J=12.4 Hz, 1H), 3.44-3.39 (m, 1H), 3.23 (d, J=14.8 Hz, 1H), 3.00-2.89 (m, 4H), 2.80-2.74 (m, 3H), 1.99 (s, 1H), 1.91-1.84 (m, 1H), 1.51-1.48 (m, 2H), 1.29-1.25 (m, 1H).

Chiral SFC: MeOH (0.2% methanol ammonia) over a chiral IG column (4.6×100 mm, 5 μm), retention time=4.05 min).

Compound 3040 and Compound 3041 were prepared following the General CP Method A for spiro-compound synthesis using enantiomerically pure (S)-1-phenyl-1,2,3,4-tetrahydroisoquinoline.

The diastereomers were separated by chiral SFC eluting with CO₂/MeOH containing 0.2% methanolic ammonia over a chiral IG column (4.6×100 mm, 5 μm) to give Compound 3040 (retention time=1.547 min) and Compound 3041 (retention time=3.919 min). Stereochemical assignment of (S) at the tetrahydroisoquinoline is absolute based on starting materials; stereochemical assignment at the quinuclidine is based on chromatographic elution order on the IG column.

Compound 3040: LCMS: (M+H)⁺=374; purity=100% (214 nm); retention time=1.519 min. LCMS CP Method C

¹H NMR (400 MHz, CD₃OD) δ 7.44-7.12 (m, 8H), 7.08 (d, J=7.4 Hz, 1H), 6.13 (d, J=19.9 Hz, 1H), 4.01-3.70 (m, 2H), 3.66-3.40 (m, 2H), 3.13-2.91 (m, 3H), 2.93-2.64 (m, 5H), 2.29-2.01 (m, 2H), 1.77-1.50 (m, 3H).

Chiral SFC: CO₂/MeOH containing 0.2% ammonia over a chiral IG column (4.6×100 mm, 5 μm), retention time=1.547 min), 100% ee.

Compound 3041: LCMS: (M+H)⁺=374; purity=100% (214 nm); retention time=1.509 min. LCMS CP Method C

¹H NMR (400 MHz, CD₃OD) δ 7.27-7.04 (m, 9H), 6.08 (d, J=11.9 Hz, 1H), 3.92 (d, J=12.0 Hz, 1H), 3.68 (dt, J=12.1, 5.9 Hz, 1H), 3.63-3.49 (m, 2H), 3.41-3.28 (m, 1H), 3.18-3.10 (m, 1H), 3.06-2.94 (m, 2H), 2.90 (dd, J=16.1, 8.1 Hz, 3H), 2.78 (dt, J=16.1, 5.6 Hz, 1H), 2.13-1.91 (m, 3H), 1.75-1.70 (m, 1H), 1.53 (d, J=7.8 Hz, 1H).

Chiral SFC: CO₂/MeOH containing 0.2% ammonia over a chiral IG column (4.6×100 mm, 5 μm), retention time=3.919 min), 100% ee.

Compounds 3042-3044 were prepared following the General CP Method B for spiro-compound synthesis using racemic 1-(2,3-difluorophenyl)-1,2,3,4-tetrahydroisoquinoline.

The diastereomers were separated by chiral SFC eluting with IPA containing 1% methanolic ammonia over a chiral AD-H column (4.6×100 mm, 5 μm) to give Compound 3042 (retention time=1.60 min), Compound 3043 (retention time=2.05 min) and Compound 3044 (retention time=3.06 min). Compound 3042 was a mixture of two isomers which could not be separated by SFC. Stereochemical assignment of the chiral centers at both the spiropiperidine and the tetrahydroisoquinoline is tentative and based on chromatographic elution order on the IG column.

Compound 3042: LCMS: (M+H)⁺=398; purity=100% (214 nm); retention time=1.149 min. LCMS CP Method A

¹H NMR (400 MHz, CDCl₃) δ 7.24-7.11 (m, 3H), 7.10-6.99 (m, 2H), 6.96-6.88 (m, 1H), 6.81-6.74 (m, 1H), 6.45 (d, J=7.0 Hz, 1H), 4.01 (ddd, J=13.1, 5.4, 3.0 Hz, 1H), 3.62 (dd, J=26.9, 12.4 Hz, 1H), 3.53-3.37 (m, 2H), 3.06 (ddd, J=16.3, 10.5, 5.8 Hz, 1H), 2.82 (dt, J=16.3, 3.5 Hz, 1H), 2.58-2.30 (m, 3H), 2.26 (d, J=9.6 Hz, 3H), 2.06-2.02 (m, 1H), 1.84-1.48 (m, 4H).

Chiral SFC: CO₂/MeOH containing 1% ammonia over a chiral IG column (4.6×100 mm, 5 μm), retention time=1.99 min), 99% de.

Compound 3043: LCMS: (M+H)⁺=398; purity=100% (214 nm); retention time=1.151 min. LCMS CP Method A

¹H NMR (400 MHz, CDCl₃) δ 7.24-7.13 (m, 3H), 7.05 (ddd, J=13.3, 8.7, 2.6 Hz, 2H), 6.98-6.90 (m, 1H), 6.81-6.72 (m, 1H), 6.47 (s, 1H), 4.04 (ddd, J=13.2, 5.6, 3.5 Hz, 1H), 3.67-3.56 (m, 1H), 3.55-3.37 (m, 2H), 3.14-3.01 (m, 1H), 2.89-2.78 (m, 1H), 2.50 (d, J=10.8 Hz, 1H), 2.37 (dd, J=16.6, 3.6 Hz, 3H), 2.26 (d, J=9.3 Hz, 3H), 1.85-1.76 (m, 1H), 1.71-1.47 (m, 3H).

Chiral SFC: CO₂/MeOH containing 1% ammonia over a chiral IG column (4.6×100 mm, 5 μm), retention time=2.54 min), 99% ee.

Compound 3044: LCMS: (M+H)⁺=398; purity=100% (214 nm); retention time=1.132 min. LCMS CP Method A

¹H NMR (400 MHz, CDCl₃) δ 7.25-7.12 (m, 3H), 7.11-6.99 (m, 2H), 6.98-6.87 (m, 1H), 6.76 (dd, J=7.7, 6.3 Hz, 1H), 6.42 (d, J=27.2 Hz, 1H), 4.13-4.00 (m, 1H), 3.73-3.62 (m, 1H), 3.54-3.38 (m, 2H), 3.14-3.01 (m, 1H), 2.84 (dt, J=16.3, 3.8 Hz, 1H), 2.52-2.20 (m, 7H), 1.86-1.45 (m, 4H).

Chiral SFC: CO₂/MeOH containing 1% ammonia over a chiral IG column (4.6×100 mm, 5 μm), retention time=2.7 min), 100% ee.

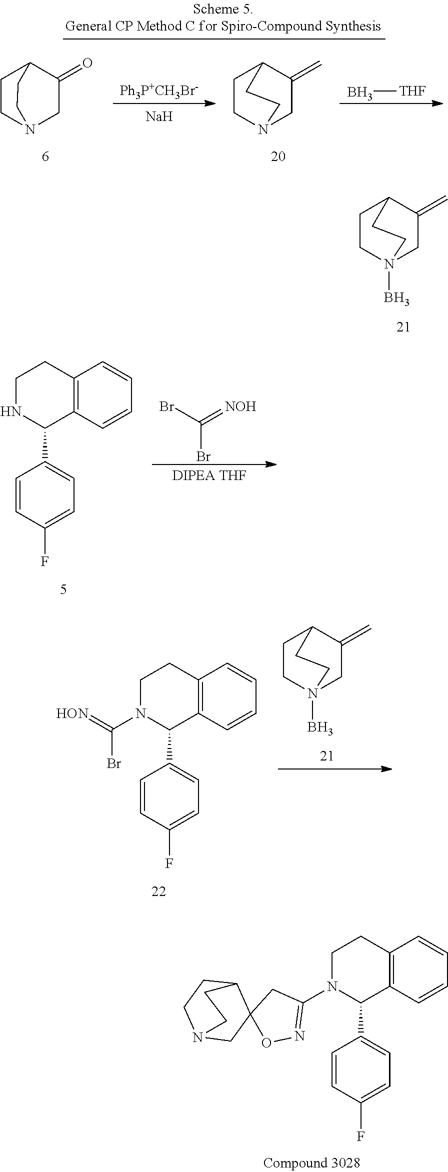

Step 1: To a solution of NaH (40 g, 1 mol) in tetrahydrofuran (100 mL) was added methyl triphenylphosphonium bromide (71.4 g 0.2 mol). The reaction was stirred at room temperature for four hours, then quinuclidin-3-one (25 g 0.2 mol) was added and the mixture was stirred until TLC analysis indicated the total consumption of the starting material. The solvent was evaporated and the residue distilled to give 3-methylenequinuclidine 20.

Step 2: To a mixture of 3-methylenequinuclidine 20 (12.3 g, 0.1 mol) in tetrahydrofuran (200 mL) at 0° C. was added dropwise 1 M borane in tetrahydrofuran (180 mL, 0.18 mol). The mixture was stirred at 0° C. for an hour. The solution was quenched by methanol, concentrated in vacuo and purified by silica gel chromatography eluting with petroleum ether:ethyl acetate=10:1) to give 1-(14-boranyl)-3-methylenequinuclidin-1-ium

Step 3: To a solution of (S)-1-(4-fluorophenyl)-1,2,3,4-tetrahydroisoquinoline 5 (2.27 g, 10 mmol), and DIPEA (3.87 g 30 mmol) in tetrahydrofuran (100 mL) cooled to 0° C. was added hydroxycarbamide dibromide (2.03 g, 10 mmol). The reaction was stirred at room temperature until TLC analysis indicated the total consumption of the starting material. The solvent was evaporated to give crude (S)-1-(4-fluorophenyl)-N-hydroxy-3,4-dihydroisoquinoline-2(1H)-carbimidoyl bromide 22 directly used in the next step without further purification.

LCMS: (M+H)⁺=349; purity=45% (UV 214 nm); retention time=1.780 min. LCMS CP Method B

Step 4: To a solution of (S)-1-(4-fluorophenyl)-N-hydroxy-3,4-dihydroisoquinoline-2(1H)-carbimidoyl bromide 22 (3.49 g, 20 mmol), and KHCO₃ (3.00 g 30 mmol) in tetrahydrofuran (100 mL) cooled to 0° C. was added olefin 21 (5.48 g, 40 mmol). The reaction was stirred at 60° C. until TLC analysis indicated the total consumption of the starting material. The solvent was evaporated to give crude compound 3028 which was further purified by preparative HPLC to give 2 [symbol] ((S)-1-(4-fluorophenyl)-3,4-dihydroisoquinolin-2(1H)-yl)-4 [symbol] -4-azaspiro[bicyclo[2.2.2]octane-2,5 [symbol] oxazole], compound 3028.

Compound 3028: LCMS: (M+H)⁺=392; purity=100% (214 nm); retention time=1.506 min. LCMS CP Method B

¹H NMR (400 MHz, CD₃OD) δ 7.33-7.10 (m, 5H), 7.06-6.91 (m, 3H), 6.46 (s, 1H), 5.06 (t, J=2.3 Hz, 1H), 4.94-4.85 (m, 5H), 4.50 (d, J=16.9 Hz, 2H), 4.15-3.70 (m, 5H), 3.33 (dt, J=3.3, 1.6 Hz, 6H), 3.18-3.03 (m, 1H), 2.94 (ddd, J=16.3, 10.4, 5.9 Hz, 1H), 2.73-2.60 (m, 2H), 2.26-1.94 (m, 4H).

Chiral SFC: CO₂/MeOH containing 0.2% ammonia over a Chiral® Column (4.6×100 mm, 5 μm), retention time=5.237 min), 100% ee.

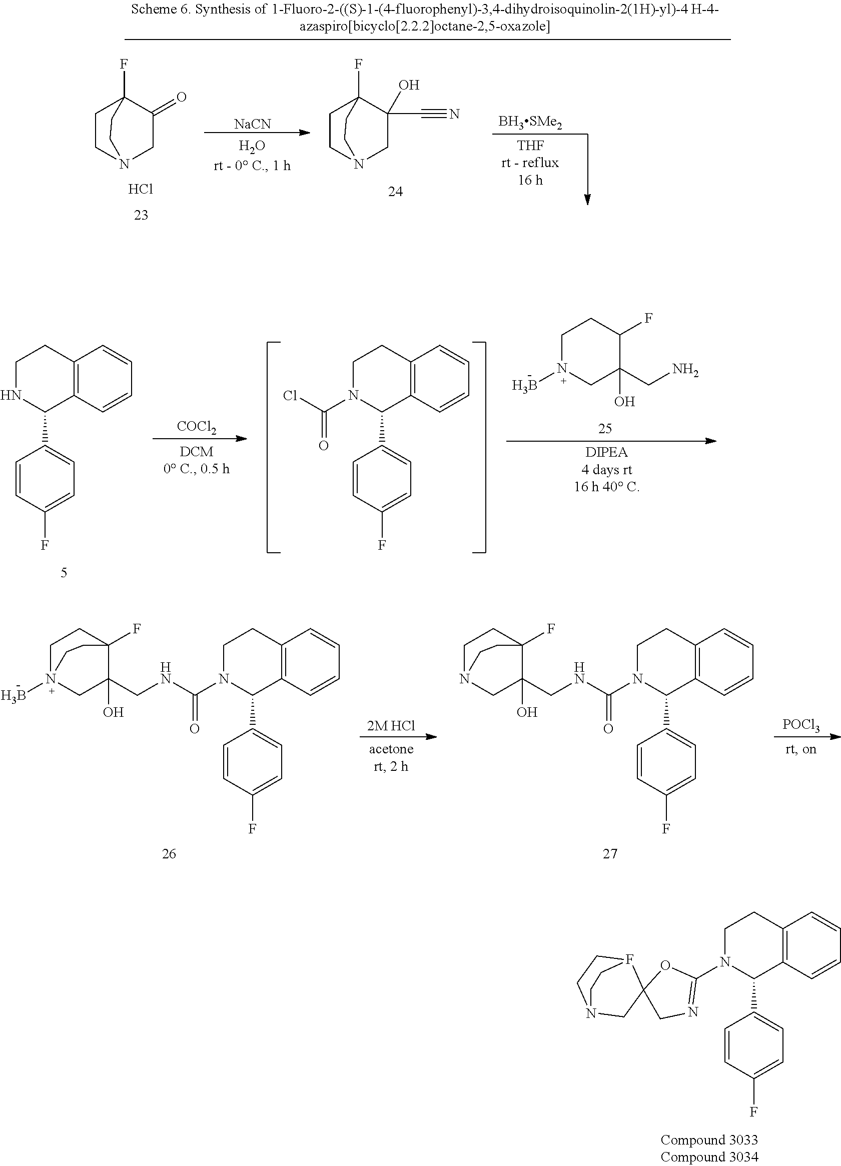

### Step 1: Synthesis of 4-fluoro-3-hydroxyquinuclidine-3-carbonitrile

A solution of NaCN (46.1 mg, 0.941 mmol) in water (0.2 mL) was added dropwise to a suspension of 4-fluoroquinuclidin-3-one hydrochloride 23 (154 mg, 0.855 mmol) in water (0.2 mL). During the addition, the reaction mixture became clear and immediately turned into a suspension again. The mixture was cooled to 0° C., stirred for 1 hour, filtered, washed with ice cold water, and dried on the filter to give 4-fluoro-3-hydroxyquinuclidine-3-carbonitrile 24 was used without further purification.

¹H NMR (400 MHz, DMSO-d₆) δ 7.14 (s, 1H), 3.35-3.28 (m, 1H), 3.01-2.80 (m, 5H), 2.09-1.95 (m, 1H), 1.92-1.73 (m, 2H), 1.66-1.55 (m, 1H).

### Step 2: Synthesis of 3-(aminomethyl)-4-fluoroquinuclidin-3-ol trihydroborate

Borane-methyl sulfide complex (2 M, 0.308 ml, 0.616 mmol) was added to a solution of 4-fluoro-3-hydroxyquinuclidine-3-carbonitrile 24 (105 mg, 0.617 mmol) in tetrahydrofuran (dry, 1 mL). After 1 hour, a second portion of borane-methyl sulfide complex (0.679 ml, 1.357 mmol) was added. Then, the reaction mixture was stirred under reflux overnight. At room temperature, methanol (few drops) was added slowly to the reaction mixture (vigorous foaming) after which the mixture was added dropwise to methanol (5 mL). The mixture was concentrated to dryness under reduced pressure and the residue was taken up into methanol (1 mL). The formed solid was filtered, washed with methanol, and dried on the filter to give a first crop of 3-(aminomethyl)-4-fluoroquinuclidin-3-ol trihydroborate 25. The mother liquor was concentrated to dryness under reduced pressure and the residue was crystallized from ethanol (2 mL) to give a second crop of 3-(aminomethyl)-4-fluoroquinuclidin-3-ol trihydroborate 25.

¹H NMR (400 MHz, DMSO-d₆) δ 5.20 (s, 1H), 3.23-3.05 (m, 4H), 3.05-2.93 (m, 1H), 2.83 (d, J=13.1 Hz, 1H), 2.78-2.69 (m, 2H), 2.40-2.26 (m, 1H), 2.18-2.05 (m, 1H), 1.93-1.73 (m, 2H), 1.45 (m, 5H).

### Step 3: Synthesis of (1S)—N-((4-fluoro-3-hydroxyquinuclidin-3-yl)methyl)-1-(4-fluorophenyl)-3,4-dihydroisoquinoline-2(1H)-carboxamide trihydroborate

At 0° C., (S)-1-(4-fluorophenyl)-1,2,3,4-tetrahydroisoquinoline 5 (98 mg, 0.431 mmol) was added to a solution of phosgene (20% in toluene, 0.302 mL, 0.574 mmol) in dichloromethane (0.5 mL). After 0.5 hour, a solution of 3-(aminomethyl)-4-fluoroquinuclidin-3-01 trihydroborate 25 (54 mg, 0.287 mmol) and N,N-diisopropylethylamine (0.251 ml, 1.436 mmol) was added at 0° C. The reaction mixture was stirred at room temperature for 4 days. Then, N,N-diisopropylethylamine (0.2 mL, 1.145 mmol) was added and the mixture was stirred at 40° C. overnight. The reaction mixture was diluted with dichloromethane (10 mL) and stirred with aqueous citric acid solution (1 M, 15 mL). The layers were separated using a phase-separator and the organic filtrate was evaporated under reduced pressure. The residue was purified by flash silica gel column chromatography eluting with 20% to 100% ethyl acetate in heptane to give (1S)—N-((4-fluoro-3-hydroxyquinuclidin-3-yl)methyl)-1-(4-fluorophenyl)-3,4-dihydroisoquinoline-2(1H)-carboxamide trihydroborate 26.

LCMS MC2: (M−H)⁻=440; purity=99%, retention time=2.057 min., MC method B).

¹H NMR (400 MHz, chloroform-d) δ 7.26-7.13 (m, 6H), 7.02-6.93 (m, 2H), 6.37 (d, J=5.1 Hz, 1H), 4.97-4.88 (m, 1H), 3.72-3.50 (m, 4H), 3.44-3.30 (m, 1H), 3.29-3.04 (m, 5H), 2.98-2.87 (m, 1H), 2.87-2.76 (m, 1H), 2.68-2.53 (m, 1H), 2.16-1.02 (m, 6H), OH not visible.

### Step 4: Synthesis of (1S)—N-((4-fluoro-3-hydroxyquinuclidin-3-yl)methyl)-1-(4-fluorophenyl)-3,4-dihydroisoquinoline-2(1H)-carboxamide

Aqueous HCl (2 M, 0.203 mL, 0.406 mmol) was added to a solution of (1 S)—N-((4-fluoro-3-hydroxyquinuclidin-3-yl)methyl)-1-(4-fluorophenyl)-3,4-dihydroisoquinoline-2(1H)-carboxamide trihydroborate (35.8 mg, 0.081 mmol) in acetone (3 mL). After 2 hours, the reaction mixture concentrated to dryness under reduced pressure. The residue was dissolved in methanol (2 mL) and brought onto an SCX-2 column (1 g) and eluted with methanol until neutral. Next, the column was eluted with ammonia in methanol (1 M). The basic fraction was concentrated to dryness under reduced pressure giving (1S)—N-((4-fluoro-3-hydroxyquinuclidin-3-yl)methyl)-1-(4-fluorophenyl)-3,4-dihydroisoquinoline-2(1H)-carboxamide 27.

LCMS MC1: (M+H)⁺=428; purity=93%, retention time=1.610 min., MC method A).

¹H NMR (400 MHz, chloroform-d) δ 7.30-7.10 (m, 6H), 7.06-6.90 (m, 2H), 6.38 (d, J=8.7 Hz, 1H), 5.00 (s, 1H), 3.86-3.71 (m, 1H), 3.66-3.50 (m, 2H), 3.50-3.41 (m, 2H), 3.22-3.10 (m, 1H), 3.10-2.75 (m, 6H), 2.49-2.28 (m, 1H), 2.00-1.86 (m, 1H), 1.86-1.74 (m, 1H), 1.58-1.44 (m, 1H).

### Step 5: Synthesis of 1-fluoro-2((S)-1-(4-fluorophenyl)-3,4-dihydroisoquinolin-2(1H)-yl)-4-4-azaspiro[bicyclo[2.2.2]octane-2,5oxazole]

Phosphorus oxychloride (32.7 μl, 0.351 mmol) was added to (1S)—N-((4-fluoro-3-hydroxyquinuclidin-3-yl)methyl)-1-(4-fluorophenyl)-3,4-dihydroisoquinoline-2(1H)-carboxamide 27 (30 mg, 0.070 mmol). The reaction mixture was stirred overnight, then quenched by the addition of methanol (1 mL) and stirred for another hour. The mixture was partitioned between chloroform (10 mL) and a mixture of water and saturated aqueous K₂CO₃ solution (1:1, 10 mL). The organic layer was passed through a phase separator and concentrated to dryness under reduced pressure. The residue was dissolved in dimethyl sulfoxide (1 mL) and purified by acidic preparative MPLC (Linear Gradient: t=0 min 5% A, t=1 min 5% A; t=16 min 30% A; t=17 min 100%; t=22 min 100% A; detection: 215/263 nm; Instrument type: Reveleris™ prep MPLC; Column: Phenomenex LUNA C18(3) (150×25 mm, 10p); Flow: 40 mL/min; Column temp: room temperature; Eluent A: 0.1% (v/v) Formic acid in acetonitrile, Eluent B: 0.1% (v/v) Formic acid in water). The product fraction of the first eluting diastereomer was lyophilized. The residue was dissolved in methanol (2 mL) and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give 1-fluoro-2 [symbol] ((S)-1-(4-fluorophenyl)-3,4-dihydroisoquinolin-2(1H)-yl)-4 [symbol] -4-azaspiro[bicyclo[2.2.2]octane-2,5 [symbol] oxazole], compound 3033. The product fractions of the second eluting diastereomer were combined and lyophilized. The residue was further purified by preparative chiral SFC (Phenomenex iAmylose-3 (100×4.6 mm 5 μm), flow: 4 mL/min; Column temp: 40° C.; BPR: 120 bar, 30% 20 mM ammonia in MeOH/CO₂). The product fractions were combined and lyophilized. The residue was dissolved in methanol (2 mL) and brought onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give 1-fluoro-2 [symbol] ((S)-1-(4-fluorophenyl)-3,4-dihydroisoquinolin-2(1H)-yl)-4 [symbol] -4-azaspiro[bicyclo[2.2.2]octane-2,5 [symbol] oxazole] compound 3034.

Compound 3033: LCMS MC1: (M+H)⁺=410; purity=100%, retention time=2.213 min., (MC method C). MC Chiral SFC: 100% retention time=1.449 min (MC method N)

¹H NMR (400 MHz, chloroform-d) δ 7.25-7.13 (m, 5H), 7.04 (d, J=7.4 Hz, 1H), 7.01-6.90 (m, 2H), 6.18 (s, 1H), 4.37 (d, J=12.4 Hz, 1H), 3.93 (ddd, J=13.1, 5.9, 4.1 Hz, 1H), 3.52 (d, J=12.4 Hz, 1H), 3.39 (ddd, J=13.2, 10.1, 4.5 Hz, 1H), 3.22 (dd, J=14.7, 4.9 Hz, 1H), 3.14 (t, J=7.9 Hz, 2H), 3.09-2.93 (m, 4H), 2.77 (dt, J=16.2, 4.3 Hz, 1H), 2.35-2.19 (m, 1H), 1.93-1.69 (m, 3H).

Compound 3034: LCMS MC1: (M+H)⁺=410; purity=99%, retention time=2.182 min., (MC method C). MC Chiral SFC: 99.5% retention time=2.177 min (MC method N)

¹H NMR (400 MHz, chloroform-d) δ 7.25-7.13 (m, 5H), 7.12-7.06 (m, 1H), 7.00-6.91 (m, 2H), 6.19 (s, 1H), 4.37 (d, J=12.5 Hz, 1H), 3.87 (dt, J=13.2, 5.2 Hz, 1H), 3.54 (d, J=12.5 Hz, 1H), 3.42-3.29 (m, 2H), 3.19-3.08 (m, 2H), 3.08-2.96 (m, 4H), 2.74 (dt, J=16.1, 4.5 Hz, 1H), 2.23-2.10 (m, 1H), 1.92-1.66 (m, 3H).

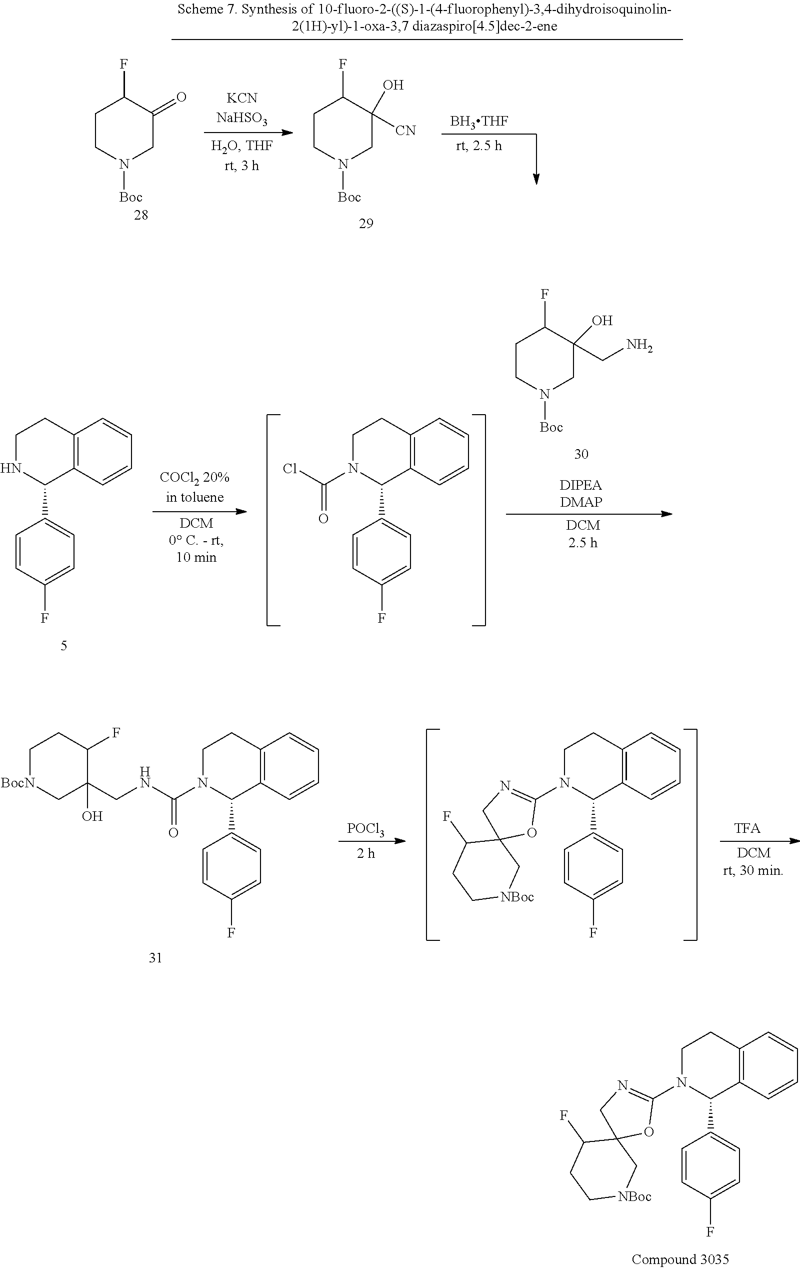

### Step 1: Synthesis of tert-butyl 3-cyano-4-fluoro-3-hydroxypiperidine-1-carboxylate

To a solution of tert-butyl 4-fluoro-3-oxopiperidine-1-carboxylate 28 as a mixture with of tert-butyl 4-fluoro-3,3-dihydroxypiperidine-1-carboxylate (1.0 g, 4.4 mmol) in tetrahydrofuran (4 mL), potassium cyanide (548 mg, 8.4 mmol) and water (5 mL) were added. Next a solution of sodium bisulfite (2.5 g, 24 mmol) in water (5 mL) was added. After stirring the reaction mixture at room temperature for 3 hours, the reaction mixture was extracted with dichloromethane (2×40 mL). The combined organic extracts were dried on Na₂SO₄ and evaporated under reduced pressure to afford tert-butyl 3-cyano-4-fluoro-3-hydroxypiperidine-1-carboxylate 29 which was used without further purification.

LCMS MC3: (M+H)⁺=245; purity=25% (ELSD), retention time=1.61 min., (MC method K).

¹H NMR (300 MHz, chloroform-d) δ 4.89 (m), 4.71 (m), 4.54 (m), 4.33-3.80 (br m), 3.79-3.56 (m), 3.55-2.79 (br m), 2.24-1.74 (br m), 1.48 (s).

### Step 2: Synthesis of tert-butyl 3-(aminomethyl)-4-fluoro-3-hydroxypiperidine-1-carboxylate

Borane-tetrahydrofuran complex (1.0 M, 15 mL, 15 mmol) was added to a solution of tert-butyl 3-cyano-4-fluoro-3-hydroxypiperidine-1-carboxylate 29 (crude, 0.45 g, 1.8 mmol) in tetrahydrofuran (15 mL). After 2.5 hours, water (10 mL) was added after which the organic solvent was removed by evaporation under reduced pressure. Dichloromethane (25 mL) was added followed by water (10 mL) and the organic solvent was removed by evaporated under reduced pressure. Aqueous NaOH solution (33%, 10 mL) was added and the mixture was extracted with dichloromethane (2×50 mL). The combined organic layers were dried on Na₂SO₄ and evaporated under reduced pressure to give the tert-butyl 3-(aminomethyl)-4-fluoro-3-hydroxypiperidine-1-carboxylate 30.

### Step 2: Synthesis of tert-butyl 3-(aminomethyl)-4-fluoro-3-hydroxypiperidine-1-carboxylate

Borane-tetrahydrofuran complex (1.0 M, 15 mL, 15 mmol) was added to a solution of tert-butyl 3-cyano-4-fluoro-3-hydroxypiperidine-1-carboxylate 29 (crude, 0.45 g, 1.8 mmol) in tetrahydrofuran (15 mL). After 2.5 hours, water (10 mL) was added after which the organic solvent was removed by evaporation under reduced pressure. Dichloromethane (25 mL) was added followed by water (10 mL) and the organic solvent was removed by evaporated under reduced pressure. Aqueous NaOH solution (33%, 10 mL) was added and the mixture was extracted with dichloromethane (2×50 mL). The combined organic layers were dried on Na₂SO₄ and evaporated under reduced pressure to give the tert-butyl 3-(aminomethyl)-4-fluoro-3-hydroxypiperidine-1-carboxylate 30.

LCMS MC3: (M+H)+=249; 1:4 mixture of isomers, retention times=0.83 min. and 1.04 min., (MC method K).

¹H NMR (300 MHz, chloroform-d) δ 4.89-4.38 (m), 3.94-3.61 (m), 3.55-3.24 (m), 3.10-2.90 (m), 2.91-2.65 (m), 2.65-2.26 (m), 2.22-1.60 (m), 1.45 (s, 9H).

### Step 3: Synthesis of tert-butyl 4-fluoro-3-(((S)-1-(4-fluorophenyl)-1,2,3,4-tetrahydroisoquinoline-2-carboxamido)methyl)-3-hydroxypiperidine-1-carboxylate

At 0° C., a solution of (S)-1-(4-fluorophenyl)-1,2,3,4-tetrahydroisoquinoline 5 (850 mg, 3.74 mmol) in dichloromethane (10 mL) was added to a solution of phosgene (20% in toluene, 21.6 mL, 41.1 mmol). After stirring for 10 minutes at room temperature, the reaction mixture was evaporated under reduced pressure (at 60° C.). The residue was taken up in dichloromethane (10 mL) and the resulting slurry was added to a solution of tert-butyl 3-(aminomethyl)-4-fluoro-3-hydroxypiperidine-1-carboxylate 30 (555 mg, 2.24 mmol), N,N-diisopropylethylamine (1.25 g, 9.67 mmol), and 4-dimethylaminopyridine (32 mg, 0.26 mmol) in dichloromethane (10 mL). After 2.5 hours, the reaction mixture was evaporated under reduced pressure (at 60° C.). The residue was purified by flash silica gel column chromatography (0% to 70% ethyl acetate in heptane) to give tert-butyl 4-fluoro-3-(((S)-1-(4-fluorophenyl)-1,2,3,4-tetrahydroisoquinoline-2-carboxamido)methyl)-3-hydroxypiperidine-1-carboxylate 31.

LCMS MC3: (M+H)+=502; purity=99.5%, retention time=2.37 min., (MC method K).

1H NMR (300 MHz, chloroform-d) δ 7.29-7.10 (m, 6H), 6.95 (t, J=8.6, 2H), 6.42 (d, J=13.3 Hz, 1H), 5.78-5.29 (br s, 1H), 4.65-4.45 (m, 1H), 3.91-3.39 (m, 5H), 3.11-2.73 (m, 6H), 2.17-1.97 (m, 1H), 1.76-1.63 (m, 1H), 1.45 (s, 9H).

### Step 4: Synthesis of 10-fluoro-2-((S)-1-(4-fluorophenyl)-3,4-dihydroisoquinolin-2(1H)-yl)-1-oxa-3,7-diazaspiro[4.5]dec-2-ene (Compound 3035)

Phosphorus oxychloride (3 mL, 30 mmol) was added to tert-butyl 4-fluoro-3-(((S)-1-(4-fluorophenyl)-1,2,3,4-tetrahydroisoquinoline-2-carboxamido)methyl)-3-hydroxypiperidine-1-carboxylate 31 (670 mg, 1.34 mmol). After 2 hours at room temperature the reaction mixture was evaporated under reduced pressure (at 60° C.). The residue was dissolved in dichloromethane (10 mL) and trifluoroacetic acid (3 mL, 40 mmol) was added. After 30 minutes, the reaction mixture was evaporated under reduced pressure (at 60° C.). The residue was taken up into dichloromethane (50 mL) and aqueous NaOH (15%, 10 mL) and the resulting mixture was mixed thoroughly. The layers were separated, the aqueous phase was diluted with aqueous ammonia (10%, 25 mL) and extracted with dichloromethane (50 ml). The combined organic layers were dried on Na₂SO₄ and evaporated under reduced pressure. The residue was purified by flash silica gel column chromatography (0% to 10% methanol in dichloromethane) to give 10-fluoro-2-((S)-1-(4-fluorophenyl)-3,4-dihydroisoquinolin-2(1H)-yl)-1-oxa-3,7-diazaspiro[4.5]dec-2-ene, compound 3035 as a mixture of diastereomers.

LCMS MC3: (M+H)+=384; purity=99.2% (1:2 mixture of diastereomers), retention times=1.475 min. and 1.493 min., (MC method E).

¹H NMR (300 MHz, chloroform-d) 1:1 mixture of diastereomers δ 7.27-7.11 (m, 5H), 7.09-7.00 (m, 1H), 7.04-6.89 (m, 2H), 6.20 (s, 1H), 4.72-4.56 (m, 1H), 3.98-3.84 (m, 2H), 3.60-3.45 (m, 1H), 3.42-3.26 (m, 1H), 3.12-2.93 (m, 3H), 2.89-2.68 (m, 3H), 2.11-1.88 (m, 2H), 1.87-1.67 (m, 1H).

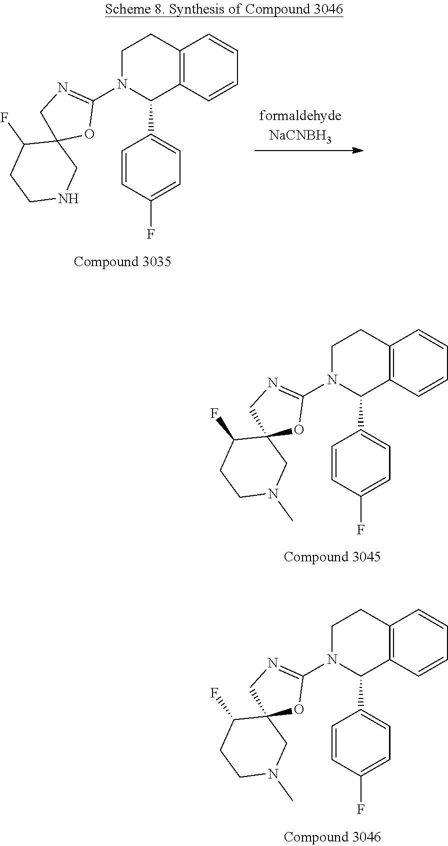 [SMILES: Fc1ccc([C@H]2c3ccccc3CCN2C2=NCC3(CNCCC3F)O2)cc1 ; CN1CC[C@@H](F)[C@]2(CN=C(N3CCc4ccccc4[C@@H]3c3ccc(F)cc3)O2)C1 ; CN1CC[C@H](F)[C@]2(CN=C(N3CCc4ccccc4[C@@H]3c3ccc(F)cc3)O2)C1]

### Synthesis of (5R,10R)-10-fluoro-2-((S)-1-(4-fluorophenyl)-3,4-dihydroisoquinolin-2(1H)-34)-7-methyl-1-oxa-3,7-diazaspiro[4.5]dec-2-ene, Compound 3045

Formaldehyde (37 wt % solution in water, stabilized with 10-15% methanol, 0.3 mL, 4 mmol) was added to a solution of 10-fluoro-2-((S)-1-(4-fluorophenyl)-3,4-dihydroisoquinolin-2(1H)-yl)-1-oxa-3,7-diazaspiro[4.5]dec-2-ene (182 mg, 0.475 mmol; mixture of diastereomers (˜1:4)) in methanol (5.0 mL). Sodium cyanoborohydride (270 mg, 4.30 mmol) was added and the resulting mixture was stirred at room temperature for 20 minutes. The reaction mixture was diluted with water (40 mL) and extracted with two 50 mL portions of ethyl acetate. The combined organics were dried on Na₂SO₄ and evaporated under reduced pressure. Part of the residue was purified by preparative chiral SFC (MC method T). The major, first eluting peak was evaporated and the residue was lyophilized from acetonitrile/water giving (5R,10R)-10-fluoro-2-((S)-1-(4-fluorophenyl)-3,4-dihydroisoquinolin-2(1H)-yl)-7-methyl-1-oxa-3,7-diazaspiro[4.5]dec-2-ene. Stereochemistry at the C5 and 010 positions are assigned arbitrarily.

LCMS MC3: (M+H)+=398; purity=100%, retention time=1.976 min.

MC Chiral SFC: 100%, retention time=1.811 min., (M+H)⁺=398 (MC method V).

¹H NMR (400 MHz, chloroform-d) δ 7.25-7.12 (m, 5H), 7.01 (d, J=7.4 Hz, 1H), 6.98-6.90 (m, 2H), 6.21 (s, 1H), 4.57 (ddd, J=49.5, 8.3, 4.2 Hz, 1H), 3.96-3.87 (m, 2H), 3.58 (d, J=12.8 Hz, 1H), 3.28 (ddd, J=13.2, 10.8, 4.5 Hz, 1H), 3.03 (ddd, J=16.7, 10.7, 6.2 Hz, 1H), 2.74 (dt, J=16.2, 4.0 Hz, 1H), 2.64-2.50 (m, 2H), 2.46-2.22 (m, 2H), 2.31 (s, 3H), 2.15-2.01 (m, 1H), 1.92-1.78 (m, 1H).

### (5R,10S)-10-fluoro-2-((S)-1-(4-fluorophenyl)-3,4-dihydroisoquinolin-2(1H)-yl)-7-methyl-1-oxa-3,7-diazaspiro[4.5]dec-2-ene, Compound 3046

Formaldehyde (37 wt % solution in water, stabilized with 10-15% methanol, 0.2 mL, 2.7 mmol) was added to a solution of 10-fluoro-2-((S)-1-(4-fluorophenyl)-3,4-dihydroisoquinolin-2(1H)-yl)-1-oxa-3,7-diazaspiro[4.5]dec-2-ene (44 mg, 0.11 mmol; mixture of diastereomers (˜3:1)) in methanol (5.0 mL). Sodium cyanoborohydride (170 mg, 2.71 mmol) was added and the resulting mixture was stirred at room temperature for 20 minutes. The reaction mixture was diluted with water (40 mL) and extracted with two 50 mL portions of ethyl acetate. The combined organics were dried on Na₂SO₄ and evaporated under reduced pressure. The residue was purified by preparative chiral SFC (MC method T). The major, second eluting peak was evaporated. The residue was further purified by preparative chiral SFC (MC method U). The first major eluting peak was evaporated and the residue was lyophilized from acetonitrile/water giving compound 3046 (5R,10S)-10-fluoro-2-((S)-1-(4-fluorophenyl)-3,4-dihydroisoquinolin-2(1H)-yl)-7-methyl-1-oxa-3,7-diazaspiro[4.5]dec-2-ene. Stereochemistry at the C5 and 010 positions are assigned arbitrarily.

LCMS MC3: (M+H)+=398; purity=95%, retention time=1.868 min

MC Chiral SFC: 98%, retention time=1.852 min., (M+H)+=398 (MC method V).

¹H NMR (400 MHz, chloroform-d) δ 7.25-7.13 (m, 5H), 7.04 (d, J=7.4 Hz, 1H), 6.98-6.90 (m, 2H), 6.20 (s, 1H), 4.55 (ddd, J=49.4, 8.4, 4.1 Hz, 1H), 3.96-3.86 (m, 2H), 3.61 (br d, J=12.8 Hz, 1H), 3.30 (ddd, J=13.2. 10.3, 4.5 Hz, 1H), 3.03 (ddd, J=16.2, 10.1, 6.0 Hz, 1H), 2.73 (dt, J=16.1, 4.1 Hz, 1H), 2.68-2.52 (m, 2H), 2.48-2.34 (m, 1H), 2.33-2.19 (m, 1H), 2.29 (s, 3H), 2.14-1.99 (m, 1H), 1.93-1.78 (m, 1H).

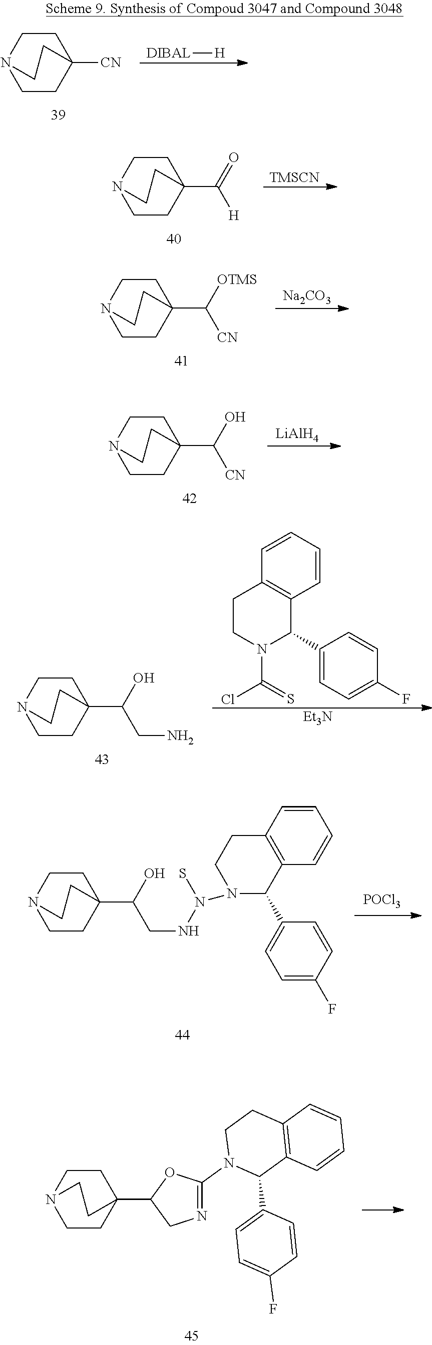

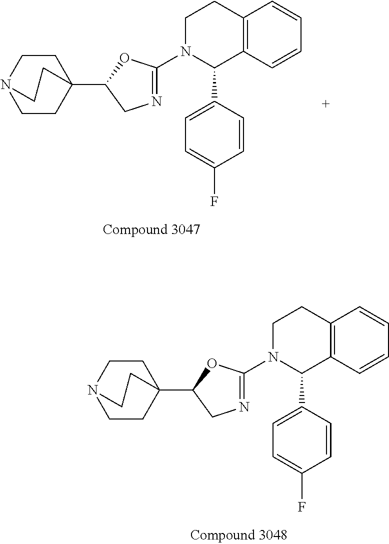 [SMILES: Fc1ccc([C@H]2c3ccccc3CCN2C2=NC[C@H](C34CCN(CC3)CC4)O2)cc1 ; Fc1ccc([C@H]2c3ccccc3CCN2C2=NC[C@@H](C34CCN(CC3)CC4)O2)cc1]

Step 1: To a solution of 4-cyano-quinuclidine 39 (2.72 g, 20 mmol) in methylene chloride (20 mL) at −78° C. was added dropwise diisobutylaluminum hydride (1 M solution in hexanes, 40 mmol, 40 mL) and the resulting reaction mixture was stirred at the same temperature for 2 hours. The reaction mixture was quenched with saturated aqueous solution of sodium potassium tartrate (30 mL) and the mixture was allowed to warm up to room temperature. The aqueous phase was extracted with methylene chloride (3×30 mL) and the combined organic phases were washed with water, then dried (Na₂SO₄) and concentrated to afford crude quinuclidine-4-carbaldehyde 40. The crude product was used without further purification. LCMS: (M+H₂O)+=158; Retention time=0.340 min. LCMS CP Method B

Step 2: To a solution of quinuclidine-4-carbaldehyde 40 (1.71 g, 12 mmol) and TMSCN (1.9 mL, 15 mmol) in dry tetrahydrofuran (30 mL) was added ZnI₂ (0.19 g, 0.6 mmol) at room temperature. The reaction mixture was heated to reflux for 16 hours, then cooled and filtered. The filtrate was concentrated to give crude 2-(quinuclidin-4-yl)-2-(trimethylsilyloxy)acetonitrile 41 which was used in the next step without any further purification. LCMS: (M+H)⁺=239; Retention time 1.239 min. LCMS CP Method B

Step 3: To an ice-cold solution of 2-(quinuclidin-4-yl)-2-(trimethylsilyloxy)acetonitrile 41 (2.5 g, 10.5 mmol) in MeOH (40 mL) was added K₂CO₃ (1.45 g, 10.5 mmol). The reaction mixture was stirred at room temperature for 1 hour, then filtered, the filtrate was concentrated to give crude 2-hydroxy-2-(quinuclidin-4-yl)acetonitrile 42. LCMS: (M+1)+=167; Retention time 0.945 min. LCMS CP Method B

Step 4: To an ice-cold solution of 2-hydroxy-2-(quinuclidin-4-yl)acetonitrile 42 (1.6 g, 9.6 mmol) in dry tetrahydrofuran (40 mL) was added LiAlH₄ (10 mL, 10 mmol, 1M in tetrahydrofuran) dropwise. The reaction mixture was stirred at 0° C. for 2 hours, then quenched by saturated aqueous sodium sulfate solution. The mixture was diluted with tetrahydrofuran (100 mL) and anhydrous Na₂SO₄ was added. The mixture was stirred for 20 min at room temperature and then filtered. The filtrate was concentrated to give crude 2-amino-1-(quinuclidin-4-yl)ethanol 43.

LCMS: (M+1)+=243; Retention time 0.889 min. LCMS CP Method B

Step 5: To a solution of (S)-1-(4-fluorophenyl)-1,2,3,4-tetrahydroisoquinoline 5 (0.5 g, 2.2 mmol) in dry CH₃CN (10 mL) was added thiophosgene (0.25 mL, 3.3 mmol) at 0° C. The reaction mixture was stirred at room temperature for 2 hours. The reaction mixture was concentrated, and the residue was dissolved in dry DMF (10 mL). 2-amino-1-(quinuclidin-4-yl)ethanol 43 (450 mg, 2.6 mmol) and TEA (0.9 mL, 6.6 mmol) were added and the reaction mixture was stirred at room temperature for 1 hour. The reaction mixture was purified by prep-HPLC to give (1S)-1-(4-fluorophenyl)-N-(2-hydroxy-2-(quinuclidin-4-yl)ethyl)-3,4-dihydroisoquinoline-2(1H)-carbothioamide 44. LCMS: (M+1)+=440; Retention time=1.527 min. LCMS CP Method A

Step 6: (1S)-1-(4-fluorophenyl)-N-(2-hydroxy-2-(quinuclidin-4-yl)ethyl)-3,4-dihydro-isoquinoline-2(1H)-carbothioamide 44 (220 mg, 0.72 mmol) was dissolved in POCl₃ (3 mL) at 0° C. The reaction mixture was heated to 120° C. for 1 hour. The reaction mixture was cooled and concentrated. The residue was dissolved in water (15 mL) and basified by NaHCO₃. Then the mixture was extracted with three 30 mL portions of DCM. The combined organic layers were dried and concentrated. The crude compound was purified by prep-HPLC to give 2-((S)-1-(4-fluorophenyl)-3,4-dihydroisoquinolin-2(1H)-yl)-5-(quinuclidin-4-yl)-4,5-dihydrooxazole 45.

LCMS: (M+1)+=406; Retention time=1.72 min. LCMS CP Method C

The diastereomers were separated by chiral SFC eluting with CO₂/EtOH containing 0.5% methanolic ammonia over an AD column (20×250 mm, 10 μm) to give compound 3047 and compound 3048. Stereochemical assignment of (S) at the 1 position of the tetrahydroisoquinoline is based on enantiomerically pure starting materials; the stereochemical assignment at the dihydrooxazole chiral center is based on chromatographic elution order as compared to related analogues of known configuration and therefore is assumed.

Compound 3047: LCMS: (M+1)+=406; Retention time=1.725 min. LCMS CP Method C

1HNMR (400 Hz, CDCl3): 7.23-7.16 (m, 5H), 7.06-6.94 (m, 3H), 6.20 (s, 1H), 4.24-4.20 (m, 1H), 3.94-3.89 (m, 1H), 3.74-3.64 (m, 2H), 3.33-3.26 (m, 1H), 3.10-3.02 (m, 1H), 2.88 (t, J=7.8H, 6H), 2.77-2.72 (m, 1H), 1.51-1.44 (m, 3H), 1.36-1.27 (m, 3H).

Chiral SFC: CO₂/MeOH containing 0.2% ammonia over a chiral IG column (4.6×100 mm, 5 μm), retention time=8.286 min), 100% ee.

Compound 3048: LCMS: (M+1)+=406; Retention time=1.728 min. LCMS CP Method C

1HNMR (400 Hz, CDCl3): 7.26-7.17 (m, 5H), 7.05-6.97 (m, 3H), 6.14 (s, 1H), 4.27 (t, J=8.4 Hz, 1H), 3.98-3.92 (m, 1H), 3.76-3.63 (m, 2H), 3.46-3.40 (m, 1H), 3.08-2.99 (m, 7H), 2.84-2.78 (m, 1H), 1.60-1.53 (m, 3H), 1.45-1.35 (m, 3H).

Chiral SFC: CO₂/MeOH containing 0.2% ammonia over a chiral IG column (4.6×100 mm, 5 μm), retention time=8.335 min), 100% ee.

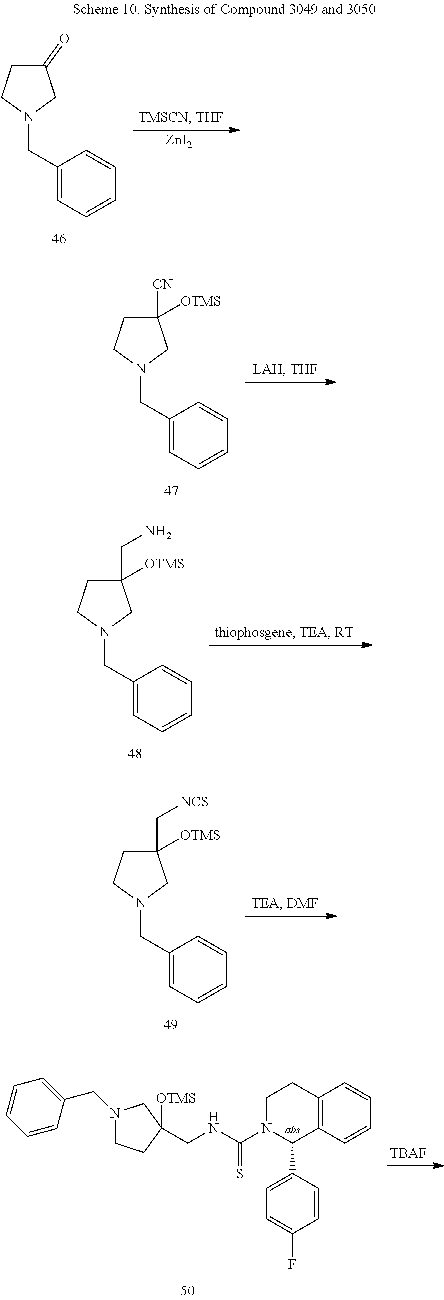

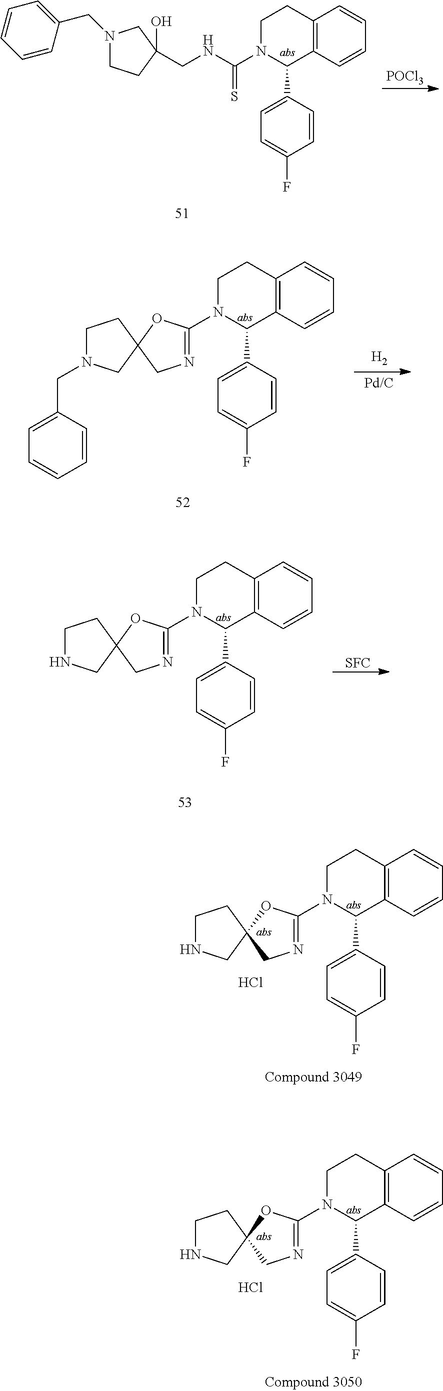

Step 1: A solution of pyrrolidinone 46 (3.5 g, 20 mmol), TMSCN (1.98 g 20 mmol) and ZnI2 (319 mg 1 mmol) in tetrahydrofuran (100 mL) was stirred at 60° C. until TLC analysis indicated the total consumption of the starting material. The solvent was evaporated to give crude compound 47.

Step 2: To a solution of 47 (5.48 g, 20 mmol) in tetrahydrofuran (100 mL) at 0° C. was added 20 mL of LAH (1M). The reaction mixture was stirred at room temperature until TLC analysis indicated the total consumption of the starting material. The reaction was quenched with saturated Na₂SO₄ to give crude 48. LCMS: (M+H)⁺=279 (No UV); MS Retention time=1.786 min. LCMS CP Method C

Step 3: To a solution of 48 (2.78 g, 10 mmol) and Et3N (3.03 g 30 mmol) in tetrahydrofuran (100 mL) at 0° C. was added thiophosgene (0.5 g, 5 mmol). The reaction mixture was stirred at room temperature until TLC analysis indicated the total consumption of the starting material. The solvent was evaporated to give crude compound 49.

Step 4: To a solution of (S)-1-(4-fluorophenyl)-1,2,3,4-tetrahydroisoquinoline 5 (2.27 g, 10 mmol) and Et₃N (3.03 g, 30 mmol) in DMF (100 mL) at 0° C. was added 49 (3.20 g, 10 mmol). The reaction mixture was stirred at 60° C. until TLC analysis indicated the total consumption of the starting material. The reaction was quenched with water and extracted with ethyl acetate. The solvent was evaporated to give crude product which was purified by prep-HPLC to give compound 50. LCMS: (M+H)⁺=548 (UV 214 nm); Retention time=1.568 min. LCMS CP Method C

Step 5: To a solution of compound 50 (1096 mg, 2.0 mmol) in tetrahydrofuran (50 mL) at 0° C. was added TBAF (3 mmol). The reaction mixture was stirred at room temperature until TLC analysis indicated the total consumption of the starting material. The solvent was evaporated to give crude product which was purified by prep-HPLC to give compound 51. LCMS: (M+H)⁺=476 (UV 214 nm); Retention time=2.197 min. LCMS CP Method C

Step 6: To a solution of 51 (475 mg, 1 mmol) in tetrahydrofuran (10 mL) at 0° C. was added phosphoryl trichloride (153 mg, 1 mmol). The reaction mixture was stirred at 60° C. until TLC analysis indicated the total consumption of the starting material. The solvent was evaporated to give crude product which was purified by prep-HPLC to give compound 52. LCMS: (M+H)⁺=442 (UV 214 nm); Retention time=1.846 min. LCMS CP Method C

Step 7: To a solution of 52(150 mg, 0.34 mmol) in methanol (10 mL) was added Pd/C (27 mg, 10% w/w) under hydrogen atmosphere. The reaction mixture was stirred at room temperature for 16 h. The solvent was evaporated to give crude product which was purified by prep-HPLC to give compound 53.

The diastereomers were separated by chiral SFC eluting with CO₂/MeOH containing 0.2% methanolic ammonia over a chiral IG column (4.6*100 mm, 5 μm) to give compound 3049 (retention time=1.030 min) and compound 3050 (retention time=1.515 min). Stereochemical assignment of (S) at the 1 position of the tetrahydroisoquinoline is based on enantiomerically pure starting materials; the stereochemical assignment at the dihydrooxazole spirocyclic chiral center is based on chromatographic elution order as compared to related analogues of known configuration and therefore is assumed.

Compound 3049: LCMS: (M+H)⁺=352; purity=100% (214 nm); retention time=1.259 min. LCMS CP Method C

¹H NMR (400 MHz, CD₃OD) δ 7.34-7.15 (m, 5H), 7.11-6.96 (m, 3H), 6.16 (s, 1H), 3.95-3.71 (m, 3H), 3.39 (ddd, J=8.6, 7.8, 3.7 Hz, 1H), 3.18-3.07 (m, 2H), 3.06-2.94 (m, 2H), 2.91-2.73 (m, 2H), 2.28-2.16 (m, 1H), 2.00-1.90 (m, 1H).

Chiral SFC: CO₂/MeOH containing 0.2% ammonia over a chiral IG column (4.6*100 mm, 5 μm), retention time=1.030 min), 100% ee.

Compound 3050: LCMS: (M+H)⁺=352; purity=100% (214 nm); retention time=1.515 min. LCMS CP Method C

¹H NMR (400 MHz, CD₃OD) δ 7.41-7.27 (m, 5H), 7.22 (d, J=7.4 Hz, 1H), 7.14 (t, J=8.7 Hz, 2H), 6.47 (s, 1H), 4.20 (q, J=10.7 Hz, 2H), 4.06 (d, J=12.6 Hz, 1H), 3.83 (dd, J=8.1, 4.9 Hz, 2H), 3.74-3.52 (m, 3H), 3.23-3.12 (m, 1H), 3.00 (dt, J=16.3, 5.1 Hz, 1H), 2.63 (s, 1H), 2.51 (dt, J=14.7, 10.0 Hz, 1H).

Chiral SFC: CO₂/MeOH containing 0.2% ammonia over a chiral IG column (4.6*100 mm 5 μm), retention time=1.515 min), 100% ee.

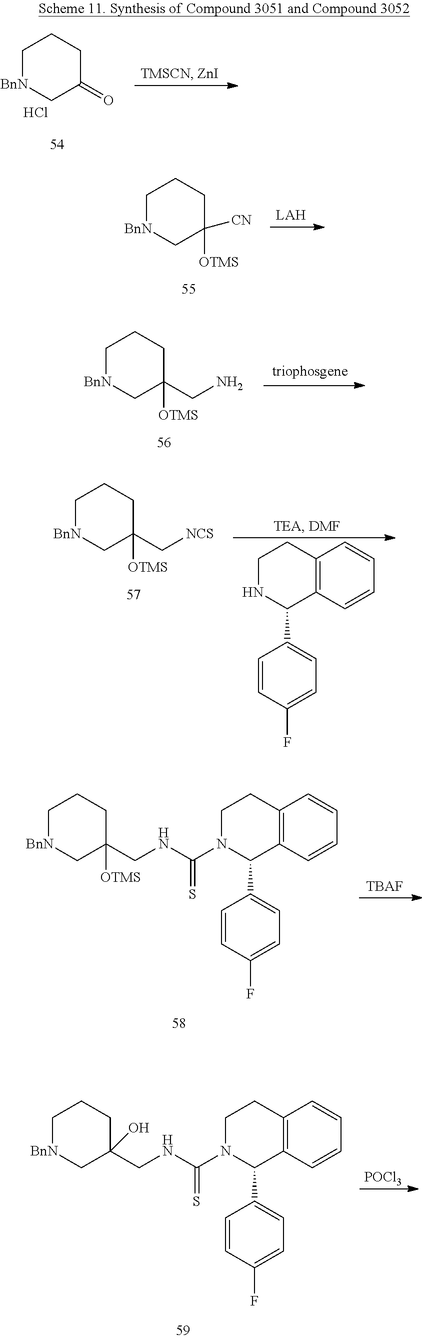

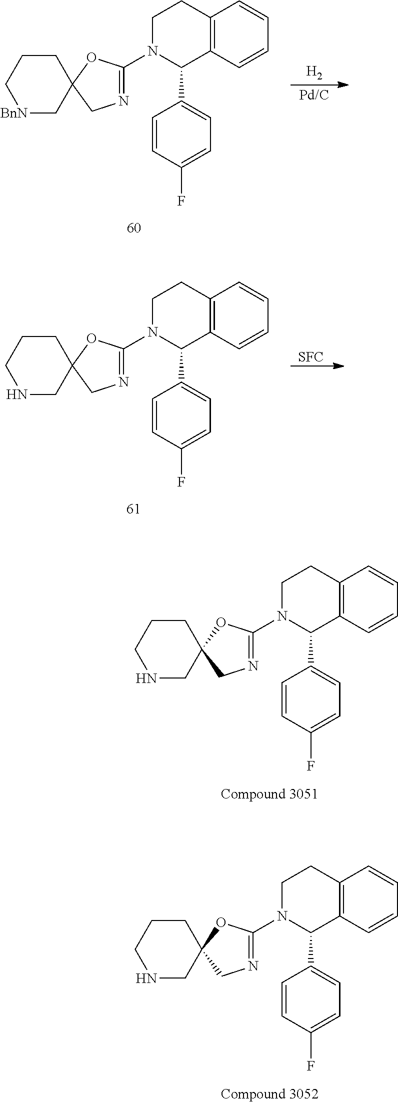

Compound 3051 and compound 3052 were prepared following a similar synthesis as for compounds 3049 and 3050 described above, except in the hydrogenation step, Pd(OH)₂ (20% w/w) was used.

The diastereomers were separated by chiral SFC eluting with n-hexane (0.1% DEA): EtOH (0.1% DEA)=70:30 over a chiral IG column (4.6*250 mm, 5 μm) to give compound 3051 (retention time=8.694 min) and compound 3052 (retention time=11.184 min). Stereochemical assignment of (S) at the 1 position of the tetrahydroisoquinoline is based on enantiomerically pure starting materials; the stereochemical assignment at the dihydrooxazole spirocyclic chiral center is based on chromatographic elution order as compared to related analogues of known configuration and therefore is assumed.

Compound 3051: LCMS: (M+H)⁺=365, purity=97.9% (214 nm), Retention time=1.704 min. LCMS CP Method C

¹H NMR (400 MHz, CDCl₃) δ 7.24-7.14 (m, 5H), 7.04 (d, J=7.4 Hz, 1H), 6.99-6.92 (m, 2H), 6.22 (s, 1H), 3.94 (ddd, J=13.2, 5.8, 3.7 Hz, 1H), 3.52 (dd, J=33.0, 12.2 Hz, 2H), 3.33 (ddd, J=13.3, 10.5, 4.5 Hz, 1H), 3.03 (ddd, J=26.7, 16.4, 9.6 Hz, 2H), 2.91 (d, J=13.5 Hz, 1H), 2.81-2.69 (m, 3H), 1.97-1.91 (m, 1H), 1.61-1.53 (m, 1H), 1.44-1.39 (m, 1H), 1.39-1.30 (m, 2H).

Compound 3052: LCMS: (M+H)⁺=365, purity=100% (214 nm), Retention time=1.737 min. LCMS CP Method C

¹H NMR (400 MHz, CDCl₃) δ 7.23-7.14 (m, 5H), 7.05-6.99 (m, 1H), 6.95 (td, J=8.8, 2.2 Hz, 2H), 6.18 (d, J=9.0 Hz, 1H), 3.92 (ddd, J=13.1, 5.9, 3.8 Hz, 1H), 3.54 (d, J=12.2 Hz, 1H), 3.47 (d, J=12.3 Hz, 1H), 3.38-3.28 (m, 1H), 3.04 (ddd, J=16.4, 12.0, 6.7 Hz, 1H), 2.88 (dd, J=22.8, 9.5 Hz, 2H), 2.80-2.66 (m, 3H), 1.93 (d, J=9.4 Hz, 1H), 1.58-1.51 (m, 1H), 1.43-1.40 (m, 1H), 1.38-1.32 (m, 2H).

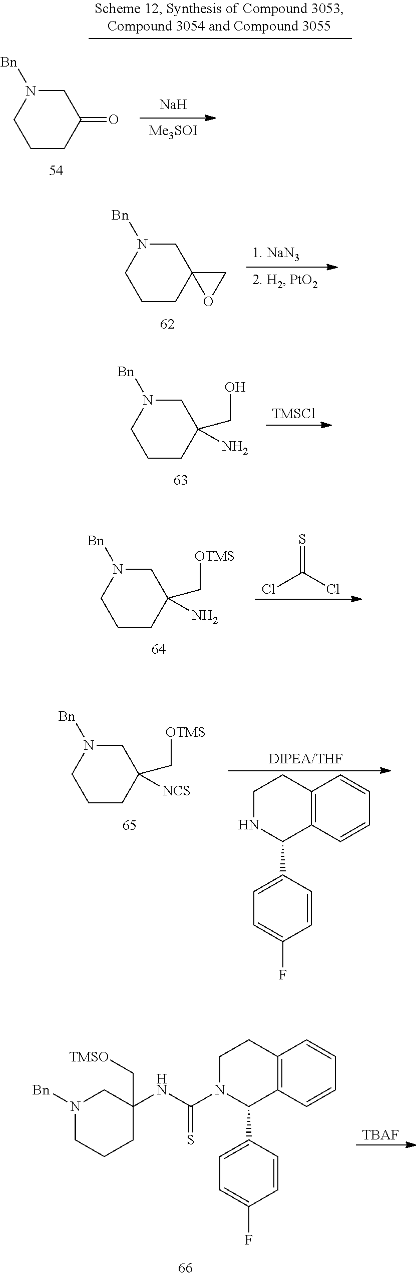

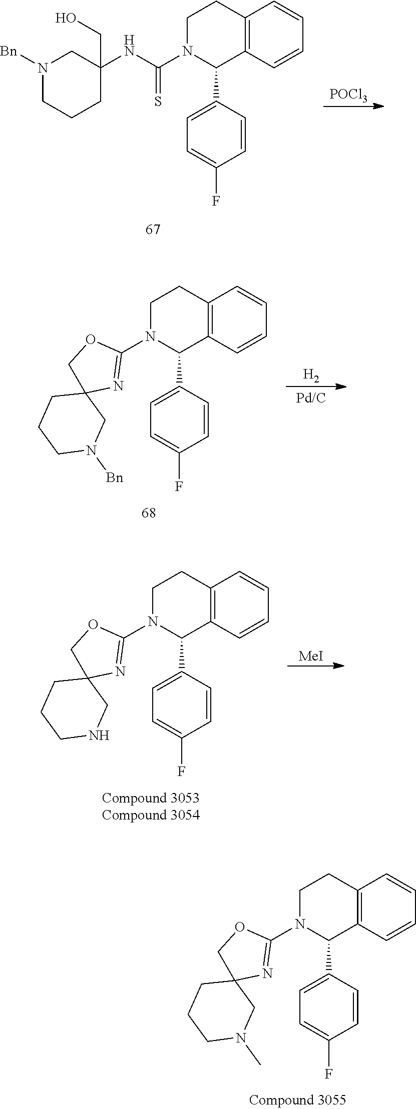

Step 1: To a solution of trimethylsulfoxonium iodide (4.13 g, 18.78 mmol) in anhydrous DMSO (60 mL) was added NaH (0.450 g, 18.75 mmol) under nitrogen. The solution was stirred until bubbling had stopped and a solution of 1 equivalent of the piperidone in DMSO was added slowly with stirring. The resulting mixture was stirred at room temperature for 6 hours, and then at 50° C. for 40 minutes. The mixture was cooled to room temperature, quenched with water (12 mL), then extracted with ether (5 times). The combined organic layers were washed with water, dried over Na2SO4, and concentrated. The crude material was purified by silica gel chromatography eluting with a gradient of EtOAc:Hex from 1:9 to 6:4 to yield epoxide 62. LCMS: (M+H)+=204; Retention time=1.672 min. LCMS CP Method C

Step 2: To the epoxide 62 (2.03 g, 0.01 mol) in DMSO/H2O (50 mL) at 0° C. was added NaN₃ (1.3 g, 0.02 mol). The mixture was stirred at 80° C. until 62 was consumed. The reaction mixture was extracted with two 100 mL portions of ethyl acetate and the combined organic layers were concentrated to give the crude product. MeOH (200 mL) was then added, followed by PtO₂ (1.0 g) under H₂. The mixture was stirred at room temperature until starting material was all converted to amino alcohol 63. The reaction was filtered and the filtrate was concentrated to give the crude product 63 which was used without further purification. LCMS: (M+H)+=221; Retention time=1.067 min. LCMS CP Method C2

Step 3: To a solution of alcohol 63 (1.1 g, 5 mmol) and DIPEA (1.30 g 20 mmol) in tetrahydrofuran (50 mL) at 0° C. was added TMSCl (1.08 g, 20 mmol). The reaction mixture was stirred at room temperature until TLC analysis indicated the total consumption of starting material. The solvent was evaporated to give crude compound 64 which was used directly in the next step reaction without further purification. LCMS: (M+H)+=293; Retention time=1.732 min. LCMS CP Method C

Step 4: To a solution of compound 64 (1.46 g, 5 mmol) and thiophosgene (1.15 g 10 mmol) in tetrahydrofuran (50 mL) at 0° C. was added DIPEA (1.30 g, 10 mmol). The reaction mixture was stirred at 0° C. until TLC analysis indicated the total consumption of the starting material. The solvent was evaporated to give crude compound 65 which was used directly in the next step reaction without further purification.

Step 5: To a solution of compound 65 (1.67 g, 5 mmol) and (S)-1-(4-fluorophenyl)-1,2,3,4-tetrahydroisoquinoline 5 (2.27 g 10 mmol) in tetrahydrofuran (60 mL) at 0° C. was added DIPEA (1.30 g, 10 mmol). The reaction mixture was stirred at 0° C. until TLC analysis indicated the total consumption of the starting material. The solvent was evaporated and the residue was purified by flash silica gel column eluting with MeOH/DCM=1:10 to give crude product 66. LCMS: (M+H)+=562; Retention time=1.940 min. LCMS CP Method C

Step 6: To a solution of compound 66 (280 mg, 0.25 mmol) in tetrahydrofuran (10 mL) at 0° C. was added TBAF (1 mL 0.25 mmol). The reaction mixture was stirred at room temperature until TLC analysis indicated the total consumption of the starting material. The solvent was evaporated to give the crude product 67 which was used directly in the next step reaction without further purification. LCMS: (M+H)+=490; Retention time=1.543 min. LCMS CP Method C1

Step 7: A solution of compound 67 (400 mg, 0.8 mmol) in POCl₃ (5 mL) was stirred at 70° C. until TLC analysis indicated the total consumption of the starting material. The solvent was evaporated and the residue was purified by prep-HPLC to give compound 68. LCMS: (M+H)+=456; Retention time=1.783 min. LCMS CP Method C

Step 8: To a solution of compound 68 (150 mg, 0.33 mmol) in MeOH (50 mL) was added Pd/C (500 mg) and the mixture was stirred at room temperature under H₂. Once the starting material was consumed, the reaction was filtered and concentrated to give the crude product which was further purified by prep-HPLC. LCMS: (M+H)⁺=366; Retention time=1.152 min. LCMS CP Method B

The diastereomers were separated by chiral SFC eluting with n-hexane (0.1% DEA):EtOH (0.1% DEA)=70:30 over a chiral IG column (4.6*250 mm, 5 μm) to give compound 3053 and compound 3054. Stereochemical assignment of (S) at the 1 position of the tetrahydroisoquinoline is based on enantiomerically pure starting materials; the stereochemical assignment at the dihydrooxazole spirocyclic chiral center is based on chromatographic elution order and therefore is assumed.

Compound 3053: LCMS: (M+H)+=366; purity=100% (214 nm); retention time=1.149 min. LCMS CP Method B

¹H NMR (400 MHz, CD₃OD) δ 7.27-7.12 (m, 5H), 7.03 (ddd, J=14.6, 8.8, 4.7 Hz, 3H), 6.26 (s, 1H), 3.88-3.77 (m, 1H), 3.50 (d, J=12.2 Hz, 1H), 3.46-3.35 (m, 2H), 2.93 (ddd, J=17.4, 9.1, 5.1 Hz, 3H), 2.83-2.60 (m, 3H), 1.92 (d, J=12.1 Hz, 1H).

Chiral SFC: CO₂/MeOH containing 0.2% ammonia over a chiral IG column (4.6*100 mm, 5 μm), retention time=1.040 min), 100% ee

Compound 3054: LCMS: (M+H)⁺=366; purity=100% (214 nm); retention time=1.150 min. LCMS CP Method B

¹H NMR (400 MHz, CD₃OD) δ 7.29-7.16 (m, 5H), 7.06 (ddd, J=17.5, 10.5, 4.7 Hz, 3H), 6.18 (s, 1H), 3.91-3.81 (m, 1H), 3.57-3.37 (m, 3H), 3.02 (ddd, J=15.9, 9.7, 6.0 Hz, 1H), 2.90-2.77 (m, 3H), 2.75-2.64 (m, 2H), 2.01-1.88 (m, 1H), 1.88-1.73 (m, 2H), 1.65-1.54 (m, 1H).

Chiral SFC: CO₂/MeOH containing 0.2% ammonia over a chiral IG column (4.6*100 mm, 5 μm), retention time=1.075 min), 100% ee

Step 9: A solution of mixture of diastereomers above (91 mg, 0.25 mmol), K₂CO₃ (138 mg, 1 mmol) and MeI (355 mg, 2.5 mmol) in DMF (5 mL) was stirred at 70° C. until TLC analysis indicated the total consumption of the starting material. The solvent was evaporated and the residue was purified by prep-HPLC and to give the desired compound.

Compound 3055: LCMS: (M+H)⁺=380; purity=95.64% (214 nm); retention time=1.617 min. LCMS CP Method C2

¹H NMR (400 MHz, DMSO-d₆) δ 11.62 (s, 1H), 10.98 (s, 1H), 7.49 (dd, J=8.5, 5.6 Hz, 2H), 7.34-7.14 (m, 7H), 3.98 (dd, J=18.7, 13.7 Hz, 2H), 3.80-3.73 (m, 2H), 3.07 (dd, J=9.3, 5.8 Hz, 1H), 2.96 (d, J=15.9 Hz, 2H), 2.73 (d, J=4.3 Hz, 3H), 2.12 (d, J=13.0 Hz, 2H), 1.99-1.82 (m, 2H), 1.24 (s, 1H).

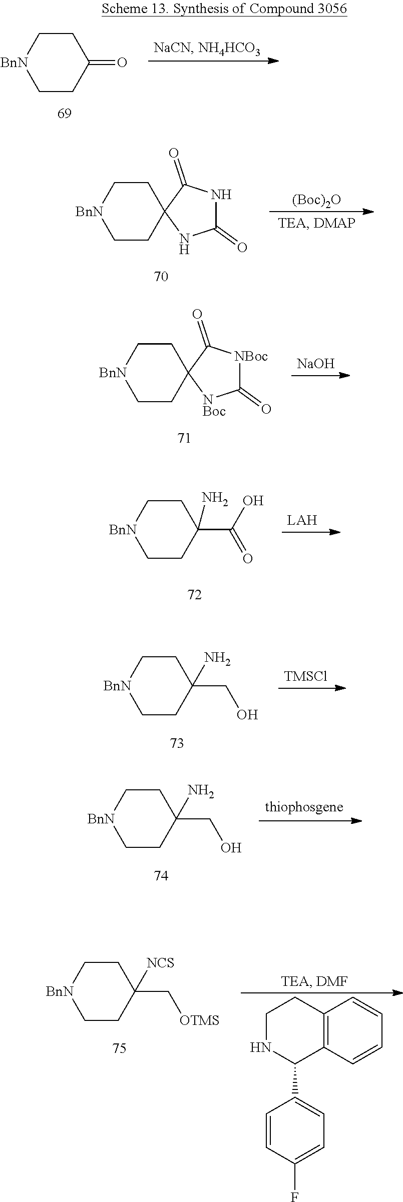

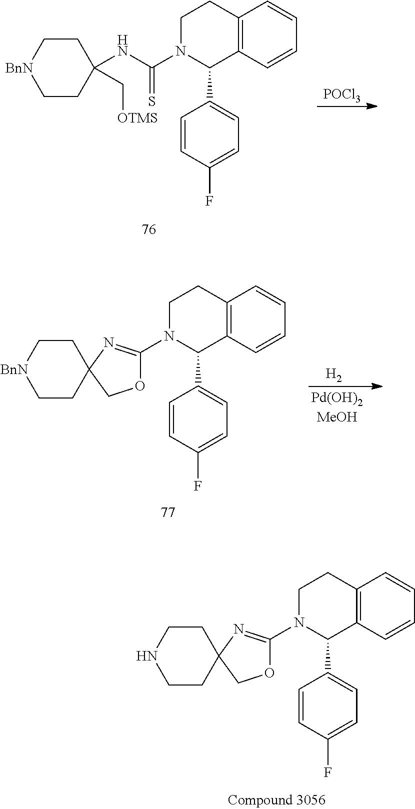

Step 1: To a solution of 1-benzylpiperidin-4-one 69 (18.9 g, 100.0 mmol) in EtOH (200 mL) and H₂O (200 mL) were added sodium cyanide (9.8 g, 200 mmol) and NH₄HCO₃ (40 g, 500 mmol). The reaction mixture was stirred at 80° C. for 16 h. Then the reaction mixture was cooled to room temperature and filtered. The solid was washed with hot water (1000 mL) and EtOH (100 mL). The solid was dried under vacuum to obtain 8-benzyl-1, 3, 8-triazaspiro[4.5]decane-2,4-dione 70. LCMS: (M+H)+=260 (UV 214 nm); Retention time=0.92 min. LCMS CP Method E

Step 2: To a solution of 8-benzyl-1,3,8-triazaspiro[4.5]decane-2,4-dione 70 (20 g, 77 mmol), DMAP (939 mg, 7.7 mmol) and triethylamine (23.33 g, 231 mmol) in tetrahydrofuran (200 mL) was added (Boc)₂O (50.4 g, 231 mmol) slowly. The mixture was stirred at room temperature for 16 h. The mixture was filtered and the solid was washed with THF (400 mL). The solid was dried under vacuum to obtain di-tert-butyl 8-benzyl-2, 4-dioxo-1,3,8-triazaspiro[4.5]decane-1,3-dicarboxylate 71. LCMS: (M+H)+=460 (UV 214 nm); Retention time=1.48 min. LCMS CP Method D

Step 3: To a solution of di-tert-butyl 8-benzyl-2, 4-dioxo-1,3,8-triazaspiro[4.5]decane-1,3-dicarboxylate 71 (26.8 g, 58.4 mmol) in water (300 mL) was added NaOH (9.34 g, 233.6 mmol). The mixture was stirred overnight at 100° C. The mixture was cooled to 25° C. and quenched with 4N HCl aqueous solution to adjust pH=6-7. A solid precipitated and was collected by filtration, then washed with water (100 mL). The solid was dried in vacuo to give 4-amino-1-benzylpiperidine-4-carboxylic acid 72. LCMS: (M+H)+=235 (214 nm); retention time=1.03 min. LCMS CP Method C

Step 4: To a solution of 4-amino-1-benzylpiperidine-4-carboxylic acid 72 (2.34 g, 10 mmol) in THF (20 mL) was added LiAlH₄ (15 ml, 15 mmol) at 0° C. The mixture was stirred at 25° C. for 12 hours and quenched with Na₂SO₄.10H₂O. The resulting mix was filtered and the filtrate concentrated in vacuo to give (4-amino-1-benzylpiperidin-4-yl)methanol 73. This material was used directly in the next reaction without further purification. LCMS: (M+H)+=221 (214 nm); retention time=1.00 min. LCMS CP Method C

Step 5: To a solution of (4-amino-1-benzylpiperidin-4-yl)methanol 73 (830 mg, 3.8 mmol) in DCM (10 mL) were added TEA (707 mg, 7 mmol) and trimethyl chlorosilane (654 mg, 6 mmol). The mixture was stirred at room temperature for 2 h. The mixture was diluted with water (40 mL) and extracted with three 50 mL portions of ethyl acetate. The combined organic layers were dried with Na₂SO₄, filtered and concentrated to give 1-benzyl-4-((trimethylsilyloxy)methyl)piperidin-4-amine 74. LCMS: (M+H)+=293 (214 nm); retention time=1.64 min. LCMS CP Method C

Step 6: To a mixture of 1-benzyl-4-((trimethylsilyloxy)methyl)piperidin-4-amine 74 (620 mg, 2.1 mmol) in DCM (5 mL) was added CSCl₂ (242 mg, 2.1 mmol) at 0° C. The resulting reaction mixture was stirred at room temperature for 1 h and concentrated at 30° C. to obtain a solid. The solid 75 was used directly in the next step without further purification.

Step 7: To a mixture of (S)-1-(4-fluorophenyl)-1,2,3,4-tetrahydroisoquinoline 5 (477 mg, 2.1 mmol) and TEA (424 mg, 4.2 mmol) in DMF (5 mL) was added the white solid 75 and the resulting mixture was heated to 60° C. for 6 h. The reaction mixture was diluted with water (50 mL) and extracted with three 30 mL portions of ethyl acetate. The combined organic layers were dried over anhydrous Na₂SO₄, filtered and concentrated to give a crude oil which was purified by prep-HPLC to give (S)—N-(1-benzyl-4-((trimethylsilyloxy)methyl)piperidin-4-yl)-1-(4-fluorophenyl)-3,4-dihydroisoquinoline-2(1H)-carbothioamide 76. LCMS: (M+H)+=561(214 nm); retention time=1.42 min. LCMS CP Method C

Step 8: A mixture of (S)—N-(1-benzyl-4-((trimethylsilyloxy)methyl)piperidin-4-yl)-1-(4-fluorophenyl)-3,4-dihydroisoquinoline-2(1H)-carbothioamide 76 (480 mg, 0.86 mmol) in POCl3 (5 mL) was stirred at 100° C. for 16 h. The reaction mixture was cooled to room temperature and quenched with cold saturated NaHCO₃ aqueous solution, then extracted with three 20 mL portions of ethyl acetate. The combined organic layers were dried over anhydrous Na₂SO₄, filtered and concentrated to give a crude oil which was purified by prep-HPLC to give (S)-8-benzyl-2-(1-(4-fluorophenyl)-3,4-dihydroisoquinolin-2(1H)-yl)-3-oxa-1,8-diazaspiro[4.5]dec-1-ene 77. LCMS: (M+H)⁺=456(214 nm); retention time=1.979 min. LCMS CP Method C1

Step 9: To a mixture of (S)-8-benzyl-2-(1-(4-fluorophenyl)-3,4-dihydroisoquinolin-2(1H)-yl)-3-oxa-1,8-diazaspiro[4.5]dec-1-ene 77 (120 mg, 0.26 mmol) in MeOH (5 mL) was added Pd(OH)₂ (24 mg, 20% w/w %) and the reaction mixture was stirred at 25° C. for 2 h under H₂. LCMS showed the reaction completed. The reaction mixture was filtered and the filtrate was concentrated to give a crude oil which was purified by prep-HPLC to give (S)-2-(1-(4-fluorophenyl)-3, 4-dihydroisoquinolin-2(1H)-yl)-3-oxa-1,8-diazaspiro[4.5]dec-1-ene Compound 3056.

Compound 3056: LCMS: (M+H)⁺=366; purity=100% (214 nm); retention time=1.702 min. LCMS CP Method C

¹H NMR (400 MHz, CDCl₃) δ 7.26-7.21 (m, 2H), 7.21-7.14 (m, 4H), 7.01-6.91 (m, 2H), 6.39 (s, 1H), 4.99 (s, 1H), 3.63-3.48 (m, 2H), 3.16 (dt, J=15.8, 5.2 Hz, 2H), 2.90 (ddd, J=23.8, 15.4, 6.5 Hz, 2H), 2.80 (dd, J=9.4, 6.8 Hz, 4H), 2.09-1.98 (m, 2H), 1.69 (ddd, J=8.1, 7.2, 5.7 Hz, 2H).

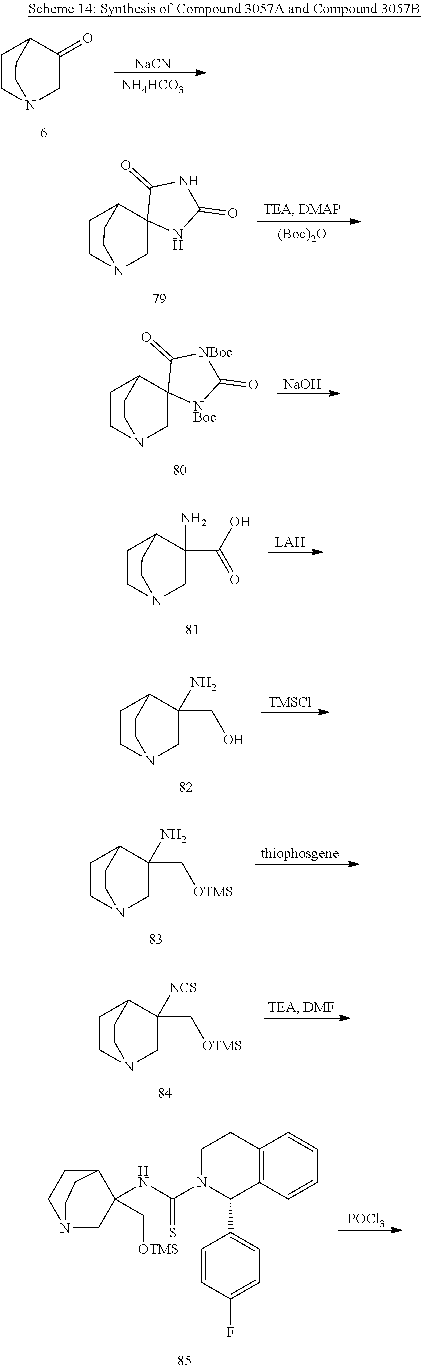

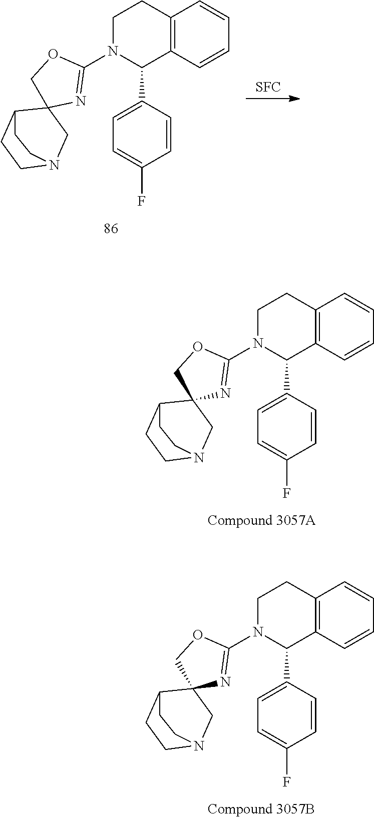

Step 1: To a solution of quinuclidin-3-one (12.5 g, 100.0 mmol) in EtOH (200 mL) and H₂O (200 mL) was added sodium cyanide (9.8 g, 200 mmol) and NH₄HCO₃ (40 g, 500 mmol). The reaction mixture was stirred at 80° C. for 16 h, cooled to ambient temperature and filtered. The solid was washed with hot water (1000 mL) and EtOH (100 mL) and dried under vacuum to afford 4-azaspiro[bicyclo[2.2.2]octane-2,4 [symbol] imidazolidine]-2 [symbol] dione (17.5 g) as a white solid. LCMS: (M+H)⁺=196 (UV 214 nm); Retention time=0.85 min. LCMS CP Method E

Step 2: To a mixture of 4-azaspiro[bicyclo[2.2.2]octane-2,4 [symbol] imidazolidine]-2 [symbol] dione (15.6 g, 80 mmol), DMAP (980 mg, 8 mmol) and triethylamine (20 g, 200 mmol) in tetrahydrofuran (200 mL) was slowly added (Boc)₂O (45 g, 200 mmol). The reaction mixture was stirred at room temperature for 16 h, filtered and the solid washed by THF (400 mL). The solid was dried under vacuum to deliver di-tert-butyl 2 [symbol] dioxo-4-azaspiro[bicyclo[2.2.2]octane-2,4 [symbol] imidazolidine]-1 [symbol] dicarboxylate. LCMS: (M+H)⁺=396 (UV 214 nm); Retention time=1.52 min. LCMS CP Method D

Step 3: To a solution of di-tert-butyl 2 [symbol] dioxo-4-azaspiro[bicyclo[2.2.2]octane-2,4 [symbol] imidazolidine]-1 [symbol] dicarboxylate (23 g, 58.4 mmol) in water (300 mL) was added NaOH (9.34 g, 233.6 mmol). The mixture was stirred overnight at 100° C., then cooled to 25° C. and quenched by the addition of 4N HCl. Acid was added until the pH reached between 6 and 7, resulting in the precipitation of a white solid. This solid was filtered, washed with water (100 mL) and dried in vacuo to give 3-aminoquinuclidine-3-carboxylic acid. LCMS: (M+H)⁺=171 (214 nm); retention time=0.78 min. LCMS CP Method C

Step 4: To a solution of 3-aminoquinuclidine-3-carboxylic acid (1.71 g, 10 mmol) in THF (20 mL) was added LAH (15 ml, 15 mmol) at 0° C. The mixture was stirred at 25° C. for 12 hours. Then NaSO₄.5H₂O was added until effervescence ceased and the mixture was filtered through celite, dried and concentrated in vacuo to give (3-aminoquinuclidin-3-yl)methanol. LCMS: (M+H)⁺=157 (214 nm); retention time=0.95 min. LCMS CP Method C

Step 5: To a solution of (3-aminoquinuclidin-3-yl)methanol (620 mg, 3.9 mmol) in DCM (10 mL) were added TEA (707 mg, 7 mmol) and TMSCl (654 mg, 6 mmol). The mixture was stirred at room temperature for 2 h, diluted with water (40 mL) and extracted with ethyl acetate (3×50 mL). The combined organic phase was dried over Na₂SO₄ and concentrated to give 3-(((trimethylsilyl)oxy)methyl)quinuclidin-3-amine. LCMS: (M+H)⁺=229 (214 nm); retention time=1.54 min. LCMS CP Method C

Steps 6 & 7: To a mixture of 3-(((trimethylsilyl)oxy)methyl)quinuclidin-3-amine (453 mg, 1.98 mmol) in DCM (5 mL) was added CSCl₂ (242 mg, 2.1 mmol) at 0° C. The resulting reaction mixture was stirred at ambient temperature for 1 h and concentrated at 30° C. to obtain a solid. The solid was used for next step without further purification.

To a mixture of THIQ (477 mg, 2.1 mmol) and TEA (424 mg, 4.2 mmol) in DMF (5 mL) was added the solid obtained above. It was heated at 60° C. for 6 h and then diluted with water (50 mL). After extraction with ethyl acetate (3×30 mL), the combined organic phase was dried over anhydrous Na₂SO₄, filtered and concentrated to obtain a crude oil, which was purified by prep-HPLC to obtain (1S)-1-(4-fluorophenyl)-N-(3-(((trimethylsilyl)oxy)methyl)quinuclidin-3-yl)-3,4-dihydroisoquinoline-2(1H)-carbothioamide. LCMS: (M+H)⁺=498(214 nm); retention time=1.55 min. LCMS CP Method C

Step 8: (1S)-1-(4-fluorophenyl)-N-(3-(((trimethylsilyl)oxy)methyl)quinuclidin-3-yl)-3,4-dihydroisoquinoline-2(1H)-carbothioamide (208 mg, 0.42 mmol) was mixed with POCl₃ (5 mL) and stirred at 100° C. for 16 h. The reaction mixture was cooled to ambient temperature, quenched by cold saturated aqueous NaHCO₃ and extracted with ethyl acetate (3×20 mL). The combined organic phase was dried over anhydrous Na₂SO₄, filtered and concentrated to obtain a crude oil, which was purified by prep-HPLC to obtain 2 [symbol] ((S)-1-(4-fluorophenyl)-3,4-dihydroisoquinolin-2(1H)-yl)-5 [symbol] -4-azaspiro[bicyclo[2.2.2]octane-2,4 [symbol] oxazole]. LCMS: (M+H)⁺=392 (214 nm); retention time=1.79 min. LCMS CP Method C1

The diastereomers were separated by chiral SFC eluting with CO₂/MeOH containing 0.2% methanolic ammonia over an EnantioPak® IG column (4.6*100 mm 5 μm) to give Compound 3057A (retention time=1.031 min) and Compound 3057B (retention time=1.122 min). Stereochemical assignment of (S) at 1 position of the tetrahydroisoquinoline is based on enantiomerically pure starting material, stereochemical configuration at the spirocyclic chiral center is arbitrarily assigned based on chromatographic elution order as compared to diastereomers of related analogues of known configuration.

Compound 3057A: LCMS: (M+H)⁺=392; purity=100% (214 nm); retention time=1.79 min. LCMS CP Method C. ¹H NMR (400 MHz, CDCl₃) δ 7.24-7.10 (m, 5H), 7.02-6.88 (m, 3H), 6.23 (s, 1H), 4.36 (t, J=11.7 Hz, 1H), 3.98-3.81 (m, 2H), 3.28 (ddd, J=13.2, 10.8, 4.3 Hz, 1H), 3.08-2.96 (m, 3H), 2.86-2.64 (m, 5H), 2.20 (t, J=10.5 Hz, 1H), 2.00 (s, 2H), 1.60-1.56 (m, 1H), 1.47-1.36 (m, 1H). Chiral SFC: CO₂/MeOH containing 0.2% MA (60%:40%) over CHIRALPAK® IG column (4.6×100 mm, 5 μm), retention time=1.031 min, 100% ee.

Compound 3057B: LCMS: (M+H)⁺=392; purity=100% (214 nm); retention time=1.77 min. LCMS CP Method C. ¹H NMR (400 MHz, CDCl₃) δ 7.25-7.13 (m, 5H), 7.06-6.90 (m, 3H), 6.22 (s, 1H), 4.36 (d, J=8.2 Hz, 1H), 4.05-3.88 (m, 2H), 3.32-3.20 (m, 2H), 3.04 (dd, J=18.3, 8.3 Hz, 3H), 2.89 (dt, J=14.0, 7.5 Hz, 4H), 2.76-2.67 (m, 1H), 2.27 (d, J=9.8 Hz, 1H), 1.76-1.59 (m, 3H). Chiral SFC: CO₂/MeOH containing 0.2% MA (60%:40%) over CHIRALPAK® IG column (4.6×100 mm, 5 μm), retention time=1.122 min, 100% ee.

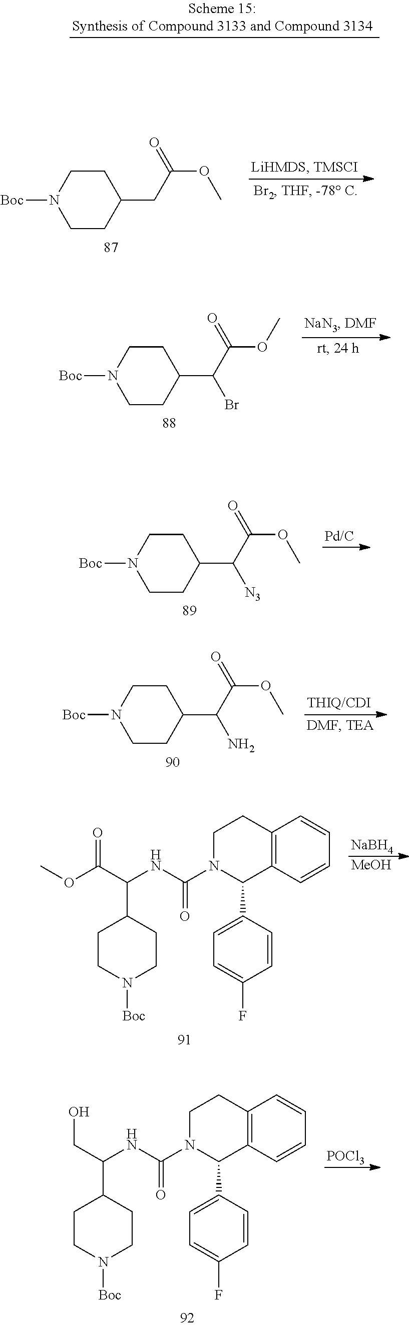

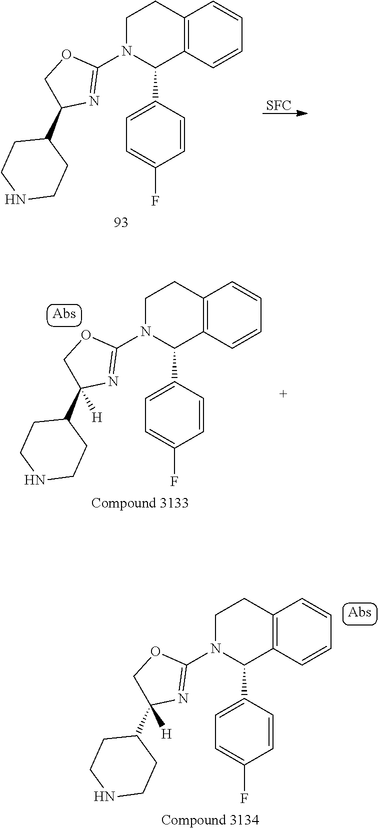

Step 1: To a solution of 4-methoxycarbonylmethyl-piperidine-1-carboxylic acid tert-butyl ester (10 g, 39 mmol) in THF (150 mL) was added to LHMDS (1.0 M in THF) (70 mL, 70 mmol) at −78° C. The reaction mixture was stirred at −78° C. for 3 before TMSCl (8.9 mL, 70 mmol) was added dropwise. The mixture was stirred for 1 h at −78° C. and then bromine (2.4 mL, 47 mmol) was added dropwise. The mixture was stirred at −78° C. for 2 h, then allowed to warm to 0° C. and stirred for an additional 30 min. The mixture was diluted with ethyl acetate and washed with saturated NaHCO₃ solution, then washed with water. The organic phase was dried over Na₂SO₄, filtered and the filtrate concentrated in vacuo to yield a yellow solid. This material was purified by flash column chromatography (50% EtOAc/hexane) to yield 4-(bromo-methoxycarbonyl-methyl)-piperidine-1-carboxylic acid tert-butyl ester. LCMS: (M+H)⁺=336 (UV 214 nm); Retention time=1.32 min. LCMS CP Method E

Step 2: Methyl 2-Azido-2-H-tert-butoxycarbonyl-4-piperidinyl)-acetate (26.3 g, 78.2 mmol) was dissolved in DMF (100 mL), sodium azide (26.2 g, 403 mmol) was then added. After stirring at ambient temperature for 24 h, the reaction mixture was filtered through a glass frit, washing once with DMF, and the filtrate concentrated in vacuo. The residue was dissolved in DCM and washed twice with water and the aqueous layers extracted with DCM. The organic phase was washed with brine, dried over anhydrous sodium sulfate, and filtered. Concentration of the filtrate in vacuo gave tert-butyl 4-(1-azido-2-methoxy-2-oxoethyl)piperidine-1-carboxylate. LCMS: (M+H)⁺=460 (UV 214 nm); Retention time=1.48 min. LCMS CP Method D. ¹H NMR (400 MHz, CDCl₃) δ: 3.81 (s, 3H), 3.73 (d, J=6.8 Hz, 1H), 2.59-2.78 (m, 2H), 1.91-2.05 (m, 1H), 1.62-1.72 (m, 1H), 1.55 (ddd, J=13.1, 2.6, 2.5 Hz, 1H), 1.42-1.48 (m, 10H), 1.25-1.42 (m, 2H).

Step 3: A solution of tert-butyl 4-(1-azido-2-methoxy-2-oxoethyl)piperidine-1-carboxylate (23.5 g, 78.2 mmol) in methanol (100 mL) was sparged with argon for 10 min, treated with 10% palladium(0) on carbon (2.3 g) under argon, evacuated under house vacuum and backfilled with hydrogen (5×). The reaction mixture was stirred vigorously under a hydrogen atmosphere at ambient temperature for 24 h and then filtered over Celite, washing with methanol. The filtrate was concentrated in vacuo giving tert-butyl 4-(1-amino-2-methoxy-2-oxoethyl)piperidine-1-carboxylate. LCMS: (M+H)⁺=273 (214 nm); retention time=1.03 min. LCMS CP Method C. ¹H NMR (400 MHz, CDCl₃) δ: 4.15 (br. s., 2H), 3.74 (s, 3H), 3.48 (s, 1H), 3.34 (d, J=5.6 Hz, 1H), 2.67 (br. s., 2H), 1.72-1.85 (m, 1H), 1.64 (d, J=13.1 Hz, 1H), 1.54 (d, J=12.9 Hz, 1H), 1.42-1.49 (m, 10H), 1.32-1.42 (m, 1H), 1.22-1.32 (m, 1H).

Step 4: To a solution of THIQ (2.27 g, 10 mmol) in DMF (5 mL) was added tert-butyl 4-(1-amino-2-methoxy-2-oxoethyl)piperidine-1-carboxylate (2.99 g, 11 mmol), CDI (1.62 mg, 30 mmol) and TEA (3.12 g, 3.08 mmol). The mixture was stirred at 70° C. for 10 min. The mixture was cooled to 25° C. and added water (20 mL). Then the mixture was extracted with ethyl acetate (3×20 mL). The combined organic phase was washed with brine (3×20 mL), dried and concentrated in vacuo to give a crude product, which was purified by prep-HPLC to give 1-(4-isopropoxyphenyl)-N—((S)-quinuclidin-3-yl)-3,4-dihydroisoquinoline-2(1H)-carboxamide. LCMS: (M+H)⁺=526; purity=100% (214 nm); retention time=1.72 min. LCMS CP Method C.

Step 5: To a solution of tert-butyl 4-(1-((S)-1-(4-fluorophenyl)-1,2,3,4-tetrahydroisoquinoline-2-carboxamido)-2-methoxy-2-oxoethyl)piperidine-1-carboxylate (1.05 g 2 mmol) in MeOH (20 mL) was added NaBH₄ (235 mg, 6.3 mmol). It was stirred at r.t for 0.5 h. The mixture was concentrated in vacuo to remove MeOH and the solid was dissolved in ethyl acetate (20 mL), washed with water (20 mL) and brine (20 mL), dried over Na₂SO₄ and concentrated giving tert-butyl 4-(1-((S)-1-(4-fluorophenyl)-1,2,3,4-tetrahydroisoquinoline-2-carboxamido)-2-hydroxyethyl)piperidine-1-carboxylate. LCMS: (M+H)⁺=498; purity=100% (214 nm); retention time=1.42 min. LCMS CP Method C

Step 6: A solution of tert-butyl 4-(1-((S)-1-(4-fluorophenyl)-1,2,3,4-tetrahydroisoquinoline-2-carboxamido)-2-hydroxyethyl)piperidine-1-carboxylate (497 mg, 1 mmol) in tetrahydrofuran (50 mL) was cooled to 0° C., then phosphoryl trichloride (612 mg, 4 mmol) was added. The reaction mixture was stirred at 60 C.° until TLC analysis indicated the total consumption of the starting material. The solvent was evaporated to give a crude product (1.2 g), which was purified by Prep-HPLC to give 2-((S)-1-(4-fluorophenyl)-3,4-dihydroisoquinolin-2(1H)-yl)-4-(piperidin-4-yl)-4,5-dihydrooxazole. LCMS: (M+H)⁺=380 (UV 214 nm); Retention time=1.77 min. LCMS CP Method C

The diastereomers were separated by chiral SFC eluting with CO₂/MeOH containing 0.2% methanolic ammonia over an EnantioPak® IG column (4.6*100 mm 5 μm) to give Compound 3133 (54.7 mg, retention time=1.625 min) and Compound 3134 (38.3 mg, retention time=1.985 min). Stereochemical assignment of (S) at 1 position of the tetrahydroisoquinoline is based on enantiomerically pure starting materials, stereochemical configuration at the spirocyclic chiral center is arbitrarily assigned based on chromatographic elution order as compared to diastereomers of related analogues of known configuration.

Compound 3133: LCMS: (M+H)⁺=380; purity=92.11% (214 nm); retention time=1.24 min. Method C. Chiral SFC: CO₂/MeOH containing 0.2% ammonia over CHIRALPAK® IG column (4.6*100 mm 5 μm), retention time=1.625 min), 100% ee.

Compound 3134: LCMS: (M+H)⁺=380; purity=92.11% (214 nm); retention time=1.24 min. Method C. Chiral SFC: CO₂/MeOH containing 0.2% ammonia over CHIRALPAK® IG column (4.6*100 mm 5 μm), retention time=1.985 min), 100% ee.

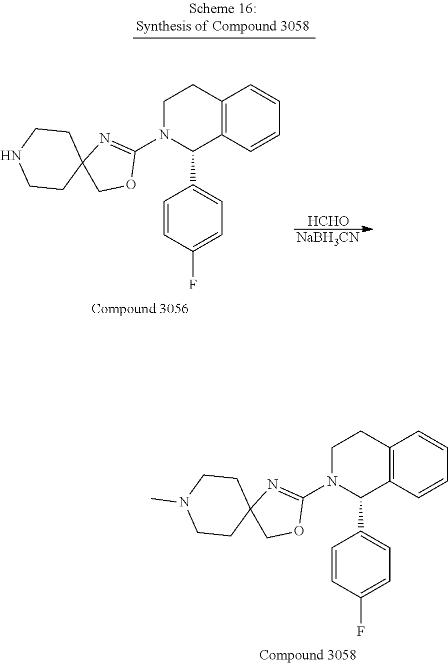 [SMILES: Fc1ccc([C@H]2c3ccccc3CCN2C2=NC3(CCNCC3)CO2)cc1 ; "HCHO" ; "NaBH3CN" ; CN1CCC2(CC1)COC(N1CCc3ccccc3[C@@H]1c1ccc(F)cc1)=N2]

Compound 3058 was prepared following a similar synthesis as for Compound 3056.

Step 10: To a mixture of (S)-2-(1-(4-fluorophenyl)-3, 4-dihydroisoquinolin-2(1H)-yl)-3-oxa-1, 8-diazaspiro[4.5]dec-1-ene (36 mg, 0.1 mmol) in MeOH (5 mL) was added 37% HCHO aqueous (1 mL, excess), followed by NaBH₃CN (12 mg, 0.2 mmol). The reaction mixture was stirred at 25° C. for 4 hr and then concentrated to give a crude solid. This was purified by prep-HPLC to give (S)-2-(1-(4-fluorophenyl)-3,4-dihydroisoquinolin-2(1H)-yl)-8-methyl-3-oxa-1,8-diazaspiro[4.5]dec-1-ene. Compound 3058: LCMS: (M+H)⁺=380.1; purity=100% (214 nm); retention time=1.76 min. Method C. ¹H NMR (400 MHz, CDCl₃) δ 7.07 (q, J=8.3 Hz, 4H), 7.01 (d, J=5.6 Hz, 2H), 6.85 (t, J=8.6 Hz, 2H), 6.45 (s, 1H), 4.90-4.82 (m, 1H), 4.73 (d, J=1.8 Hz, 1H), 4.55 (d, J=10.9 Hz, 1H), 3.78 (s, 1H), 3.53 (s, 2H), 3.38 (dd, J=8.1, 1.2 Hz, 3H), 3.04 (s, 3H), 2.93-2.83 (m, 1H), 2.55 (d, J=16.0 Hz, 1H), 2.30 (d, J=11.4 Hz, 2H), 2.18 (s, 2H). Chiral SFC: CO₂/MeOH containing 0.2% Methanol Ammonia=65:35 over CHIRALPAK IG column (4.6*100 mm 5 μm), retention time=1.32 min, 100% ee.

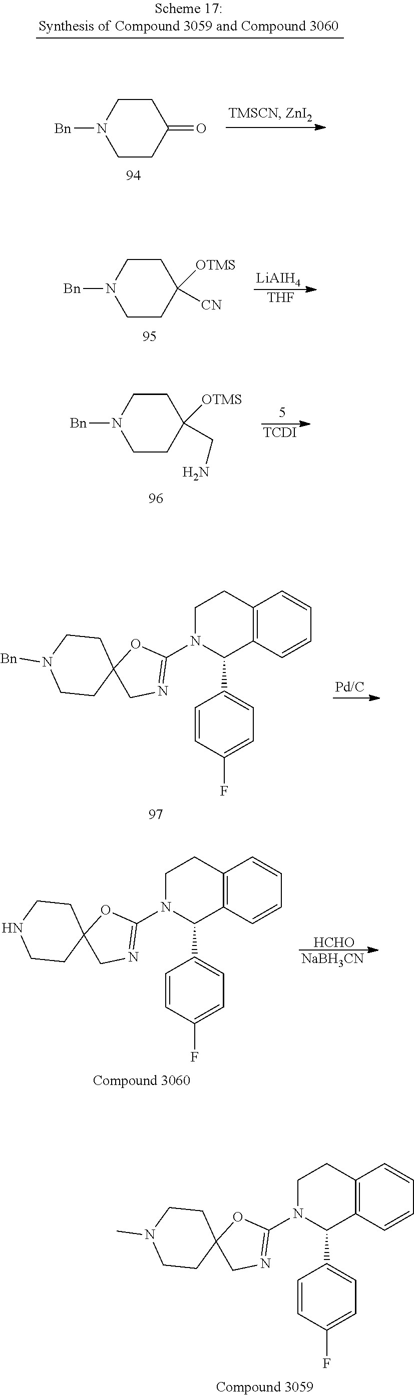

Step 1: To a solution of 1-benzylpiperidin-4-one (22.5 g, 100 mmol) and ZnI₂ (7.36 g, 20 mmol) in THF (200 mL) was added TMSCN (19.8 g, 200 mmol) at 25° C. The reaction mixture was stirred at 80° C. for 16 h and then filtered and concentrated to give a 1-benzyl-4-(trimethylsilyloxy)piperidine-4-carbonitrile which was used directly in the next step reaction without further purification. LCMS: (M+H)⁺=289.1, purity=54% (214 nm), Retention time=1.44 min. LCMS CP method C

Step 2: To a solution of 1-benzyl-4-(trimethylsilyloxy)piperidine-4-carbonitrile (14.4 g, 50 mmol) in THF (250 mL) was added LAH (150 mL, 150 mmol) dropwise at 0° C. under nitrogen atmosphere. The reaction mixture was stirred for 16 h at 0° C. The reaction mixture was quenched with saturated aqueous Na₂SO₄ solution, filtered and concentrated to give (1-benzyl-4-(trimethylsilyloxy) piperidin-4-yl)methanamine which was used directly in the next step reaction without further purification. LCMS: (M+H)⁺=293.2, purity=47.8% (214 nm), Retention time=0.98 min. LCMS CP method C

Step 3: To a solution of (S)-1-(4-fluorophenyl)-1,2,3,4-tetrahydroisoquinoline (2.27 g, 10 mmol) and TEA (4.04 g, 40 mmol) in DMF (150 mL) was added TCDI (2.67 g, 15 mmol) at room temperature under nitrogen atmosphere. After stirring the reaction mixture for 30 min at 120° C., (1-benzyl-4-(trimethylsilyloxy) piperidin-4-yl)methanamine (11.68 g, 40 mmol) and TEA (3.03 g, 30 mmol) in DMF (50 mL) were added. The reaction mixture was heated to 140° C. for 1 h, cooled to ambient temperature, diluted with brine (300 mL) and extracted with EA (300 mL). The organic layer was washed with brine (200 mL), dried over anhydrous Na₂SO₄, filtered and concentrated to give a residue. Purification by Prep-HPLC afforded (S)-8-benzyl-2-(1-(4-fluorophenyl)-3,4-dihydroisoquinolin-2(1H)-yl)-1-oxa-3,8-diazaspiro[4.5]dec-2-ene. LCMS: (M+H)⁺=456.1, purity=98.4% (214 nm), Retention time=1.44 min. LCMS CP method C

Step 4: To a solution of (S)-8-benzyl-2-(1-(4-fluorophenyl)-3,4-dihydroisoquinolin-2(1H)-yl)-1-oxa-3,8-diazaspiro[4.5]dec-2-ene (210 mg, 0.46 mmol) in MeOH (3 mL) was added Pd/C (20 mg, 10% w/w %) at 25° C. The reaction mixture was stirred at 25° C. under a hydrogen atmosphere for 16 h, filtered through celite and concentrated. The residue which was purified by Prep-HPLC and SFC to afford (S)-2-(1-(4-fluorophenyl)-3,4-dihydroisoquinolin-2(1H)-yl)-1-oxa-3,8-diazaspiro[4.5]dec-2-ene. Compound 3060: LCMS: (M+H)⁺=366.1, purity=100% (214 nm), Retention time=1.55 min. LCMS CP method C. ¹H NMR (400 MHz, CDCl₃) δ 7.23-7.13 (m, 5H), 7.04 (d, J=7.4 Hz, 1H), 6.95 (t, J=8.7 Hz, 2H), 6.19 (s, 1H), 3.93 (ddd, J=13.1, 5.7, 3.7 Hz, 1H), 3.59-3.49 (m, 2H), 3.37-3.27 (m, 1H), 3.08-2.94 (m, 3H), 2.90-2.80 (m, 2H), 2.75 (dt, J=16.1, 3.9 Hz, 1H), 1.88-1.78 (m, 2H), 1.65 (d, J=8.6 Hz, 2H). Chiral SFC: CO₂/MeOH containing 0.2% Methanol Ammonia=80:20 over CHIRALPAK® OD column (4.6*100 mm 5 μm), retention time=1.98 min), 98.2% ee.

Step 5: A solution of (S)-2-(1-(4-fluorophenyl)-3,4-dihydroisoquinolin-2(1H)-yl)-1-oxa-3,8-diazaspiro[4.5]dec-2-ene (70 mg, 0.19 mmol), HCHO (49 mg, 0.57 mmol, 37% w/w aqueous) and NaBH₃CN (36 mg, 3 mmol) in MeOH (3 mL) was stirred for 16 h under nitrogen atmosphere. The reaction mixture was concentrated to remove the solvent and diluted with water (10 mL). The mixture was extracted with EA (3×10 mL), the combined organic layers washed with brine, dried over anhydrous Na₂SO₄, and concentrated. The residue was purified by Prep-HPLC and SFC to give (S)-2-(1-(4-fluorophenyl)-3,4-dihydroisoquinolin-2(1H)-yl)-8-methyl-1-oxa-3,8-diazaspiro[4.5]dec-2-ene. Compound 3059: LCMS: (M+H)⁺=380.1, purity=100% (214 nm), Retention time=1.76 min. LCMS CP method C. ¹H NMR (400 MHz, CDCl₃) δ 7.23-7.12 (m, 5H), 7.04 (d, J=7.3 Hz, 1H), 6.95 (t, J=8.5 Hz, 2H), 6.19 (s, 1H), 3.93 (dd, J=8.1, 4.3 Hz, 1H), 3.53 (s, 2H), 3.39-3.23 (m, 1H), 3.04 (ddd, J=16.4, 10.5, 6.0 Hz, 1H), 2.75 (d, J=16.2 Hz, 1H), 2.46 (s, 4H), 2.32 (s, 3H), 1.91 (t, J=12.4 Hz, 2H), 1.78 (d, J=4.7 Hz, 2H). Chiral SFC: CO₂/MeOH containing 0.2% Methanol Ammonia=65:35 CHIRALPAK® IG column (4.6*100 mm 5 μm), retention time=0.99 min, 100% ee.

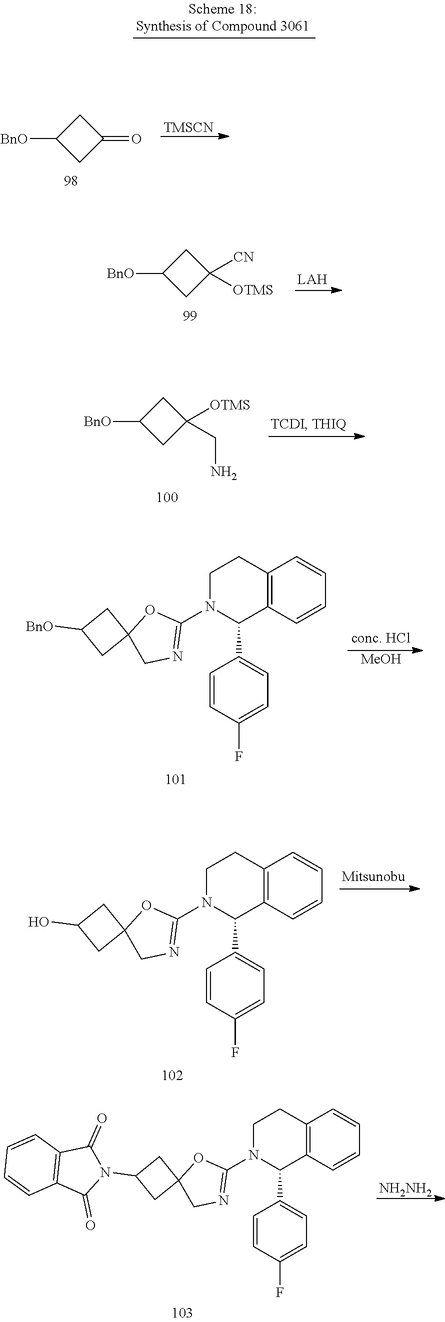

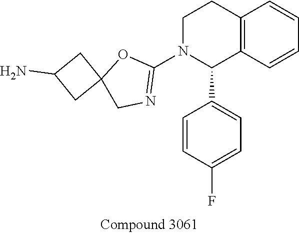 [SMILES: NC1CC2(CN=C(N3CCc4ccccc4[C@@H]3c3ccc(F)cc3)O2)C1]

Step 1: To a solution of 3-(benzyloxy)cyclobutan-1-one (3.52 g, 20.0 mmol) in anhydrous DCM (60 mL) were added TMSCN (2.38 g, 24.0 mmol) and ZnI₂ (638 mg, 2.0 mmol). The reaction mixture was stirred at room temperature overnight and then filtered. The filtrate was concentrated to give the crude product 3-(benzyloxy)-1-((trimethylsilyl)oxy)cyclobutane-1-carbonitrile which was used directly in the next step without further purification. LCMS: (M+H)⁺=276.1; Retention time=1.42 min. LCMS CP Method E

Step 2: To a suspension of tert-butyl 3-(benzyloxy)-1-((trimethylsilyl)oxy)cyclobutane-1-carbonitrile (5.5 g, 20 mmol) in THF (20 mL) was added LAH (1M/L 40 mL). The reaction mixture was stirred at room temperature overnight and then quenched with saturated Na₂SO₄ aqueous solution (50 ml). The mixture was filtered and concentrated to give (3-(benzyloxy)-1-((trimethylsilyl)oxy)cyclobutyl)methanamine which was used directly in the next step without further purification. LCMS: (M+H)⁺=280; Retention time=0.76 min. LCMS CP Method E

Step 3: To a solution of (3-(benzyloxy)-1-((trimethylsilyl)oxy)cyclobutyl) methanamine (2.79 g, 10 mmol) in DMF (50 mL) were added (S)-1-(4-fluorophenyl)-1,2,3,4-tetrahydroisoquinoline (4.54 g, 20 mmol), N,N [symbol] Carbonyldiimidazole (3.24 g, 20 mmol) and Et₃N (3.03 g, 30 mmol) at 0° C. The resulting reaction mixture was heated to 160° C. for 2 h, cooled and diluted with EA (100 mL). The organic phase was washed with saturated NH₄Cl (2×50 mL), brine (2×500 mL) and dried over Na₂SO₄. After filtration and concentration, the residue which was purified by Prep-HPLC to yield the product. LCMS: (M+H)⁺=443.1; Retention time=1.42 min. LCMS CP Method E

Step 4: A suspension of (S)-2-(benzyloxy)-6-(1-(4-fluorophenyl)-3,4-dihydroisoquinolin-2(1H)-yl)-5-oxa-7-azaspiro[3.4]oct-6-ene (2.0 g, 4.5 mmol) in conc. HCl (20 mL) was stirred at 100° C. overnight and then concentrated to give (S)-6-(1-(4-fluorophenyl)-3,4-dihydroisoquinolin-2(1H)-yl)-5-oxa-7-azaspiro[3.4]oct-6-en-2-ol which was used directly in the next step without further purification. LCMS: (M+H)⁺=353.2; Retention time=1.50 min. LCMS CP Method E

Step 5: To a solution of (S)-6-(1-(4-fluorophenyl)-3,4-dihydroisoquinolin-2(1H)-yl)-5-oxa-7-azaspiro[3.4]oct-6-en-2-ol (352 mg, 1.0 mmol), isoindoline-1,3-dione (294 mg, 2 mmol) and Ph₃P (524 mg, 2 mmol) in THF (25 mL) under a nitrogen atmosphere was added DEAD (348 mg, 2.0 mmol). The resulting reaction mixture was stirred at 60° C. for 2 h then diluted with ethyl acetate (100 mL) and water (130 mL) and the aqueous layer extracted with ethyl acetate (3×60 mL). The combined organic phase was washed with brine (60 mL), dried over Na₂SO₄, filtered and concentrated. The residue was purified by column chromatography to give (S)-2-(6-(1-(4-fluorophenyl)-3,4-dihydroisoquinolin-2(1H)-yl)-5-oxa-7-azaspiro[3.4]oct-6-en-2-yl)isoindoline-1,3-dione. LCMS: (M+H)⁺=482.1; Retention time=1.62 min. LCMS CP Method E

Step 6: A solution of ((S)-2-(6-(1-(4-fluorophenyl)-3,4-dihydroisoquinolin-2(1H)-yl)-5-oxa-7-azaspiro[3.4]oct-6-en-2-yl)isoindoline-1,3-dione (420 mg, 0.87 mmol) and NH₂NH₂.H₂O (1.0 g, 20 mmol) in EtOH (10 mL) was stirred at 65° C. for 2 h and then concentrated under reduced pressure. Purification of the residue by Prep-HPLC afforded (S)-6-(1-(4-fluorophenyl)-3,4-dihydroisoquinolin-2(1H)-yl)-5-oxa-7-azaspiro[3.4]oct-6-en-2-amine.

The cyclobutene isomers were isolated as a mixture. Stereochemistry at the 1 position of the tetrahydroisoquinoline is assigned based on enantiomerically pure starting material. Compound 3061: LCMS: (M+H)⁺=352.1; Retention time=1.48 min. LCMS CP Method C2. ¹H NMR (400 MHz, DMSO) δ 7.26-7.02 (m, 8H), 6.11 (s, 1H), 3.81-3.69 (m, 1H), 3.47 (dd, J=13.9, 6.6 Hz, 1H), 3.31-3.17 (m, 2H), 2.90 (ddd, J=16.1, 10.1, 6.0 Hz, 1H), 2.75 (dt, J=16.3, 4.1 Hz, 1H), 2.12 (s, 2H), 1.84 (ddd, J=18.3, 12.1, 6.3 Hz, 2H), 1.39 (s, 1H)

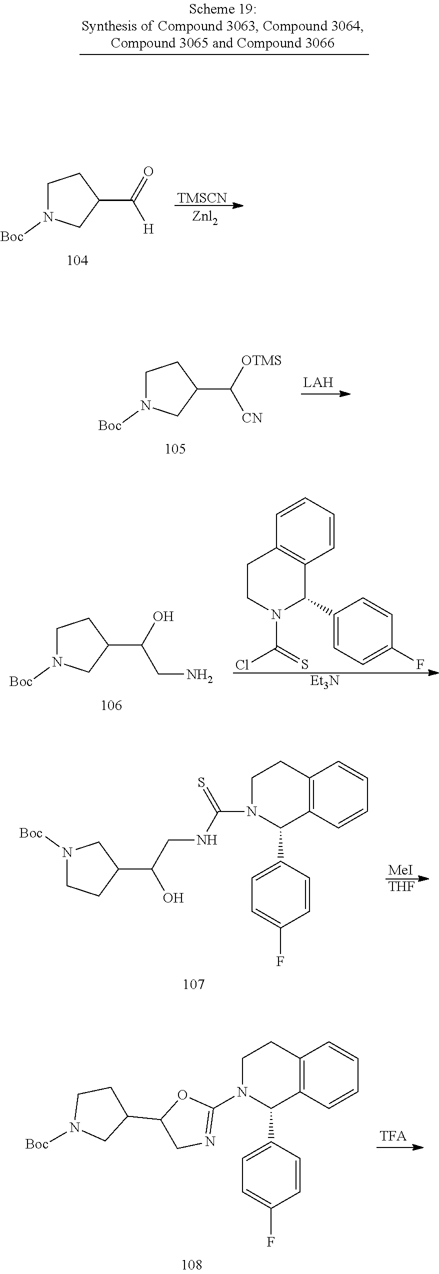

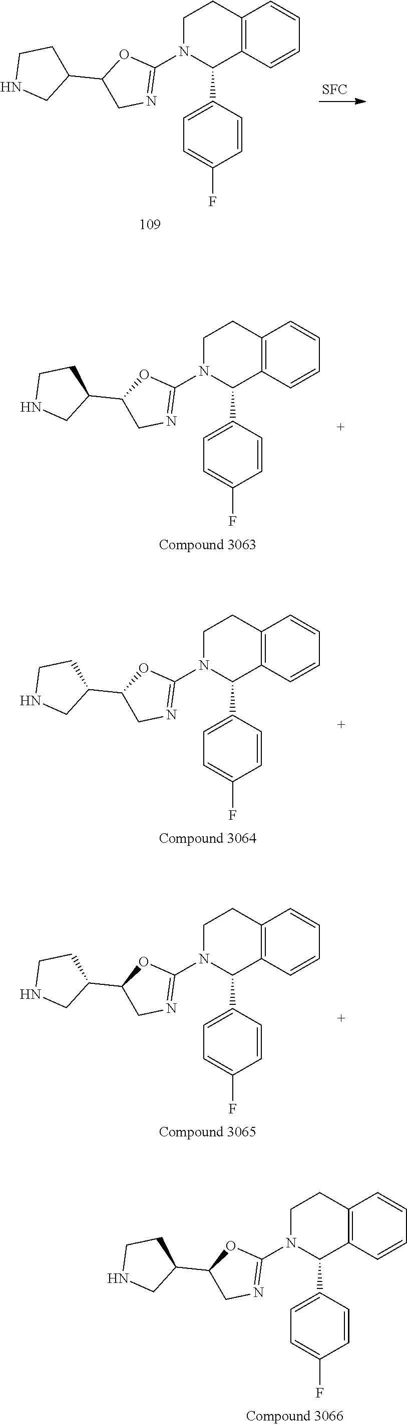

Step 1: To a solution of tert-butyl 3-formylpyrrolidine-1-carboxylate (1 g, 5 mmol) and TMSCN (0.75 mL, 6 mmol) in dried DCM (20 mL) was added ZnI₂ (80 mg, 0.25 mmol) at room temperature. The reaction mixture was stirred at room temperature for 16 h. The reaction mixture was filtered and the filtrate was concentrated to give the crude tert-butyl 3-(cyano((trimethylsilyl)oxy)methyl)pyrrolidine-1-carboxylate which was used directly in the next step reaction without further purification. LCMS: (M−TMS+Na)+=249.2; Retention time=1.49 min. LCMS CP Method E

Step 2: To a solution of tert-butyl 3-(cyano((trimethylsilyl)oxy)methyl)pyrrolidine-1-carboxylate (1.48 g, 5 mmol) in dry THF (20 mL) was slowly added LiAlH₄ (0.28 g, 7.4 mmol) at 0° C. After addition, the reaction mixture was stirred at room temperature for 16 hand then cooled to 0° C. and quenched with saturated sodium sulfate solution. The mixture was diluted with THF (100 mL) and dried over anhydrous Na₂SO₄. After filtration, the filtrate was concentrated to give tert-butyl 3-(2-amino-1-hydroxyethyl)pyrrolidine-1-carboxylate which was used directly in the next step reaction without further purification. LCMS: (M−55)+=175.1; Retention time=1.05 min. LCMS CP Method E

Step 3: To a solution of (S)-1-(4-fluorophenyl)-1,2,3,4-tetrahydroisoquinoline (0.7 g, 3.08 mmol) in dry CH₃CN (15 mL) was added thiophosgene (0.36 mL, 6.47 mmol) at 0° C. The reaction mixture was warmed to room temperature and stirred for 2 before being concentration in vacuo. The residue was dissolved in dry DMF (10 mL) and a solution of tert-butyl 3-(2-amino-1-hydroxyethyl)pyrrolidine-1-carboxylate (0.71 g, 3.08 mmol) in dry DMF (2 mL) followed by TEA (1.5 mL, 10.8 mmol) was added to the reaction mixture at 0° C. The reaction mixture was stirred at room temperature for 16 h then poured into water and extracted with DCM (3×20 mL). The combined organic phase was dried over anhydrous Na₂SO₄, filtered and concentrated. The residue was purified by Prep-HPLC to give tert-butyl 3-(2-((S)-1-(4-fluorophenyl)-1,2,3,4-tetrahydroisoquinoline-2-carbothioamido)-1-hydroxyethyl)pyrrolidine-1-carboxylate. LCMS: (M−55)+=444.0; Retention time=2.01 min. LCMS CP Method C

Step 4: To a solution of tert-butyl 3-(2-((S)-1-(4-fluorophenyl)-1,2,3,4-tetrahydroisoquinoline-2-carbothioamido)-1-hydroxyethyl)pyrrolidine-1-carboxylate (220 mg, 0.44) in THF (1 mL) was added CH₃I (316 mg, 2.22 mmol) at room temperature and the reaction mixture was stirred for 16 h. Concentration under reduced pressure gave the crude tert-butyl 3-(2-((S)-1-(4-fluorophenyl)-3,4-dihydroisoquinolin-2(1H)-yl)-4,5-dihydrooxazol-5-yl)pyrrolidine-1-carboxylate. This was used directly in the next step reaction without further purification. LCMS: (M+H)⁺=466.2; Retention time=1.63 min. LCMS CP Method B1

Step 5: To a solution of tert-butyl 3-(2-((S)-1-(4-fluorophenyl)-3,4-dihydroisoquinolin-2(1H)-yl)-4,5-dihydrooxazol-5-yl)pyrrolidine-1-carboxylate (204 mg, 0.44 mmol) in DCM (5 mL) was added TFA (1 mL) at 0° C. The resulting reaction mixture was stirred at room temperature for 3 h and then concentrated. The residue was redissolved in water, adjusted to alkaline pH with 1 N NaOH and extracted with DCM (3×50 mL). The combined organic phase was concentrated to give a residue which was purified by Prep-HPLC to give 2-((S)-1-(4-fluorophenyl)-3,4-dihydroisoquinolin-2(1H)-yl)-5-(pyrrolidin-3-yl)-4,5-dihydrooxazole. LCMS: (M+H)⁺=366.2; Retention time=1.39 min. LCMS CP Method B1

The diastereomers (80 mg) were separated by chiral HPLC eluting with n-Hexane (0.1% DEA):Ethanol (0.1% DEA)=80:20 over a Daicel® OD column (20×250 mm, 10 μm) to give Compound 3063, Compound 3064, Compound 3065 and Compound 3066. Stereochemical assignment of (S) at the 1 position of the tetrahydroisoquinoline is based on enantiomerically pure starting material; the stereochemistry of the chiral centers in the five membered rings is arbitrarily assigned based on chromatographic elution order as compared to related analogues of known configuration. Compound 3063: LCMS: (M+H)⁺=366.2; Retention time=1.20 min. LCMS CP Method A1. ¹H NMR (400 Hz, CDCl3): 7.24-7.14 (m, 5H), 7.02 (d, J=7.6 Hz, 1H), 6.97-6.93 (m, 2H), 6.16 (s, 1H), 4.31-4.45 (m, 1H), 3.93-3.87 (m, 2H), 3.51-3.46 (m, 1H), 3.37-3.30 (m, 1H), 3.09-2.71 (m, 6H), 2.37-2.27 (m, 1H), 1.91-1.86 (m, 1H), 1.41-1.35 (m, 1H). Chiral SFC: CO₂/MeOH (65%:35%) containing 0.2% ammonia over CHIRALPAK® IG column (4.6×100 mm, 5 μm), retention time=1.528 min), 97.94% ee.

Compound 3064: LCMS: (M+H)⁺=366.3; Retention time=1.22 min. LCMS CP Method A1. ¹H NMR (400 Hz, CDCl3): 7.23-7.15 (m, 5H), 7.03 (d, J=7.6 Hz, 1H), 6.98-6.93 (m, 2H), 6.18 (s, 1H), 4.57-4.51 (m, 1H), 3.92-3.84 (m, 2H), 3.50-3.45 (m, 1H), 3.32-3.25 (m, 1H), 3.09-3.01 (m, 2H), 2.92-2.87 (m, 2H), 2.76-2.63 (m, 2H), 2.37-2.31 (m, 1H), 1.94-1.87 (m, 1H), 1.65-1.56 (m, 1H). Chiral SFC: CO₂/MeOH (65%:35%) containing 0.2% ammonia over CHIRALPAK® IG column (4.6×100 mm, 5 μm), retention time=1.233 min), 100% ee.

Compound 3065: LCMS: (M+H)⁺=366.2; Retention time=1.21 min. LCMS CP Method A1. ¹H NMR (400 Hz, CDCl3): 7.24-7.15 (m, 5H), 7.02 (d, J=7.6 Hz, 1H), 6.95 (t, J=8.8 Hz, 2H), 6.17 (s, 1H), 4.48 (dd, J=8.4, 15.6 Hz, 1H), 3.92-3.85 (m, 2H), 3.52-3.47 (m, 1H), 3.31-3.24 (m, 1H), 3.12-2.70 (m, 6H), 2.36-2.30 (m, 1H), 1.94-1.87 (m, 1H), 1.45-1.36 (m, 1H). Chiral SFC: CO₂/MeOH (65%:35%) containing 0.2% ammonia over CHIRALPAK® IG column (4.6×100 mm, 5 μm), retention time=1.243 min), 97.29% ee.

Compound 3066: LCMS: (M+H)⁺=366.3; Retention time=1.22 min. LCMS CP Method A1. ¹H NMR (400 Hz, CDCl3): 7.24-7.15 (m, 5H), 7.03 (d, J=7.6 Hz, 1H), 6.98-6.93 (m, 2H), 6.17 (s, 1H), 4.57-4.52 (m, 1H), 3.93-3.85 (m, 2H), 3.48-3.32 (m, 2H), 3.06-2.98 (m, 2H), 2.95-2.89 (m, 2H), 2.78-2.72 (m, 1H), 2.67-2.63 (m, 1H), 2.35 (dd, J=8, 15.2 Hz, 1H), 1.94-1.86 (m, 1H), 1.66-1.57 (m, 1H). Chiral SFC: CO₂/MeOH (65%:35%) containing 0.2% ammonia over CHIRALPAK® IG column (4.6×100 mm, 5 μm), retention time=1.502 min), 100% ee.

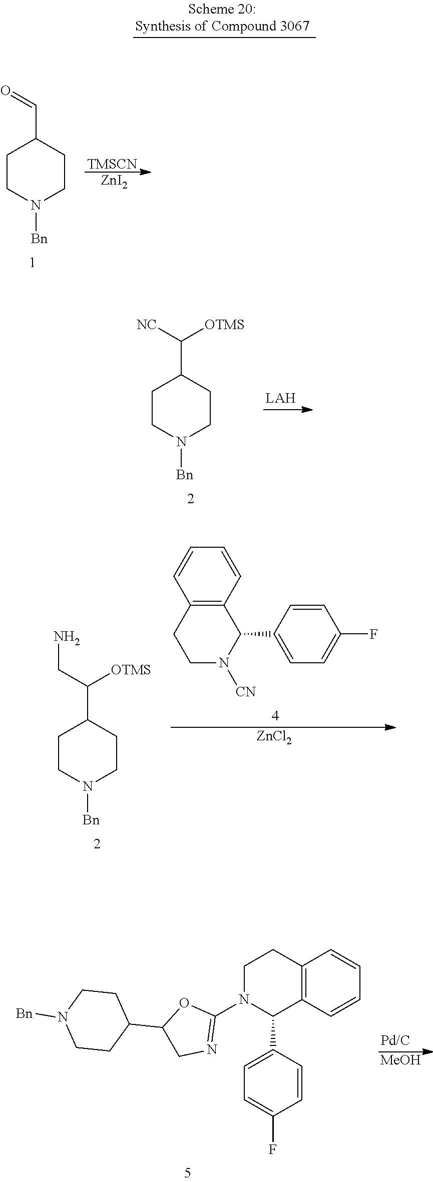

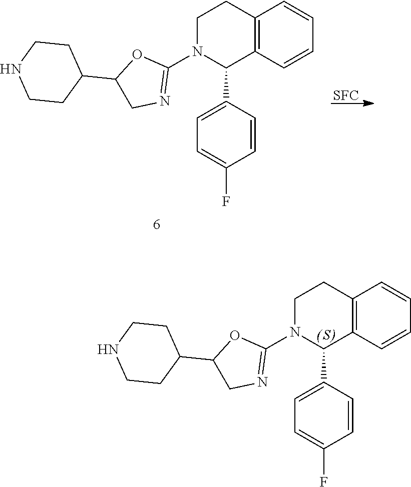

Compound 3067 was prepared following a similar synthesis as for TGT-277 with the following exception:

Step 3: To a solution of 2-(1-benzylpiperidin-4-yl)-2-(trimethylsilyloxy)ethanamine (918 mg, 3 mmol) and (S)-1-(4-fluorophenyl)-3,4-dihydroisoquinoline-2(1H)-carbonitrile (756 mg, 3 mmol) in anhydrous THF (20 mL) was added anhydrous ZnCl₂ (80 mg, 0.6 mmol). The reaction mixture was stirred at 80° C. for 16 h and then filtered. The filtrate was concentrated to give a residue which was purified by Prep-HPLC to give 5-(1-benzylpiperidin-4-yl)-2-((S)-1-(4-fluorophenyl)-3,4-dihydroisoquinolin-2(1H)-yl)-4,5-dihydrooxazole. LCMS: (M+H)⁺=380.1, purity=100% (214 nm), Retention time=1.57 min. Method C1. Compound 3067: (Mixture of diastereomers) LCMS: (M+H)⁺=380.1, purity=100% (214 nm), Retention time=1.57 min. Method C1. ¹H NMR (400 MHz, CDCl₃) δ 7.19 (ddd, J=15.3, 8.4, 3.5 Hz, 5H), 7.03 (dd, J=7.2, 3.5 Hz, 1H), 6.99-6.91 (m, 2H), 6.19 (d, J=4.1 Hz, 1H), 4.33 (dd, J=15.2, 7.8 Hz, 1H), 3.95-3.87 (m, 1H), 3.82 (ddd, J=12.5, 8.8, 4.0 Hz, 1H), 3.53 (ddd, J=12.3, 7.3, 4.9 Hz, 1H), 3.38-3.25 (m, 1H), 3.16-2.97 (m, 3H), 2.78-2.69 (m, 1H), 2.58 (ddd, J=12.1, 7.5, 2.6 Hz, 2H), 1.82 (d, J=15.3 Hz, 1H), 1.56 (dd, J=23.2, 8.4 Hz, 2H), 1.24 (ddd, J=12.3, 10.6, 6.5 Hz, 2H).

Chiral SFC: CO₂/MeOH containing 0.2% Methanol Ammonia=65:35 over CHIRALPAK® IG column (4.6*100 mm 5 μm), retention time=1.785 min), 96.52% ee.

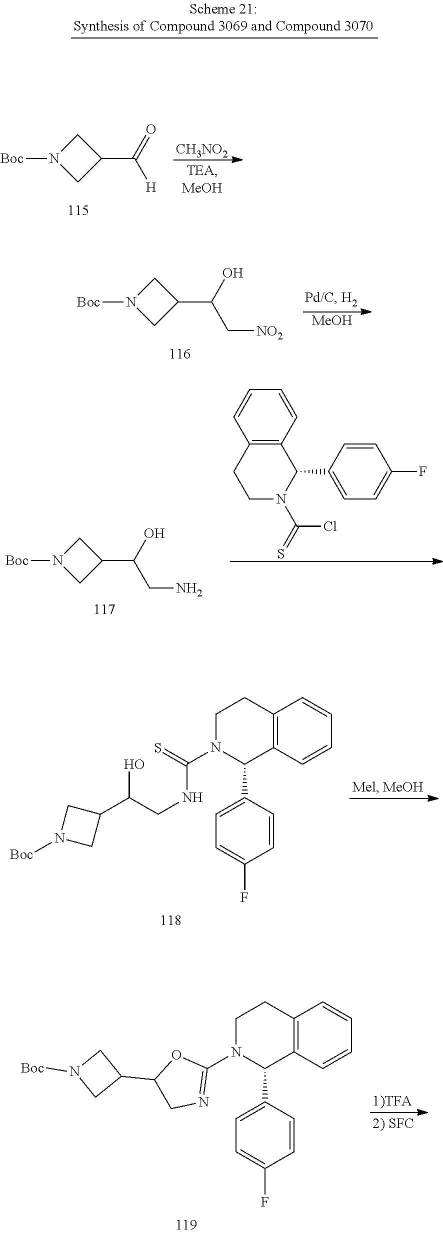

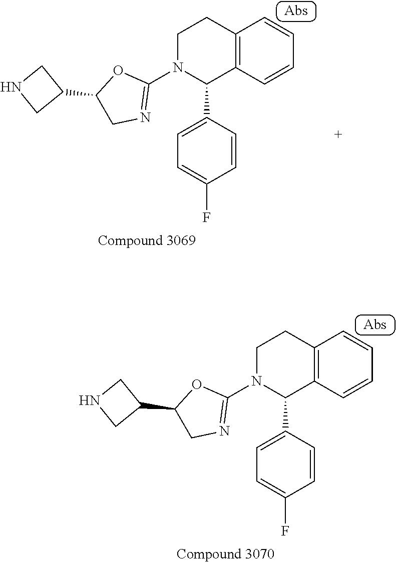 [SMILES: Fc1ccc([C@H]2c3ccccc3CCN2C2=NC[C@H](C3CNC3)O2)cc1 ; Fc1ccc([C@H]2c3ccccc3CCN2C2=NC[C@@H](C3CNC3)O2)cc1]

Step 1: To a solution of tert-butyl 3-formylazetidine-1-carboxylate (3 g, 16.2 mmol) in MeOH (30 mL) were added CH₃NO₂ (2.6 mL, 48.6 mmol) and TEA (4.5 mL, 32.4 mmol) at 0° C. The mixture was stirred at room temperature for 2 h and then concentrated to give the crude product tert-butyl 3-(1-hydroxy-2-nitroethyl)azetidine-1-carboxylate which was used directly in the next step reaction without further purification. LCMS: (M−55)⁺=191.1; Retention time=1.45 min. LCMS CP Method F

Step 2: To a solution of tert-butyl 3-(1-hydroxy-2-nitroethyl)azetidine-1-carboxylate (3.6 g, 14.6 mmol) in MeOH (40 mL) was added Pd/C (1 g, 10 wt %). The resulting reaction mixture was stirred at room temperature overnight under a hydrogen atmosphere. The mixture was filtered and the filtrate was concentrated to give tert-butyl 3-(2-amino-1-hydroxyethyl)azetidine-1-carboxylate. LCMS: (M−55)⁺=217.2; Retention time=1.14 min. LCMS CP Method D

Step 3: To a mixture of tert-butyl 3-(2-amino-1-hydroxyethyl)azetidine-1-carboxylate (1.6 g, 7.4 mmol) and TEA (2 mL, 14.8 mmol) in DMF (20 mL) was added a solution of (S)-1-(4-fluorophenyl)-3,4-dihydroisoquinoline-2(1H)-carbothioyl chloride (1.5 g, 4.9 mmol) in DMF (10 mL) 0° C. The resulting reaction mixture was stirred at 60° C. for 2 h and then diluted with ethyl acetate (100 mL) and water (100 mL). The mixture was extracted with ethyl acetate (3×80 mL). The combined organic phase was washed with brine (60 mL), dried over Na₂SO₄, filtered and concentrated under reduced pressure. The residue was purified by flash column chromatography to give tert-butyl 3-(2-((S)-1-(4-fluorophenyl)-1,2,3,4-tetrahydroisoquinoline-2-carbothioamido)-1-hydroxyethyl)azetidine-1-carboxylate. LCMS: (M+H)⁺=486.0; Retention time=1.98 min. LCMS CP Method C

Step 4: To a solution of tert-butyl 3-(2-((S)-1-(4-fluorophenyl)-1,2,3,4-tetrahydroisoquinoline-2-carbothioamido)-1-hydroxyethyl)azetidine-1-carboxylate (300 mg, 0.6 mmol) in MeOH (8 mL) was added MeI (0.2 mL, 3.1 mmol) at 0° C. The reaction mixture was stirred at room temperature overnight. The resulting reaction mixture was concentrated under reduced pressure. The residue was purified by flash column chromatography to give the product tert-butyl 3-(2-((S)-1-(4-fluorophenyl)-3,4-dihydroisoquinolin-2(1H)-yl)-4,5-dihydrooxazol-5-yl)azetidine-1-carboxylate. LCMS: (M+H)⁺=452.1; Retention time=2.02 min. LCMS CP Method C

Step 5: To a solution of tert-butyl 3-(2-((S)-1-(4-fluorophenyl)-3,4-dihydroisoquinolin-2(1H)-yl)-4,5-dihydrooxazol-5-yl)azetidine-1-carboxylate (150 mg, 0.3 mmol) in DCM (2 mL) was added TFA (1 mL) and the mixture was stirred at room temperature for 2 h. Then the mixture were concentrated under reduced pressure and the residue was purified by Prep-HPLC to give 5-(azetidin-3-yl)-2-((S)-1-(4-fluorophenyl)-3,4-dihydroisoquinolin-2(1H)-yl)-4,5-dihydrooxazole. LCMS: (M+H)⁺=352.2; Retention time=1.33 min. LCMS CP Method D

The diastereomers were separated by chiral SFC eluting with CO₂/EtOH containing 0.2% MA (80%:20%) over an EnantioPak®IG column (20*250 mm 10 μm) to give Compound 3069 (retention time=1.297 min) and Compound 3070 (retention time=1.718 min) both as white solids. Stereochemical assignment of (S) at the 1 position of the tetrahydroisoquinoline is based on enantiomerically pure starting material; the stereochemistry at the chiral center of the four-membered ring is arbitrarily assigned based on chromatographic elution order as compared to related analogues of known configuration.

Compound 3069: LCMS: (M+H)⁺=352.2; purity=95.80% (214 nm); Retention time=1.36 min. LCMS CP Method F. ¹H NMR (400 Hz, CD₃OD): δ 7.28-7.18 (m, 5H), 7.12-7.00 (m, 3H), 6.22 (s, 1H), 4.90-4.85 (m, 1H), 3.91-3.82 (m, 2H), 3.73 (t, J=8.8 Hz, 1H), 3.66-3.56 (m, 1H), 3.45-3.34 (m, 3H), 3.10-2.97 (m, 2H), 2.81 (dt, J=12.4, 4.4 Hz, 2H). Chiral SFC: CO₂/EtOH containing 1% MA (70%:30%) over CHIRALPAK® IG column (4.6×100 mm, 5 μm), retention time=1.297 min, 100% ee.

Compound 3070: LCMS: (M+H)⁺=352.1; purity=100% (214 nm); Retention time=1.34 min. LCMS CP Method F. ¹H NMR (400 Hz, CD₃OD): δ 7.25-7.17 (m, 5H), 7.09-7.01 (m, 3H), 6.15 (s, 1H), 4.90-4.85 (m, 1H), 3.95-3.80 (m, 4H), 3.75-3.66 (m, 2H), 3.49-3.41 (m, 1H), 3.38-3.33 (m, 1H), 3.10-2.97 (m, 2H), 2.81 (dt, J=12.4, 4.4 Hz, 1H). Chiral SFC: CO₂/EtOH containing 1% MA (70%:30%) over CHIRALPAK® IG column (4.6×100 mm, 5 μm), retention time=1.718 min, 88.84% ee.

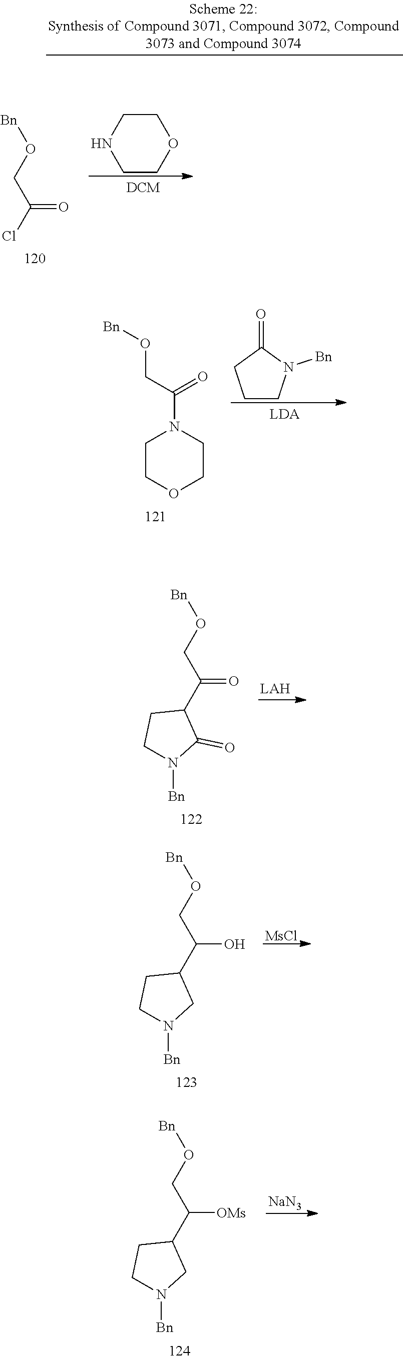

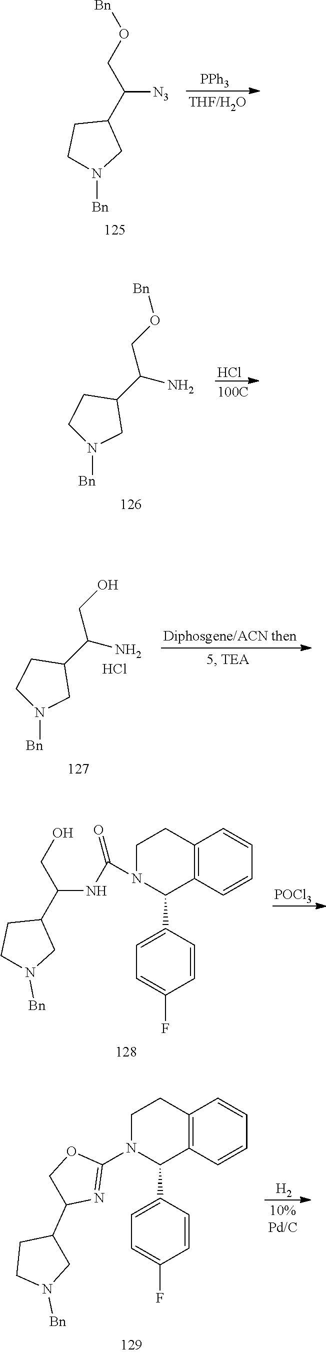

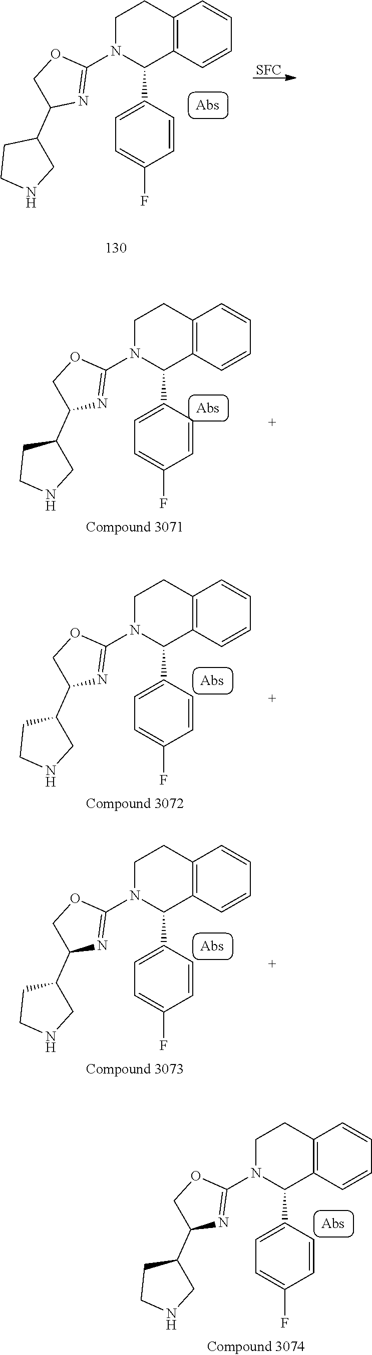

Step 1: To a solution of morpholine (5.9 g, 67.7 mmol) in dry DCM (50 mL) was added a solution of 2-(benzyloxy)acetyl chloride (5 g, 27 mmol) in dry DCM (10 mL) dropwise over 30 min and the reaction mixture was allowed to stir overnight at room temperature. Water (100 mL) was added and the aqueous layer was extracted with DCM (2×50 mL). The combined organic phase was washed with 1 N HCl (50 mL), water (50 mL) and brine (50 mL). Concentration yielded 2-(benzyloxy)-1-morpholinoethan-1-one which was used in the next step without further purification. LCMS: (M+1)⁺=236.2; Retention time=1.44 min. LCMS CP Method A1

Step 2: To a solution of 1-benzylpyrrolidin-2-one (3.5 g, 20 mmol) in dried THF (35 mL) was added LDA (15 mL, 30 mmol, 2 M in THF) at −10° C. After 30 min of stirring, 2-(benzyloxy)-1-morpholinoethan-1-one (5.2 g, 22 mmol) was added over 10 min while keeping the temperature below −5° C. After the red colored reaction mixture became a pale yellow, it was allowed to warm to 10° C. and then quenched by the addition of saturated NaHCO₃ solution (50 mL). The mixture was diluted with THF (30 mL) and extracted with EtOAc (3×50 mL). The combined organic phase was washed with brine, dried over Na₂SO₄, and concentrated in vacuo. The residue was purified by silica gel column chromatography (PE/EA, 10:1 to 2:1) to give 1-benzyl-3-(2-(benzyloxy)acetyl)pyrrolidin-2-one. LCMS: (M+1)⁺=324.1; Retention time=1.70 min. LCMS CP Method B1

Step 3: To a solution of 1-benzyl-3-(2-(benzyloxy)acetyl)pyrrolidin-2-one (1.3 g, 4.3 mmol) in dried THF (15 mL) was added LiAlH₄ (0.33 g, 8.7 mmol) at 0° C. The reaction mixture was stirred at room temperature for 2 h then cooled with an ice bath and quenched sequentially with H₂O (1 mL), 15% aqueous NaOH (1 mL). The mixture was stirred at 0° C. for 30 min and then treated with Na₂SO₄ filtered. The solid was washed with THF (50 mL) and the filtrate concentrated in vacuo to afford 2-(benzyloxy)-1-(1-benzylpyrrolidin-3-yl)ethan-1-ol. LCMS: (M+1)⁺=312.1; Retention time=1.39 min. LCMS CP Method B1

Step 4: To a solution of 2-(benzyloxy)-1-(1-benzylpyrrolidin-3-yl)ethan-1-ol (1.12 g, 3.6 mmol) and TEA (1 mL, 7.2 mmol) in DCM (15 mL) was added dropwise MsCl (0.33 mL, 4.3 mmol) at 0° C. The resulting reaction mixture was stirred at room temperature for 16 h and then diluted with DCM (50 mL). This solution was washed with water, brine, dried over Na₂SO₄ and concentrated to give crude 2-(benzyloxy)-1-(1-benzylpyrrolidin-3-yl)ethyl methanesulfonate. LCMS: (M+H)⁺=390.1; Retention time=1.47 min. LCMS CP Method B1

Step 5: To a solution of 2-(benzyloxy)-1-(1-benzylpyrrolidin-3-yl)ethyl methanesulfonate (1.4 g, 3.6 mmol) in DMSO (15 mL) and H₂O (3 mL) was added NaN₃ (0.93 g, 14.4 mmol) at room temperature. The reaction mixture was heated to 80° C. for 16 hand then poured into water (50 mL) and extracted with DCM (3×30 mL). The combined organic phase was washed with brine, dried over anhydrous Na₂SO₄ and concentrated to give 3-(1-azido-2-(benzyloxy)ethyl)-1-benzylpyrrolidine which was used directly in the next step reaction without purification. LCMS: (M+H)⁺=337.1; Retention time=1.54 min. LCMS CP Method B1

Step 6: To a solution of 3-(1-azido-2-(benzyloxy)ethyl)-1-benzylpyrrolidine (2.7 g, 8 mmol) in MeOH (30 mL) was added Raney-Ni (2 g, in water) at room temperature. The reaction mixture was evacuated, backfilled with hydrogen and stirred at room temperature for 24 h. The reaction mixture was filtered, and the filtrate concentrated to give a residue which was dissolved in 1 N HCl (30 mL) and extracted with DCM (3×20 mL). The pH value was adjusted to 10 with 10% NaOH. The mixture was extracted with DCM (5×30 mL). The combined organic phase was dried and concentrated to give 2-(benzyloxy)-1-(1-benzylpyrrolidin-3-yl)ethan-1-amine which was used directly in the next step reaction without purification. LCMS: (M+H)⁺=311.2; Retention time=1.16 min. LCMS CP Method A1

Step 7: A solution of 2-(benzyloxy)-1-(1-benzylpyrrolidin-3-yl)ethan-1-amine (2 g, 6.45 mmol) in HCl (5 mL, conc.) was heated to 100° C. and stirred at the same temperature for 1 h. The reaction mixture was cooled the pH adjusted to about 10 by the addition of 20% NaOH. The mixture was concentrated and the residue was suspended in DCM/MeOH (200 mL, 5:1). The mixture was filtered and the filtrate dried and concentrated to give 2-amino-2-(1-benzylpyrrolidin-3-yl)ethan-1-ol. LCMS: (M+H)⁺=221.2; Retention time=0.95 min. LCMS CP Method B1

Step 8: To a solution of 2-amino-2-(1-benzylpyrrolidin-3-yl)ethan-1-ol (0.5 g, 2.2 mmol) in dry CH₃CN (10 mL) was added diphosgene (0.4 mL, 3.3 mmol) at 0° C. The reaction mixture was stirred at room temperature for 2 h and then concentrated. The residue was dissolved in dry DMF (10 mL) and 2-amino-2-(1-benzylpyrrolidin-3-yl)ethan-1-ol (0.63 g, 2.9 mmol) and TEA (1.07 mL, 7.7 mmol) were added. The reaction mixture was stirred at room temperature for 16 h and then poured into water and extracted with DCM (3×30 mL). The combined organic phase was washed with brine, dried over anhydrous Na₂SO₄, filtered and concentrated to give a residue. After purified by Prep-HPLC to give (1S)—N-(1-(1-benzylpyrrolidin-3-yl)-2-hydroxyethyl)-1-(4-fluorophenyl)-3,4-dihydroisoquinoline-2(1H)-carboxamide. LCMS: (M+H)⁺=474.3; Retention time=1.55 min. LCMS CP Method B1

Step 9: To a solution of (1S)—N-(1-(1-benzylpyrrolidin-3-yl)-2-hydroxyethyl)-1-(4-fluorophenyl)-3,4-dihydroisoquinoline-2(1H)-carboxamide (370 mg, 0.78 mmol) in dry THF (10 mL) was added POCl₃ (0.36 mL, 3.9 mmol) at room temperature under N₂ atmosphere. The reaction mixture was heated to 65° C. for 1.5 h and then concentrated. The residue was dissolved in water (20 mL), adjusted to alkaline pH with 2 M NaOH and extracted with DCM (4×30 mL). The combined organic phase was washed with brine, dried, filtered and concentrated. The residue was purified by Prep-HPLC to give 4-(1-benzylpyrrolidin-3-yl)-2-((S)-1-(4-fluorophenyl)-3,4-dihydroisoquinolin-2(1H)-yl)-4,5-dihydrooxazole. LCMS: (M+H)⁺=456.1; Retention time=1.87 min. LCMS CP Method C

Step 10: To a solution of 4-(1-benzylpyrrolidin-3-yl)-2-((S)-1-(4-fluorophenyl)-3,4-dihydroisoquinolin-2(1H)-yl)-4,5-dihydrooxazole (166 mg, 0.36 mmol) in MeOH (5 mL) was added 10% Pd/C (50 mg, 33% water). The reaction system was evacuated, backfilled with hydrogen and stirred for 16 h at room temperature. The mixture was filtered and the filtrate concentrated. Purification of the residue by Prep-HPLC afforded 2-((S)-1-(4-fluorophenyl)-3,4-dihydroisoquinolin-2(1H)-yl)-4-(pyrrolidin-3-yl)-4,5-dihydrooxazole. LCMS: (M+H)⁺=366.3; Retention time=1.46 min. LCMS CP Method B1

The diastereomers (70 mg) were separated by chiral HPLC eluting with n-Hexane (0.1% DEA):EtOH (0.1% DEA)=65:35 over a Daicel® IC column (20×250 mm, 10 μm) to give Compound 3071, Compound 3072, Compound 3073 and Compound 3074. Stereochemical assignment of (S) at the 1 position of the tetrahydroisoquinoline is based on enantiomerically pure starting material; the stereochemistry at the chiral center of the four-membered ring is arbitrarily assigned based on chromatographic elution order as compared to related analogues of known configuration.

Compound 3071: LCMS: (M+H)⁺=366.1; Retention time=1.64 min. LCMS CP Method C. ¹H NMR (400 Hz, CDCl3): 7.24-7.14 (m, 6H), 7.00-6.94 (m, 2H), 6.31 (s, 1H), 4.56 (d, J=4.4 Hz, 1H), 4.18-4.12 (m, 1H), 3.59 (t, J=6.2 Hz, 2H), 3.25-3.19 (m, 1H), 2.95-2.75 (m, 4H), 2.64 (d, J=9.6 Hz, 1H), 2.50-2.43 (m, 2H), 1.88-1.83 (m, 1H), 1.55-1.50 (m, 2H). Chiral HPLC: n-Hexane (0.1% DEA):EtOH (0.1% DEA)=60:40 over CHIRALPAK® IG column (4.6×100 mm, 5 μm), retention time=12.805 min), 100% ee.

Compound 3072: LCMS: (M+H)⁺=366.1; Retention time=1.64 min. LCMS CP Method C. ¹H NMR (400 Hz, CDCl3): 7.24-7.14 (m, 6H), 7.00-6.93 (m, 2H), 6.33 (s, 1H), 4.41 (d, J=6 Hz, 1H), 3.61-3.54 (m, 3H), 3.00-2.71 (m, 4H), 2.49-2.28 (m, 5H), 1.64-1.56 (m, 2H). Chiral HPLC: n-Hexane (0.1% DEA):EtOH (0.1% DEA)=60:40 over CHIRALPAK® IG column (4.6×100 mm, 5 μm), retention time=20.907 min), 100% ee.

Compound 3073: LCMS: (M+H)⁺=366.1; Retention time=1.63 min. LCMS CP Method C. ¹H NMR (400 Hz, CDCl3): 7.26-7.15 (m, 6H), 7.00 (t, J=8.4 Hz, 2H), 6.31 (s, 1H), 4.55 (s, 1H), 4.20-4.16 (m, 1H), 3.67-3.57 (m, 2H), 3.28 (t, J=11.6 Hz, 1H), 2.97-2.65 (m, 5H), 2.46 (d, J=7.6 Hz, 2H), 1.97-1.94 (m, 1H), 1.78-1.70 (m, 1H). Chiral HPLC: n-Hexane (0.1% DEA):EtOH (0.1% DEA)=60:40 over CHIRALPAK® IG column (4.6×100 mm, 5 μm), retention time=10.883 min), 100% ee.

Compound 3074: LCMS: (M+H)⁺=366.1; Retention time=1.64 min. LCMS CP Method C. ¹H NMR (400 Hz, CDCl3): 7.23-7.13 (m, 5H), 6.96 (t, J=8.6 Hz, 1H), 6.34 (s, 1H), 4.41 (d, J=4.8 Hz, 1H), 3.58-3.51 (m, 3H), 2.97-2.75 (m, 4H), 2.52-2.32 (m, 5H), 1.67-1.60 (m, 2H). Chiral HPLC: n-Hexane (0.1% DEA):EtOH (0.1% DEA)=60:40 over CHIRALPAK® IG column (4.6×100 mm, 5 μm), retention time=21.358 min), 100% ee.

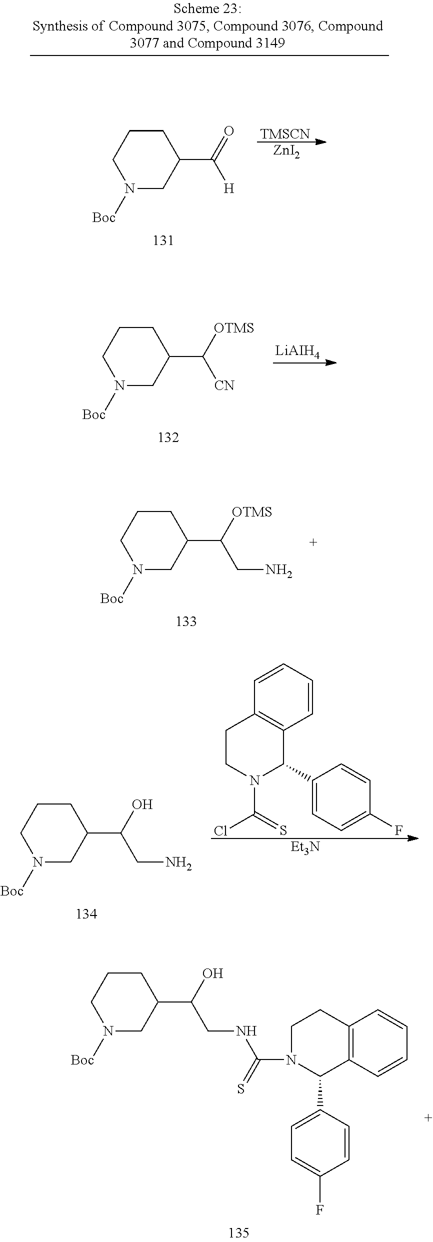

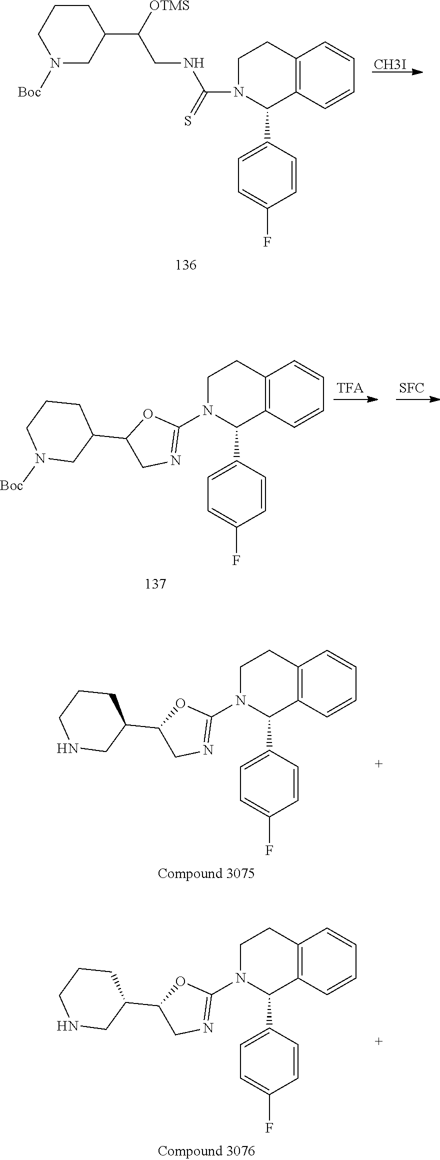

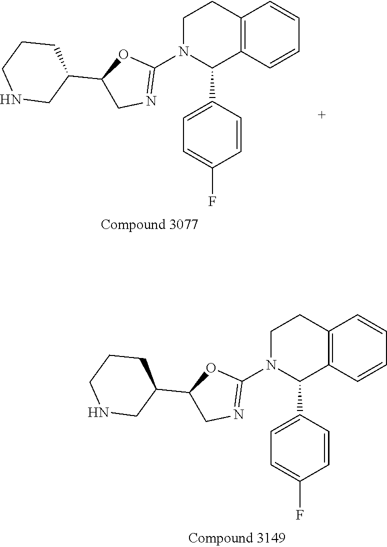 [SMILES: Fc1ccc([C@H]2c3ccccc3CCN2C2=NC[C@@H]([C@H]3CCCNC3)O2)cc1 ; Fc1ccc([C@H]2c3ccccc3CCN2C2=NC[C@@H]([C@@H]3CCCNC3)O2)cc1]

Step 1: To a solution of tert-butyl 3-formylpiperidine-1-carboxylate (4.26, 20 mmol) in anhydrous DCM (60 mL) were added TMSCN (2.38 g, 24 mmol) and ZnI₂ (0.32 g, 1 mmol). The reaction mixture was stirred at room temperature overnight, filtered and the filtrate concentrated to give the crude product tert-butyl 3-(cyano(trimethylsilyloxy)methyl)piperidine-1-carboxylate (6.24 g) which was used in the next step reaction without further purification. LCMS: (M−TMS+Na)+=263.1; Retention time=1.67 min. LCMS CP Method E

Step 2: To a solution of tert-butyl 3-(cyano(trimethylsilyloxy)methyl)piperidine-1-carboxylate (6.24 g, 20 mmol) in THF (30 mL) at 0° C. was added dropwise LiAlH₄ (20 mL, 20 mmol, 1M solution in THF). The mixture was allowed to warm to room temperature and stirred for 1 h. Solid Na₂SO₄*10 H₂O was carefully added, and the mixture was filtered. The filter cake was washed with THF (2×30 mL) and the filtrates concentrated to give tert-butyl 3-(2-amino-1-(trimethylsilyloxy)ethyl)piperidine-1-carboxylate which was used directly in the next step reaction without purification. LCMS: (M+H)⁺=245.2; Retention time=1.18 min. LCMS CP Method E

Step 3: To a solution of tert-butyl 3-(2-amino-1-hydroxyethyl)piperidine-1-carboxylate (732 mg, 3 mmol) and Et₃N (606 mg, 6 mmol) in DMF (6 mL) was slowly added a solution of (S)-1-(4-fluorophenyl)-3,4-dihydroisoquinoline-2(1H)-carbothioyl chloride (903 mg, 3 mmol) in DMF (4 mL). The mixture was stirred at room temperature for 24 h before water (20 mL) was added and the mixture was extracted with EA (3×15 mL). The combined organic phase was dried over Na₂SO₄, filtered and concentrated. The residue was purified by Prep-HPLC to give tert-butyl 3-(2-((S)-1-(4-fluorophenyl)-1,2,3,4-tetrahydroisoquinoline-2-carbothioamido)-1-hydroxyethyl)piperidine-1-carboxylate and tert-butyl 3-(2-((S)-1-(4-fluorophenyl)-1,2,3,4-tetrahydroisoquinoline-2-carbothioamido)-1-(trimethylsilyloxy)ethyl)piperidine-1-carboxylate. LCMS: (M+H)⁺=514.1; Retention time=2.12 min. (M+H)⁺=586.1; Retention time=2.64 min. LCMS CP Method C

Step 4: To a solution of tert-butyl 3-(2-((S)-1-(4-fluorophenyl)-1,2,3,4-tetrahydroisoquinoline-2-carbothioamido)-1-hydroxyethyl)piperidine-1-carboxylate (284 mg, 0.55 mmol) in THF (3 mL) was added CH₃I (393 mg, 2.77 mmol). The mixture was stirred at room temperature overnight and then concentrated to give tert-butyl 3-(2-((S)-1-(4-fluorophenyl)-3,4-dihydroisoquinolin-2(1H)-yl)-4,5-dihydrooxazol-5-yl)piperidine-1-carboxylate which was used directly in the next step reaction without purification. LCMS: (M+H)⁺=480.2; Retention time=2.21 min. LCMS CP Method C

Step 5: To a solution of tert-butyl 3-(2-((S)-1-(4-fluorophenyl)-3,4-dihydroisoquinolin-2(1H)-yl)-4,5-dihydrooxazol-5-yl)piperidine-1-carboxylate (265 mg, 0.55 mmol) in DCM (2 mL) was added TFA (1 mL). The reaction mixture was stirred at room temperature for 2 h, concentrated and the residue purified by Prep-HPLC to give 2-((S)-1-(4-fluorophenyl)-3,4-dihydroisoquinolin-2(1H)-yl)-5-(piperidin-3-yl)-4,5-dihydrooxazole. LCMS: (M+H)⁺=380.1; Retention time=1.60 min. LCMS CP Method C

The diastereomers were separated by chiral SFC eluting with CO₂/EtOH containing 0-2% methanolic ammonia over a Daicel® AD column (20×250 mm, 10 μm) to give Compound 3075, Compound 3149 and mixed fractions containing Compound 3076 & Compound 3077. The isomeric mixture was further separated by chiral SFC eluting with CO₂/EtOH containing 0-2% methanolic ammonia over a Daicel® OD column (20×250 mm, 10 μm) to give Compound 3076 and Compound 3149. Stereochemical assignment of (S) at the 1 position of the tetrahydroisoquinoline is based on enantiomerically pure starting material; the stereochemistry at the chiral center of the four-membered ring is arbitrarily assigned based on chromatographic elution order as compared to related analogues of known configuration. Compound 3075: LCMS: (M+H)⁺=380.2; Retention time=1.23 min. LCMS CP Method B. ¹H NMR (400 Hz, CDCl3): 7.25-7.16 (m, 5H), 7.04-7.00 (d, J=7.2 Hz, 1H), 6.94 (t, J=7.2 Hz, 2H), 6.18 (s, 1H), 4.41-3.38 (m, 1H), 3.92-3.81 (m, 2H), 3.57-3.52 (m, 1H), 3.38-3.34 (m, 1H), 3.06-2.95 (m, 3H), 2.79-2.74 (m, 1H), 2.55-2.42 (m, 2H), 1.91-1.87 (m, 1H), 1.74-1.70 (m, 2H), 1.48-1.44 (m, 1H), 1.30-1.26 (m, 1H). Chiral SFC: CO₂/MeOH containing 0.2% ammonia=60:40 over CHIRALPAK® IG column (4.6×100 mm, 5 μm), retention time=2.51 min), 97.27% ee.

Compound 3076: LCMS: (M+H)⁺=380.3; Retention time=1.24 min. LCMS CP Method B. ¹H NMR (400 Hz, CDCl3): 7.27-7.15 (m, 5H), 7.04-7.00 (d, J=7.2 Hz, 1H), 6.95 (t, J=8.8 Hz, 2H), 6.20 (s, 1H), 4.33-4.29 (m, 1H), 3.94-3.88 (m, 2H), 3.59-3.53 (m, 1H), 3.34-3.30 (m, 2H), 3.09-3.02 (m, 2H), 2.78-2.73 (m, 1H), 2.60-2.40 (m, 2H), 1.75-1.66 (m, 3H), 1.49-1.44 (m, 1H), 1.30-1.25 (m, 1H). Chiral SFC: CO₂/MeOH containing 0.2% ammonia=60:40 over CHIRALPAK® IG column (4.6×100 mm, 5 μm), retention time=1.05 min), 100% ee.

Compound 3077: LCMS: (M+H)⁺=380.1; Retention time=1.26 min. LCMS CP Method B. 1H NMR (400 Hz, CDCl3): 7.25-7.17 (m, 5H), 7.05-7.00 (d, J=7.2 Hz, 1H), 6.97 (t, J=7.2 Hz, 2H), 6.21 (s, 1H), 4.40-3.37 (m, 1H), 3.86-3.80 (m, 2H), 3.59-3.54 (m, 1H), 3.39-3.35 (m, 1H), 3.07-2.96 (m, 3H), 2.79-2.75 (m, 1H), 2.54-2.43 (m, 2H), 1.74-1.68 (m, 3H), 1.48-1.43 (m, 1H), 1.30-1.25 (m, 1H). Chiral SFC: CO₂/MeOH containing 0.2% ammonia=60:40 over CHIRALPAK® IG column (4.6×100 mm, 5 μm), retention time=1.09 min), 100% ee.

Compound 3149: LCMS: (M+H)⁺=380.2; Retention time=1.24 min. LCMS CP Method B. ¹H NMR (400 Hz, CDCl3): 7.26-7.17 (m, 5H), 7.04-7.00 (d, J=7.2 Hz, 1H), 6.94 (t, J=8.8 Hz, 2H), 6.19 (s, 1H), 4.34-4.30 (m, 1H), 3.92-3.82 (m, 2H), 3.57-3.52 (m, 1H), 3.36-3.21 (m, 2H), 3.07-3.01 (m, 2H), 2.78-2.74 (m, 1H), 2.56-2.38 (m, 2H), 1.80-1.70 (m, 3H), 1.50-1.45 (m, 1H), 1.31-1.26 (m, 1H). Chiral SFC: CO₂/MeOH containing 0.2% ammonia=60:40 over CHIRALPAK® IG column (4.6×100 mm, 5 μm), retention time=1.42 min), 96.05% ee.

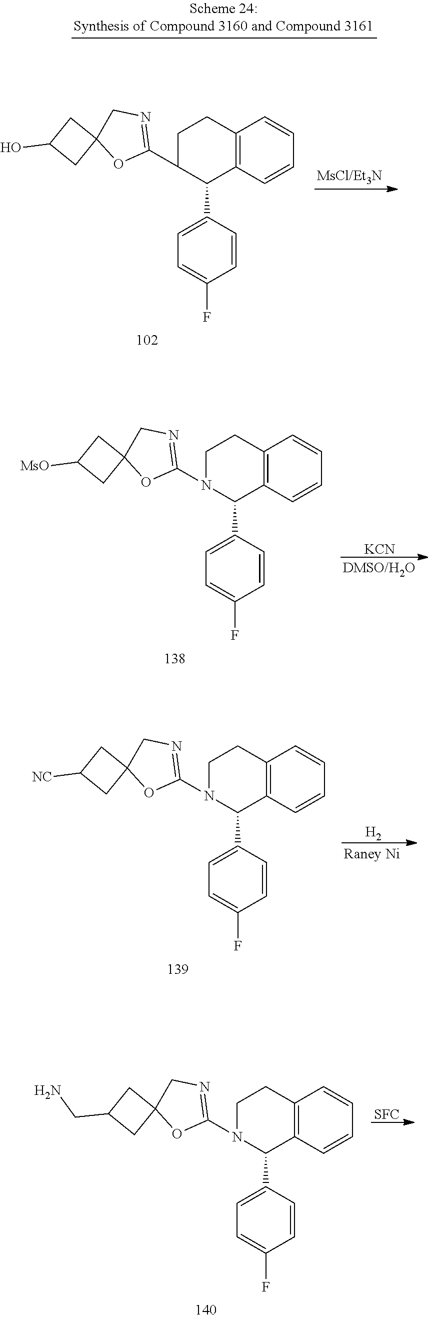

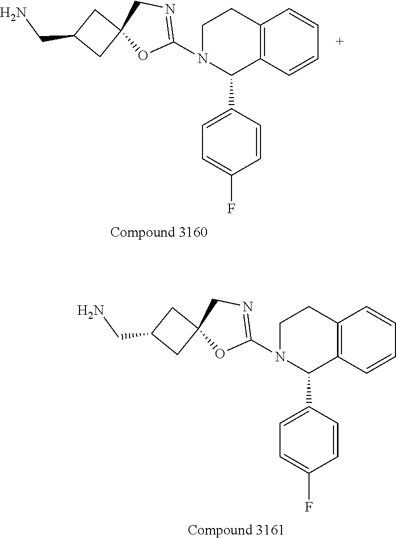 [SMILES: NC[C@H]1C[C@@]2(CN=C(N3CCc4ccccc4[C@@H]3c3ccc(F)cc3)O2)C1 ; NC[C@H]1C[C@]2(CN=C(N3CCc4ccccc4[C@@H]3c3ccc(F)cc3)O2)C1]

Step 1: A mixture of (S)-6-(1-(4-fluorophenyl)-3,4-dihydroisoquinolin-2(1H)-yl)-5-oxa-7-azaspiro[3.4]oct-6-en-2-ol (352 mg, 1.0 mmol), methanesulfonyl chloride (230 mg, 2 mmol) and TEA (303 mg, 3.0 mmol) was stirred at 0° C. for 2 h. The mixture was concentrated in vacuo and the residue was purified by column chromatography to give (S)-6-(1-(4-fluorophenyl)-3,4-dihydroisoquinolin-2(1H)-yl)-5-oxa-7-azaspiro[3.4]oct-6-en-2-yl methanesulfonate. LCMS: (M+H)⁺=431.1; Retention time=1.52 min. LCMS CP Method C1

Step 2: A mixture of (S)-6-(1-(4-fluorophenyl)-3,4-dihydroisoquinolin-2(1H)-yl)-5-oxa-7-azaspiro[3.4]oct-6-en-2-yl methanesulfonate (380 mg, 0.88 mmol), KCN (65 mg, 1 mmol) and DMSO (10 mL) was stirred at 80° C. for 2 h. The reaction mixture was cooled to room temperature diluted with water and extracted with DCM. The combined organic phase was dried over Na₂SO₄, filtered, and concentrated to give a residue which was purified by Prep-HPLC to give (S)-6-(1-(4-fluorophenyl)-3,4-dihydroisoquinolin-2(1H)-yl)-5-oxa-7-azaspiro[3.4]oct-6-ene-2-carbonitrile. LCMS: (M+H)⁺=362.1; Retention time=1.70 min. LCMS CP Method C

Step 3: To a solution of (S)-2-(1-benzylpiperidin-4-yloxy)-1-(1-(4-fluorophenyl)-3,4-dihydroisoquinolin-2(1H)-yl)-2-methylpropan-1-one (80 mg, 0.16 mmol) in MeOH (5 mL) was added Ranney (200 mg). The reaction mixture was stirred at room temperature overnight under hydrogen atmosphere, filtered and the filtrate concentrated under reduced pressure. The residue was purified by Prep-HPLC to give (S)-1-(1-(4-fluorophenyl)-3,4-dihydroisoquinolin-2(1H)-yl)-2-methyl-2-(piperidin-4-yloxy)propa-n-1-one. LCMS: (M+H)⁺=366; Retention time=1.60 min. LCMS CP Method C

The diastereomers were separated by chiral SFC eluting with CO₂/MeOH containing 0.2% MA (70%:30%) over an EnantioPak®IG column (20*250 mm 10 μm) to give Compound 3160 (retention time=1.420 min) and Compound 3161 (retention time=1.362 min). Stereochemical assignment of (S) at the 1 position of the tetrahydroisoquinoline is based on enantiomerically pure starting material; the stereochemistry at the four-membered ring is arbitrarily assigned based on chromatographic elution order as compared to related analogues of known configuration. Compound 3160: LCMS: (M+H)⁺=366; purity=100% (214 nm); Retention time=1.61 min. LCMS CP Method C2. ¹H NMR (400 MHz, MeOD) δ 8.51 (s, 2H), 7.27 (qd, J=8.5, 6.5 Hz, 5H), 7.17-7.02 (m, 3H), 6.27 (d, J=6.7 Hz, 1H), 3.96 (d, J=37.1 Hz, 2H), 3.83 (dt, J=9.7, 4.9 Hz, 1H), 3.67-3.53 (m, 1H), 3.07 (dd, J=21.6, 6.3 Hz, 3H), 2.94-2.66 (m, 2H), 2.63-2.53 (m, 1H), 2.53-2.18 (m, 3H). Chiral SFC: CO₂/MeOH containing 1% MA (65%:35%) over CHIRALPAK® IG column (4.6×100 mm, 5 μm), retention time=1.420 min, 70% ee.

Compound 3161: LCMS: (M+H)⁺=366; purity=100% (214 nm); Retention time=1.60 min. LCMS CP Method C2. ¹H NMR (400 MHz, MeOD) δ 8.54 (s, 2H), 7.42-7.18 (m, 5H), 7.18-7.02 (m, 3H), 6.31 (s, 1H), 3.94 (s, 2H), 3.89-3.79 (m, 1H), 3.74-3.60 (m, 1H), 3.15-2.99 (m, 3H), 2.83 (ddt, J=18.2, 11.4, 4.8 Hz, 4H), 2.33 (d, J=16.4 Hz, 2H). Chiral SFC: CO₂/MeOH containing 1% MA (65%:35%) over CHIRALPAK® IG column (4.6×100 mm, 5 μm), retention time=1.462 min, 70% ee.

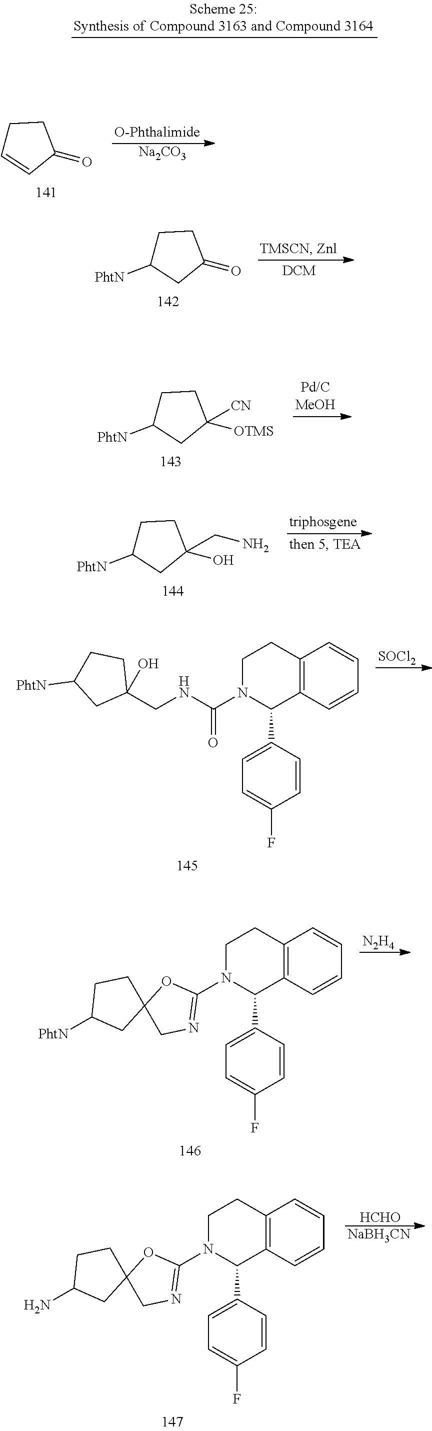

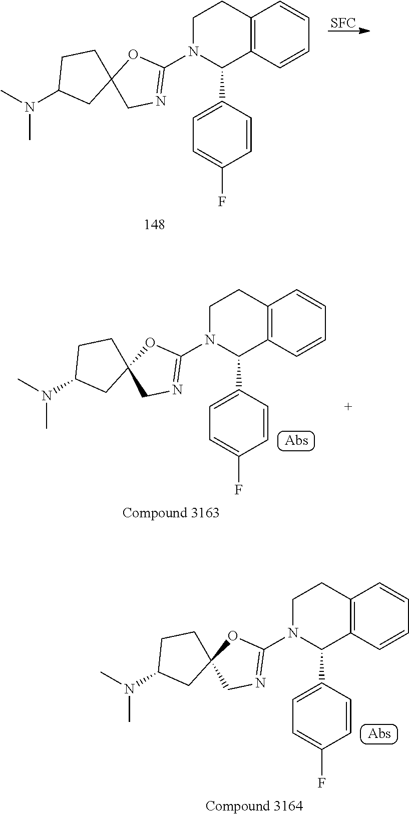

Step 1: To a suspension of cyclopent-2-enone (16.4 g, 200 mmol) and Na₂CO₃ (31.8 g, 300 mmol) in THF (500 mL) was added Phthalimide (29.6 g, 200 mmol). The resulting reaction mixture was stirred for 16 h at 80° C., the solvent removed under reduced pressure and the residue diluted with water (300 mL). The mixture was extracted with EA (3×200 mL) and the combined organic phase was washed with brine (500 mL), dried over anhydrous Na₂SO₄, filtered and concentrated. The residue was purified by flash chromatography (silica, DCM:MeOH=100:1) to give 2-(3-oxocyclopentyl)isoindoline-1,3-dione. LCMS: (M+H)⁺=230.1; Retention time=1.48 min. LCMS CP Method C

Step 2: To a suspension of 2-(3-oxocyclopentyl)isoindoline-1,3-dione (32 g, 140 mmol) and ZnI₂ (5.38 g, 28 mmol) in DCM (400 mL) was added TMSCN (15.2 g, 154 mmol). The resulting reaction mixture was stirred for 2 h at 25° C., filtered and the filtrate concentrated. The residue was purified by column flash (silica, DCM:MeOH=100:1) to give 3-(1,3-dioxoisoindolin-2-yl)-1-(trimethylsilyloxy)cyclopentanecarbonitrile. LCMS: (M+H)⁺=329.1; Retention time=1.66 min. LCMS CP Method C

Step 3: To a solution of 3-(1,3-dioxoisoindolin-2-yl)-1-(trimethylsilyloxy) cyclopentanecarbonitrile (21 g, 64 mmol) in MeOH (200 mL) were added Pd/C (2 g, 10% w/w %) and conc. HCl (1 mL). The reaction mixture was stirred under a hydrogen atmosphere for 16 h at 25° C., filtered and the filtrate concentrated. The residue was purified by flash chromatography (silica, DCM:MeOH=10:1) to give 2-(3-(aminomethyl)-3-hydroxycyclopentyl)isoindoline-1,3-dione. LCMS: (M+H)⁺=261.1; Retention time=1.21 min. LCMS CP Method C

Step 4: To a solution of THIQ (13.6 g, 60 mmol) in DCM (150 mL) was added triphosgene (5.94 g, 20 mmol). The resulting reaction mixture was stirred for 45 min at 0° C. before being concentrated to give a white solid. This was added to a solution of 2-(3-(aminomethyl)-3-hydroxycyclopentyl)isoindoline-1,3-dione (15.6 g, 60 mmol) and TEA (18.2 g, 180 mmol) in DMF (150 mL). The reaction mixture was stirred at 60° C. for 3 h and then water (500 mL) was added. The mixture was extracted with EA (3×300 mL) and the combined organic phase was washed with brine (500 mL), dried over anhydrous Na₂SO₄, filtered and concentrated. The residue was purified by prep-HPLC to give (1S)—N-((3-(1,3-dioxoisoindolin-2-yl)-1-hydroxycyclopentyl)methyl)-1-(4-fluorophenyl)-3,4-dihydroisoquinoline-2(1H)-carboxamide. LCMS: (M+H)⁺=514.1; Retention time=1.77 min. LCMS CP Method C

Step 5: To a solution of (1S)—N-((3-(1,3-dioxoisoindolin-2-yl)-1-hydroxycyclopentyl) methyl)-1-(4-fluorophenyl)-3,4-dihydroisoquinoline-2(1H)-carboxamide (4.1 g, 8 mmol) in DCM (50 mL) was added SOCl₂ (1.07 g, 9 mmol). The reaction mixture was stirred for 5 min at 25° C., concentrated and the residue diluted with water (50 mL). After extraction with EA (3×30 mL), the combined organic phase was washed with brine (100 mL), dried over Na₂SO₄, filtered and concentrated. The residue was purified by prep-HPLC to give 2-(2-((S)-1-(4-fluorophenyl)-3,4-dihydroisoquinolin-2(1H)-yl)-1-oxa-3-azaspiro[4.4]non-2-en-7-yl)isoindoline-1,3-dione. LCMS: (M+H)⁺=496.2; Retention time=1.59 min. LCMS CP Method C

Step 6: To a solution of 2-(2-((S)-1-(4-fluorophenyl)-3,4-dihydroisoquinolin-2(1H)-yl)-1-oxa-3-azaspiro[4.4]non-2-en-7-yl)isoindoline-1,3-dione (2 g, 4 mmol) in EtOH (20 mL) was added N₂H₄—H₂O (470 mg, 8 mmol). The resulting reaction mixture was stirred for 3 h at 75° C., filtered and the filtrate concentrated. The residue was treater with water (50 mL) and the mixture extracted with EA (3×30 mL). The combined organic phase was washed with brine (100 mL), dried over Na₂SO₄, filtered and concentrated. The residue was purified by prep-HPLC to give 2-((S)-1-(4-fluorophenyl)-3,4-dihydroisoquinolin-2(1H)-yl)-1-oxa-3-azaspiro[4.4]non-2-en-7-amine. LCMS: (M+H)⁺=366.2; Retention time=1.64 min. LCMS CP Method C

Step 7: To a solution of 2-((S)-1-(4-fluorophenyl)-3,4-dihydroisoquinolin-2(1H)-yl)-1-oxa-3-azaspiro[4.4]non-2-en-7-amine (680 mg, 1.9 mmol) and HCHO (648 mg, 6 mmol, 37% w/w aqueous) in MeOH (5 mL) was added NaBH₃CN (310 mg, 5 mmol). The reaction mixture was stirred at room temperature for 6 h and concentrated. The residue which was purified by prep-HPLC to give 2-((S)-1-(4-fluorophenyl)-3,4-dihydroisoquinolin-2(1H)-yl)-N,N-dimethyl-1-oxa-3-azaspiro[4.4]non-2-en-7-amine.

The diastereomers were separated by chiral SFC eluting with CO₂/MeOH (0.2% Methanol Ammonia)=80/20 over an EnantioPak® OD column (20*250 mm 10 μm) to give Compound 3163 (retention time=1.213 min) and Compound 3164 (retention time=1.651 min). Stereochemical assignment at 1 position of the tetrahydroisoquinoline is assigned based on enantiomerically pure starting material; the stereochemistry at the pyrrolidine chiral center is arbitrarily assigned based on chromatographic elution order as compared to related analogues of known configuration.

Compound 3163: LCMS: (M+H)⁺=394.2; Retention time=1.21 min. LCMS CP Method C. ¹H NMR (400 MHz, CDCl₃) δ 7.25-7.15 (m, 5H), 7.09-6.96 (m, 3H), 6.18 (s, 1H), 4.01 (d, J=13.6 Hz, 1H), 3.88 (q, J=11.9 Hz, 2H), 3.58-3.49 (m, 1H), 3.17 (s, 1H), 3.07 (dd, J=16.7, 10.0 Hz, 1H), 2.86 (d, J=16.2 Hz, 1H), 2.59-2.46 (m, 6H), 2.18 (s, 3H), 2.06 (dd, J=13.6, 10.4 Hz, 3H). Chiral SFC: CO₂/MeOH containing 0.2% Methanol Ammonia=65:35 over CHIRALPAK® IG column (4.6*100 mm 3 μm), retention time=1.14 min, 100% ee.

Compound 3164: LCMS: (M+H)⁺=394.2; Retention time=1.22 min. LCMS CP Method C. 1H NMR (400 MHz, CDCl₃) δ 7.25-7.14 (m, 5H), 7.03 (d, J=7.4 Hz, 1H), 7.00-6.93 (m, 2H), 6.17 (s, 1H), 3.99-3.87 (m, 1H), 3.76 (q, J=12.3 Hz, 2H), 3.39-3.26 (m, 1H), 3.04 (ddd, J=16.6, 10.6, 6.1 Hz, 1H), 2.94-2.83 (m, 1H), 2.82-2.72 (m, 1H), 2.38-2.29 (m, 6H), 2.30-2.21 (m, 2H), 2.13-2.04 (m, 2H), 2.03-1.94 (m, 2H). Chiral SFC: CO₂/MeOH containing 0.2% Methanol Ammonia=65:35 over CHIRALPAK® IG column (4.6*100 mm 3 μm), retention time=1.23 min, 100% ee.

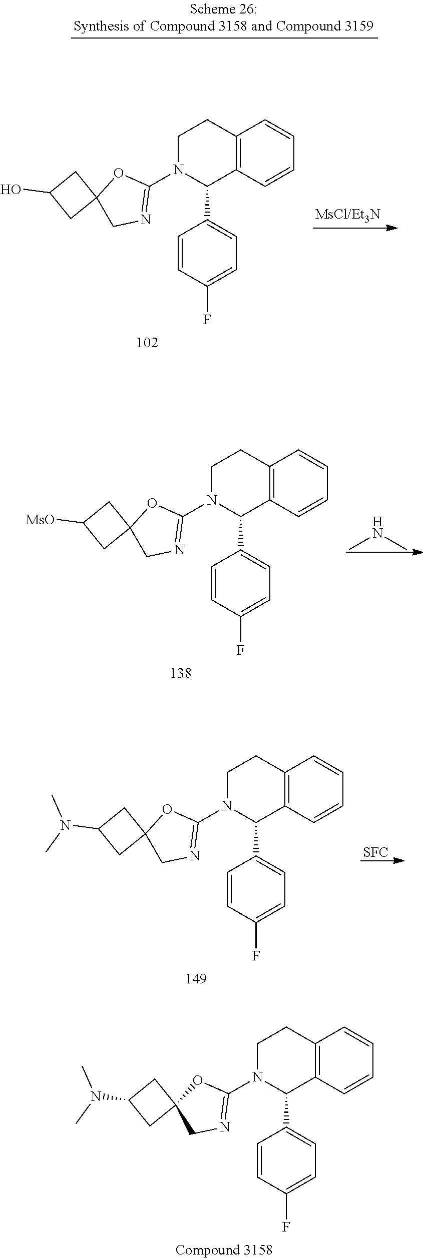

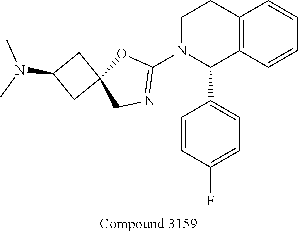 [SMILES: CN(C)[C@H]1C[C@@]2(CN=C(N3CCc4ccccc4[C@@H]3c3ccc(F)cc3)O2)C1]

Step 1: To a solution of (S)-6-(1-(4-fluorophenyl)-3,4-dihydroisoquinolin-2(1H)-yl)-5-oxa-7-azaspiro[3.4]oct-6-en-2-ol (352 mg, 1 mmol) and TEA (202 mg, 2 mmol) in DCM (5 mL) was added MsCl (114 mg, 1 mmol). The reaction mixture was stirred at 25° C. for 2 h and then water (10 mL) was added. The mixture was extracted with DCM (3×10 mL) and the combined organic phase washed with brine (20 mL), dried over anhydrous Na₂SO₄, filtered and concentrated. The residue was purified by prep-HPLC to give (S)-6-(1-(4-fluorophenyl)-3,4-dihydroisoquinolin-2(1H)-yl)-5-oxa-7-azaspiro[3.4]oct-6-en-2-yl methanesulfonate. LCMS: (M+H)⁺=353.1, purity=100% (214 nm), Retention time=1.79 min. Method C

Step 2: A solution of (S)-6-(1-(4-fluorophenyl)-3,4-dihydroisoquinolin-2(1H)-yl)-5-oxa-7-azaspiro[3.4]oct-6-en-2-yl methanesulfonate (215 mg, 0.5 mmol) in aqueous dimethylamine (5 mL) was stirred at 100° C. for 6 h before the reaction mixture was extracted with EA (3×10 mL). The combined organic phase was washed with brine (20 mL), dried over anhydrous Na₂SO₄, filtered and concentrated. The residue was purified by Prep-HPLC to give (S)-6-(1-(4-fluorophenyl)-3,4-dihydroisoquinolin-2(1H)-yl)-N,N-dimethyl-5-oxa-7-azaspiro[3.4]oct-6-en-2-amine. LCMS: (M+H)⁺=380.1, purity=100% (214 nm), Retention time=1.65 min. Method C

The diastereomers were separated by chiral SFC eluting with CO₂/MeOH (0.2%/Methanol Ammonia)=70/30 over an EnantioPak® OZ column (20*250 mm 10 μm) to give Compound 3158 (retention time=2.68 min) and Compound 3159 (retention time=2.03 min) both as white solids. Stereochemical assignment at 1 position of the tetrahydroisoquinoline is assigned based on enantiomerically pure starting material; the stereochemistry at the cyclobutane center is arbitrarily assigned based on chromatographic elution order as compared to related analogues of known configuration.

Compound 3158: LCMS: (M+H)⁺=380.1, purity=100% (214 nm), Retention time=1.65 min. Method C. ¹H NMR (400 MHz, CDCl₃) δ 7.25-7.14 (m, 5H), 7.03 (d, J=7.5 Hz, 1H), 6.96 (t, J=8.7 Hz, 2H), 6.22 (s, 1H), 4.00-3.88 (m, 1H), 3.73 (d, J=12.9 Hz, 2H), 3.36-3.26 (m, 1H), 3.05 (ddd, J=16.6, 10.5, 6.1 Hz, 1H), 2.88-2.79 (m, 1H), 2.75 (dt, J=16.3, 4.0 Hz, 1H), 2.59-2.44 (m, 2H), 2.16 (s, 6H), 2.12-2.04 (m, 2H). Chiral SFC: CO₂/MeOH containing 0.2% Methanol Ammonia=80:20 over CHIRALPAK® IG column (4.6*100 mm 5 μm), retention time=2.191 min), 100% ee.

Compound 3159: LCMS: (M+H)⁺=380.1, purity=100% (214 nm), Retention time=1.67 min. Method C. ¹H NMR (400 MHz, CDCl₃) δ 7.23-7.14 (m, 5H), 7.03 (d, J=7.5 Hz, 1H), 6.96 (t, J=8.7 Hz, 2H), 6.21 (s, 1H), 3.98-3.89 (m, 1H), 3.73 (s, 2H), 3.36-3.26 (m, 1H), 3.04 (ddd, J=16.5, 10.5, 6.2 Hz, 1H), 2.86-2.79 (m, 1H), 2.74 (dt, J=16.3, 4.0 Hz, 1H), 2.58-2.45 (m, 2H), 2.14 (d, J=10.1 Hz, 6H), 2.12-2.03 (m, 2H). Chiral SFC: CO₂/MeOH containing 0.2% Methanol Ammonia=80:20 over CHIRALPAK® IG column (4.6*100 mm 5 μm), retention time=3.123 min), 100% ee.

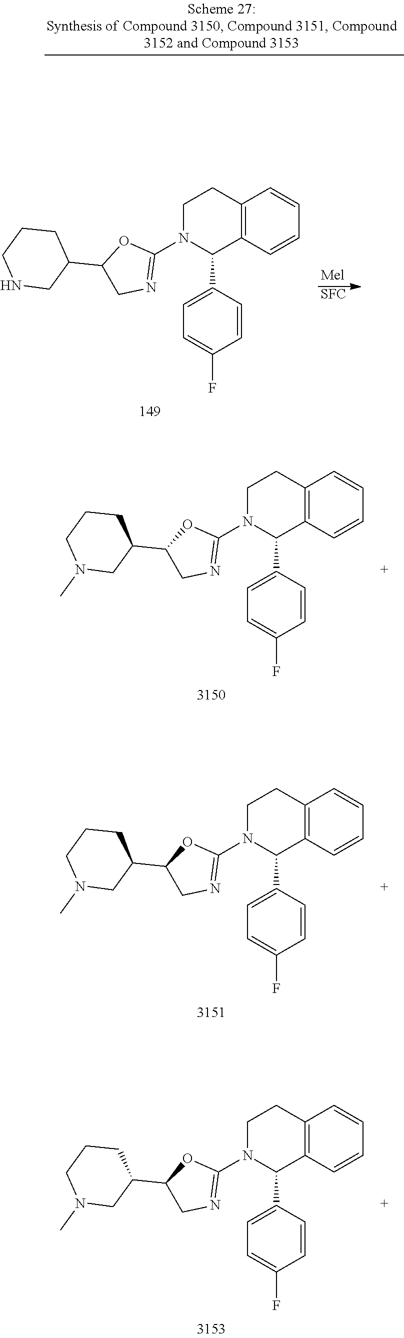

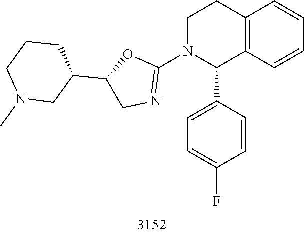 [SMILES: CN1CCC[C@H]([C@H]2CN=C(N3CCc4ccccc4[C@@H]3c3ccc(F)cc3)O2)C1]

Compound 3150, Compound 3151, Compound 3152 and Compound 3153 were prepared by methylation of intermediate 149 prepared as described in Scheme 23.

The diastereomers (Compound 3151 and Compound 3153) were separated by chiral HPLC eluting with n-Hexane (0.1% DEA):IPA (0.1% DEA)=70:30 over a Chiral® IG column (20×250 mm, 10 μm) to give Compound 3151 and Compound 3153. Stereochemical assignment at 1 position of the tetrahydroisoquinoline is assigned based on enantiomerically pure starting material; the stereochemistry at the dihydrooxazole and piperidine centers is arbitrarily assigned based on chromatographic elution order as compared to related analogues of known configuration.

Compound 3151: LCMS: (M+H)⁺=394.1; Retention time=1.16 min. LCMS CP Method A. ¹H NMR (400 Hz, CDCl₃): 7.26-7.17 (m, 5H), 7.04-7.02 (d, J=7.6 Hz, 1H), 6.95 (t, J=8.8 Hz, 2H), 6.20 (s, 1H), 4.35-4.31 (m, 1H), 3.91-3.80 (m, 2H), 3.58-3.53 (m, 1H), 3.31-3.25 (m, 1H), 3.04-2.96 (m, 2H), 2.80-2.70 (m, 2H), 2.26 (s, 3H), 1.86-1.71 (m, 2H), 1.70-1.58 (m, 4H), 1.00-0.95 (m, 1H). Chiral SFC: n-Hexane (0.1% DEA):IPA (0.1% DEA)=80:20 over CHIRALPAK® IG column (4.6×250 mm, 5 μm), retention time=16.532 min), 100% ee.

Compound 3153: LCMS: (M+H)⁺=394.1; Retention time=1.17 min. LCMS CP Method A. ¹H NMR (400 Hz, CDCl₃): 7.26-7.17 (m, 5H), 7.05 (d, J=7.2 Hz, 1H), 6.98 (t, J=8.8 Hz, 2H), 6.19 (s, 1H), 4.37-4.32 (m, 1H), 3.95-3.83 (m, 2H), 3.58-3.53 (m, 1H), 3.38-3.32 (m, 1H), 3.06-2.96 (m, 2H), 2.84-2.75 (m, 2H), 2.30 (s, 3H), 1.92-1.83 (m, 2H), 1.75-1.62 (m, 4H), 1.01-0.97 (m, 1H). Chiral SFC: n-Hexane (0.1% DEA):IPA (0.1% DEA)=80:20 over CHIRALPAK® IG column (4.6×250 mm, 5 μm), retention time=9.073 min), 100% ee.

The diastereomers (Compound 3150 and Compound 3152) were separated by chiral SFC eluting with CO₂/EtOH (0.5% Methanol Ammonia)=50/50 over a Daicel® IG column (20×250 mm, 10 μm) to give Compound 3150 and Compound 3152. Stereochemical assignment at 1 position of the tetrahydroisoquinoline is assigned based on enantiomerically pure starting material; the stereochemistry at the dihydrooxazole and piperidine centers is arbitrarily assigned based on chromatographic elution order as compared to related analogues of known configuration.

Compound 3150: LCMS: (M+H)⁺=394.1; Retention time=1.16 min. LCMS CP Method A. ¹H NMR (400 Hz, CDCl₃): 7.27-7.18 (m, 5H), 7.04 (d, J=7.6 Hz, 1H), 6.98 (t, J=8.4 Hz, 2H), 6.18 (s, 1H), 4.45-4.41 (m, 1H), 3.88-3.82 (m, 2H), 3.60-3.54 (m, 1H), 3.31-3.26 (m, 1H), 2.88-2.75 (m, 3H), 2.28 (s, 3H), 1.88-1.73 (m, 5H), 1.63-1.57 (m, 1H), 1.01-0.97 (m, 1H). Chiral SFC: CO₂/30% EtOH containing 1% ammonia over CHIRALPAK® IG column (4.6×100 mm, 5 μm), retention time=1.841 min), 100% ee.

Compound 3152: LCMS: (M+H)⁺=394.1; Retention time=1.16 min. LCMS CP Method A. ¹H NMR (400 Hz, CDCl₃): 7.27-7.17 (m, 5H), 7.05 (d, J=7.2 Hz, 1H), 6.97 (t, J=8.8 Hz, 2H), 6.22 (s, 1H), 4.43-4.39 (m, 1H), 3.94-3.81 (m, 2H), 3.60-3.54 (m, 1H), 3.33-3.27 (m, 1H), 3.07-3.02 (m, 1H), 2.80-2.68 (m, 3H), 2.26 (s, 3H), 1.89-1.71 (m, 5H), 1.60-1.55 (m, 1H), 1.03-0.99 (m, 1H). Chiral SFC: CO₂/EtOH containing 1% ammonia=70:30 over CHIRALPAK® IG column (4.6×100 mm, 5 μm), retention time=1.373 min), 100% ee.

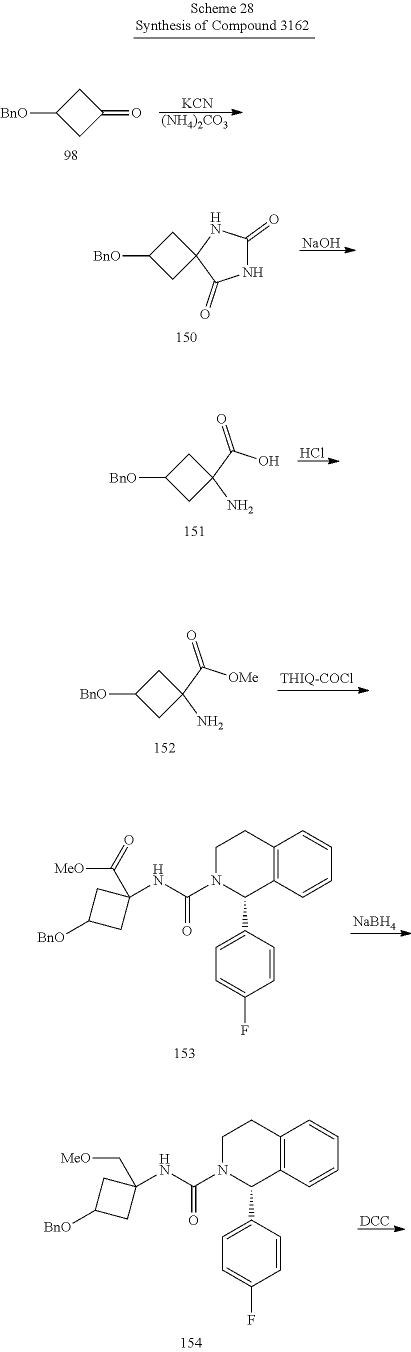

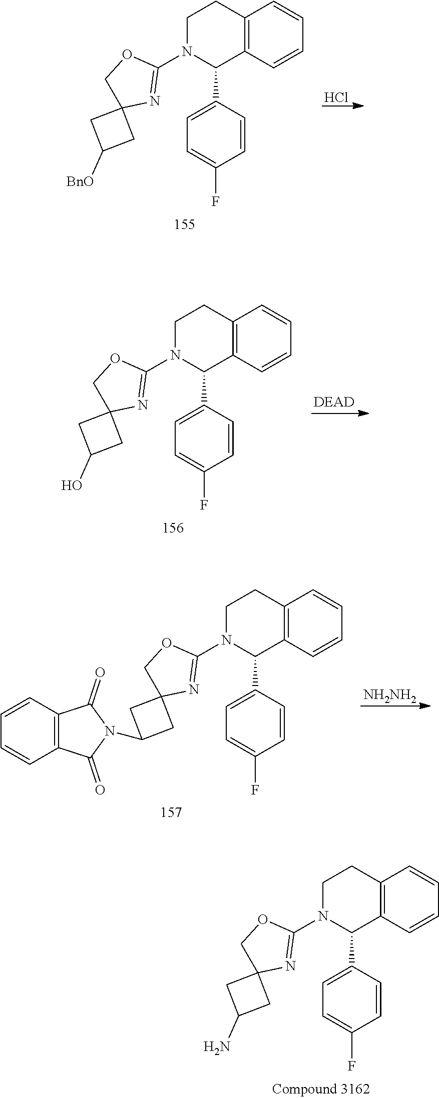 [SMILES: Fc1ccc([C@H]2c3ccccc3CCN2C2=NC3(CO2)CC(OCc2ccccc2)C3)cc1 ; Cl ; OC1CC2(COC(N3CCc4ccccc4[C@@H]3c3ccc(F)cc3)=N2)C1 ; O=C1c2ccccc2C(=O)N1C1CC2(COC(N3CCc4ccccc4[C@@H]3c3ccc(F)cc3)=N2)C1 ; "NH2NH2" ; NC1CC2(COC(N3CCc4ccccc4[C@@H]3c3ccc(F)cc3)=N2)C1]

Step 1: To a solution of 3-(benzyloxy)cyclobutan-1-one (35.2 g, 0.2 mol) in mixed EtOH and H₂O (500 mL, EtOH:H₂O=1:1) were added KCN (39 g, 0.6 mol) and ammonium carbonate (192 g, 2 mol). The reaction mixture was heated to reflux for 16 h, filtered and concentrated in vacuo to give the crude product which was used directly without further purification. ¹H NMR (400 MHz, DMSO,) δ 10.63 (s, 1H. NH), 8.24 (s, 1H, NH), 7.38-7.27 (m, 5H), 4.32 (s, 1H), 4.06-3.98 (m, 1H), 2.68-2.61 (m, 2H). LCMS: (M+H)⁺=247.1; Retention time=1.24 min. LCMS CP Method B

Step 2: To a solution of 2-(benzyloxy)-5,7-diazaspiro[3.4]octane-6,8-dione (24.6 g, 0.1 mol) in water (200 mL) at 0° C. was added NaOH (16 g 0.4 mol). The reaction mixture was heated at reflux for 24 h and then cooled to room temperature and quenched by adjusting the pH to between 5 and 6 through the addition of conc. HCl. The solvent was removed in vacuo and the residue treated with THF (250 mL), filtered and concentrated in vacuo to give 1-amino-3-benzyloxycyclobutane-1-carboxylic acid. ¹H NMR (d4-methanol) δ 2.2-2.9 (m, 4H, CH₂), 4.3 (t, J=6.9 Hz, 1H, OCH), 4.5 (s, 2H, OCH₂), 7.23 (br s, 5H, phenyl). LCMS: (M+H)⁺=222.1; Retention time=0.65 min. LCMS CP Method C

Step 3: To a refluxing solution of 1-amino-3-(benzyloxy)cyclobutane-1-carboxylic acid (11 g, 50 mmol) in MeOH (100 mL) was added dropwise SOCl₂(5.9 g, 50 mmol) over 3 min. The reaction mixture was stirred for an additional 3 h at reflux and then concentrated under reduced pressure to give methyl 1-amino-3-(benzyloxy)cyclobutane-1-carboxylate. LCMS: (M+H)⁺=236.1; Retention time=1.21 min. LCMS CP Method C

Step 4: To a suspension of (S)-1-(4-fluorophenyl)-1,2,3,4-tetrahydroiso-quinoline (477 mg, 2.0 mmol) and pyridine (120 mg, 4.2 mmol) in DMF (5 mL) at 0° C. was added Triphosgene (197 mg, 0.6 mmol). The reaction mixture was stirred at room temperature for 30 min before methyl 1-amino-3-(benzyloxy)cyclobutane-1-carboxylate (470 mg, 2 mmol) was introduced. The resulting mixture was heated to 40° C. for 6 h, diluted with water (50 mL) and extracted with EA (3×30 mL). The combined organic phase was dried over anhydrous Na₂SO₄, filtered and concentrated and the residue purified by prep-HPLC to give methyl (S)-3-(benzyloxy)-1-(1-(4-fluorophenyl)-1,2,3,4-tetrahydroisoquinoline-2-carboxamido) cyclobutane-1-carboxylate. LCMS: (M+H)⁺=489.2; Retention time=1.56 min. LCMS CP Method C

Step 5: To a solution of methyl (S)-3-(benzyloxy)-1-(1-(4-fluorophenyl)-1,2,3,4-tetrahydroisoquinoline-2-carboxamido) cyclobutane-1-carboxylate (122 mg, 0.25 mmol) in MeOH (4 mL) was added NaBH₄ (152 mg, 4 mmol) at 0° C. The reaction mixture was stirred at room temperature for 2 h and then concentrated under reduced pressure. Purification of the residue by Prep-HPLC afforded (S)—N-(3-(benzyloxy)-1-(hydroxymethyl)cyclobutyl)-1-(4-fluorophenyl)-3,4-dihydroisoquinoline-2(1H)-carboxamide. LCMS: (M+H)⁺=461.1; Retention time=1.26 min. LCMS CP Method C

Step 6: A solution of (S)—N-(3-(benzyloxy)-1-(hydroxymethyl)cyclobutyl)-1-(4-fluorophenyl)-3,4-dihydroisoquinoline-2(1H)-carboxamide (230 mg, 0.5 mmol) and DCC (206 mg, 1.0 mmol) in THF (30 mL) was stirred at 65° C. for 2 h and then concentrated under reduced pressure. Purification of the residue by prep-HPLC afforded (S)-2-(benzyloxy)-6-(1-(4-fluorophenyl)-3, 4-dihydroisoquinolin-2(1H)-yl)-7-oxa-5-azaspiro[3.4]oct-5-ene. LCMS: (M+H)⁺=442.1; Retention time=1.45 min. LCMS CP Method C

Step 7: A solution of (S)-2-(benzyloxy)-6-(1-(4-fluorophenyl)-3,4-dihydroisoquinolin-2(1H)-yl)-7-oxa-5-azaspiro[3.4]oct-5-ene (221 mg, 0.5 mmol) in conc. HCl (10 mL) was stirred at 100° C. for 1 h. After cooling to room temperature, the mixture was diluted with ethyl acetate (60 mL) and water (30 mL). The aqueous layer was extracted with ethyl acetate (3×40 mL). The combined organic phase was washed with brine (30 mL), dried over Na₂SO₄, filtered and concentrated. The residue was purified by column chromatography to give (S)-6-(1-(4-fluorophenyl)-3,4-dihydroisoquinolin-2(1H)-yl)-7-oxa-5-azaspiro[3.4]oct-5-en-2-ol. LCMS: (M+H)⁺=353.0; Retention time=1.45 min. LCMS CP Method B

Step 8: To a solution of (S)-6-(1-(4-fluorophenyl)-3,4-dihydroisoquinolin-2(1H)-yl)-7-oxa-5-azaspiro[3.4]oct-5-en-2-ol (88 mg, 0.25 mmol), isoindoline-1,3-dione (73.5 mg, 0.5 mmol) and Ph₃P (131 mg, 0.5 mmol) in THF (5 mL) was added DEAD (174 mg, 1.0 mmol) under a nitrogen atmosphere. The resulting reaction mixture was stirred at 60° C. for 2 h and then diluted with ethyl acetate (60 mL) and water (30 mL). The aqueous layer was extracted with ethyl acetate (3×40 mL). The combined organic phase was washed with brine (30 mL), dried over Na₂SO₄, filtered and concentrated. The residue was purified by column chromatography to give (S)-2-(6-(1-(4-fluorophenyl)-3,4-dihydroisoquinolin-2(1H)-yl)-7-oxa-5-azaspiro[3.4]oct-5-en-2-yl)isoindoline-1,3-dione. LCMS: (M+H)⁺=482.1; Retention time=1.38 min. LCMS CP Method C

Step 9: A solution of (S)-2-(6-(1-(4-fluorophenyl)-3,4-dihydroisoquinolin-2(1H)-yl)-7-oxa-5-azaspiro[3.4]oct-5-en-2-yl)isoindoline-1,3-dione (102 mg, 0.21 mmol) and NH₂NH₂.H₂O (500 mg, 10 mmol) in EtOH (10 mL) was stirred at 65° C. for 2 h and then concentrated under reduced pressure. Purification of the residue by Prep-HPLC afforded (S)-2-(benzyloxy)-6-(1-(4-fluorophenyl)-3,4-dihydroisoquinolin-2(1H)-yl)-7-oxa-5-azaspiro[3.4]oct-5-ene, mixture of isomers.

Stereochemical assignment at the 1 position of the tetrahydroisoquinoline is assigned based on enantiomerically pure starting material.

Compound 3162: LCMS: (M+H)⁺=352; Retention time=1.56 min. LCMS CP Method C2. 1H NMR (400 MHz, CDCl₃) δ 9.73 (s, 2H), 7.23-7.12 (m, 6H), 6.96 (dd, J=17.6, 9.0 Hz, 2H), 6.35 (s, 1H), 4.04 (s, 1H), 3.61-3.36 (m, 4H), 2.97-2.86 (m, 1H), 2.76 (dt, J=11.4, 5.9 Hz, 1H), 2.11 (dd, J=34.5, 27.9 Hz, 4H). Chiral SFC: CO₂/MeOH containing 0.2% MA (80%:20%) over CHIRALPAK® IG column (4.6×100 mm, 3 μm), retention time=2.707 min, 94% ee.

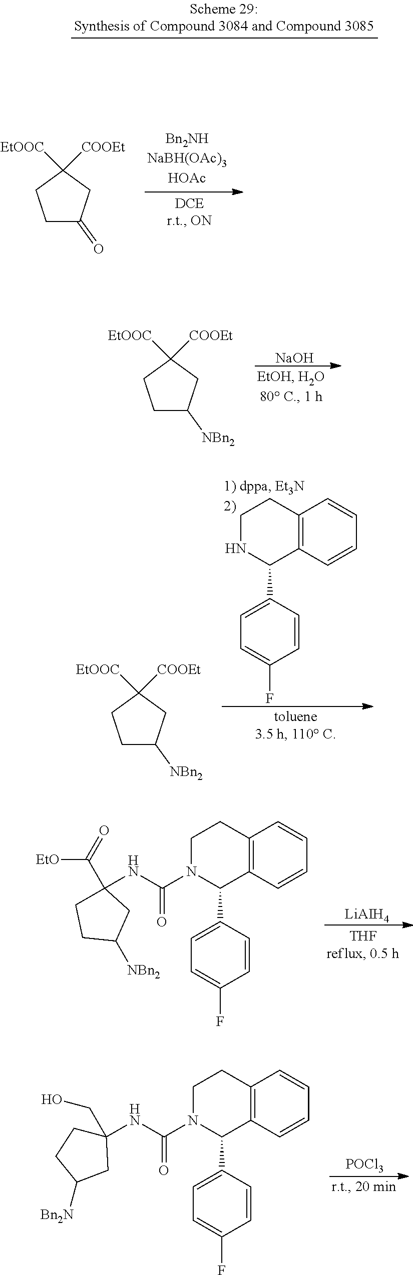

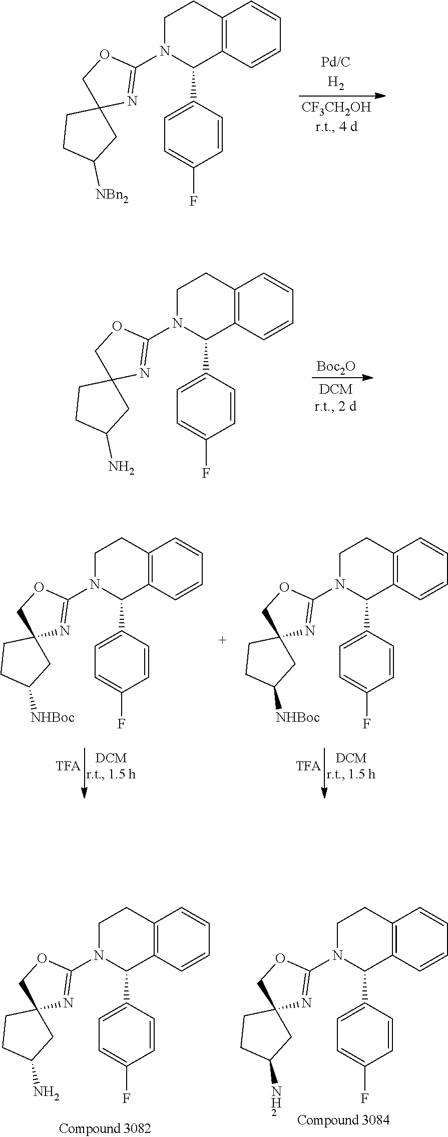

### Synthesis of diethyl 3-(dibenzylamino)cyclopentane-1,1-dicarboxylate

Sodium triacetoxyborohydride (574 mg, 2.71 mmol) was added portionwise to a solution of diethyl 3-oxocyclopentane-1,1-dicarboxylate (412 mg, 1.81 mmol), dibenzylamine (237 μL, 2.17 mmol), and acetic acid (155 μL, 2.71 mmol) in 1,2-dichloroethane (8 mL). After stirring at room temperature overnight, the reaction mixture was diluted with dichloromethane and washed with saturated aqueous NaHCO₃ (15 mL). The aqueous phase was extracted with dichloromethane (2×10 mL). The combined organic phase was dried over Na₂SO₄ and concentrated under reduced pressure. The residue was purified by flash column chromatography (silica, 0 to 20% ethyl acetate in heptane) to give diethyl 3-(dibenzylamino)cyclopentane-1,1-dicarboxylate. ¹H NMR (300 MHz, chloroform-d) δ 7.39-7.16 (m, 10H), 4.24-4.06 (m, 4H), 3.63 (s, 4H), 3.42-3.25 (m, 1H), 2.50 (dd, J=13.5, 7.7 Hz, 1H), 2.29 (ddd, J=12.8, 8.4, 4.1 Hz, 1H), 2.22-1.98 (m, 2H), 1.90-1.59 (m, 2H), 1.32-1.15 (m, 6H).

### Synthesis of 3-(dibenzylamino)-1-(ethoxycarbonyl)cyclopentane-1-carboxylic acid

Aqueous NaOH (1 M, 1.06 mL, 1.06 mmol) was added to a solution of diethyl 3-(dibenzylamino)cyclopentane-1,1-dicarboxylate (433 mg, 1.06 mmol) in ethanol (2 mL) and the reaction mixture was stirred at 80° C. for 1 hour. After cooling to ambient temperature, the reaction mixture was diluted with water and aqueous HCl (1 M, 1 mL) and extracted with dichloromethane (3×10 mL). The combined extracts were dried on Na₂SO₄ and concentrated under reduced pressure to give 3-(dibenzylamino)-1-(ethoxycarbonyl)cyclopentane-1-carboxylic acid which was used without further purification. LCMS: 57%, RT=1.98 min., (M+H)⁺=382; 34%, RT=2.03 min., (M+H)⁺=382 (method K). ¹H NMR (300 MHz, chloroform-d) δ 7.45-7.15 (m, 10H), 4.28-4.10 (m, 2H), 4.08-3.59 (m, 4H), 3.47-3.29 (m, 1H), 2.78-2.61 (m, 1H), 2.59-2.41 (m, 1H), 2.30-1.61 (m, 4H), 1.34-1.18 (m, 3H).

### Synthesis of ethyl 3-(dibenzylamino)-1-((S)-1-(4-fluorophenyl)-1,2,3,4-tetrahydroisoquinoline-2-carboxamido)cyclopentane-1-carboxylate

Under a nitrogen atmosphere, diphenylphosphoryl azide (179 μL, 0.830 mmol) was added to a mixture of crude 3-(dibenzylamino)-1-(ethoxycarbonyl)cyclopentane-1-carboxylic acid (288 mg, 0.755 mmol) and triethylamine (526 μL, 3.77 mmol) in toluene (10 mL). The reaction mixture was stirred at 110° C. for 1.5 hours, cooled to room temperature and (S)-1-phenyl-1,2,3,4-tetrahydroisoquinoline (172 mg, 0.755 mmol) was added. The mixture was warmed again to 110° C. for 1 hour then cooled to room temperature and concentrated to dryness under reduced pressure. The residue was purified by flash column chromatography (silica, 0 to 40% ethyl acetate in heptane) to give ethyl 3-(dibenzylamino)-1-((S)-1-(4-fluorophenyl)-1,2,3,4-tetrahydroisoquinoline-2-carboxamido)cyclopentane-1-carboxylate. LCMS: 100%, RT=2.81 min., (M+H)⁺=606 (method K).

### Synthesis of (1S)—N-(3-(dibenzylamino)-1-(hydroxymethyl)cyclopentyl)-1-(4-fluorophenyl)-3,4-dihydroisoquinoline-2(1H)-carboxamide

Under a nitrogen atmosphere, a solution of ethyl 3-(dibenzylamino)-1-((S)-1-(4-fluorophenyl)-1,2,3,4-tetrahydroisoquinoline-2-carboxamido)cyclopentane-1-carboxylate (390 mg, 0.644 mmol) in tetrahydrofuran (10 mL) was added with a pipette in 4 portions to a suspension of lithium aluminum hydride (100 mg, 2.64 mmol) in tetrahydrofuran (10 mL) in 1 minute. The reaction mixture was warmed to reflux and after 0.5 hour before being allowed to cool to room temperature. After 0.5 hour, the reaction mixture was cooled in a water bath and quenched by the addition of water (0.1 mL), aqueous NaOH (15%, 0.1 mL), and water (0.3 mL). The mixture was filtered through a thin layer of Celite, washed with tetrahydrofuran and the filtrate concentrated to dryness under reduced pressure to give (1S)—N-(3-(dibenzylamino)-1-(hydroxymethyl)cyclopentyl)-1-(4-fluorophenyl)-3,4-dihydro-isoquinoline-2(1H)-carboxamide. LCMS: 88%, RT=2.68 min., (M+H)⁺=564 (method K).

### Synthesis of N,N-dibenzyl-2-((S)-1-(4-fluorophenyl)-3,4-dihydroisoquinolin-2(1H)-yl)-3-oxa-1-azaspiro[4.4]non-1-en-7-amine

Phosphorus oxychloride (2.00 mL, 21.5 mmol) was added to (1S)—N-(3-(dibenzylamino)-1-(hydroxymethyl)cyclopentyl)-1-(4-fluorophenyl)-3,4-dihydroisoquinoline-2(1H)-carboxamide (360 mg, 0.639 mmol). The resulting solution was stirred at room temperature for 20 minutes and then concentrated to dryness under reduced pressure (50° C.). The residue was taken up in ethyl acetate (25 mL) and saturated aqueous NaHCO₃ (25 mL) and vigorously stirred for 10 minutes. The layers were separated and the aqueous phase extracted with ethyl acetate (25 mL). The combined organic phase was dried over Na₂SO₄ and evaporated under reduced pressure. The residue was purified by flash column chromatography (silica, 0 to 25% ethyl acetate in heptane) to give N,N-dibenzyl-2-((S)-1-(4-fluorophenyl)-3,4-dihydroisoquinolin-2(1H)-yl)-3-oxa-1-azaspiro[4.4]non-1-en-7-amine. LCMS: 97%, RT=3.14 min., (M+H)⁺=546 (method K). ¹H NMR (300 MHz, chloroform-d) δ 7.50-7.09 (m, 15H), 7.09-6.83 (m, 3H), 6.27-6.18 (m, 1H), 4.05-3.83 (m, 3H), 3.75-3.55 (m, 4H), 3.35-3.14 (m, 2H), 3.13-2.97 (m, 1H), 2.73 (dt, J=16.2, 3.7 Hz, 1H), 2.04-1.48 (m, 6H).

### Synthesis of 2-((S)-1-(4-fluorophenyl)-3,4-dihydroisoquinolin-2(1H)-yl)-3-oxa-1-azaspiro[4.4]non-1-en-7-amine

Palladium (10 wt % on carbon, 50 mg, 0.047 mmol) was added to a solution of N,N-dibenzyl-2-((S)-1-(4-fluorophenyl)-3,4-dihydroisoquinolin-2(1H)-yl)-3-oxa-1-azaspiro[4.4]non-1-en-7-amine (130 mg, 0.238 mmol) in 2,2,2-trifluoroethanol (10 mL). The resulting mixture was stirred under a hydrogen atmosphere for 4 days, filtered through a layer of Celite and washed with ethanol. The filtrate was evaporated under reduced pressure. The residue was purified by flash column chromatography (silica, 0 to 15% methanol in dichloromethane) to give 2-((S)-1-(4-fluorophenyl)-3,4-dihydroisoquinolin-2(1H)-yl)-3-oxa-1-azaspiro[4.4]non-1-en-7-amine. LCMS: 48%, RT=1.97 min., (M+H)⁺=366; 47%, RT=2.00 min., (M+H)⁺=366 (method K).

### Synthesis of tert-butyl ((5S,7R)-2-((S)-1-(4-fluorophenyl)-3,4-dihydroisoquinolin-2(1H)-yl)-3-oxa-1-azaspiro[4.4]non-1-en-7-yl)carbamate and tert-butyl ((5S,7S)-2-((S)-1-(4-fluorophenyl)-3,4-dihydroisoquinolin-2(1H)-yl)-3-oxa-1-azaspiro[4.4]non-1-en-7-yl)carbamate

Di-tert-butyl dicarbonate (53.9 mg, 0.247 mmol) was added to a solution of 2-((S)-1-(4-fluorophenyl)-3,4-dihydroisoquinolin-2(1H)-yl)-3-oxa-1-azaspiro[4.4]non-1-en-7-amine (80 mg, 0.219 mmol) in dichloromethane (2 mL). After 1 day, another portion of di-tert-butyl dicarbonate (9.9 mg, 0.045 mmol) was added and stirring was continued for another day. The reaction mixture was concentrated to dryness under reduced pressure. The residue was purified by basic preparative MPLC (Linear Gradient: t=0 min 5% A; t=1 min 5% A; t=2 min 50% A; t=17 min 70% A; t=18 min 100% A; t=23 min 100% A; detection: 220/235 nm) and preparative chiral SFC (method W) to give tert-butyl ((5S,7R)-2-((S)-1-(4-fluorophenyl)-3,4-dihydroisoquinolin-2(1H)-yl)-3-oxa-1-azaspiro[4.4]non-1-en-7-yl)carbamate as the first eluting SFC isomer, and tert-butyl ((5S,7S)-2-((S)-1-(4-fluorophenyl)-3,4-dihydroisoquinolin-2(1H)-yl)-3-oxa-1-azaspiro[4.4]non-1-en-7-yl)carbamate as second eluting SFC isomer. Configuration of the spirocylic center and primary amine are arbitrarily assigned.

First eluting isomer: LCMS: 98%, RT=1.80 min., (M+H)⁺=466 (method A). Chiral SFC: 100% d.e., RT=1.85 min., (M+H)⁺=466 (method \A/). ¹H NMR (400 MHz, chloroform-d) δ 7.25-7.12 (m, 5H), 7.01 (d, J=7.5 Hz, 1H), 6.96 (t, J=8.6 Hz, 2H), 6.18 (s, 1H), 5.87 (br d, J=9.0 Hz, 1H), 4.20-4.05 (m, 3H), 3.89 (ddd, J=13.2, 6.0, 3.4 Hz, 1H), 3.31 (ddd, J=13.1, 10.7, 4.3 Hz, 1H), 3.04 (ddd, J=16.6, 10.7, 6.0 Hz, 1H), 2.74 (dt, J=16.2, 3.9 Hz, 1H), 2.15-2.01 (m, 1H), 2.00-1.89 (m, 1H), 1.86 (dd, J=13.0, 6.9 Hz, 1H), 1.81-1.70 (m, 1H), 1.70-1.57 (m, 2H), 1.46 (s, 9H). Second eluting isomer: LCMS: 68%, RT=1.81 min., (M+H)⁺=466 (method A). Chiral SFC: 95% d.e., RT=2.12 min., (M+H)⁺=466 (method W). ¹H NMR (400 MHz, chloroform-d) δ 7.25-7.12 (m, 5H), 7.00 (d, J=7.6 Hz, 1H), 6.95 (t, J=8.7 Hz, 2H), 6.17 (s, 1H), 5.80 (br d, J=9.0 Hz, 1H), 4.19-4.05 (m, 3H), 3.88 (ddd, J=13.1, 6.0, 3.4 Hz, 1H), 3.29 (ddd, J=13.1, 10.5, 4.4 Hz, 1H), 3.05 (ddd, J=16.4, 10.6, 5.9 Hz, 1H), 2.75 (dt, J=16.3, 3.9 Hz, 1H), 2.13-2.01 (m, 1H), 1.97-1.81 (m, 2H), 1.80-1.57 (m, 3H), 1.44 (s, 9H).

### Synthesis of (5S,7S)-2-((S)-1-(4-fluorophenyl)-3,4-dihydroisoquinolin-2(1H)-yl)-3-oxa-1-azaspiro[4.4]non-1-en-7-amine (Compound 3084)

Trifluoroacetic acid (0.5 mL, 6.49 mmol) was added to a solution of tert-butyl ((5S,7S)-2-((S)-1-(4-fluorophenyl)-3,4-dihydroisoquinolin-2(1H)-yl)-3-oxa-1-azaspiro[4.4]non-1-en-7-yl)carbamate (18 mg, 0.039 mmol) in dichloromethane (2 mL). After 1.5 hours, the reaction mixture was concentrated to dryness under reduced pressure. The residue was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure and the residue lyophilized from a mixture of acetonitrile and water (1:1, 4 mL) to give (5S,7S)-2-((S)-1-(4-fluorophenyl)-3,4-dihydroisoquinolin-2(1H)-yl)-3-oxa-1-azaspiro[4.4]non-1-en-7-amine (Compound 3084). LCMS: 98%, RT=1.83 min., (M+H)⁺=366 (method AK). ¹H NMR (400 MHz, chloroform-d) δ 7.25-7.12 (m, 5H), 7.02 (d, J=7.5 Hz, 1H), 6.94 (t, J=8.7 Hz, 2H), 6.20 (s, 1H), 4.09 (d, J=7.8 Hz, 1H), 4.05 (d, J=7.8 Hz, 1H), 3.88 (ddd, J=13.2, 6.3, 3.7 Hz, 1H), 3.36-3.21 (m, 2H), 3.05 (ddd, J=16.4, 10.5, 6.1 Hz, 1H), 2.72 (dt, J=16.2, 4.0 Hz, 1H), 2.06-1.95 (m, 2H), 1.92 (dd, J=13.0, 6.7 Hz, 1H), 1.74-1.55 (m, 5H).

### Synthesis of (5S,7R)-2-((S)-1-(4-fluorophenyl)-3,4-dihydroisoquinolin-2(1H)-yl)-3-oxa-1-azaspiro[4.4]non-1-en-7-amine (Compound 3082)

Trifluoroacetic acid (0.5 mL, 6.49 mmol) was added to a solution of tert-butyl ((5S,7R)-2-((S)-1-(4-fluorophenyl)-3,4-dihydroisoquinolin-2(1H)-yl)-3-oxa-1-azaspiro[4.4]non-1-en-7-yl)carbamate (22 mg, 0.047 mmol) in dichloromethane (2 mL). After 1.5 hours, the reaction mixture was concentrated to dryness under reduced pressure. The residue was dissolved in methanol (1 mL) and loaded onto an SCX-2 column (1 g) and eluted with methanol until neutral. Next, the column was eluted with ammonia in methanol (1 M), the basic fraction concentrated to dryness under reduced pressure and the residue lyophilized from a mixture of acetonitrile and water (1:1, 4 mL) to give (5S,7S)-2-((S)-1-(4-fluorophenyl)-3,4-dihydroisoquinolin-2(1H)-yl)-3-oxa-1-azaspiro[4.4]non-1-en-7-amine (Compound 3082). LCMS: 99%, RT=1.83 min., (M+H)⁺=366 (method AK). ¹H NMR (400 MHz, chloroform-d) δ 7.24-7.12 (m, 5H), 7.02 (d, J=7.5 Hz, 1H), 6.94 (t, J=8.7 Hz, 2H), 6.21 (s, 1H), 4.10 (d, J=7.8 Hz, 1H), 4.04 (d, J=7.8 Hz, 1H), 3.88 (ddd, J=13.2, 6.2, 3.6 Hz, 1H), 3.35-3.22 (m, 2H), 3.04 (ddd, J=16.5, 10.6, 5.9 Hz, 1H), 2.72 (dt, J=16.1, 4.0 Hz, 1H), 2.07-1.95 (m, 2H), 1.92 (dd, J=13.0, 6.9 Hz, 1H), 1.72-1.53 (m, 5H).

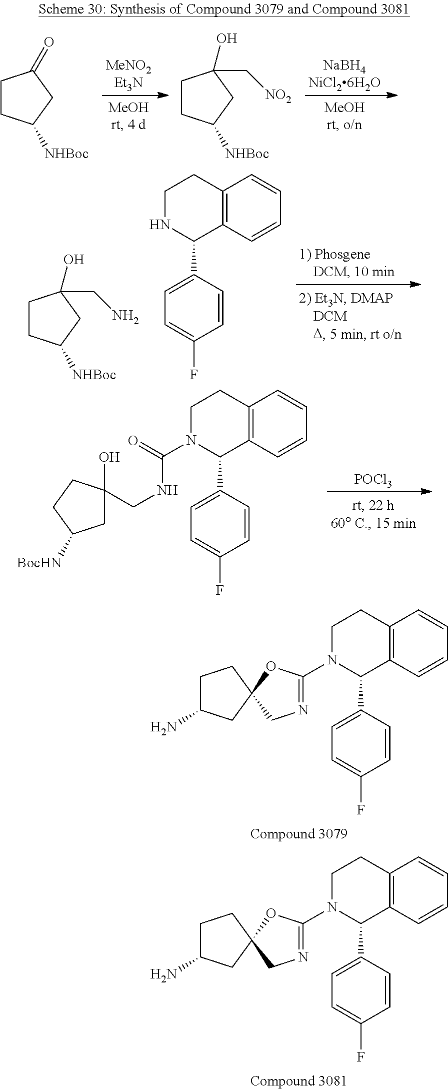

### Synthesis of tert-butyl ((1R)-3-hydroxy-3-(nitromethyl)cyclopentyl)carbamate

Nitromethane (1.25 mL, 22.9 mmol) and triethylamine (2.61 mL, 18.8 mmol) were added sequentially to a solution of tert-butyl (R)-(3-oxocyclopentyl)carbamate (500 mg, 2.51 mmol) in methanol (10 mL). After stirring for 4 days, the mixture was concentrated to dryness under reduced pressure. The residue was purified by flash column chromatography (silica, 0 to 50% ethyl acetate in heptane) to give tert-butyl ((1R)-3-hydroxy-3-(nitromethyl)cyclopentyl)carbamate. LCMS: 50.0%, RT=1.50 min., (M−H)⁻=259; 38.4%, RT=1.56 min., (M−H)⁻=259 (method K). ¹H NMR (300 MHz, chloroform-d) δ 4.56 (br s, 1H), 4.52 (d, J=5.0 Hz, 1H), 4.29-4.14 (m, 2H), 2.62 (dd, J=18.5, 7.3 Hz, 1H), 2.35 (dtd, J=14.3, 9.9, 8.6, 5.2 Hz, 2H), 2.28-2.16 (m, 1H), 2.11 (dd, J=18.5, 7.5 Hz, 2H), 1.82 (dt, J=12.1, 7.9 Hz, 1H), 1.45 (s, 9H).

### Synthesis of tert-butyl ((1R)-3-(aminomethyl)-3-hydroxycyclopentyl)carbamate

Sodium borohydride (950 mg, 25.1 mmol) was added in three portions over 5 minutes to a solution of tert-butyl ((1R)-3-hydroxy-3-(nitromethyl)cyclopentyl)carbamate (276 mg, 1.06 mmol) and nickel(II) chloride hexahydrate (270 mg, 1.14 mmol) in methanol (15 mL). The reaction mixture was stirred overnight, diluted with water (25 mL), stirred for 15 minutes, and extracted with ethyl acetate (50 mL). The layers were separated and the aqueous phase was diluted with saturated aqueous NaHCO₃ (25 mL) and extracted with ethyl acetate (2×50 mL). The combined organic phase was dried over Na₂SO₄ and evaporated under reduced pressure to give tert-butyl ((1R)-3-(aminomethyl)-3-hydroxycyclopentyl)carbamate that was used as such.

### Synthesis of tert-butyl ((1R)-3-(((S)-1-(4-fluorophenyl)-1,2,3,4-tetrahydroisoquinoline-2-carboxamido)methyl)-3-hydroxycyclopentyl)carbamate

A cold (˜5° C.) solution of phosgene (5 mL, 9.50 mmol, 20 wt % in toluene) was added to a solution of (S)-1-(4-fluorophenyl)-1,2,3,4-tetrahydroisoquinoline (280 mg, 1.23 mmol) in dichloromethane (4 mL) at room temperature. After 10 minutes, the reaction mixture was concentrated to dryness under reduced pressure (at 60° C.). A solution of tert-butyl ((1R)-3-(aminomethyl)-3-hydroxycyclopentyl)carbamate (240 mg, 1.04 mmol), triethylamine (1.6 mL, 12 mmol), and 4-dimethylaminopyridine (21 mg, 0.17 mmol) in dichloromethane (5 mL) was added to the residue and the resulting solution was heated to reflux for 5 minutes and then stirred overnight at room temperature. The reaction mixture was concentrated to dryness under reduced pressure, the residue diluted with saturated aqueous NaHCO₃ (20 mL) and extracted with ethyl acetate (3×40 mL). The combined organic phase was dried over Na₂SO₄ and evaporated under reduced pressure. The residue was purified by flash column chromatography (silica, 0 to 100% ethyl acetate in heptane) to give tert-butyl ((1R)-3-(((S)-1-(4-fluorophenyl)-1,2,3,4-tetrahydroisoquinoline-2-carboxamido)methyl)-3-hydroxycyclopentyl) carbamate. LCMS: 92%, RT=2.22 min., (M+H)⁺=484 (method K).

### Synthesis of (5R,7R)-2-((S)-1-(4-fluorophenyl)-3,4-dihydroisoquinolin-2(1H)-yl)-1-oxa-3-azaspiro[4.4]non-2-en-7-amine (Compound 3079) and (5S,7R)-2-((S)-1-(4-fluorophenyl)-3,4-dihydroisoquinolin-2(1H)-yl)-1-oxa-3-azaspiro[4.4]non-2-en-7-amine (Compound 3081)

Phosphoryl trichloride (4 mL, 43 mmol) was added to tert-butyl ((1R)-3-(((S)-1-(4-fluorophenyl)-1,2,3,4-tetrahydroisoquinoline-2-carboxamido)methyl)-3-hydroxycyclopentyl)carbamate (110 mg, 0.227 mmol) and the resulting solution was stirred at room temperature for 22 hours. Then, the mixture was warmed to 60° C. for 15 minutes after which it was concentrated to dryness under reduced pressure. The residue was diluted with saturated aqueous NaHCO₃ (10 mL) and extracted with ethyl acetate (3×15 mL). The combined organic phase was dried over Na₂SO₄ and evaporated under reduced pressure. The residue was purified by acidic preparative MPLC (Linear Gradient: t=0 min 2% A; t=1 min 2% A; t=16 min 30% A; t=17 min 100%; t=22 min 100% A; detection: 220/245/270 nm) to give (5R,7R)-2-((S)-1-(4-fluorophenyl)-3,4-dihydroisoquinolin-2(1H)-yl)-1-oxa-3-azaspiro[4.4]non-2-en-7-amine (Compound 3079) and (5S,7R)-2-((S)-1-(4-fluorophenyl)-3,4-dihydroisoquinolin-2(1H)-yl)-1-oxa-3-azaspiro[4.4]non-2-en-7-amine (Compound 3081) after lyophilization. Absolute stereochemistry of the spirocyclic center was arbitrarily assigned.

Compound 3079: LCMS: 86%, RT=0.68 min., (M+H)⁺=366 (method P). ¹H NMR (400 MHz, methanol-d₄) δ 7.24-7.16 (m, 5H), 7.08-6.99 (m, 3H), 6.18 (s, 1H), 3.89-3.78 (m, 1H), 3.74-3.63 (m, 2H), 3.50-3.36 (m, 2H), 3.03-2.93 (m, 1H), 2.83-2.74 (m, 1H), 2.26-2.01 (m, 3H), 1.98-1.92 (m, 1H), 1.80-1.57 (m, 2H).

Compound 3081: LCMS: 81%, RT=0.71 min., (M+H)⁺=366 (method P). ¹H NMR (400 MHz, methanol-d₄) δ 7.24-7.17 (m, 5H), 7.08-7.01 (m, 3H), 6.11 (s, 1H), 3.84-3.70 (m, 4H), 3.46-3.38 (m, 1H), 3.04-2.95 (m, 1H), 2.83-2.75 (m, 1H), 2.46-2.26 (m, 2H), 2.16-2.07 (m, 2H), 1.89-1.76 (m, 2H).

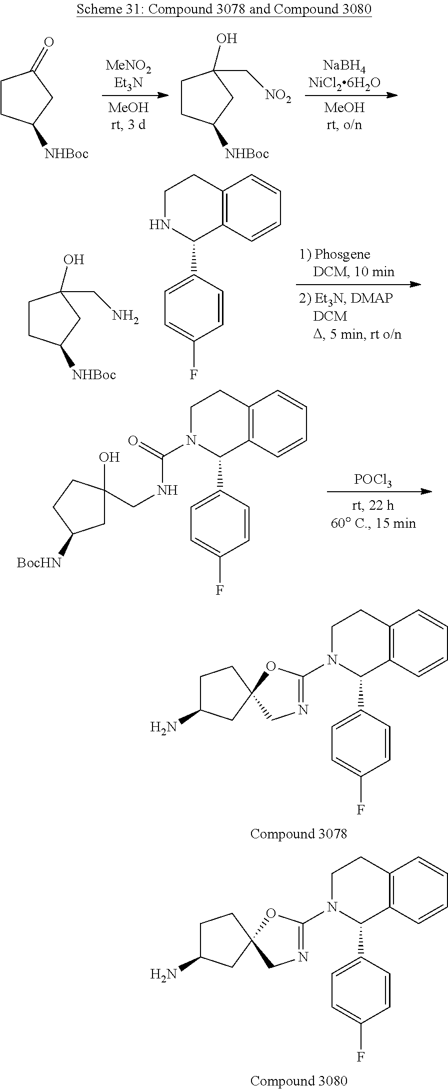

### Synthesis of tert-butyl ((1S)-3-hydroxy-3-(nitromethyl)cyclopentyl)carbamate

Starting from tert-butyl (S)-(3-oxocyclopentyl)carbamate (1.0 g, 5.0 mmol), tert-butyl ((1S)-3-hydroxy-3-(nitromethyl)cyclopentyl)carbamate was prepared as described for tert-butyl ((1R)-3-hydroxy-3-(nitromethyl)cyclopentyl)carbamate (see Compound 3079). LCMS: 55.4%, RT=1.42 min., (M-tBuO)⁺=187; 44.0%, RT=1.49 min., (M-tBuO)⁺=187 (method K). ¹H NMR (300 MHz, chloroform-d) δ 5.23-5.01+4.63-4.37+4.31-3.80 (m, 4H), 2.41-2.03 (m, 2H), 2.02-1.79 (m, 2H), 1.72-1.49 (m, 2H), 1.48-1.35 (m, 9H).

### Synthesis of tert-butyl ((1S)-3-(aminomethyl)-3-hydroxycyclopentyl)carbamate

Starting from tert-butyl ((1S)-3-hydroxy-3-(nitromethyl)cyclopentyl)carbamate (240 mg, 0.922 mmol), tert-butyl ((1S)-3-(aminomethyl)-3-hydroxycyclopentyl)carbamate was prepared as described for tert-butyl ((1R)-3-(aminomethyl)-3-hydroxycyclopentyl)carbamate (see Compound 3079).

### Synthesis of tert-butyl ((1S)-3-(((S)-1-(4-fluorophenyl)-1,2,3,4-tetrahydroisoquinoline-2-carboxamido)methyl)-3-hydroxycyclopentyl)carbamate

Starting from (S)-1-(4-fluorophenyl)-1,2,3,4-tetrahydroisoquinoline (280 mg, 1.23 mmol) and tert-butyl ((1S)-3-(aminomethyl)-3-hydroxycyclopentyl)carbamate (190 mg, 0.825 mmol), tert-butyl ((1S)-3-(((S)-1-(4-fluorophenyl)-1,2,3,4-tetrahydroisoquinoline-2-carboxamido)methyl)-3-hydroxycyclopentyl)carbamate was prepared as described for tert-butyl ((1R)-3-(((S)-1-(4-fluorophenyl)-1,2,3,4-tetrahydroisoquinoline-2-carboxamido)methyl)-3-hydroxycyclopentyl)carbamate (see Compound 3079). LCMS: 41%, RT=1.66 min., (M+H)⁺=484 (method K).

### Synthesis of (5R,7S)-2-((S)-1-(4-fluorophenyl)-3,4-dihydroisoquinolin-2(1H)-yl)-1-oxa-3-azaspiro[4.4]non-2-en-7-amine (Compound 3078) and (5S,7S)-2-((S)-1-(4-fluorophenyl)-3,4-dihydroisoquinolin-2(1H)-yl)-1-oxa-3-azaspiro[4.4]non-2-en-7-amine (Compound 3080)

Starting from tert-butyl ((1S)-3-(((S)-1-(4-fluorophenyl)-1,2,3,4-tetrahydroisoquinoline-2-carboxamido)methyl)-3-hydroxycyclopentyl)carbamate (184 mg, 0.38 mmol), (5R,7S)-2-((S)-1-(4-fluorophenyl)-3,4-dihydroisoquinolin-2(1H)-yl)-1-oxa-3-azaspiro[4.4]non-2-en-7-amine (Compound 3078) and (5S,7S)-2-((S)-1-(4-fluorophenyl)-3,4-dihydroisoquinolin-2(1H)-yl)-1-oxa-3-azaspiro[4.4]non-2-en-7-amine (Compound 3080) were prepared as described for (5R,7R)-2-((S)-1-(4-fluorophenyl)-3,4-dihydroisoquinolin-2(1H)-yl)-1-oxa-3-azaspiro[4.4]non-2-en-7-amine (Compound 3079). Absolute stereochemistry of the spirocyclic center was arbitrarily assigned.

Compound 3078 LCMS: 99%, RT=0.67 min., (M+H)⁺=366 (method P). ¹H NMR (400 MHz, methanol-d₄) δ 7.29-7.14 (m, 5H), 7.12-6.96 (m, 3H), 6.18 (s, 1H), 3.86-3.76 (m, 1H), 3.75-3.66 (m, 2H), 3.57-3.51 (m, 1H), 3.43-3.34 (m, 1H), 3.05-2.93 (m, 1H), 2.81-2.71 (m, 1H), 2.33-2.10 (m, 3H), 2.04-1.98 (m, 1H), 1.87-1.70 (m, 2H).

Compound 3080: LCMS: 91%, RT=0.72 min., (M+H)⁺=366 (method P). ¹H NMR (400 MHz, methanol-d₄) δ 7.25-7.14 (m, 5H), 7.06-6.97 (m, 3H), 6.11 (s, 1H), 3.85-3.62 (m, 4H), 3.42-3.36 (m, 1H), 3.04-2.93 (m, 1H), 2.83-2.76 (m, 1H), 2.46-2.38 (m, 1H), 2.23-2.14 (m, 1H), 2.13-1.97 (m, 2H), 1.74-1.58 (m, 2H).

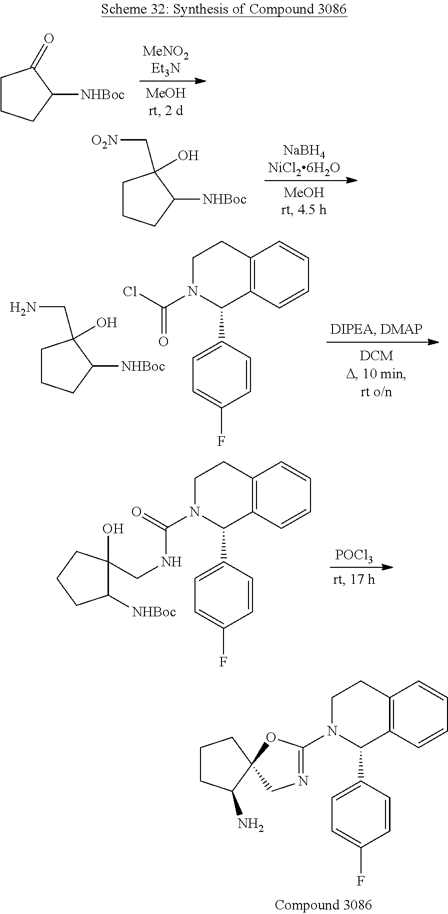

### Synthesis of tert-butyl (2-hydroxy-2-(nitromethyl)cyclopentyl)carbamate

Starting from tert-butyl (2-oxocyclopentyl)carbamate (4.0 g, 20.0 mmol), tert-butyl (2-hydroxy-2-(nitromethyl)cyclopentyl)carbamate was prepared as described for tert-butyl ((1R)-3-hydroxy-3-(nitromethyl)cyclopentyl)carbamate (see Compound 3079). LCMS: 76%, RT=1.64 min., (M−H)⁻=259 (method K).

### Synthesis of tert-butyl (2-(aminomethyl)-2-hydroxycyclopentyl)carbamate

Starting from tert-butyl (2-hydroxy-2-(nitromethyl)cyclopentyl)carbamate (3.3 g, 13 mmol), tert-butyl (2-(aminomethyl)-2-hydroxycyclopentyl)carbamate was prepared as described for tert-butyl ((1R)-3-(aminomethyl)-3-hydroxycyclopentyl)carbamate (see Compound 3079).

### Synthesis of (1S)—N-((2-amino-1-hydroxycyclopentyl)methyl)-1-(4-fluorophenyl)-3,4-dihydroisoquinoline-2(1H)-carboxamide

N,N-diisopropylethylamine (1.7 mL, 9.52 mmol) was added to a solution of tert-butyl (2-(aminomethyl)-2-hydroxycyclopentyl)carbamate (0.79 g, 3.5 mmol), (S)-1-(4-fluorophenyl)-3,4-dihydroisoquinoline-2(1H)-carbonyl chloride (1.0 g, 3.5 mmol), and dimethylaminopyridine (40 mg, 0.33 mmol) in dichloromethane (40 mL). The resulting mixture was heated to reflux for 10 minutes and then stirred overnight at room temperature. The reaction mixture was concentrated to dryness under reduced pressure. The residue was purified by flash column chromatography (silica, 0 to 100% ethyl acetate in heptane) to give (1S)—N-((2-amino-1-hydroxycyclopentyl)methyl)-1-(4-fluorophenyl)-3,4-dihydroisoquinoline-2(1H)-carboxamide. LCMS: 97%, RT=2.38 min., (M+H)⁺=484 (method K). ¹H NMR (300 MHz, chloroform-d) δ 7.32-7.07 (m, 6H), 7.02-6.84 (m, 2H), 6.44 (s, 1H), 5.64 (br s, 1H), 5.02-4.84 (m, 1H), 4.24-3.89 (m, 1H), 3.76-3.21 (m, 6H), 2.98-2.66 (m, 2H), 2.08-1.44 (m, 4H), 1.40-1.30 (m, 9H).

### Synthesis of (55,65)-2-((S)-1-(4-fluorophenyl)-3,4-dihydroisoquinolin-2(1H)-yl)-1-oxa-3-azaspiro[4.4]non-2-en-6-amine (Compound 3086)

Phosphoryl trichloride (5 mL, 54 mmol) was added to (1S)—N-((2-amino-1-hydroxycyclopentyl)methyl)-1-(4-fluorophenyl)-3,4-dihydroisoquinoline-2(1H)-carboxamide (510 mg, 1.05 mmol) and the resulting solution was stirred at room temperature for 17 hours. The mixture was concentrated to dryness under reduced pressure at 60° C. The residue was purified by acidic preparative MPLC (Linear Gradient: t=0 min 2% A; t=2 min 2% A; t=17 min 20% A; t=18 min 100%; t=22 min 100% A; detection: 220/245/270 nm), preparative LCMS (method AX), and preparative chiral HPLC (method AY) to give (5S,6S)-2-((S)-1-(4-fluorophenyl)-3,4-dihydroisoquinolin-2(1H)-yl)-1-oxa-3-azaspiro[4.4]non-2-en-6-amine (Compound 3086) as a white solid after lyophilization. Absolute stereochemistry of the spirocyclic and cyclopentylamine centers were arbitrarily assigned. LCMS: 87%, RT=0.70 min., (M+H)⁺=366 (method P). ¹H NMR (400 MHz, chloroform-d) δ 7.25-7.12 (m, 5H), 7.05-6.94 (m, 3H), 6.13 (s, 1H), 3.98 (dt, J=12.9, 5.1 Hz, 1H), 3.74-3.64 (m, 2H), 3.47 (ddd, J=13.6, 9.8, 4.4 Hz, 1H), 3.04 (ddd, J=15.9, 9.9, 5.8 Hz, 1H), 2.91-2.77 (m, 2H), 2.21-2.11 (m, 1H), 2.07-1.96 (m, 1H), 1.83-1.37 (m, 4H).

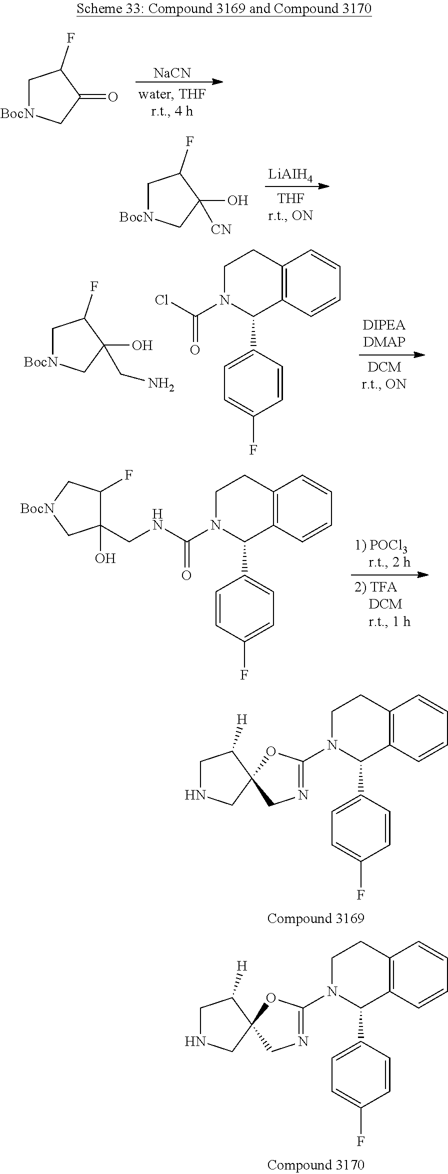

### Synthesis of tert-butyl 3-cyano-4-fluoro-3-hydroxypyrrolidine-1-carboxylate

Sodium cyanide (613 mg, 12.5 mmol) was added to a mixture of tert-butyl 3-fluoro-4-oxopyrrolidine-1-carboxylate (508 mg, 2.50 mmol) and water (3 mL). After stirring for 5 minutes, tetrahydrofuran (1 mL) was added and stirring was continued for 4 hours. The reaction mixture was diluted with ethyl acetate (50 mL) and washed with water (25 mL). The aqueous phase was extracted with ethyl acetate (2×25 mL). The combined organic phase was dried over Na₂SO₄ and evaporated under reduced pressure to give tert-butyl 3-cyano-4-fluoro-3-hydroxypyrrolidine-1-carboxylate which was used as such in the next step.

### Synthesis of tert-butyl 3-(aminomethyl)-4-fluoro-3-hydroxypyrrolidine-1-carboxylate

Under nitrogen atmosphere, lithium aluminium hydride (141 mg, 3.72 mmol) was added to a solution of tert-butyl 3-cyano-4-fluoro-3-hydroxypyrrolidine-1-carboxylate (389 mg, 1.69 mmol) in dry tetrahydrofuran (15 mL) and stirred at room temperature overnight. The reaction mixture was quenched with water (0.66 mL), aqueous NaOH (15%, 0.14 mL) and stirred for 30 minutes. The mixture was further diluted with ethyl acetate (50 mL) and water (25 mL) and the layers were separated. The aqueous phase was extracted with ethyl acetate (2×30 mL) and the combined extracts were dried over Na₂SO₄ and evaporated under reduced pressure. The residue was purified by flash column chromatography (silica, 0 to 30% methanol in dichloromethane) to give tert-butyl 3-(aminomethyl)-4-fluoro-3-hydroxypyrrolidine-1-carboxylate which was used as such.

### Synthesis of tert-butyl 4-fluoro-3-(((S)-1-(4-fluorophenyl)-1,2,3,4-tetrahydroisoquinoline-2-carboxamido)methyl)-3-hydroxypyrrolidine-1-carboxylate

Under nitrogen atmosphere, a solution of tert-butyl 3-(aminomethyl)-4-fluoro-3-hydroxypyrrolidine-1-carboxylate (207 mg, 0.884 mmol), (S)-1-(4-fluorophenyl)-3,4-dihydroisoquinoline-2(1H)-carbonyl chloride (256 mg, 0.884 mmol) and N,N-diisopropylethylamine (0.462 mL, 2.65 mmol) in dichloromethane (5 mL) was stirred overnight at room temperature. The reaction mixture was partly concentrated under reduced pressure and purified by flash column chromatography (silica, 0 to 100% ethyl acetate in heptane) to give tert-butyl 4-fluoro-3-(((S)-1-(4-fluorophenyl)-1,2,3,4-tetrahydroisoquinoline-2-carboxamido)methyl)-3-hydroxypyrrolidine-1-carboxylate. LCMS: 89%, RT=2.25 min., (M−H)⁻=486 (method K).

### Synthesis of (5S,9S)-9-fluoro-2-((S)-1-(4-fluorophenyl)-3,4-dihydroisoquinolin-2(1H)-yl)-1-oxa-3,7-diazaspiro[4.4]non-2-ene (Compound 3169) and (5R,9S)-9-fluoro-2-((S)-1-(4-fluorophenyl)-3,4-dihydroisoquinolin-2(1H)-yl)-1-oxa-3,7-diazaspiro[4.4]non-2-ene (Compound 3170)

A mixture of tert-butyl 4-fluoro-3-(((S)-1-(4-fluorophenyl)-1,2,3,4-tetrahydroisoquinoline-2-carboxamido)methyl)-3-hydroxypyrrolidine-1-carboxylate (180 mg, 0.369 mmol) and phosphoryl trichloride (3.00 mL, 32.2 mmol) was stirred at room temperature for 2 hours. The reaction mixture was concentrated to dryness under reduced pressure. The residue was taken up in dichloromethane (3 mL) and trifluoroactic acid (1 mL) was added. After 1 hour, the reaction mixture was concentrated to dryness under reduced pressure. The residue was taken up in dichloromethane (20 mL) and washed with aqueous NaOH (1 M, 10 mL). The aqueous phase was extracted with dichloromethane (10 mL). The combined organic phase was dried over Na₂SO₄ and concentrated under reduced pressure. The residue was purified by flash column chromatography (silica, 0 to 15% (3.5 M ammonia in methanol) in dichloromethane) and preparative SFC (method Z) to give (5R,9S)-9-fluoro-2-((S)-1-(4-fluorophenyl)-3,4-dihydroisoquinolin-2(1H)-yl)-1-oxa-3,7-diazaspiro[4.4]non-2-ene (Compound 3170) as the first eluting SFC isomer and (5S,9S)-9-fluoro-2-((S)-1-(4-fluorophenyl)-3,4-dihydroisoquinolin-2(1H)-yl)-1-oxa-3,7-diazaspiro[4.4]non-2-ene (Compound 3169) as the second eluting SFC isomer after lyophilization from a mixture of acetonitrile and water (1:1, 4 mL). Configuration of the pyrrolidine moiety is absolute unknown and arbitrarily assigned.

Compound 3169: LCMS: 96%, RT=0.66 min., (M+H)⁺=370 (method P). ¹H NMR (400 MHz, chloroform-d) mixture of rotamers (˜10:1); major rotamer: δ 7.25-7.12 (m, 5H), 7.01 (d, J=7.5 Hz, 1H), 6.96 (t, J=8.7 Hz, 2H), 6.12 (s, 1H), 4.67 (dd, J=52.4, 3.2 Hz, 1H), 4.13 (dd, J=13.3, 1.9 Hz, 1H), 3.89 (ddd, J=13.2, 6.0, 3.9 Hz, 1H), 3.67 (d, J=13.4 Hz, 1H), 3.46-3.17 (m, 4H), 3.13-2.95 (m, 2H), 2.77 (dt, J=16.3, 4.3 Hz, 1H).

Compound 3170: LCMS: 97%, RT=0.66 min., (M+H)⁺=370 (method P). ¹H NMR (400 MHz, chloroform-d) mixture of rotamers (˜20:1); major rotamer: δ 7.25-7.13 (m, 5H), 7.01 (d, J=7.5 Hz, 1H), 6.96 (t, J=8.7 Hz, 2H), 6.15 (s, 1H), 4.82 (dd, J=52.3, 3.7 Hz, 1H), 4.17 (dd, J=13.3, 1.9 Hz, 1H), 3.88 (ddd, J=13.3, 6.1, 3.5 Hz, 1H), 3.66 (d, J=13.3 Hz, 1H), 3.42-3.17 (m, 4H), 3.12-2.97 (m, 2H), 2.74 (dt, J=16.4, 4.0 Hz, 1H).

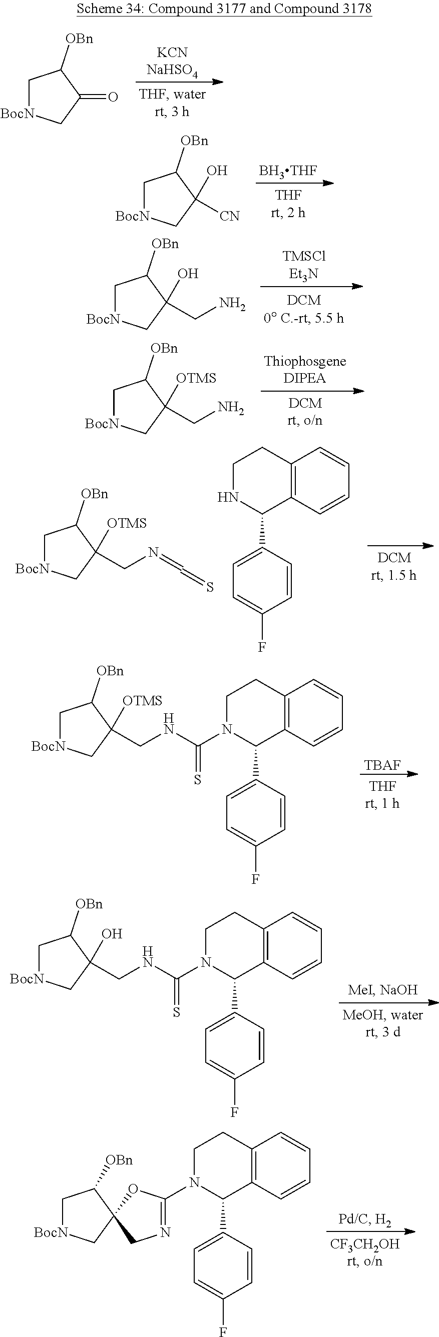

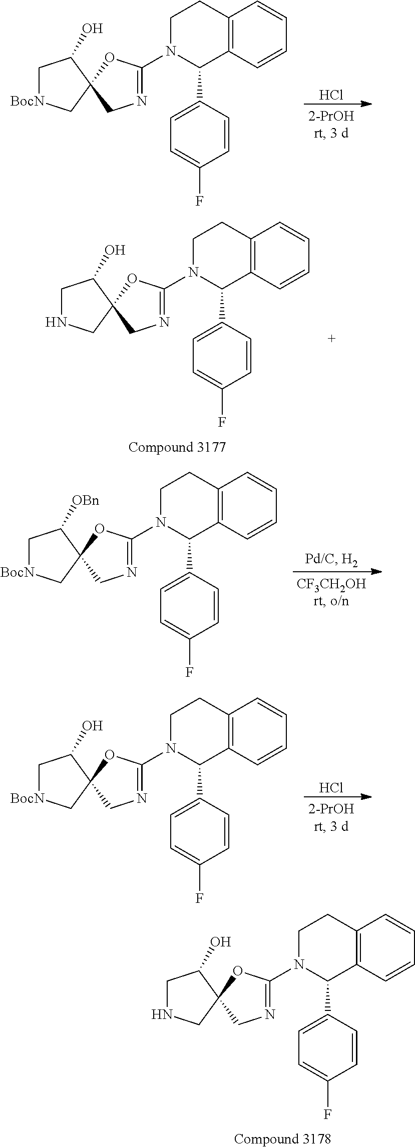

### Synthesis of tert-butyl 4-(benzyloxy)-3-cyano-3-hydroxypyrrolidine-1-carboxylate

A solution of potassium cyanide (1.019 g, 15.65 mmol) in water (7.8 mL) followed by a solution of sodium bisulfate monohydrate (6.20 g, 44.9 mmol) in water (7.8 mL) were added to a solution of tert-butyl 3-(benzyloxy)-4-oxopyrrolidine-1-carboxylate (2.40 g, 8.24 mmol) in tetrahydrofuran (9.35 mL). After stirring for 3 hours, the reaction mixture was extracted with dichloromethane (2×20 mL). The combined organic extracts were dried over Na₂SO₄ and evaporated under reduced pressure to give tert-butyl 4-(benzyloxy)-3-cyano-3-hydroxypyrrolidine-1-carboxylate. ¹H NMR (400 MHz, chloroform-d) δ 7.48-7.29 (m, 5H), 4.93-4.54 (m, 2H), 4.10-3.97 (m, 1H), 3.88-3.43 (m, 4H), 1.45 (s, 9H).

### Synthesis of tert-butyl 3-(aminomethyl)-4-(benzyloxy)-3-hydroxypyrrolidine-1-carboxylate

Under argon atmosphere, borane-THF complex (1M solution in THF, 69.2 mL, 69.2 mmol) was added to a solution of tert-butyl 4-(benzyloxy)-3-cyano-3-hydroxypyrrolidine-1-carboxylate (2.46 g, 7.72 mmol) in tetrahydrofuran (65 mL). After stirring for 2 hours, the reaction was quenched by the addition of water (15 mL). The volatiles were removed under reduced pressure and once more after dilution of the mixture with dichloromethane (25 mL) and water (15 mL). The mixture was diluted with aqueous NaOH (2 M, 20 mL) and extracted with dichloromethane (2×30 mL). The combined organic phase was dried over Na₂SO₄ and evaporated under reduced pressure. The residue was purified by flash column chromatography (silica, 0 to 7% (7M ammonia in methanol) in dichloromethane) to give tert-butyl 3-(aminomethyl)-4-(benzyloxy)-3-hydroxypyrrolidine-1-carboxylate. LCMS: 99%, RT=1.89 min., (M+H)⁺=323 (method B). ¹H NMR (400 MHz, chloroform-d) δ 7.45-7.28 (m, 5H), 4.74-4.62 (m, 1H), 4.39 (dd, J=21.4, 12.0 Hz, 1H), 3.77-3.45 (m, 3H), 3.38-3.26 (m, 2H), 3.20 (t, J=12.8 Hz, 1H), 2.63 (dd, J=27.8, 12.7 Hz, 1H), 1.46 (s, 9H).

### Synthesis of tert-butyl 3-(aminomethyl)-4-(benzyloxy)-3-((trimethylsilyl)oxy)pyrrolidine-1-carboxylate

At 0° C. under argon atmosphere, trimethylsilyl chloride (0.951 mL, 7.44 mmol) was added dropwise to a solution of tert-butyl 3-(aminomethyl)-4-(benzyloxy)-3-hydroxypyrrolidine-1-carboxylate (0.80 g, 2.481 mmol) and triethylamine (1.725 mL, 12.41 mmol) in dichloromethane (20 mL). The reaction mixture was allowed to reach room temperature and stirred for 5.5 hour. Then, the mixture was diluted with dichloromethane (15 mL) and saturated aqueous NaHCO₃ (25 mL), the layers separated, and the aqueous phase extracted with dichloromethane (3×20 mL). The combined organic phase was dried over Na₂SO₄ and evaporated under reduced pressure. The residue was purified by flash column chromatography (silica, 0 to 5% methanol in dichloromethane) to give tert-butyl 3-(aminomethyl)-4-(benzyloxy)-3-((trimethylsilyl)oxy)pyrrolidine-1-carboxylate. LCMS: 97%, RT=2.23 min., (M+H)⁺=395 (method B). ¹H NMR (400 MHz, chloroform-d) δ 7.45-7.27 (m, 5H), 4.65 (d, J=11.7 Hz, 1H), 4.41 (dd, J=22.7, 11.9 Hz, 1H), 3.76-3.69 (m, 1H), 3.63 (d, J=11.9 Hz, 0.5H), 3.56-3.45 (m, 2H), 3.42 (d, J=11.8 Hz, 0.5H), 3.30 (dd, J=11.9, 7.2 Hz, 1H), 3.03 (dd, J=13.6, 1.7 Hz, 1H), 2.75 (dd, J=13.6, 3.2 Hz, 1H), 1.46 (s, 9H), 0.12 (2×s, 9H).

### Synthesis of tert-butyl 4-(benzyloxy)-3-(isothiocyanatomethyl)-3-((trimethylsilyl)oxy)pyrrolidine-1-carboxylate

Under argon atmosphere, a solution of tert-butyl 3-(aminomethyl)-4-(benzyloxy)-3-((trimethylsilyl)oxy)pyrrolidine-1-carboxylate (0.621 g, 1.574 mmol) and N,N-diisopropylethylamine (0.330 mL, 1.889 mmol) in dichloromethane (20 mL) was added to a solution of thiophosgene (0.142 mL, 1.574 mmol) in dichloromethane (20 ml). The reaction mixture was stirred overnight and quenched with saturated aqueous NaHCO₃ (25 mL). The layers were separated and the aqueous phase was extracted with dichloromethane (3×20 mL). The combined organic phase was dried over Na₂SO₄ and evaporated under reduced pressure. The residue was purified by flash column chromatography (silica, 0 to 30% ethyl acetate in heptane) to give tert-butyl 4-(benzyloxy)-3-(isothiocyanatomethyl)-3-((trimethylsilyl)oxy)pyrrolidine-1-carboxylate. LCMS: 85%, RT=2.43 min., (M+Na)⁺=459 (method A). ¹H NMR (400 MHz, chloroform-d) δ 7.41-6.92 (m, 5H), 4.62 (d, J=11.6 Hz, 1H), 4.55-4.43 (m, 1H), 3.89-3.79 (m, 2H), 3.69-3.42 (m, 4H), 3.34-3.26 (m, 1H), 1.46 (s, 9H), 0.17 (s, 9H).

### Synthesis of tert-butyl 4-(benzyloxy)-3-(((S)-1-(4-fluorophenyl)-1,2,3,4-tetrahydroisoquinoline-2-carbothioamido)methyl)-3-((trimethylsilyl)oxy)pyrrolidine-1-carboxylate

Under argon atmosphere, (S)-1-(4-fluorophenyl)-1,2,3,4-tetrahydroisoquinoline (0.286 g, 1.260 mmol) was added to a solution of tert-butyl 4-(benzyloxy)-3-(isothiocyanatomethyl)-3-((trimethylsilyl)oxy)pyrrolidine-1-carboxylate (0.550 g, 1.260 mmol) in dichloromethane (20 mL). After 1.5 hour, the reaction mixture was concentrated to dryness under reduced pressure. The residue was purified by flash column chromatography (silica, 0 to 30% ethyl acetate in heptane) to give tert-butyl 4-(benzyloxy)-3-(((S)-1-(4-fluorophenyl)-1,2,3,4-tetrahydroisoquinoline-2-carbothioamido)methyl)-3-((trimethylsilyl)oxy)pyrrolidine-1-carboxylate. LCMS: 99%, RT=2.52 min., (method A). ¹H NMR (400 MHz, chloroform-d) δ mixture of diastereomers δ 7.58-7.04 (m, 11H), 6.95-6.85 (m, 2H), 6.47-6.28 (m, 1H), 4.65-4.29 (m, 3H), 3.83-2.94 (m, 8H), 2.58-2.36 (m, 2H), 1.51-1.38 (m, 9H), 0.29-0.06 (m, 9H).

### Synthesis of tert-butyl 4-(benzyloxy)-3-(((S)-1-(4-fluorophenyl)-1,2,3,4-tetrahydroisoquinoline-2-carbothioamido)methyl)-3-hydroxypyrrolidine-1-carboxylate

Under argon atmosphere, tetrabutylammonium fluoride (1.0M solution in tetrahydrofuran, 0.813 mL, 0.813 mmol) was added dropwise to a solution of tert-butyl 4-(benzyloxy)-3-(((S)-1-(4-fluorophenyl)-1,2,3,4-tetrahydroisoquinoline-2-carbothioamido)methyl)-3-((trimethylsilyl)oxy)pyrrolidine-1-carboxylate (0.54 g, 0.813 mmol) in tetrahydrofuran (dry, 12 mL). After stirring for 1 hour, the reaction mixture was diluted with ethyl acetate (40 mL) and washed with brine (30 mL). The organic layer was dried on Na₂SO₄ and evaporated under reduced pressure. The residue was purified by flash column chromatography (silica, 0 to 35% ethyl acetate in heptane) to give tert-butyl 4-(benzyloxy)-3-(((S)-1-(4-fluorophenyl)-1,2,3,4-tetrahydroisoquinoline-2-carbothioamido)methyl)-3-hydroxypyrrolidine-1-carboxylate. LCMS: 98%, RT=2.32 min., (M+H)⁺=592 (method A). ¹H NMR (400 MHz, chloroform-d) δ 7.44-7.19 (m, 8H), 7.19-7.05 (m, 3H), 6.98-6.86 (m, 2H), 6.14-5.92 (m, 1H), 4.70-2.93 (m, 12H), 2.78-2.45 (m, 2H), 1.48-1.43 (m, 9H).

### Synthesis of tert-butyl 9-(benzyloxy)-2-((S)-1-(4-fluorophenyl)-3,4-dihydroisoquinolin-2(1H)-yl)-1-oxa-3,7-diazaspiro[4.4]non-2-ene-7-carboxylate

Aqueous NaOH (1 M, 0.656 mL, 0.656 mmol) was added to a solution of tert-butyl 4-(benzyloxy)-3-(((S)-1-(4-fluorophenyl)-1,2,3,4-tetrahydroisoquinoline-2-carbothioamido)methyl)-3-hydroxypyrrolidine-1-carboxylate (0.388 g, 0.656 mmol) in methanol (13 ml) at room temperature. After 10 minutes, iodomethane (0.123 mL, 1.967 mmol) was added and the reaction mixture was stirred for 3 days. The mixture was diluted with dichloromethane (30 mL) and washed with brine (25 mL). The layers were separated and the aqueous phase was extracted with dichloromethane (3×30 mL). The combined organic phase was dried over Na₂SO₄ and evaporated under reduced pressure. The residue was purified by flash column chromatography (silica, 0 to 50% ethyl acetate in heptane) and preparative chiral SFC (method BD) to give tert-butyl (5S,9S)-9-(benzyloxy)-2-((S)-1-(4-fluorophenyl)-3,4-dihydroisoquinolin-2(1H)-yl)-1-oxa-3,7-diazaspiro[4.4]non-2-ene-7-carboxylate (44 mg) as the first eluting SFC isomer as a white solid and tert-butyl (5R,9S)-9-(benzyloxy)-2-((S)-1-(4-fluorophenyl)-3,4-dihydroisoquinolin-2(1H)-yl)-1-oxa-3,7-diazaspiro[4.4]non-2-ene-7-carboxylate as the second eluting SFC isomer. Configuration of the spirocyclic center and benzyloxy moiety are arbitrarily assigned.

First eluting SFC isomer: LCMS: 99%, RT=1.91 min., (M+H)⁺=558 (method A). SFC: 100%, RT=2.29 min., (M+H)⁺=558 (method V). ¹H NMR (400 MHz, chloroform-d) δ 7.39-7.11 (m, 10H), 7.06-6.98 (m, 1H), 6.93 (t, J=8.7 Hz, 2H), 6.11 (s, 1H), 4.66-4.54 (m, 1H), 4.52-4.41 (m, 1H), 4.14 (d, J=13.4 Hz, 1H), 3.95-3.81 (m, 1H), 3.80-3.55 (m, 4H), 3.55-3.28 (m, 3H), 3.08-2.92 (m, 1H), 2.82-2.70 (m, 1H), 1.48 and 1.46 (2×s, 9H). Second eluting SFC isomer: LCMS: 100%, RT=1.90 min., (M+H)⁺=558 (method A). SFC: 97%, RT=2.52 min., (M+H)⁺=558 (method V). ¹H NMR (400 MHz, chloroform-d) δ 7.38-7.08 (m, 10H), 7.02-6.89 (m, 3H), 6.15-6.07 (m, 1H), 4.63 (t, J=11.9 Hz, 1H), 4.53-4.42 (m, 1H), 4.13 (dd, J=13.4, 3.4 Hz, 1H), 3.93-3.83 (m, 2H), 3.81-3.42 (m, 5H), 3.33-3.19 (m, 1H), 3.10-2.95 (m, 1H), 2.80-2.69 (m, 1H), 1.50 (s, 9H).

### Synthesis of tert-butyl (5R,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate

A solution of tert-butyl (5R,9S)-9-(benzyloxy)-2-((S)-1-(4-fluorophenyl)-3,4-dihydroisoquinolin-2(1H)-yl)-1-oxa-3,7-diazaspiro[4.4]non-2-ene-7-carboxylate (42 mg, 0.075 mmol) in trifluoroethanol (2 mL) was hydrogenated at atmospheric pressure in the presence of palladium (10% on activated carbon, 50% wet, 176 mg, 0.083 mmol) at room temperature overnight. The reaction mixture was filtered through celite and the filtrate was concentrated under reduced pressure to give tert-butyl (5R,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate. Configuration of the spirocyclic center and hydroxy moiety are arbitrarily assigned. LCMS: 90%, RT=1.75 min., (M+H)⁺=468 (method A). ¹H NMR (400 MHz, chloroform-d) δ 7.26-7.08 (m, 5H), 7.08-6.88 (m, 3H), 6.11 (s, 1H), 4.14-3.81 (m, 4H), 3.77-3.44 (m, 5H), 3.38-3.20 (m, 1H), 3.11-2.94 (m, 1H), 2.85-2.68 (m, 1H), 1.50 (s, 9H).

### Synthesis of (5S,9S)-2-((S)-1-(4-fluorophenyl)-3,4-dihydroisoquinolin-2(1H)-yl)-1-oxa-3,7-diazaspiro[4.4]non-2-en-9-ol (Compound 3177)

HCl (6 M in 2-propanol, 0.167 mL, 1.00 mmol) was added to a solution of tert-butyl (5R,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate (38 mg, 0.081 mmol) in 2-propanol (1 mL). An additional portion of HCl (6 M in 2-propanol, 0.167 mL, 1.00 mmol) was added after 1 day and a second portion of HCl (6 M in 2-propanol, 0.167 mL, 1.00 mmol) was added after 2 days. After 3 days, the reaction mixture was diluted with dichloromethane and washed with saturated aqueous K₂CO₃ (5 mL). The aqueous phase was extracted with dichloromethane (3×10 mL). The combined organic phase was dried over Na₂SO₄ and evaporated under reduced pressure. The residue was dissolved in methanol (1 mL) and loaded onto an SCX-2 column (1 g) and eluted with methanol until neutral. Next, the column was eluted with ammonia in methanol (7 M). The basic fraction was concentrated to dryness and the residue purified by acidic preparative MPLC (Linear Gradient: t=0 min 5% A, t=1 min 5% A; t=16 min 30% A; t=17 min 100%; t=22 min 100% A; detection: 220/254 nm). The product containing fractions were basified with saturated aqueous K₂CO₃ till pH-10 and extracted with dichloromethane (3×20 mL). The combined organic phase was dried over Na₂SO₄ and evaporated under reduced pressure. The residue was lyophilized from a mixture of acetonitrile and water (1:1, 4 mL) to give (5S,9S)-2-((S)-1-(4-fluorophenyl)-3,4-dihydroisoquinolin-2(1H)-yl)-1-oxa-3,7-diazaspiro[4.4]non-2-en-9-ol (Compound 3177). LCMS: 99%, RT=0.68 min., (M+H)⁺=368 (method BB). ¹H NMR (400 MHz, chloroform-d) δ 7.26-7.12 (m, 5H), 7.01 (d, J=7.5 Hz, 1H), 6.95 (t, J=8.7 Hz, 2H), 6.14 (s, 1H), 4.13 (d, J=13.2 Hz, 1H), 4.08-4.02 (m, 1H), 3.93-3.82 (m, 1H), 3.61 (d, J=13.2 Hz, 1H), 3.40-3.24 (m, 2H), 3.20 (d, J=12.7 Hz, 1H), 3.10-2.92 (m, 3H), 2.79-2.68 (m, 1H).

### Synthesis of tert-butyl (5S,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate

Starting from tert-butyl (5S,9S)-9-(benzyloxy)-2-((S)-1-(4-fluorophenyl)-3,4-dihydroisoquinolin-2(1H)-yl)-1-oxa-3,7-diazaspiro[4.4]non-2-ene-7-carboxylate (44 mg, 0.079 mmol), tert-butyl (5S,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate was prepared according to the procedure described for tert-butyl (5R,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate. LCMS: 100%, RT=1.76 min., (M+H)⁺=468 (method A). ¹H NMR (400 MHz, chloroform-d) δ 7.26-7.10 (m, 5H), 7.06-6.99 (m, 1H), 6.94 (t, J=8.5 Hz, 2H), 6.12 (s, 1H), 4.10-3.79 (m, 4H), 3.78-3.22 (m, 6H), 3.03-2.87 (m, 1H), 2.85-2.66 (m, 1H), 1.48 and 1.46 (2×s, 9H).

### Synthesis of (5R,9S)-2-((S)-1-(4-fluorophenyl)-3,4-dihydroisoquinolin-2(1H)-yl)-1-oxa-3,7-diazaspiro[4.4]non-2-en-9-ol (Compound 3178)

Starting from tert-butyl (5S,9S)-2-((S)-1-(4-fluorophenyl)-3,4-dihydroisoquinolin-2(1H)-yl)-9-hydroxy-1-oxa-3,7-diazaspiro[4.4]non-2-ene-7-carboxylate (43 mg, 0.093 mmol), (5R,9S)-2-((S)-1-(4-fluorophenyl)-3,4-dihydroisoquinolin-2(1H)-yl)-1-oxa-3,7-diazaspiro[4.4]non-2-en-9-ol (Compound 3178) was prepared according to the procedure described for (5S,9S)-2-((S)-1-(4-fluorophenyl)-3,4-dihydroisoquinolin-2(1H)-yl)-1-oxa-3,7-diazaspiro[4.4]non-2-en-9-ol (Compound 3177). LCMS: 92%, RT=0.69 min., (M+H)⁺=368 (method BB). ¹H NMR (400 MHz, chloroform-d) δ 7.26-7.11 (m, 5H), 7.01 (d, J=7.5 Hz, 1H), 6.94 (t, J=8.7 Hz, 2H), 6.14 (s, 1H), 4.09 (d, J=13.2 Hz, 1H), 3.95-3.79 (m, 2H), 3.60 (d, J=13.2 Hz, 1H), 3.39-3.18 (m, 3H), 3.06 (d, J=12.7 Hz, 1H), 3.02-2.90 (m, 2H), 2.82-2.68 (m, 1H).

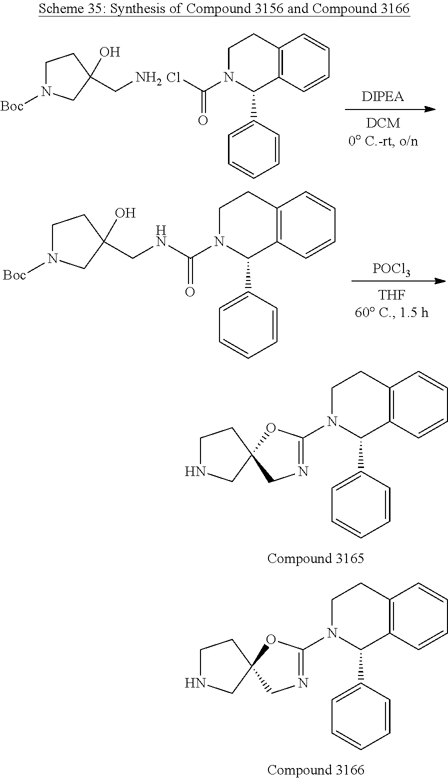

### Synthesis of tert-butyl 3-hydroxy-3-(((S)-1-phenyl-1,2,3,4-tetrahydroisoquinoline-2-carboxamido)methyl)pyrrolidine-1-carboxylate

Under an argon atmosphere at 0° C., a solution of tert-butyl 3-(aminomethyl)-3-hydroxypyrrolidine-1-carboxylate (398 mg, 1.840 mmol) and N,N-diisopropylethylamine (0.353 mL, 2.02 mmol) in dichloromethane (5 mL) was added to a solution of (S)-1-phenyl-3,4-dihydroisoquinoline-2(1H)-carbonyl chloride (0.50 g, 1.84 mmol) in dichloromethane (5 mL). The reaction mixture was allowed to warm to room temperature, stirred overnight, and concentrated to dryness under reduced pressure. The residue was purified by flash column chromatography (silica, 50 to 100% ethyl acetate in heptane) to give tert-butyl 3-hydroxy-3-(((S)-1-phenyl-1,2,3,4-tetrahydroisoquinoline-2-carboxamido)methyl)pyrrolidine-1-carboxylate. LCMS: 99%, RT=2.04 min., (M+Na)⁺=474 (method A). ¹H NMR (400 MHz, chloroform-d) δ 7.34-7.12 (m, 9H), 6.38-6.24 (m, 1H), 5.31-5.01 (m, 1H), 4.97-4.53 (m, 1H), 3.73-3.43 (m, 4H), 3.42-3.18 (m, 4H), 2.98-2.75 (m, 2H), 1.95-1.57 (m, 2H), 1.47-1.40 (2×s, 9H).

### Synthesis of (R)-2-((S)-1-(4-fluorophenyl)-3,4-dihydroisoquinolin-2(1H)-yl)-1-oxa-3,7-diazaspiro[4.4]non-2-ene (Compound 3165) and (S)-2-((S)-1-(4-fluorophenyl)-3,4-dihydroisoquinolin-2(1H)-yl)-1-oxa-3,7-diazaspiro[4.4]non-2-ene (Compound 3166)

Phosphorus oxychloride (0.826 mL, 8.86 mmol) was added to a solution of tert-butyl 3-hydroxy-3-(((S)-1-phenyl-1,2,3,4-tetrahydroisoquinoline-2-carboxamido)methyl)pyrrolidine-1-carboxylate (0.80 g, 1.77 mmol) in dry tetrahydrofuran (9 mL) under an argon atmosphere. The reaction mixture was transferred into a pre-heated oil bath (60° C.) and stirred for 1.5 hour. After cooling to room temperature, the reaction mixture was concentrated to dryness under reduced pressure and stored overnight before further use. The residue was dissolved in methanol and treated with Amberlyst A-21 resin (10 g) for 30 minutes, filtered, and washed with methanol. The combined filtrates were concentrated under reduced pressure. The residue was taken up in dichloromethane (25 mL) and washed with aqueous NaOH (2M, 30 mL). The layers were separated and the aqueous phase was extracted with dichloromethane (3×25 mL). The combined organic phase was dried over Na₂SO₄ and evaporated under reduced pressure. The residue was purified by flash column chromatography (silica, 0 to 10% (7M ammonia in methanol) in dichloromethane) and preparative chiral SFC (method AN and BA) to give (R)-2-((S)-1-phenyl-3,4-dihydroisoquinolin-2(1H)-yl)-1-oxa-3,7-diazaspiro[4.4]non-2-ene (Compound 3166) as the first eluting SFC isomer and (S)-2-((S)-1-phenyl-3,4-dihydroisoquinolin-2(1H)-yl)-1-oxa-3,7-diazaspiro[4.4]non-2-ene (Compound 3165) as the second eluting SFC isomer. Configuration of the spirocyclic center is arbitrarily assigned.

Compound 3165: LCMS: 96%, RT=0.70 min., (M+H)⁺=334 (method BB). SFC: 97%, RT=4.15 min., (M+H)⁺=334 (method AZ). ¹H NMR (400 MHz, chloroform-d+D₂O) δ 7.37-7.12 (m, 8H), 7.11-7.02 (m, 1H), 6.26-6.14 (m, 1H), 4.01-3.87 (m, 1H), 3.86-3.69 (m, 2H), 3.45-3.22 (m, 2H), 3.22-3.10 (m, 1H), 3.10-2.94 (m, 2H), 2.94-2.65 (m, 2H), 2.32-2.09 (m, 1H), 2.06-1.79 (m, 1H).

Compound 3166: LCMS: 95%, RT=0.70 min., (M+H)⁺=334 (method BB). SFC: 100%, RT=3.58 min., (M+H)⁺=334 (method AZ). ¹H NMR (400 MHz, chloroform-d+D₂O) mixture of rotamers δ 7.32-7.12 (m, 8H), 7.11-6.97 (m, 1H), 6.26-6.12 (m, 1H), 3.98-3.87 (m, 1H), 3.87-3.69 (m, 2H), 3.44-2.85 (m, 5H), 2.84-2.66 (m, 2H), 2.34-2.16 (m, 1H), 2.06-1.82 (m, 1H).

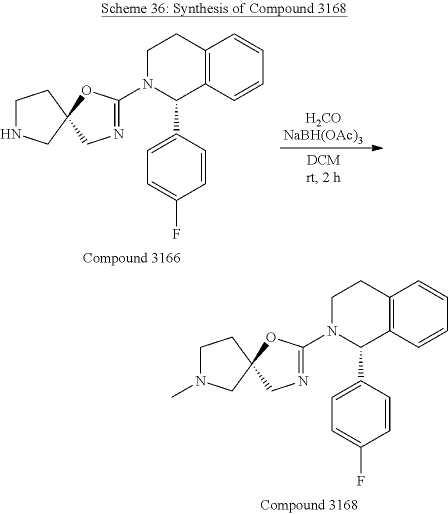 [SMILES: Fc1ccc([C@H]2c3ccccc3CCN2C2=NC[C@]3(CCNC3)O2)cc1 ; CN1CC[C@]2(CN=C(N3CCc4ccccc4[C@@H]3c3ccc(F)cc3)O2)C1]

### Synthesis of (R)-2-((S)-1-(4-fluorophenyl)-3,4-dihydroisoquinolin-2(1H)-yl)-7-methyl-1-oxa-3,7-diazaspiro[4.4]non-2-ene (Compound 3168)

Formaldehyde (37 wt % solution in water stabilized with 10-15% MeOH, 0.043 mL, 0.574 mmol) followed by sodium triacetoxyborohydride (91 mg, 0.430 mmol) were added to a solution of (R)-2-((S)-1-(4-fluorophenyl)-3,4-dihydroisoquinolin-2(1H)-yl)-1-oxa-3,7-diazaspiro[4.4]non-2-ene (Compound 3166, 50.4 mg, 0.143 mmol) in dichloromethane (1.5 mL). After 2 hours, water (2 mL) was added and the layers separated using a phase separator. The aqueous phase was extracted with dichloromethane and the combined organic phase was evaporated under reduced pressure. The residue was purified by acidic preparative MPLC (Linear Gradient: t=0 min 2% A; t=1 min 2% A; t=16 min 30% A; t=17 min 100%; t=22 min 100% A; detection: 220/245 nm). The product was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give (R)-2-((S)-1-(4-fluorophenyl)-3,4-dihydroisoquinolin-2(1H)-yl)-7-methyl-1-oxa-3,7-diazaspiro[4.4]non-2-ene (Compound 3168). LCMS: 99%, RT=0.67 min., (M+H)⁺=366 (method P). ¹H NMR (400 MHz, chloroform-d) δ 7.25-7.12 (m, 5H), 7.02 (d, J=7.4 Hz, 1H), 6.94 (t, J=8.6 Hz, 2H), 6.24 (s, 1H), 3.98-3.89 (m, 1H), 3.86-3.72 (m, 2H), 3.34-3.24 (m, 1H), 3.11-2.97 (m, 2H), 2.95-2.85 (m, 1H), 2.72 (dt, J=16.3, 4.0 Hz, 1H), 2.48-2.34 (m, 2H), 2.37 (s, 3H), 2.35-2.24 (m, 1H), 2.10-1.99 (m, 1H).

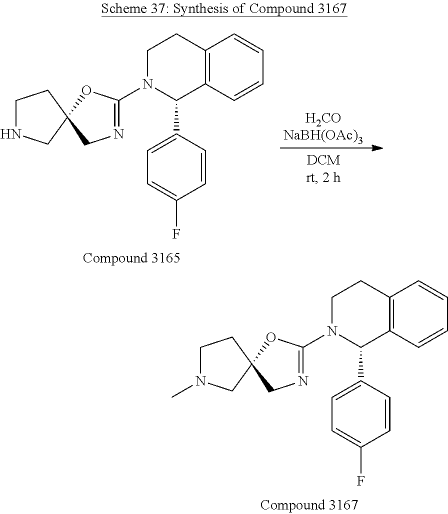 [SMILES: Fc1ccc([C@H]2c3ccccc3CCN2C2=NC[C@@]3(CCNC3)O2)cc1 ; CN1CC[C@@]2(CN=C(N3CCc4ccccc4[C@@H]3c3ccc(F)cc3)O2)C1]

### Synthesis of (S)-2-((S)-1-(4-fluorophenyl)-3,4-dihydroisoquinolin-2(1H)-yl)-7-methyl-1-oxa-3,7-diazaspiro[4.4]non-2-ene (Compound 3167)

Starting from (S)-2-((S)-1-(4-fluorophenyl)-3,4-dihydroisoquinolin-2(1H)-yl)-1-oxa-3,7-diazaspiro[4.4]non-2-ene (Compound 3165, 68 mg, 0.193 mmol), (S)-2-((S)-1-(4-fluorophenyl)-3,4-dihydroisoquinolin-2(1H)-yl)-7-methyl-1-oxa-3,7-diazaspiro[4.4]non-2-ene (Compound 3167) was prepared as described for (R)-2-((S)-1-(4-fluorophenyl)-3,4-dihydroisoquinolin-2(1H)-yl)-7-methyl-1-oxa-3,7-diazaspiro[4.4]non-2-ene (Compound 3168). LCMS: 97%, RT=0.67 min., (M+H)⁺=366 (method P). ¹H NMR (400 MHz, chloroform-d) δ 7.25-7.12 (m, 5H), 7.05-6.90 (m, 3H), 6.25 (s, 1H), 4.04-3.91 (m, 1H), 3.88-3.72 (m, 2H), 3.36-3.21 (m, 1H), 3.12-2.94 (m, 2H), 2.94-2.83 (m, 1H), 2.73 (dt, J=16.2, 3.9 Hz, 1H), 2.50-2.28 (m, 6H), 2.10-1.99 (m, 1H).

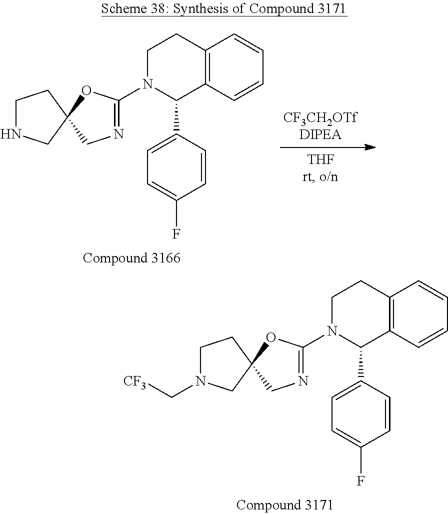 [SMILES: Fc1ccc([C@H]2c3ccccc3CCN2C2=NC[C@]3(CCNC3)O2)cc1 ; Fc1ccc([C@H]2c3ccccc3CCN2C2=NC[C@]3(CCN(CC(F)(F)F)C3)O2)cc1]

### Synthesis of (R)-2-((S)-1-(4-fluorophenyl)-3,4-dihydroisoquinolin-2(1H)-yl)-7-(2,2,2-trifluoroethyl)-1-oxa-3,7-diazaspiro[4.4]non-2-ene (Compound 3171)

N,N-diisopropylethylamine (0.052 mL, 0.299 mmol) and 2,2,2-trifluoroethyl trifluoromethanesulfonate (0.032 mL, 0.224 mmol) were added to a solution of (R)-2-((S)-1-(4-fluorophenyl)-3,4-dihydroisoquinolin-2(1H)-yl)-1-oxa-3,7-diazaspiro[4.4]non-2-ene (Compound 3166, 52.2 mg, 0.149 mmol) in dry tetrahydrofuran (2 mL). The mixture was stirred overnight, diluted with water (2 mL) and brine (2 mL), and extracted with dichloromethane (2×5 mL). The combined organic phase was dried over Na₂SO₄ and evaporated under reduced pressure. The residue was purified by acidic preparative MPLC (Linear Gradient: t=0 min 5% A; t=1 min 5% A; t=2 min 10% A; t=17 min 50% A; t=18 min 100%; t=23 min 100% A; detection: 220/250 nm). The product was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give (R)-2-((S)-1-(4-fluorophenyl)-3,4-dihydroisoquinolin-2(1H)-yl)-7-(2,2,2-trifluoroethyl)-1-oxa-3,7-diazaspiro[4.4]non-2-ene (Compound 3171). LCMS: 98%, RT=1.90 min., (M+H)⁺=434 (method P). ¹H NMR (400 MHz, chloroform-d) δ 7.24-7.13 (m, 5H), 7.03 (d, J=7.4 Hz, 1H), 6.95 (t, J=8.7 Hz, 2H), 6.19 (s, 1H), 3.97-3.87 (m, 1H), 3.79 (q, J=12.7 Hz, 2H), 3.36-3.25 (m, 1H), 3.21-2.95 (m, 5H), 2.93-2.82 (m, 2H), 2.74 (dt, J=16.3, 4.1 Hz, 1H), 2.26-2.16 (m, 1H), 2.00 (dt, J=13.6, 7.6 Hz, 1H).

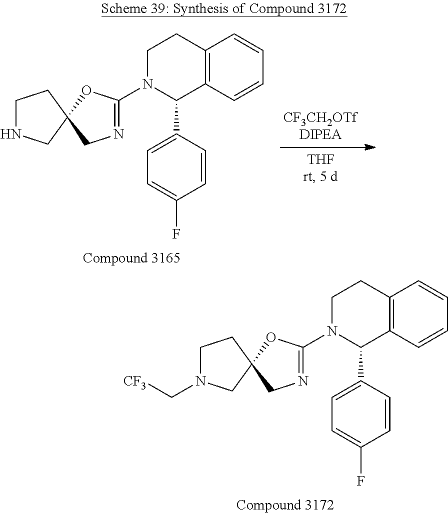 [SMILES: Fc1ccc([C@H]2c3ccccc3CCN2C2=NC[C@@]3(CCNC3)O2)cc1 ; Fc1ccc([C@H]2c3ccccc3CCN2C2=NC[C@@]3(CCN(CC(F)(F)F)C3)O2)cc1]

### Synthesis of (S)-2-((S)-1-(4-fluorophenyl)-3,4-dihydroisoquinolin-2(1H)-yl)-7-(2,2,2-trifluoroethyl)-1-oxa-3,7-diazaspiro[4.4]non-2-ene (Compound 3172)

Starting from (S)-2-((S)-1-(4-fluorophenyl)-3,4-dihydroisoquinolin-2(1H)-yl)-1-oxa-3,7-diazaspiro[4.4]non-2-ene (Compound 3165, 50.2 mg, 0.143 mmol), (S)-2-((S)-1-(4-fluorophenyl)-3,4-dihydroisoquinolin-2(1H)-yl)-7-(2,2,2-trifluoroethyl)-1-oxa-3,7-diazaspiro[4.4]non-2-ene (Compound 3172) was prepared as described for (R)-2-((S)-1-(4-fluorophenyl)-3,4-dihydroisoquinolin-2(1H)-yl)-7-(2,2,2-trifluoroethyl)-1-oxa-3,7-diazaspiro[4.4]non-2-ene (Compound 3171). LCMS: 100%, RT=1.28 min., (M+H)⁺=434 (method P). ¹H NMR (400 MHz, chloroform-d) δ 7.25-7.13 (m, 5H), 7.00 (d, J=7.5 Hz, 1H), 6.95 (t, J=8.7 Hz, 2H), 6.19 (s, 1H), 4.00-3.90 (m, 1H), 3.78 (d, J=1.7 Hz, 2H), 3.31 (ddd, J=13.2, 10.8, 4.4 Hz, 1H), 3.22-2.99 (m, 5H), 2.86-2.71 (m, 3H), 2.31 (ddd, J=13.5, 7.1, 5.2 Hz, 1H), 2.03 (ddd, J=14.3, 8.2, 6.5 Hz, 1H).

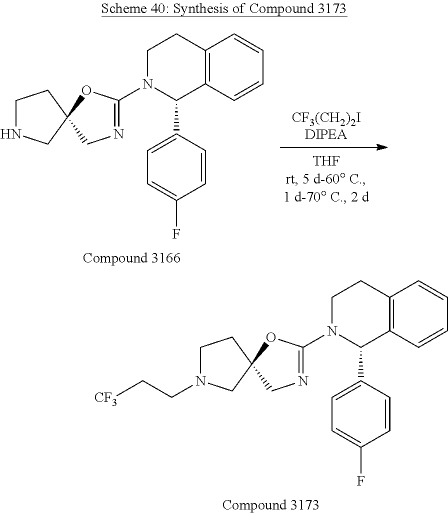 [SMILES: Fc1ccc([C@H]2c3ccccc3CCN2C2=NC[C@]3(CCNC3)O2)cc1 ; Fc1ccc([C@H]2c3ccccc3CCN2C2=NC[C@]3(CCN(CCC(F)(F)F)C3)O2)cc1]

### Synthesis of (R)-2-((S)-1-(4-fluorophenyl)-3,4-dihydroisoquinolin-2(1H)-yl)-7-(3,3,3-trifluoropropyl)-1-oxa-3,7-diazaspiro[4.4]non-2-ene (Compound 3173)

N,N-diisopropylethylamine (0.050 mL, 0.289 mmol) and 1,1,1-trifluoro-3-iodopropane (0.020 mL, 0.173 mmol) were added to a solution of (R)-2-((S)-1-(4-fluorophenyl)-3,4-dihydroisoquinolin-2(1H)-yl)-1-oxa-3,7-diazaspiro[4.4]non-2-ene (Compound 3166, 50.8 mg, 0.145 mmol) in dry tetrahydrofuran (2 mL). The mixture was stirred at room temperature for 5 days, at 60° C. for 1 day, and at 70° C. for 2 days. At day 7, additional 1,1,1-trifluoro-3-iodopropane (0.034 mL, 0.289 mmol) and N,N-diisopropylethylamine (0.050 mL, 0.289 mmol) were added. The reaction mixture was diluted with acetonitrile (2 mL) and purified by acidic preparative MPLC (Linear Gradient: t=0 min 5% A; t=1 min 5% A; t=16.6 min 40% A; t=17.6 min 100%; t=22.8 min 100% A; detection: 220/250/280 nm). The product was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 4 mL) to give (R)-2-((S)-1-(4-fluorophenyl)-3,4-dihydroisoquinolin-2(1H)-yl)-7-(3,3,3-trifluoropropyl)-1-oxa-3,7-diazaspiro[4.4]non-2-ene (Compound 3173). LCMS: 98%, RT=0.98 min., (M+H)⁺=448 (method P). ¹H NMR (400 MHz, chloroform-d) δ 7.25-7.11 (m, 5H), 7.03 (d, J=7.4 Hz, 1H), 6.95 (t, J=8.7 Hz, 2H), 6.21 (s, 1H), 3.93 (ddd, J=13.2, 6.1, 3.5 Hz, 1H), 3.79 (q, J=12.6 Hz, 2H), 3.35-3.24 (m, 1H), 3.09-2.84 (m, 3H), 2.81-2.63 (m, 3H), 2.61-2.50 (m, 2H), 2.40-2.20 (m, 3H), 2.03 2.03 (ddd, J=13.9, 8.0, 5.8 Hz, 1H).

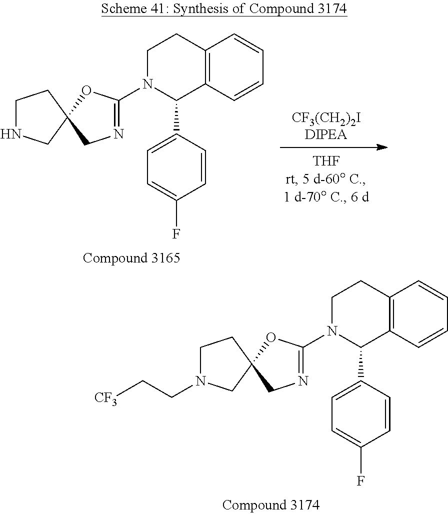 [SMILES: Fc1ccc([C@H]2c3ccccc3CCN2C2=NC[C@@]3(CCNC3)O2)cc1 ; Fc1ccc([C@H]2c3ccccc3CCN2C2=NC[C@@]3(CCN(CCC(F)(F)F)C3)O2)cc1]

### Synthesis of (S)-2-((S)-1-(4-fluorophenyl)-3,4-dihydroisoquinolin-2(1H)-yl)-7-(3,3,3-trifluoropropyl)-1-oxa-3,7-diazaspiro[4.4]non-2-ene (Compound 3174)

Starting from (R)-2-((S)-1-(4-fluorophenyl)-3,4-dihydroisoquinolin-2(1H)-yl)-1-oxa-3,7-diazaspiro[4.4]non-2-ene (Compound 3165, 53.9 mg, 0.153 mmol), (S)-2-((S)-1-(4-fluorophenyl)-3,4-dihydroisoquinolin-2(1H)-yl)-7-(3,3,3-trifluoropropyl)-1-oxa-3,7-diazaspiro[4.4]non-2-ene (Compound 3174) was prepared as described for (R)-2-((S)-1-(4-fluorophenyl)-3,4-dihydroisoquinolin-2(1H)-yl)-7-(3,3,3-trifluoropropyl)-1-oxa-3,7-diazaspiro[4.4]non-2-ene (Compound 3173). LCMS: 99%, RT=1.00 min., (M+H)⁺=448 (method P). ¹H NMR (400 MHz, chloroform-d) δ 7.25-7.13 (m, 5H), 7.01 (d, J=7.5 Hz, 1H), 6.95 (t, J=8.7 Hz, 2H), 6.21 (s, 1H), 3.94 (ddd, J=13.4, 6.2, 3.5 Hz, 1H), 3.83-3.73 (m, 2H), 3.30 (ddd, J=13.2, 10.6, 4.4 Hz, 1H), 3.04 (ddd, J=16.6, 10.6, 6.0 Hz, 1H), 2.96 (d, J=10.4 Hz, 1H), 2.89 (td, J=8.3, 5.7 Hz, 1H), 2.80-2.66 (m, 3H), 2.59-2.50 (m, 2H), 2.41-2.25 (m, 3H), 2.03 (ddd, J=13.9, 8.1, 5.7 Hz, 1H).

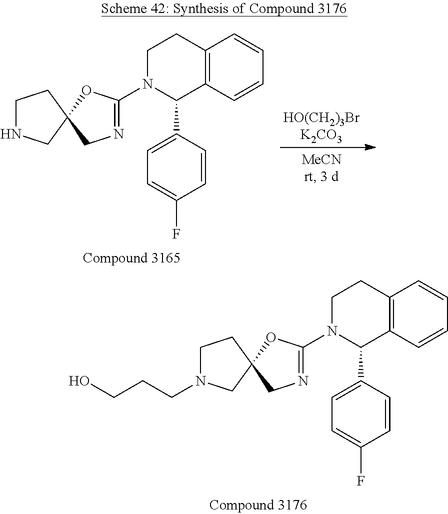 [SMILES: Fc1ccc([C@H]2c3ccccc3CCN2C2=NC[C@@]3(CCNC3)O2)cc1 ; OCCCN1CC[C@@]2(CN=C(N3CCc4ccccc4[C@@H]3c3ccc(F)cc3)O2)C1]

### Synthesis of 3-((S)-2-((S)-1-(4-fluorophenyl)-3,4-dihydroisoquinolin-2(1H)-yl)-1-oxa-3,7-diazaspiro[4.4]non-2-en-7-yl)propan-1-01(Compound 3176)

K₂CO₃ (88 mg, 0.640 mmol) and 3-bromopropan-1-ol (21 μL, 0.235 mmol) were added to a solution of (S)-2-((S)-1-(4-fluorophenyl)-3,4-dihydroisoquinolin-2(1H)-yl)-1-oxa-3,7-diazaspiro[4.4]non-2-ene (Compound 3165, 75 mg, 0.213 mmol) in dry acetonitrile (5 mL). The mixture was stirred at room temperature for 3 days, filtered and the filtrate evaporated under reduced pressure. The residue was purified by preparative LCMS (method BC) and the product was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure to give 3-((S)-2-((S)-1-(4-fluorophenyl)-3,4-dihydroisoquinolin-2(1H)-yl)-1-oxa-3,7-diazaspiro[4.4]non-2-en-7-yl)propan-1-ol (Compound 3176) after lyophilization from a mixture of acetonitrile and water (1:1, 4 mL). LCMS: 98%, RT=0.68 min., (M+H)⁺=410 (method P). ¹H NMR (400 MHz, chloroform-d+D₂O) δ 7.24-7.11 (m, 5H), 7.01 (d, J=7.6 Hz, 1H), 6.96 (t, J=8.6 Hz, 2H), 6.18 (s, 1H), 3.96-3.87 (m, 1H), 3.83 (t, J=5.2 Hz, 2H), 3.80-3.70 (m, 2H), 3.34-3.24 (m, 1H), 3.10-2.95 (m, 3H), 2.82-2.68 (m, 3H), 2.56-2.44 (m, 2H), 2.29 (dt, J=14.3, 7.3 Hz, 1H), 2.06-1.94 (m, 1H), 1.74 (p, J=5.4 Hz, 2H).

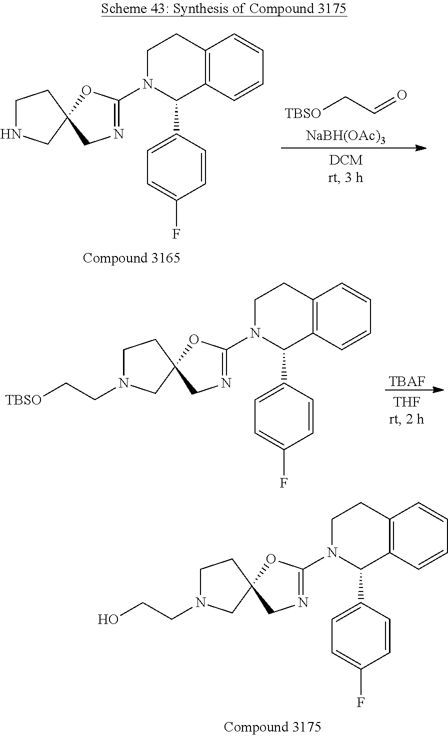

### Synthesis of (S)-7-(2-((tert-butyldimethylsilyl)oxy)ethyl)-2-((S)-1-(4-fluorophenyl)-3,4-dihydroisoquinolin-2(1H)-yl)-1-oxa-3,7-diazaspiro[4.4]non-2-ene

2-((Tert-butyldimethylsilyl)oxy)acetaldehyde (0.032 mL, 0.167 mmol) followed by sodium triacetoxyborohydride (35.5 mg, 0.167 mmol) were added to a solution of (S)-2-((S)-1-(4-fluorophenyl)-3,4-dihydroisoquinolin-2(1H)-yl)-1-oxa-3,7-diazaspiro[4.4]non-2-ene (Compound 3165, 49 mg, 0.139 mmol) in dichloromethane (3 mL). After 3 hours, water was added and the layers separated using a phase separator. The aqueous phase was extracted with dichloromethane and the combined organic layers were evaporated under reduced pressure. The residue was purified by acidic preparative MPLC (Linear Gradient: t=0 min 5% A; t=1 min 5% A; t=16.6 min 40% A; t=17.6 min 100%; t=22.8 min 100% A; detection: 220/250/280 nm). The product fractions were combined and lyophilized to give (S)-7-(2-((tert-butyldimethylsilyl)oxy)ethyl)-2-((S)-1-(4-fluorophenyl)-3,4-dihydroisoquinolin-2(1H)-yl)-1-oxa-3,7-diazaspiro[4.4]non-2-ene. ¹H NMR (400 MHz, chloroform-d) δ 7.24-7.14 (m, 5H), 7.02 (d, J=7.5 Hz, 1H), 6.95 (t, J=8.7 Hz, 2H), 6.24 (s, 1H), 4.02-3.92 (m, 1H), 3.86-3.75 (m, 4H), 3.38-3.28 (m, 1H), 3.11 (d, J=11.2 Hz, 1H), 3.09-2.92 (m, 2H), 2.80-2.62 (m, 5H), 2.30 (dt, J=14.1, 7.1 Hz, 1H), 2.09-1.99 (m, 1H), 0.90 (s, 9H), 0.07 (s, 6H).

### Synthesis of 2-((S)-2-((S)-1-(4-fluorophenyl)-3,4-dihydroisoquinolin-2(1H)-yl)-1-oxa-3,7-diazaspiro[4.4]non-2-en-7-yl)ethan-1-ol (Compound 3175)

Tetrabutylammonium fluoride (1.0 M solution in tetrahydrofuran (0.109 mL, 0.109 mmol) was added to a solution of (S)-7-(2-((tert-butyldimethylsilyl)oxy)ethyl)-2-((S)-1-(4-fluorophenyl)-3,4-dihydroisoquinolin-2(1H)-yl)-1-oxa-3,7-diazaspiro[4.4]non-2-ene (37 mg, 0.073 mmol) in dry tetrahydrofuran (0.5 mL). After 2 hours, the reaction mixture was concentrated to dryness under reduced pressure and the residue purified by acidic preparative MPLC (Linear Gradient: t=0 min 2% A; t=1 min 2% A; t=16 min 30% A; t=17 min 100%; t=22 min 100% A; detection: 210/220/280 nm). The product was dissolved in methanol (1 mL) and loaded onto an SCX-2 column (1 g) and eluted with methanol until neutral. Next, the column was eluted with ammonia in methanol (1 M). The basic fraction was concentrated to dryness under reduced pressure and the residue lyophilized from a mixture of acetonitrile and water (1:1, 4 mL) to give 2-((S)-2-((S)-1-(4-fluorophenyl)-3,4-dihydroisoquinolin-2(1H)-yl)-1-oxa-3,7-diazaspiro[4.4]non-2-en-7-yl)ethan-1-ol (Compound 3175). LCMS: 100%, RT=0.68 min., (M+H)⁺=396 (method P). ¹H NMR (400 MHz, chloroform-d) δ 7.25-7.13 (m, 5H), 7.01 (d, J=7.5 Hz, 1H), 6.96 (t, J=8.7 Hz, 2H), 6.20 (s, 1H), 3.94 (ddd, J=13.4, 6.2, 3.4 Hz, 1H), 3.83-3.73 (m, 2H), 3.66 (t, J=5.4 Hz, 2H), 3.30 (ddd, J=13.3, 10.7, 4.4 Hz, 1H), 3.10-2.88 (m, 3H), 2.80-2.56 (m, 5H), 2.31 (dt, J=14.1, 7.1 Hz, 1H), 2.03 (ddd, J=13.9, 8.1, 5.9 Hz, 1H).

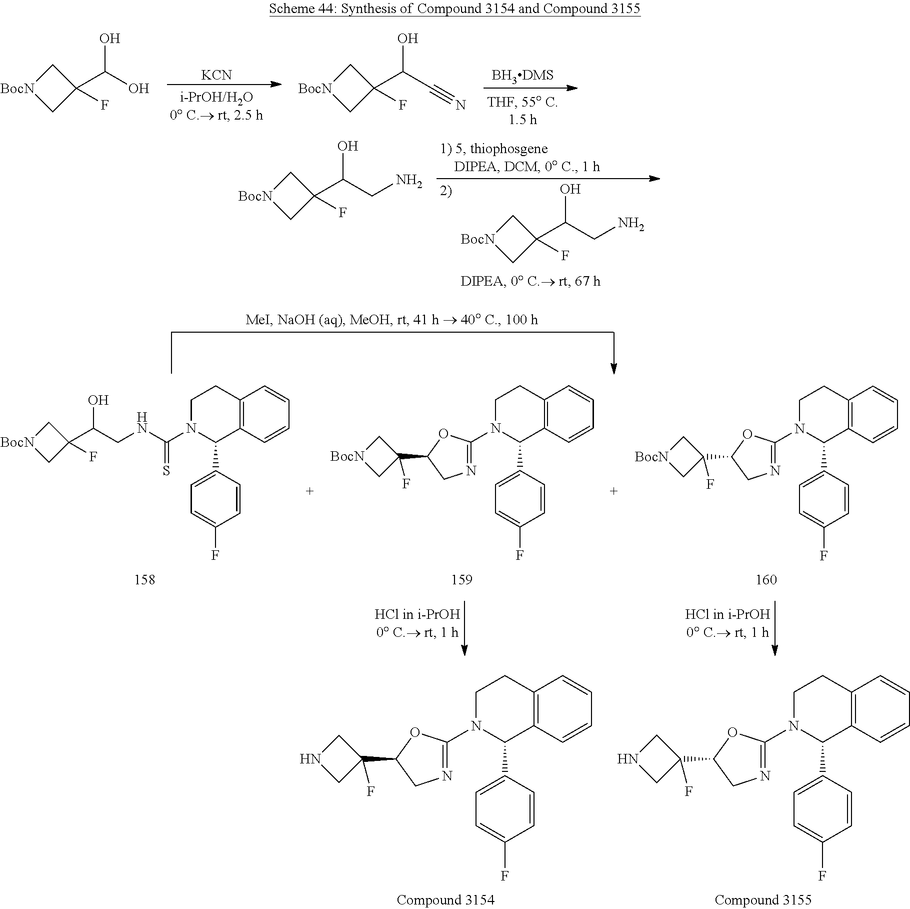

### Synthesis of tert-butyl 3-(cyano(hydroxy)methyl)-3-fluoroazetidine-1-carboxylate

At 0° C., a solution of tert-butyl 3-(dihydroxymethyl)-3-fluoroazetidine-1-carboxylate (1.027 g, 4.36 mmol) in 2-propanol (5.0 mL) was added dropwise to a solution of KCN (313 mg, 4.80 mmol) in water (3.5 mL). The mixture was allowed to warm to room temperature and stirred for 2.5 hours then diluted with ethyl acetate (30 mL), washed with saturated aqueous NaHCO₃ (10 mL) and the aqueous layer extracted with ethyl acetate (2×15 mL). The combined organic phase was dried over Na₂SO₄ and evaporated under reduced pressure. The residue was purified by flash column chromatography (silica, 10 to 50% ethyl acetate in heptane) to give tert-butyl 3-(cyano(hydroxy)methyl)-3-fluoroazetidine-1-carboxylate. ¹H NMR (400 MHz, chloroform-d) δ 4.65 (d, J=13.2 Hz, 1H), 4.49 (br s, 1H), 4.30-4.07 (m, 4H), 1.46 (s, 9H).

### Synthesis of tert-butyl 3-(2-amino-1-hydroxyethyl)-3-fluoroazetidine-1-carboxylate

Borane dimethyl sulfide complex (2M in tetrahydrofuran, 4.27 mL, 8.53 mmol) was added to a solution of tert-butyl 3-(cyano(hydroxy)methyl)-3-fluoroazetidine-1-carboxylate (655 mg, 2.84 mmol). The resulting mixture was stirred at 60° C. for 1.5 hours, cooled in an ice/water bath and quenched by the addition of water (6 mL). Once foam formation ceased, saturated aqueous NaHCO₃ (15 mL) was added followed by ethyl acetate (30 mL). After stirring for 5 minutes, the layers were separated and the aqueous phase was extracted with ethyl acetate (2×30 mL). The combined organic phase was dried over Na₂SO₄ and evaporated under reduced pressure. The residue was purified by flash column chromatography (silica, 3 to 10% (7M ammonia in methanol) in dichloromethane) to give tert-butyl 3-(2-amino-1-hydroxyethyl)-3-fluoroazetidine-1-carboxylate. ¹H NMR (400 MHz, chloroform-d) δ 4.25-4.13 (m, 1H), 4.11-3.92 (m, 3H), 3.71 (ddd, J=18.0, 7.4, 4.1 Hz, 1H), 2.95 (dd, J=12.9, 4.1 Hz, 1H), 2.76 (dd, J=12.9, 7.4 Hz, 1H), 1.44 (s, 9H).

### Synthesis of tert-butyl 3-fluoro-3-((S)-2-((S)-1-(4-fluorophenyl)-3,4-dihydroisoquinolin-2(1H)-yl)-4,5-dihydrooxazol-5-yl)azetidine-1-carboxylate and tert-butyl 3-fluoro-3-((R)-2-((S)-1-(4-fluorophenyl)-3,4-dihydroisoquinolin-2(1H)-yl)-4,5-dihydrooxazol-5-yl)azetidine-1-carboxylate

Under nitrogen atmosphere at 0° C., a solution of (S)-1-(4-fluorophenyl)-1,2,3,4-tetrahydroisoquinoline (128 mg, 0.470 mmol) and N,N-diisopropylethylamine (98 μL, 0.470 mmol) in dry dichloromethane (1.7 mL) was added dropwise to a solution of thiophosgene (51 μL, 0.470 mmol) in dry dichloromethane (1.7 mL). The reaction mixture was stirred for 75 minutes before a suspension of tert-butyl 3-(2-amino-1-hydroxyethyl)-3-fluoroazetidine-1-carboxylate (120 mg, 0.427 mmol) and N,N-diisopropylethylamine (98 μL, 0.470 mmol) in dichloromethane (dry, 1.7 mL) was added. After the addition the reaction mixture was allowed to slowly warm to room temperature and stir for 67 hours. The reaction mixture was diluted with dichloromethane (20 mL), washed with aqueous citric acid (0.5 M, 5 mL) and saturated aqueous NaHCO₃ (5 mL), dried over Na₂SO₄, and evaporated under reduced pressure. The residue was purified by flash column chromatography (silica, 0 to 75% ethyl acetate in heptane) to give tert-butyl 3-fluoro-3-(2-((S)-1-(4-fluorophenyl)-1,2,3,4-tetrahydroisoquinoline-2-carbothioamido)-1-hydroxyethyl)azetidine-1-carboxylate (158, 72 mg) as a colorless amorphous solid, tert-butyl 3-fluoro-3-((S)-2-((S)-1-(4-fluorophenyl)-3,4-dihydroisoquinolin-2(1H)-yl)-4,5-dihydrooxazol-5-yl)azetidine-1-carboxylate (159), and tert-butyl 3-fluoro-3-((R)-2-((S)-1-(4-fluorophenyl)-3,4-dihydroisoquinolin-2(1H)-yl)-4,5-dihydrooxazol-5-yl)azetidine-1-carboxylate (160).

Another portion of 159 and 160 was prepared by adding a solution of sodium hydroxide (6.12 mg, 0.153 mmol) in water (25 μL) to a solution of tert-butyl 3-fluoro-3-(2-((S)-1-(4-fluorophenyl)-1,2,3,4-tetrahydroisoquinoline-2-carbothioamido)-1-hydroxyethyl)azetidine-1-carboxylate (158, 70 mg, 0.139 mmol) in methanol (2.5 mL). The solution was cooled to 0° C. and iodomethane (0.030 mL, 0.486 mmol) was added. The reaction mixture was allowed to warm to room temperature and stirred for 41 hours and then at 40° C. for 100 hours. Afterwards, the reaction mixture was concentrated to half of its original volume under reduced pressure and the residue was partitioned between brine (1 mL) and dichloromethane (4 mL). The aqueous layer was extracted with dichloromethane (2×3 mL) and the combined organic phase was dried over Na₂SO₄ and evaporated under reduced pressure. The residue was purified by flash column chromatography (silica, 40 to 80% ethyl acetate in heptane) to give 159 and upon further elution of the column (0 to 10% (7M ammonia in methanol) in dichloromethane) a mixture of 159 and 160 (ratio 12:88, SFC, method W). Stereochemistry on the oxazoline moiety was arbitrarily assigned.

158: LCMS: 88%, RT=2.19 min., (M+H)⁺=504 (method A). ¹H NMR (400 MHz, chloroform-d) mixture of diastereomers δ 7.36 (s, 1H), 7.32-7.27 (m, 3H), 7.25-7.13 (m, 3H), 7.01-6.92 (m, 2H), 6.01 (t, J=5.7 Hz, 1H), 4.32-3.90 (m, 6H), 3.89-3.67 (m, 3H), 2.97-2.87 (m, 1H), 2.86-2.75 (m, 1H), 1.45 and 1.44 (2×s, 9H). 159: LCMS: 93%, RT=1.79 min., (M+H)⁺=470 (method A). SFC: 100%, RT=1.66 min., (M+H)⁺=470 (method W). ¹H NMR (400 MHz, chloroform-d) δ 7.26-7.12 (m, 5H), 7.08-6.91 (m, 3H), 6.19 (s, 1H), 4.85-8.73 (m, 1H), 4.16-3.82 (m, 6H), 3.72 (dd, J=12.8, 6.7 Hz, 1H), 3.39-3.26 (m, 1H), 3.09-2.97 (m, 1H), 2.75 (dt, J=16.2, 4.1 Hz, 1H), 1.45 (s, 9H). 160: LCMS: 96%, RT=1.78 min., (M+H)⁺=470 (method A). SFC: 99%, RT=2.06 min., (M+H)⁺=470 (method W). ¹H NMR (400 MHz, chloroform-d) mixture of rotamers. δ 7.26-6.90 (m, 8H), 6.18 (s, 0.8H), 5.77 (s, 0.2H), 4.89-4.74 (m, 1H), 4.18-3.86 (m, 6.2H), 3.78-3.65 (m, 1.2H), 3.52-3.46 (m, 0.2H), 3.39-3.27 (m, 0.8H), 3.09-2.96 (m, 0.8H), 2.76 (dt, J=16.2, 4.0 Hz, 0.8H), 1.45 (s, 9H).

### Synthesis of (S)-5-(3-fluoroazetidin-3-yl)-2-((S)-1-(4-fluorophenyl)-3,4-dihydroisoquinolin-2(1H)-yl)-4,5-dihydrooxazole (Compound 3154)

At 0° C., HCl (5-6 M in 2-propanol, 749 μL, 4.12 mmol) was added to tert-butyl 3-fluoro-3-(((S)-2-((S)-1-(4-fluorophenyl)-3,4-dihydroisoquinolin-2(1H)-yl)-4,5-dihydrooxazol-5-yl)azetidine-1-carboxylate (40.7 mg, 0.082 mmol). After 15 minutes, the reaction mixture was allowed to warm to room temperature and stirred for 45 minutes. The mixture was diluted with dichloromethane (5 mL) and washed with saturated aqueous Na₂CO₃ (3 mL). The aqueous layer was extracted with dichloromethane (2×3 mL) and the combined organic phase was dried over Na₂SO₄ and evaporated under reduced pressure. The residue was dissolved in methanol (2 mL) and loaded onto an SCX-2 column (1 g) and eluted with methanol until neutral. Next, the column was eluted with ammonia in methanol (1 M). The basic fraction was concentrated to dryness under reduced pressure. The residue was lyophilized from a mixture of acetonitrile and water (1:1, 2 mL) to give (S)-5-(3-fluoroazetidin-3-yl)-2-((S)-1-(4-fluorophenyl)-3,4-dihydroisoquinolin-2(1H)-yl)-4,5-dihydrooxazole (Compound 3154). Stereochemistry on the oxazoline moiety was arbitrarily assigned. LCMS: 98%, RT=0.70 min., (M+H)⁺=370 (method P). SFC: 100%, RT=2.32 min., (M+H)⁺=370 (method W). ¹H NMR (400 MHz, chloroform-d) δ 7.25-7.12 (m, 5H), 7.07-6.90 (m, 3H), 6.22 (s, 1H), 4.93-4.81 (m, 1H), 3.99-3.77 (m, 5H), 3.71-3.52 (m, 2H), 3.37-3.26 (m, 1H), 3.11-2.99 (m, 1H), 2.74 (dt, J=16.4, 4.1 Hz, 1H).

### Synthesis of (R)-5-(3-fluoroazetidin-3-yl)-2-((S)-1-(4-fluorophenyl)-3,4-dihydroisoquinolin-2(1H)-yl)-4,5-dihydrooxazole (Compound 3155)

Starting from tert-butyl 3-fluoro-3-((R)-2-((S)-1-(4-fluorophenyl)-3,4-dihydroisoquinolin-2(1H)-yl)-4,5-dihydrooxazol-5-yl)azetidine-1-carboxylate (27.8 mg, 0.057 mmol), (R)-5-(3-fluoroazetidin-3-yl)-2-((S)-1-(4-fluorophenyl)-3,4-dihydroisoquinolin-2(1H)-yl)-4,5-dihydrooxazole (Compound 3155) was prepared as described for (S)-5-(3-fluoroazetidin-3-yl)-2-((S)-1-(4-fluorophenyl)-3,4-dihydroisoquinolin-2(1H)-yl)-4,5-dihydrooxazole (Compound 3154). Stereochemistry on the oxazoline moiety was arbitrarily assigned. LCMS: 98%, RT=0.69 min., (M+H)⁺=370 (method P). SFC: 100%, RT=2.65 min., (M+H)⁺=370 (method W). ¹H NMR (400 MHz, chloroform-d) δ 7.25-7.13 (m, 5H), 7.06-6.90 (m, 3H), 6.19 (s, 1H), 4.94-4.81 (m, 1H), 4.01-3.76 (m, 5H), 3.71-3.49 (m, 2H), 3.40-3.29 (m, 1H), 3.11-2.99 (m, 1H), 2.76 (dt, J=16.3, 4.1 Hz, 1H).

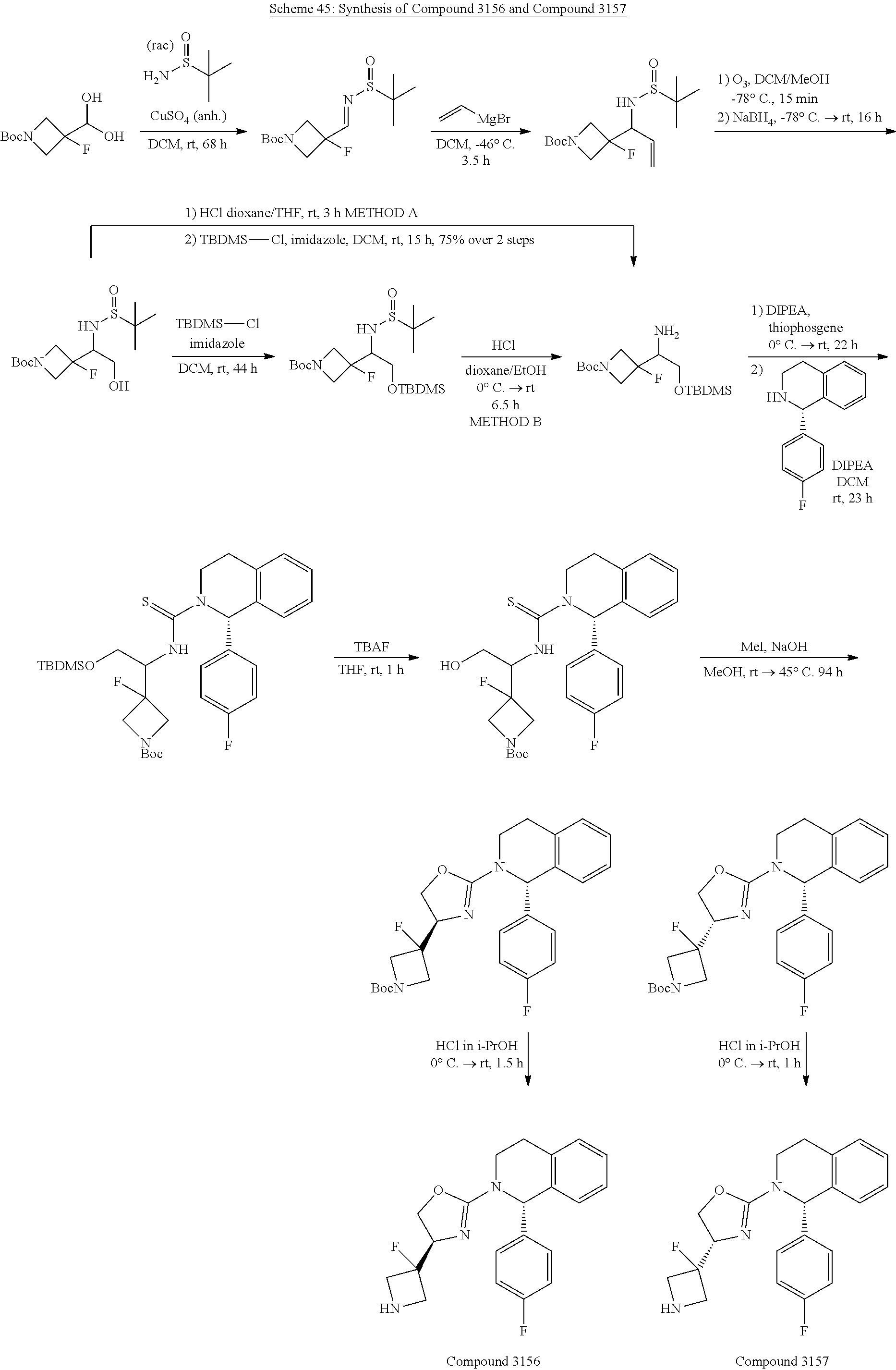

### Synthesis of tert-butyl 3-(((tert-butylsulfinyl)imino)methyl)-3-fluoroazetidine-1-carboxylate

Under nitrogen atmosphere, copper(II) sulfate (anhydrous, 2.10 g, 13.2 mmol) was added to a solution of tert-butyl 3-(dihydroxymethyl)-3-fluoroazetidine-1-carboxylate (910 mg, 4.11 mmol) in dichloromethane (dry, 9.0 mL). After 15 minutes, 2-methyl-2-propanesulfinamide (499 mg, 4.11 mmol) was added and the reaction mixture was stirred at room temperature for 68 hours. The mixture was filtered through Celite® and washed with dichloromethane. The filtrate was concentrated under reduced pressure and purified by flash column chromatography (silica, 0 to 50% ethyl acetate in heptane) to give tert-butyl 3-(((tert-butylsulfinyl)imino)methyl)-3-fluoroazetidine-1-carboxylate. LCMS: 99%, RT=1.97 min., (M+Na)⁺=329 (method A). ¹H NMR (400 MHz, chloroform-d) mixture of diastereomers δ 8.25 and 8.23 (2×s, 1H), 4.40-4.19 (m, 4H), 1.46 (s, 9H), 1.24 (s, 9H).

### Synthesis of tert-butyl 3-(1-((tert-butylsulfinyl)amino)allyl)-3-fluoroazetidine-1-carboxylate

Under nitrogen atmosphere at −46° C. (dry ice/acetonitrile), vinylmagnesium bromide (0.7M solution in tetrahydrofuran, 3.15 mL, 2.20 mmol) was added dropwise to a solution of tert-butyl 3-(((tert-butylsulfinyl)imino)methyl)-3-fluoroazetidine-1-carboxylate (540 mg, 1.76 mmol) in dry dichloromethane (8.6 mL). The reaction mixture was stirred for 3.5 hours and quenched by the addition of saturated aqueous NH₄Cl (8 mL) followed by dichloromethane (10 mL). The mixture was stirred vigorously for 5 minutes after which the layers were separated. The aqueous layer was extracted with dichloromethane (2×10 mL) and the combined organic phase was dried over Na₂SO₄ and evaporated under reduced pressure. The residue was purified by flash column chromatography (silica, 15 to 80% ethyl acetate in heptane) to give tert-butyl 3-(1-((tert-butylsulfinyl)amino)allyl)-3-fluoroazetidine-1-carboxylate. LCMS: 98%, RT=1.89 min. (34%) and 1.92 min. (66%), (M−H)⁻=333 (method A). ¹H NMR (400 MHz, chloroform-d) mixture of diastereomers δ 5.95-5.84 (m, 0.4H), 5.80-5.67 (m, 0.6H), 5.53-5.37 (m, 2H), 4.22-3.90 (m, 5H), 3.59 (d, J=5.6 Hz, 0.6H), 3.44 (d, J=9.2 Hz, 0.4H), 1.45 and 1.44 (2×s, 9H), 1.23 (s, 9H).

### Synthesis of tert-butyl 3-(1-((tert-butylsulfinyl)amino)-2-hydroxyethyl)-3-fluoroazetidine-1-carboxylate

At −78° C., ozone gas was passed through a solution of tert-butyl 3-(1-((tert-butylsulfinyl)amino)allyl)-3-fluoroazetidine-1-carboxylate (210 mg, 0.628 mmol) in a mixture of dichloromethane (stabilised with methanol, 6 mL) and methanol (6 mL) for 15 minutes. NaBH₄ (119 mg, 3.14 mmol) was added and the reaction mixture was allowed to warm to room temperature and stirred for 16 hours. The reaction mixture was diluted with dichloromethane (15 mL) and washed with brine (4.5 mL). The aqueous layer was extracted with dichloromethane (2×10 mL) and the combined organic phase was dried over Na₂SO₄ and evaporated under reduced pressure. The residue was purified by flash column chromatography (silica, 15 to 90% ethyl acetate in heptane) to give tert-butyl 3-(1-((tert-butylsulfinyl)amino)-2-hydroxyethyl)-3-fluoroazetidine-1-carboxylate. LCMS: 100%, RT=1.70 min. (53%) and 1.77 min. (47%), (M-(t-Bu)+H)+=283 (method A). ¹H NMR (400 MHz, chloroform-d) mixture of diastereomers δ 4.26 (dd, J=20.3, 10.8 Hz, 0.5H), 4.16-3.95 (m, 3.5H), 3.93-3.75 (m, 2H), 3.75-3.46 (m, 2.5H), 3.10-2.50 (br s, 0.5H), 1.45 and 1.44 (2×s, 9H), 1.28 and 1.25 (2×s, 9H).

### Synthesis of tert-butyl 3-(2-((tert-butyldimethylsilyl)oxy)-1-((tert-butylsulfinyl)amino)ethyl)-3-fluoroazetidine-1-carboxylate

Under nitrogen atmosphere, imidazole (70.8 mg, 1.040 mmol) was added to a solution of tert-butyldimethylsilyl chloride (78 mg, 0.520 mmol) in dichloromethane (0.7 mL) followed by the addition of a solution of tert-butyl 3-(1-((tert-butylsulfinyl)amino)-2-hydroxyethyl)-3-fluoroazetidine-1-carboxylate (160 mg, 0.473 mmol) in dichloromethane (2.8 mL). The reaction mixture was stirred at room temperature for 44 hours during which additional imidazole (25.7 mg, 0.378 mmol) and tert-butyldimethylsilyl chloride (28.5 mg, 0.189 mmol) were added. The reaction mixture was then diluted with dichloromethane (5 mL) and washed with a mixture of brine and saturated aqueous NaHCO₃ (1:1, 2 mL). The aqueous layer was extracted with dichloromethane (2×2 mL) and the combined organic phase was dried over Na₂SO₄ and evaporated under reduced pressure. The residue was purified by flash column chromatography (silica, 0 to 100% ethyl acetate in heptane) to give tert-butyl 3-(2-((tert-butyldimethylsilyl)oxy)-1-((tert-butylsulfinyl)amino)ethyl)-3-fluoroazetidine-1-carboxylate. LCMS: 100%, RT=2.31 min. (39%) and 2.32 min. (61%), (M-(t-Bu)+H)+=397 (method A). ¹H NMR (400 MHz, chloroform-d) mixture of diastereomers δ 4.38-3.86 (m, 5H), 3.85-3.45 (m, 3H), 1.44 and 1.43 (2×s, 9H), 1.24 and 1.22 (2×s, 9H), 0.89 (2×s, 9H), 0.09-0.04 (m, 6H).

### Synthesis of tert-butyl 3-(1-amino-2-((tert-butyldimethylsilyl)oxy)ethyl)-3-fluoroazetidine-1-carboxylate (Route A)

At 0° C., HCl (4 M in 1,4-dioxane, 47.0 μL, 0.188 mmol) was added to a solution of tert-butyl 3-(1-((tert-butylsulfinyl)amino)-2-hydroxyethyl)-3-fluoroazetidine-1-carboxylate (35.3 mg, 0.099 mmol) in tetrahydrofuran (dry, 0.5 mL). After 5 minutes, the reaction mixture was allowed to warm to room temperature and stirred for 3 hours. The mixture was diluted with brine (0.25 mL), stirred for 1 minute, further diluted with saturated aqueous Na₂CO₃ (0.40 mL) and extracted with ethyl acetate (5 mL and 3×0.5 mL). The combined organic phase was dried over Na₂SO₄ and evaporated under reduced pressure. Under nitrogen atmosphere, the residue was dissolved in dichloromethane (0.5 mL) and added to a suspension of tert-butyldimethylsilyl chloride (23.9 mg, 0.158 mmol) and imidazole (21.6 mg, 0.317 mmol) in dichloromethane (0.5 mL). The mixture was stirred for 15 hours, diluted with dichloromethane (4 mL), and washed with saturated aqueous NaHCO₃ (1 mL). The aqueous layer was extracted with dichloromethane (2 mL) and the combined organic phase was dried over Na₂SO₄ and evaporated under reduced pressure. The residue was purified by flash column chromatography (silica, 3 to 10% (7M ammonia in methanol) in dichloromethane) to give tert-butyl 3-(1-amino-2-hydroxyethyl)-3-fluoroazetidine-1-carboxylate. ¹H NMR (400 MHz, chloroform-d) mixture of diastereomers and rotamers δ 4.39-3.87 (m, 4H), 3.84-3.40 (m, 2.4H), 3.05 (dt, J=19.6, 5.7 Hz, 0.6H), 1.47-1.40 (m, 9H), 0.93-0.84 (m, 9H), 0.10-0.03 (m, 6H).

### Synthesis of tert-butyl 3-(1-amino-2-((tert-butyldimethylsilyl)oxy)ethyl)-3-fluoroazetidine-1-carboxylate (Route B)

At 0° C., HCl (81 μL, 0.325 mmol, 4 M in 1,4-dioxane) was added dropwise to a solution of tert-butyl 3-(2-((tert-butyldimethylsilyl)oxy)-1-((tert-butylsulfinyl)amino)ethyl)-3-fluoroazetidine-1-carboxylate (147 mg, 0.325 mmol) in dry ethanol (3.0 mL). The reaction mixture was allowed to gradually warm to room temperature and stirred for 6.5 hours. Next, the mixture was diluted with dichloromethane (10 mL) and washed with saturated aqueous Na₂CO₃ (5 mL). The aqueous layer was extracted with dichloromethane (3×5 mL) and the combined organic phase was dried over Na₂SO₄ and evaporated under reduced pressure. The residue was purified by flash column chromatography (silica, 0.1 to 5% (7M ammonia in methanol) in dichloromethane) to give tert-butyl 3-(1-amino-2-((tert-butyldimethylsilyl)oxy)ethyl)-3-fluoroazetidine-1-carboxylate. ¹H NMR (400 MHz, chloroform-d) mixture of diasteromers δ 4.22-4.09 (m, 2H), 4.06-3.94 (m, 2H), 3.93-3.86 (m, 0.1H), 3.84-3.75 (m, 0.1H), 3.72-3.58 (m, 1.8H), 3.57-3.45 (m, 0.1H), 2.99 (dt, J=19.7, 5.8 Hz, 0.9H), 1.45 and 1.43 (2×s, 9H), 0.90 (s, 9H), 0.08 and 0.06 (2×s, 6H).

### Synthesis of tert-butyl 3-(2-((tert-butyldimethylsilyl)oxy)-1-((S)-1-(4-fluorophenyl)-1,2,3,4-tetrahydroisoquinoline-2-carbothioamido)ethyl)-3-fluoroazetidine-1-carboxylate

Under nitrogen atmosphere at 0° C., a solution of tert-butyl 3-(1-amino-2-((tert-butyldimethylsilyl)oxy)ethyl)-3-fluoroazetidine-1-carboxylate (prepared via route A and B, 106 mg, 0.304 mmol) and N,N-diisopropylethylamine (56 μL, 0.319 mmol) in dry dichloromethane (1.0 mL) was added dropwise to a solution of thiophosgene (29 μL, 0.319 mmol) in dry dichloromethane (1.0 mL). After 4.5 hours, the reaction mixture was allowed to warm to room temperature and stirred for 17.5 hours. Next, a solution of (S)-1-(4-fluorophenyl)-1,2,3,4-tetrahydroisoquinoline (90 mg, 0.395 mmol) and N,N-diisopropylethylamine (69 μL, 0.395 mmol) in dry dichloromethane (1.0 mL) was added and stirring was continued for 23 hours. The mixture was diluted with dichloromethane (10 mL) and washed with citric acid (0.5 M, 3 mL) and saturated aqueous NaHCO₃ (3 mL). The organic phase was dried over Na₂SO₄ and evaporated under reduced pressure. The residue was purified by flash column chromatography (silica, 0 to 50% ethyl acetate in heptane) to give tert-butyl 3-(2-((tert-butyldimethylsilyl)oxy)-1-((S)-1-(4-fluorophenyl)-1,2,3,4-tetrahydroisoquinoline-2-carbothioamido)ethyl)-3-fluoroazetidine-1-carboxylate. LCMS: 98%, RT=2.69 min., (M+H)⁺=618 (method B). ¹H NMR (400 MHz, chloroform-d) mixture of diastereomers δ 7.36-7.10 (m, 7H), 7.01-6.93 (m, 2H), 6.03 (br s, 1H), 5.38-5.24 (m, 1H), 4.43-4.21 (m, 1.5H), 4.14-3.70 (m, 6H), 3.66-3.59 (m, 0.5H), 2.97-2.76 (m, 2H), 1.42 and 1.38 (2×s, 9H), 0.89 and 0.86 (2×s, 9H), 0.10-0.00 (m, 6H).

### Synthesis of tert-butyl 3-fluoro-3-(1-((S)-1-(4-fluorophenyl)-1,2,3,4-tetrahydroisoquinoline-2-carbothioamido)-2-hydroxyethyl)azetidine-1-carboxylate

Under nitrogen atmosphere, tetrabutylammonium fluoride (1 M in tetrahydrofuran, 226 μl, 0.226 mmol) was added to tert-butyl 3-(2-((tert-butyldimethylsilyl)oxy)-1-((S)-1-(4-fluorophenyl)-1,2,3,4-tetrahydroisoquinoline-2-carbothioamido)ethyl)-3-fluoroazetidine-1-carboxylate (93 mg, 0.151 mmol). After stirring the reaction mixture for 75 minutes, the mixture was diluted with ethyl acetate (3 mL) and washed with brine (1 mL). The aqueous phase was extracted with ethyl acetate (1 mL) and the combined organic phase was dried over Na₂SO₄ and evaporated under reduced pressure. The residue was purified by flash column chromatography (silica, 5 to 55% ethyl acetate in heptane) to give tert-butyl 3-fluoro-3-(1-((S)-1-(4-fluorophenyl)-1,2,3,4-tetrahydroisoquinoline-2-carbothioamido)-2-hydroxyethyl)azetidine-1-carboxylate. LCMS: 98%, RT=2.18 min., (M+H)⁺=504 (method A). ¹H NMR (400 MHz, chloroform-d) mixture of diastereomers δ 7.34-7.27 (m, 3H), 7.24-7.14 (m, 3H), 7.03-6.93 (m, 2H), 6.03 (dd, J=23.7, 9.0 Hz, 1H), 5.52-5.34 (m, 1H), 4.33-4.00 (m, 4H), 4.00-3.72 (m, 5H), 3.00-2.78 (m, 2H), 2.07 (br s, 1H), 1.44 and 1.41 (2×s, 9H).

### Synthesis of tert-butyl 3-fluoro-3-((R)-2-((S)-1-(4-fluorophenyl)-3,4-dihydroisoquinolin-2(1H)-yl)-4,5-dihydrooxazol-4-yl)azetidine-1-carboxylate and tert-butyl 3-fluoro-3-((S)-2-((S)-1-(4-fluorophenyl)-3,4-dihydroisoquinolin-2(1H)-yl)-4,5-dihydrooxazol-4-yl)azetidine-1-carboxylate

A solution of NaOH (5.00 mg, 0.124 mmol) in water (50 μL) was added to a suspension of tert-butyl 3-fluoro-3-(1-((S)-1-(4-fluorophenyl)-1,2,3,4-tetrahydroisoquinoline-2-carbothioamido)-2-hydroxyethyl)azetidine-1-carboxylate (60 mg, 0.112 mmol) in methanol (1.95 mL). Methyl iodide (25 μL, 0.394 mmol) was added and the reaction mixture was stirred at room temperature for 3 hours and at 45° C. for 91 hours. The reaction mixture was diluted with dichloromethane (8 mL) and washed with a mixture of saturated aqueous NaHCO₃ and brine (1:1, 2 mL). The aqueous layer was extracted with dichloromethane (3×2 mL) and the combined organic phase was dried over Na₂SO₄ and evaporated under reduced pressure. The residue was purified by flash column chromatography (silica, 0.1 to 5% (7M ammonia in methanol) in dichloromethane) and preparative chiral SFC (method U) to give tert-butyl 3-fluoro-3-((R)-2-((S)-1-(4-fluorophenyl)-3,4-dihydroisoquinolin-2(1H)-yl)-4,5-dihydrooxazol-4-yl)azetidine-1-carboxylate (the first eluting isomer) and tert-butyl 3-fluoro-3-((S)-2-((S)-1-(4-fluorophenyl)-3,4-dihydroisoquinolin-2(1H)-yl)-4,5-dihydrooxazol-4-yl)azetidine-1-carboxylate (the second eluting isomer). Stereochemistry on oxazoline moiety was arbitrarily assigned.

First eluting SFC isomer: LCMS: 100%, RT=1.81 min., (M+H)⁺=470 (method A). SFC: 100%, RT=1.76 min., (M+H)⁺=470 (method V). ¹H NMR (400 MHz, chloroform-d) δ 7.25-7.13 (m, 5H), 7.01 (d, J=7.5 Hz, 1H), 6.99-6.89 (m, 2H), 6.31 (s, 1H), 4.44-4.28 (m, 2H), 4.28-4.10 (m, 2H), 4.10-3.86 (m, 4H), 3.33-3.20 (m, 1H), 3.09-2.96 (m, 1H), 2.74 (dt, J=16.2, 3.7 Hz, 1H), 1.46 (s, 9H). Second eluting SFC isomer: LCMS: 100%, RT=1.81 min., (M+H)⁺=470 (method A). SFC: 93.5%, RT=1.90 min., (M+H)⁺=470 (method V). ¹H NMR (400 MHz, chloroform-d) δ 7.24-7.13 (m, 5H), 7.02 (d, J=7.5 Hz, 1H), 7.00-6.91 (m, 2H), 6.18 (s, 1H), 4.42-4.26 (m, 2H), 4.26-4.06 (m, 2H), 4.06-3.86 (m, 4H), 3.40-3.23 (m, 1H), 3.11-2.97 (m, 1H), 2.75 (dt, J=16.3, 4.2 Hz, 1H), 1.39 (s, 9H).

### Synthesis of (R)-4-(3-fluoroazetidin-3-yl)-2-((S)-1-(4-fluorophenyl)-3,4-dihydroisoquinolin-2(1H)-yl)-4,5-dihydrooxazole (Compound 3156)

Starting from tert-butyl 3-fluoro-3-((R)-2-((S)-1-(4-fluorophenyl)-3,4-dihydroisoquinolin-2(1H)-yl)-4,5-dihydrooxazol-4-yl)azetidine-1-carboxylate, (R)-4-(3-fluoroazetidin-3-yl)-2-((S)-1-(4-fluorophenyl)-3,4-dihydroisoquinolin-2(1H)-yl)-4,5-dihydrooxazole (Compound 3156) was prepared as described for (S)-5-(3-fluoroazetidin-3-yl)-2-((S)-1-(4-fluorophenyl)-3,4-dihydroisoquinolin-2(1H)-yl)-4,5-dihydrooxazole (Compound 3154). Stereochemistry on oxazoline moiety was arbitrarily assigned. LCMS: 99%, RT=0.77 min., (M+H)⁺=370 (method P). SFC: 100%, RT=3.45 min., (M+H)⁺=370 (method BE). ¹H NMR (400 MHz, chloroform-d) δ 7.25-7.13 (m, 5H), 7.03 (d, J=7.5 Hz, 1H), 6.99-6.91 (m, 2H), 6.25 (s, 1H), 4.44-4.29 (m, 2H), 4.25-4.16 (m, 1H), 3.99-3.89 (m, 1H), 3.85-3.65 (m, 3H), 3.56 (dd, J=15.2, 9.9 Hz, 1H), 3.44-3.33 (m, 1H), 3.08-2.97 (m, 1H), 2.79 (dt, J=16.3, 4.2 Hz, 1H).

### Synthesis of (S)-4-(3-fluoroazetidin-3-yl)-2-((S)-1-(4-fluorophenyl)-3,4-dihydroisoquinolin-2(1H)-yl)-4,5-dihydrooxazole (Compound 3157)

Starting from tert-butyl 3-fluoro-3-(((S)-2-((S)-1-(4-fluorophenyl)-3,4-dihydroisoquinolin-2(1H)-yl)-4,5-dihydrooxazol-4-yl)azetidine-1-carboxylate, (S)-4-(3-fluoroazetidin-3-yl)-2-((S)-1-(4-fluorophenyl)-3,4-dihydroisoquinolin-2(1H)-yl)-4,5-dihydrooxazole (Compound 3157) was prepared and purified as described for (S)-5-(3-fluoroazetidin-3-yl)-2-((S)-1-(4-fluorophenyl)-3,4-dihydroisoquinolin-2(1H)-yl)-4,5-dihydrooxazole (Compound 3156). Stereochemistry on oxazoline moiety was arbitrarily assigned. LCMS: 99%, RT=0.77 min., (M+H)⁺=370 (method P). SFC: 94.5%, RT=3.71 min., (M+H)⁺=370 (method BE). ¹H NMR (400 MHz, chloroform-d) δ 7.25-7.13 (m, 5H), 7.03 (d, J=7.5 Hz, 1H), 7.00-6.91 (m, 2H), 6.22 (s, 1H), 4.42-4.31 (m, 2H), 4.31-4.22 (m, 1H), 4.00-3.91 (m, 1H), 3.84-3.66 (m, 3H), 3.58-3.48 (m, 1H), 3.36-3.25 (m, 1H), 3.12-3.00 (m, 1H), 2.75 (dt, J=16.2, 4.0 Hz, 1H).

Cellular Assays: To measure the efficacy of compounds, a progranulin induction cellular assay in mouse primary microglia (pMG), primary cortical neurons, and BV-2 cell lines is used. BV-2 cells are split the day before plating into a 96well plate format at approximately 80%. Cells should be plated the day before and allowed for 1 hour attachment period and for 16 hour incubation. Levels of progranulin secreted into the cell culture medium or retained in the cell lysate can be quantified using an ELISA-based readout and measurement of secreted mouse PGRN in the medium was assessed by the methodology published by Ghidoni et al. 2012. Standard ELISA kits to measure PGRN are available from vendors such as Adipogen, R&D, and Biovendor.

In vivo Assays: A mouse ELISA protocol to detect progranulin in brain, plasma, or cerebrospinal fluid (CSF) can be used, with GRN+/− mice or GRN+/+ mice (available from TACONIC). The mouse is administered a compound as disclosed herein and the amount of progranulin in the brain is assessed after a specific amount of time. Mice treated with a test compound or compounds are compared to control mice which are not treated with the compound. Treatment can be done with a single or multiple dosing of compounds. Control samples are assigned a relative value of 100%.

Other in vivo assays can be performed using a GRN+/− and GRN+/+ rats, non-human primates (e.g., monkey, dog) using a similar protocol.

Treatment with the test compound increases the progranulin secretion relative to the control is at least about 110%, at least about 130%, at least about 150%, at least about 180%, at least about 200%, at least about 250%, or at least about 300%.

Table D below presents the results of a PGRN assay as described above.

TABLE D

| Compound No. | EC₅₀ μM |
|---|---|
| 3000 | 0.25 |
| 3001 | 0.10 |
| 3002 | 5.08 |
| 3003 | 4.28 |
| 3004 | >10.0 |
| 3005 | >10.0 |
| 3006 | 1.00 |
| 3007 | 0.37 |
| 3008 | >10.0 |
| 3009 | 7.02 |
| 3010 | 6.39 |
| 3011 | 7.64 |
| 3012 | >10.0 |
| 3013 | 1.59 |
| 3014 | 1.68 |
| 3015 | 0.88 |
| 3016 | 0.86 |
| 3017 | 0.26 |
| 3018 | >10.0 |
| 3019 | 0.30 |
| 3020 | 9.98 |
| 3021 | >10.0 |
| 3022 | 1.49 |
| 3023 | 2.17 |
| 3024 | 3.96 |
| 3025 | 4.06 |
| 3026 | 0.31 |
| 3027 | 2.08 |
| 3028 | >3.16 |
| 3029 | 4.02 |
| 3030 | 9.65 |
| 3031 | 4.31 |
| 3032 | 3.43 |
| 3033 | 2.38 |
| 3034 | 0.45 |
| 3035 | 0.62 |
| 3036 | 3.75 |
| 3037 | 1.00 |
| 3038 | 3.91 |
| 3039 | 1.05 |
| 3040 | 1.13 |
| 3041 | 1.72 |
| 3042 | 1.03 |
| 3043 | 3.86 |
| 3044 | 5.00 |
| 3051 | 0.98 |
| 3052 | 0.44 |
| 3045 | 4.49 |
| 3046 | 3.37 |
| 3053 | 0.25 |
| 3054 | 0.11 |
| 3055 | 1.77 |
| 3047 | >10.0 |
| 3048 | 0.11 |
| 3049 | 0.019 |
| 3050 | 0.015 |
| 3056 | 0.11 |
| 3058 | 0.38 |
| 3059 | 0.11 |
| 3060 | 0.11 |
| 3061 | 0.115 |
| 3063 | 0.208 |
| 3064 | 0.0847 |
| 3065 | 0.244 |
| 3066 | 0.055 |
| 3067 | 0.309 |
| 3069 | 0.109 |
| 3070 | 0.0966 |
| 3071 | 0.429 |
| 3072 | 0.33 |
| 3073 | 0.0669 |
| 3074 | 0.338 |
| 3075 | 0.222 |
| 3076 | >10 |
| 3077 | 0.475 |
| 3078 | 0.127 |
| 3079 | 0.236 |
| 3080 | 0.11 |
| 3081 | 0.119 |
| 3082 | 0.16 |
| 3084 | 0.204 |
| 3086 | 0.757 |
| 3133 | 0.612 |
| 3134 | 0.312 |
| 3147 | 0.0501 |
| 3148 | 0.0853 |
| 3149 | 0.481 |
| 3150 | 0.266 |
| 3151 | 3.1 |
| 3152 | 0.406 |
| 3153 | 0.94 |
| 3154 | 0.132 |
| 3155 | 0.192 |
| 3156 | 0.0998 |
| 3157 | 0.0777 |
| 3158 | 3.58 |
| 3159 | 0.209 |
| 3160 | 0.993 |
| 3161 | 0.206 |
| 3162 | 0.639 |
| 3163 | 0.237 |
| 3164 | 0.137 |
| 3165 | 0.118 |
| 3166 | 0.163 |
| 3167 | 0.176 |
| 3168 | 0.0687 |
| 3169 | 0.2 |
| 3170 | 0.538 |
| 3171 | 0.836 |
| 3172 | 1.52 |
| 3173 | 1.04 |
| 3174 | 1.38 |
| 3175 | 0.181 |
| 3176 | 0.318 |
| 3177 | 0.181 |
| 3178 | 0.249 |
| 3057A | 0.231 |
| 3057B | 0.126 |

In view of the many possible embodiments to which the principles of the disclosure may be applied, it should be recognized that the illustrated embodiments are only examples and should not be taken as limiting the scope of the invention.

What is claimed:

1. A compound, or pharmaceutically acceptable salt thereof, having a structure of Formula (I): 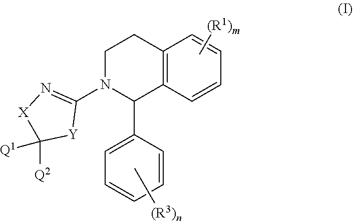
wherein
one of X and Y is O and the other is CH₂ or CH₂CH₂, or X is null or CH₂ and Y is CH₂O or (C═O)NH;
one of Q¹ and Q² is H and the other is C₀₋₃alkylene-NR⁶₂ or ring A,
or Q¹ and Q² together with the atom to which they are attached form ring A;
ring A comprises a 4- to 8-membered monocyclic or bicyclic carbocycle or heterocycle optionally substituted with 1-3 R² groups, wherein the heterocycle comprises a ring nitrogen or oxygen, or both;
each R¹ is independently C₁₋₆alkyl, O—C₁₋₆alkyl, O—C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SOₚR⁵, O—C₀₋₆alkylene-Ar, oxo, and C₀₋₆alkylene-Ar;
each R³ is independently halo, C₁₋₆alkyl, C₀₋₃alkylene-halo, O—C₁₋₃alkylene-halo, C₀₋₃alkylene-CN, C₀₋₃alkylene-NR⁴₂, C₀₋₆alkylene-OR⁴, C₀₋₆alkylene-C(O)OR⁶, C(O)N(R⁶)₂, SO₂R⁵, O—C₀₋₆alkylene-Ar, and C₀₋₆alkylene-Ar;
each R² is independently halo, OH, C₁₋₆alkyl, C₁₋₆hydroxyalkyl, or C₀₋₆alkylene-N(R^{N})₂;
each R^{N} is independently H or C₁₋₆alkyl;
each R⁴ is independently H, C₁₋₆alkyl, or C(O)C₁₋₆alkyl;
each R⁵ is independently C₁₋₆alkyl, C₁₋₆haloalkyl, or Ar;
each R⁶ is independently H or C₁₋₆alkyl;
Ar is 3-8-membered carbocycle or heterocycle, wherein the heterocycle comprises 1-4 ring heteroatoms selected from N, O, and S; C₆₋₁₀aryl; or 5-10 membered heteroaryl comprising 1-4 ring heteroatoms selected from N, O, and S and Ar is optionally substituted with 1-3 groups independently selected from halo, C₁₋₆alkyl, C₁₋₆alkoxy, C₁₋₆haloalkoxy, CN, and SO₂C₁₋₃alkyl;
m is 0-2;
n is 0-3; and
p is 0-2.

2. The compound or salt of claim 1, wherein one of X and Y is O and the other is CH₂ or CH₂CH₂, or X is null or CH₂ and Y is CH₂O; and each R² is independently halo, C₁₋₆alkyl, or —N(R^{N})₂.

3. The compound or salt of claim 1 or 2, having a structure of Formula (II):

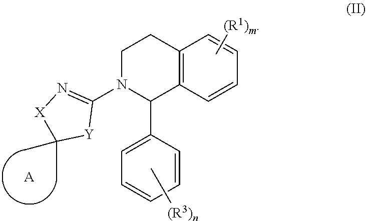

4. The compound or salt of claim 3, having a structure of Formula (IIa):

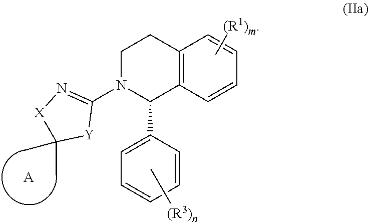

5. The compound or salt of claim 3, having a structure of Formula (IIb):

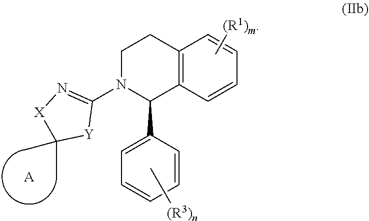

6. The compound or salt of any one of claims 1 to 5, wherein Y is O.

7. The compound or salt of any one of claims 1 to 6, wherein X is CH₂.

8. The compound or salt of any one of claims 1 to 6, wherein X is CH₂CH₂.

9. The compound or salt of any one of claims 1 to 5, wherein X is O.

10. The compound or salt of claim 9, wherein Y is CH₂.

11. The compound or salt of any one of claims 1 to 5, wherein X is CH₂ and Y is CH₂O.

12. The compound or salt of any one of claims 1 to 5, wherein X is null and Y is CH₂O.

13. The compound or salt of any one of claims 1 to 5, wherein X is null and Y is (C═O)NH.

14. The compound or salt of any one of claims 1 to 13, wherein R² is halo.

15. The compound or salt of claim 14, wherein R² is F.

16. The compound or salt of any one of claims 1 to 13, wherein R² is C₁₋₆alkyl.

17. The compound or salt of claim 16, wherein R² is methyl.

18. The compound or salt of any one of claims 1 to 13, wherein R² is N(R^{N})₂.

19. The compound or salt of claim 18, wherein R² is NH₂.

20. The compound or salt of any one of claims 1 to 19, wherein R³ is halo.

21. The compound or salt of claim 20, wherein R³ is F.

22. The compound or salt of any one of claims 1 to 21, wherein n is 0.

23. The compound or salt of any one of claims 1 to 22, wherein n is 1, 2, or 3.

24. The compound or salt of claim 23, wherein n is 1.

25. The compound or salt of claim 23, wherein n is 2.

26. The compound or salt of claim 23, wherein n is 3.

27. The compound or salt of any one of claims 1 and 6 to 26, having a structure of Formula (III):

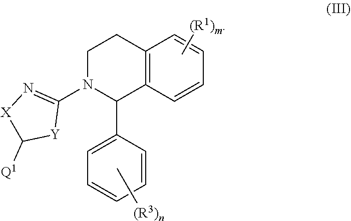

28. The compound or salt of claim 27, wherein Q¹ is C₀₋₃alkylene-NR⁶₂.

29. The compound or salt of claim 28, wherein Q¹ is C₁₋₃alkylene-NR⁶₂.

30. The compound or salt of claim 29, wherein Q¹ is CH₂NH₂.

31. The compound or salt of claim 27, wherein Q¹ is a 4- to 8-membered monocyclic or bicyclic carbocycle or heterocycle optionally substituted with 1-3 R² groups, wherein the heterocycle comprises a ring nitrogen or oxygen.

32. The compound or salt of claim 31, wherein Q¹ comprises a quinuclidine, piperidine, pyrrolidine, azetidine, or cyclobutane moiety.

33. The compound or salt of claim 32, wherein Q¹ is substituted with 1-3 R² groups.

34. The compound or salt of claim 27, wherein Q¹ is

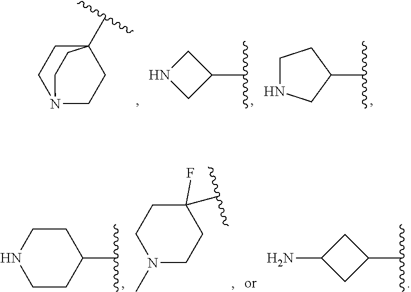

35. The compound or salt of claim 34, wherein Q² is

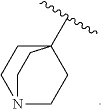 [SMILES: *C12CCN(CC1)CC2]

36. The compound or salt of claim 34, wherein Q² is

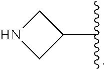 [SMILES: *C1CNC1]

37. The compound or salt of claim 34, wherein Q² is

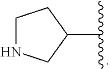 [SMILES: *C1CCNC1]

38. The compound or salt of claim 34, wherein Q² is

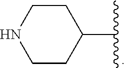 [SMILES: *C1CCNCC1]

39. The compound or salt of any one of claims 3 to 26, wherein ring A comprises a quinuclidine, piperidine, pyrrolidine, 8-azabicyclo[3.2.1]octane, 6-azabicyclo[3.1.1]heptane, cyclobutane, cyclopentane, cyclohexane, cycloheptane, furan, pyran, oxepin, morpholine, or thiomorpholine moiety.

40. The compound or salt of claim 39, wherein ring A is substituted with 1-3 R² groups.

41. The compound or salt of claim 39 or 40, wherein ring A is 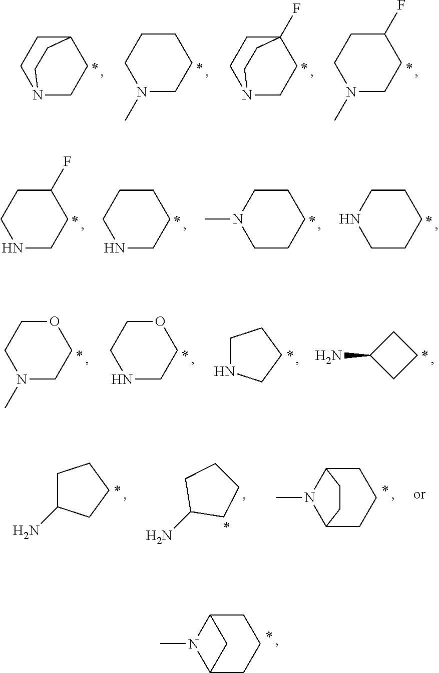
wherein * indicates the point of attachment.

42. The compound or salt of claim 41, wherein ring A is

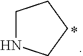

43. The compound or salt of claim 41, wherein ring A is

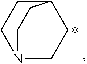

44. The compound or salt of claim 41, wherein ring A is

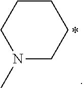

45. The compound or salt of claim 41, wherein ring A is

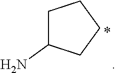

46. The compound or salt of claim 41, wherein ring A is

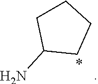

47. The compound or salt of any one of claims 1 to 46, wherein R¹ is F, Cl, OH, OMe, OiPr, OBn, O-cyclopropyl, CF₃, OCF₃, CN, SO₂Me, SO₂-cyclopropyl, SO₂iPr, oxo, imidazolyl, triazolyl, pyrrolidinyl, pyrrolidinonyl, thiadiazolyl, methyl-thiadiazolyl, trifluoromethyl-thiadiazolyl, oxadiazolyl, methyl-oxadiazolyl, trifluoromethyl-oxadiazolyl, or phenyl.

48. The compound or salt of claim 47, wherein R¹ is F or Cl.

49. The compound or salt of claim 48, wherein R¹ is F.

50. The compound or salt of any one of claims 1 to 49, wherein p is 0.

51. The compound or salt of any one of claims 1 to 49, wherein p is 1.

52. The compound or salt of any one of claims 1 to 49, wherein p is 2.

53. A compound, or pharmaceutically acceptable salt thereof, having a structure as shown in Table A.

54. A compound, or pharmaceutically acceptable salt thereof, having a structure as shown in Table B.

55. A compound, or pharmaceutically acceptable salt thereof, having a structure as shown in Table C.

56. The compound or salt of any one of claims 1 to 53, wherein the compound is compound 3000, 3001, 3049, 3050, 3057A, 3057B, 3064, 3073, 3147, 3148, 3154, 3155, 3156, or 3157.

57. The compound or salt of any one of claims 1 to 56 in the form of a salt.

58. A pharmaceutical composition comprising the compound of salt of any one of claims 1 to 57 and a pharmaceutically acceptable excipient.

59. Use of the compound or salt of any one of claims 1 to 57 as a medicament for the modulation of progranulin.

60. The use of claim 59, wherein progranulin secretion is increased.

61. A method of modulating progranulin in a subject in need thereof comprising administering to the subject the compound or salt of any one of claims 1 to 57 in an amount effective to increase the level of progranulin or granulin in the subject.

62. A method of treating a progranulin-associated disorder in a subject in need thereof comprising administering a therapeutically effective amount of the compound or salt of any one of claims 1 to 57 to the subject.

63. The method of claim 62, wherein the progranulin-associated disorder is Alzheimer [symbol] disease (AD), Parkinson [symbol] disease (PD), Amyotrophic lateral sclerosis (ALS), Frontotemporal dementia (FTD), Frontotemporal dementia-Granulin subtype (FTD-GRN), Lewy body dementia (LBD), Prion disease, Motor neuron diseases (MND), Huntington [symbol] disease (HD), Spinocerebellar ataxia (SCA), Spinal muscular atrophy (SMA), a lysosomal storage disease, nephropathy, a disease associated with inclusions and/or misfunction of C9orf72, TDP-43, FUS, UBQLN2, VCP, CHMP28, and/or MAPT, an acute neurological disorder, glioblastoma, or neuroblastoma.

64. The method of claim 63, wherein the lysosomal storage disease is Paget's disease, Gaucher's disease, Nieman's Pick disease, Tay-Sachs Disease, Fabry Disease, Pompes disease, or Naso-Hakula disease.

65. The method of claim 63, wherein the acute neurological disorder is stroke, cerebral hemorrhage, traumatic brain injury or head trauma.

66. The method of claim 63, wherein the progranulin-associated disorder is Frontotemporal dementia (FTD).

67. The method of claim 63, wherein the progranulin-associated disorder is Frontotemporal dementia-Granulin subtype (FTD-GRN).
